(19) United States
(12) Patent Application Publication
(10) Pub. No.: US 2026/0015428 A1
(43) Pub. Date: Jan. 15, 2026

(54) TARGETING IL17 SIGNALING TO TREAT CANCER AND TO PREVENT AND TREAT ICI INDUCED IMMUNE RELATED ADVERSE EVENTS (IRAES)

(71) Applicant: The Trustees of Columbia University in the City of New York, New York, NY (US)

(72) Inventors: Adam MOR, Old Tappan, NJ (US); Xizi HU, New York, NY (US); Brian HENICK, Englewood, NJ (US)

(21) Appl. No.: 19/332,600

(22) Filed: Sep. 18, 2025

Related U.S. Application Data
(63) Continuation-in-part of application No. PCT/US24/24519, filed on Apr. 12, 2024.
(60) Provisional application No. 63/496,006, filed on Apr. 13, 2023.
(60) Provisional application No. 63/599,432, filed on Nov. 15, 2023.
(60) Provisional application No. 63/696,322, filed on Sep. 18, 2024.
(60) Provisional application No. 63/809,300, filed on May 20, 2025.

(51) Int. Cl. C07K 16/28

(52) U.S. Cl. CPC C07K 16/2866; C07K 2317/21

(57) ABSTRACT

The subject matter described here relates to a method for treating cancer in a subject in need thereof comprising administering to the subject a therapeutically effective amount of an anti-IL17RA antibody or antigen binding fragment thereof. The method can also further comprise administering to the subject a therapeutically effective amount of at least one immune checkpoint inhibitor (ICI). The anti-IL17RA antibody or antigen binding fragment thereof treats, reduces, or prevents immune-related adverse events. In some embodiments, the ICI comprises an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312.

This application is a continuation in part of PCT/2024/024519, filed on Apr. 12, 2024, which claims benefit and priority to U.S. Provisional Application No. 63/496,006, filed Apr. 13, 2023; and U.S. Provisional Application No. 63/599,432, filed Nov. 15, 2023, the contents of each of which are hereby incorporated by reference in their entiretics. This application also claims the benefit of and priority to U.S. Provisional Application 63/696,322, filed on Sep. 18, 2024, and U.S. Provisional Application No. 63/809,300 filed May 20, 2025, the contents of each of which are hereby incorporated by reference in their entireties.

### STATEMENT REGARDING FEDERALLY SPONSORED RESEARCH OR DEVELOPMENT

This invention was made with government support under grants CA231277, AI150597, AI125640, and A1175498 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### INCORPORATION BY REFERENCE

All documents cited herein are incorporated herein by reference in their entireties.

### TECHNICAL FIELD

The present invention relates generally to the treatment or prevention of cancer with anti-IL-17RA antibodies in combination with an immune checkpoint inhibitor (ICI). More particularly, the present invention relates to anti-IL17RA antibodies combined with PD-1, CTLA-4, or both PD-1 and CTLA-4 blockade for cancer treatment and to prevent immune-related adverse events (irAEs) associated with ICI treatments without interfering with the anti-tumor effects induced by the ICI. In some embodiments, the ICI comprises an anti-PD-1 antibody, or antigen-binding fragment thereof, such as anti-PD1 antibodies described herein (clones 01, 02, 03, 04, 07, 09, 51, humanized 51, 55, 79, 80, SEQ ID NOS: 311-312).

### BACKGROUND

Cancer immunotherapies, e.g., immune checkpoint inhibitors (ICIs), T cell transfer therapies, tumor vaccines, and immune system modulators, produce long-term survival in many cancer patients. ICIs are approved for indications across different cancer types and stages, with an estimated 233,790 cases eligible for treatment each year. Despite their powerful advance, there remain many challenges to the use of ICIs that must be met to best advance the next generation of therapies including, but not limited to, the need to increase responsiveness to PD-1 and CTLA-4 blockade, uncover new targets to optimize pathway blockade, and not less importantly, predict and effectively manage irAEs. In some cases, ICIs produce an array of sometimes life-threatening immune-related adverse events (irAEs). There are currently no ways to predict those patients who will develop irAEs in response to ICIs. Although irAEs differ according to the ICIs used, there are common clinical features. First, the T cell infiltration of irAEs tends to be specific for different organs in different individuals rather than involving multiple organs concurrently. Second, infiltration onset could be delayed, in some cases months into ICIs treatment. Third, irAEs are not dose associated and, therefore, dose reduction doesn't prevent their occurrence and permanent ICIs discontinuation is required.

Severe or life-threatening irAEs occur in 28-72% of treated patients, resulting in up to 25% discontinuing treatment. IrAEs treatment paradigms largely empirically use corticosteroids at the time of onset; moreover, refractory irAEs management is mainly based on retrospective data and literature for phenotypically similar spontaneous autoimmune and inflammatory diseases that may be mechanistically disparate.

There remain major challenges to ICIs use that must be met to best advance these therapies. There is a need for a combination therapy that will be effective for the treatment of cancer with ICI while minimizing the irAEs of conventional ICI treatments. There is also a need for effective therapies for cancer treatment.

### SEQUENCE LISTING

The instant application contains a Sequence Listing which has been submitted electronically in XML format and is hereby incorporated by reference in its entirety. Said XML copy, created on Sep. 8, 2025, is named 0019240_01312US5_SL.xml and is 368,787 bytes in size.

### SUMMARY

In certain aspects, the present disclosure provides a method for treating cancer in a subject in need thereof comprising administering to the subject a therapeutically effective amount of an anti-IL-17RA antibody or antigen binding fragment thereof. In some embodiments, the treatment method further comprises administering to the subject a therapeutically effective amount of at least one immune checkpoint inhibitor (ICI). In certain embodiments, the present disclosure also provides a method for treating, reducing, or preventing iRAEs in a subject in need thereof comprising administering to the subject a composition comprising a therapeutically effective amount of an anti-IL-17RA antibody or antigen binding fragment thereof, wherein the iRAEs are caused by administration of at least one ICI to the subject.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof. In some embodiments, the anti-PD-1 antibody or antigen binding fragment thereof comprises Nivolumab, Pembrolizumab, Cemiplimab, Retifanlimab, Dostarlimab, Zimberelimab, Tiselelizumab, Camrelizumab, Sintilimab, Penpulimab or antigen binding fragment thereof. In some embodiments, the anti-PDL-1 antibody or antigen binding fragment thereof comprises Atezolimumab, Durvalumab and Avelumab, or a combination thereof. In some embodiments, the anti-CTLA-4 antibody or antigen binding fragment thereof comprises ipilimumab, tremelimumab, or a combination thereof. In some embodiments, the anti-LAG-3 antibody or antigen binding fragment thereof comprises BMS-986016, Relatimab, INCAGN02385, GSK2831781, or a combination thereof. In some embodiments, the anti-IL-17RA antibody or antigen binding fragment thereof is a monoclonal antibody or antigen binding fragment thereof.

In some embodiments, the at least one ICI comprises an anti-CTLA-4 antibody or antigen binding fragment thereof, anti-PD-1 antibody or antigen bindidng fragment thereof, anti-PDL-1 antibody or antigen binding fragment thereof, anti-LAG-3 antibody or antigen binding fragment thereof or a combination thereof or a therapeutically effective amount of at least one immune checkpoint inhibitor (ICI) comprising an anti-PD1 antibody, or antigen-binding fragment thereof, comprising the CDRs or variable domains of clones 01, 02, 03, 04, 07, 09, 51, humanized 51, 55, 79, 80.

In some embodiments, the anti-IL-17RA antibody or antigen binding fragment thereof, comprises: a first arm comprising a first variable heavy chain domain and a first variable light chain domain, wherein a portion of the first arm is capable of binding to a portion of an IL-17RA; and a second arm comprising a second variable heavy chain domain and a second variable light chain domain, wherein a portion of the second arm is capable of binding to a portion of the IL-17RA protein wherein the first and second arms each further comprise a fragment, crystallizable (Fc) domain. In some embodiments, the first and second arms each further comprise a CH1 domain, a hinge domain, and a CL domain. In some embodiments, the portion of IL-17RA bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-17RA binding site and wherein the second variable heavy chain domain and second variable light chain domain form a second IL-17RA binding site.

In some embodiments, the first and second IL-17RA binding sites are the same. In some embodiments, the first and third polypeptide chain each further encode a hinge domain, a CHI domain, and the Fc domain, and wherein the second and fourth polypeptide chain each further encode a CL domain.

In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 146, HCDR2 comprising SEQ ID NO: 147, and HCDR3 comprising SEQ ID NOs: 148 and wherein the first and second variable light chain domain each comprises LCDR1 comprising SEQ ID NO: 224, LCDR2 comprising SEQ ID NO: 225, and LCDR3 comprising SEQ ID NO: 226.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

In some embodiments, the first and second variable heavy chain domain each comprises an amino acid sequence of SEQ ID NO: 300 and wherein the first and second variable light chain domain each comprises an amino acid sequence of SEQ ID NO: 301.

In some embodiments, the first and third polypeptide chain each comprises an amino acid sequence comprising SEQ ID NO: 300 and the second and fourth polypeptide chain each comprises an amino acid sequence comprising SEQ ID NO: 301.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In some embodiments, the anti-IL17RA antibody is a human or humanized antibody. In some embodiments, the anti-IL17RA monoclonal antibody is Brodalumab.

In some embodiments, the subject has a solid tumor. In some embodiments, the tumor is head-neck squamous cell carcinoma, sarcoma, liver hepatocellular carcinoma, gastric cancer, colorectal cancer, and breast cancer. In some embodiments, the cancer is breast cancer. In some embodiments, the cancer is melanoma. In some embodiments, the cancer is colon cancer.

In some embodiments, immune-related adverse events are treated, reduced, or prevented in the subject. In some embodiments, the immune-related adverse events comprise colitis, diarrhea, rash, pruritis, esophagitis, duodenitis, ileitis, neuritis, arthrhtis, vasculitis, nephritis, adrenal insufficiency, hepatitis, thrombocytopenia, anemia, pneumonitis, thyroiditis, hypophysitis, encephalitis, meningitis, uveitis, mucositis, rash, myocarditis, pericarditis, pancreatitis, colitis, enteritis, or any combination thereof.

In some embodiments, off-target immune infiltration of one or more untargeted organs in the subject is reduced or prevented. In some embodiments, CD3⁺ T cells are not detected or are not present at elevated levels in one or more untargeted organs in the subject.

In some embodiments, a tumor of the subject is reduced in volume. In some embodiments, growth of a tumor or cancer cells of the subject is inhibited.

In some embodiments, the combination of the anti-IL17RA antibody or antigen-binding fragment thereof and the at least one ICI exhibits a synergistic effect on reducing a tumor volume, cancer treatment, or inhibiting tumor growth compared to the tumor volume reduction, cancer treatment effect, or tumor growth inhibition exhibited by administering a therapeutic dose of the one or more ICI alone or a therapeutic dose of the anti-IL-17RA antibody or antigen binding fragment thereof alone.

In some embodiments, the anti-IL17RA antibody or antigen binding fragment thereof and the one or more ICI is administered concurrently as a single composition or as separate compositions.

In some embodiments, the anti-IL17RA antibody or antigen binding fragment thereof and the one or more ICI is administered sequentially.

In certain aspects, the present disclosure provides a method of determining a cancer prognosis in a subject in need thereof comprising determining IL17RA gene expression levels in a sample from the subject.

In some embodiments, the IL17RA gene expression levels are IL17RA gene expression levels of T cells of the subject. In some embodiments, the T cells are CD4 T cells, CD8 T cells, or both CD4 T cells and CD8 T cells. In some embodiments, the subject is determined to have a poor prognosis if the subject has an increased level of IL17RA gene expression as compared to the level of IL17RA gene expression in a healthy subject or cohort of healthy subjects.

In some embodiments, the sample is biopsy tissue or blood. In some embodiments, the IL-17RA gene expression is measured using RNA-seq, gene chip data, or q-PCR. In some embodiments, the subject is a human.

In certain aspects, the present disclosure provides a composition comprising therapeutically effect amounts of an anti-IL-17RA antibody or antigen binding fragment thereof and one or more ICIs

In certain aspects, the present disclosure provides a composition comprising therapeutically effect amounts of an anti-IL-17RA antibody or antigen binding fragment thereof and one or more ICIs comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312.

In certain aspects, the present disclosure provides a combination in the form of a kit comprising two or more compositions, the first composition comprising a therapeutically effect amount of an anti-IL-17RA antibody or antigen binding fragment thereof and the second composition comprising a therapeutically effect amount of one or more ICIs.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof. In some embodiments, the compositions further comprises one or more pharmaceutically acceptable excipients.

In some embodiments, the at least one ICI comprises an anti-PD-1 antibody or antigen binding fragment thereof, comprising the CDRs or variable domains of clones 01, 02, 03, 04, 07, 09, 51, humanized 51, 55, 79, 80 or SEQ ID NOS: 311-312. In some embodiments, the compositions further comprises one or more pharmaceutically acceptable excipients.

In some embodiments, the compositions further comprises a package insert or label providing directions for administering the compositions simultaneously, separately or sequentially.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

In some embodiments, the anti-PD-1 antibody or antigen binding fragment thereof comprises Nivolumab, Pembrolizumab, Cemiplimab, Retifanlimab, Dostarlimab, Zimberelimab, Tiselelizumab, Camrelizumab, Sintilimab, Penpulimab or antigen binding fragment thereof. In some embodiments, the anti-PDL-1 antibody or antigen binding fragment thereof comprises Atezolimumab, Durvalumab and Avelumab, or antigen binding fragment thereof. In some embodiments, the anti-CTLA-4 antibody or antigen binding fragment thereof comprises ipilimumab, tremelimumab, or a combination thereof.

In some embodiments, the anti-LAG-3 antibody or antigen binding fragment thereof comprises BMS-986016, Relatimab, INCAGN02385, GSK2831781, or a combination thereof. In some embodiments, the anti-IL-17RA antibody or antigen binding fragment thereof is a monoclonal antibody or antigen binding fragment thereof.

In some embodiments, the at least one ICI comprises an anti-PD-1 antibody or antigen binding fragment thereof, comprising the CDRs or variable domains of clones 01, 02, 03, 04, 07, 09, 51, humanized 51, 55, 79, 80, or SEQ ID NOS: 311-312.

In some embodiments, the anti-IL-17RA antibody or antigen binding fragment thereof, comprises: a first arm comprising a first variable heavy chain domain and a first variable light chain domain, wherein a portion of the first arm is capable of binding to a portion of an IL-17RA; and a second arm comprising a second variable heavy chain domain and a second variable light chain domain, wherein a portion of the second arm is capable of binding to a portion of the IL-17RA protein and the first and second arms each further comprise a fragment, crystallizable (Fc) domain.

In some embodiments, the first and second arms each further comprise a CHI domain, a hinge domain, and a CL domain. In some embodiments, the portion of IL-17RA bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-17RA binding site and the second variable heavy chain domain and second variable light chain domain form a second IL-17RA binding site.

In some embodiments, the first and second IL-17RA binding sites are the same. In some embodiments, the first and third polypeptide chain each further encode a hinge domain, a CHI domain, and the Fc domain, and wherein the second and fourth polypeptide chain each further encode a CL domain. In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 146, HCDR2 comprising SEQ ID NO: 147, and HCDR3 comprising SEQ ID NOs: 148 and wherein the first and second variable light chain domain each comprises LCDR1 comprising SEQ ID NO: 224, LCDR2 comprising SEQ ID NO: 225, and LCDR3 comprising SEQ ID NO: 226.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

In some embodiments, the first and second variable heavy chain domain each comprises an amino acid sequence of SEQ ID NO: 300 and wherein the first and second variable light chain domain each comprises an amino acid sequence of SEQ ID NO: 301.

In some embodiments, the first and third polypeptide chain each comprises an amino acid sequence comprising SEQ ID NO: 300 and the second and fourth polypeptide chain each comprises an amino acid sequence comprising SEQ ID NO: 301.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds. In some embodiments, the anti-IL17RA antibody is a human or humanized antibody. In some embodiments, the anti-IL17RA monoclonal antibody is Brodalumab.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-F show induction of irAEs by anti-PD-1 and anti-CTLA-4 antibodies therapy in multiple organs. FIG. 1A shows a mouse model for immune checkpoint blockade-induced immune-related adverse events. Anti-PD1 (200 ug) and anti-CTLA4 (200 ug) were given biweekly through intraperitoneal injections starting on day 1 for six weeks or until sacrifice. FIG. 1Bi shows each curve representing a treatment group. Tumor growth of aPD1+aCTLA4 treated mice and untreated mice are shown as indicated by symbols and legend. FIG. 1Bii shows average tumor volumes on day 18. Left hand bar is untreated and right hand bar is aPDla/CTLA4. FIG. 1C shows immune infiltration gradings of H&E-stained liver, lung, heart, colon, and pancreas harvested upon mouse euthanasia. For each organ, left hand bar is untreated and right hand bar is aPDla/CTLA4. FIG. 1Di-Diii show spectral flow analysis reveals multiple CD45+t-cell clusters composing liver and tumor. FIGS. 1E-F show differences between liver and tumor CD45+t-cell populations. Left hand and middle clusters are enriched in the liver, and the right hand cluster is unique to the tumor.

FIGS. 2A-I show treatments for irAEs counteracting ICI's anti-tumor effect. FIG. 2A shows the experiment design for examining the effect of prednisolone on tumor and immune-related adverse event development. Prednisolone was given daily via oral gavage from day 8 to day 12 (5 doses total) at 1 mg/kg body weight in addition to regular aPDI and aCTLA4 administration. FIG. 2B shows immune infiltration gradings of H&E-stained organs harvested upon mouse euthanasia reveal levels of organ-specific immune infiltration. For each organ, left hand bar is untreated, middle bar is aPD1/aCTLA4, and right hand bar is aPD1/aCTLA4/Prednisolone. FIG. 2Ci shows each curve representing a treatment group illustrating tumor growth. FIG. 2Cii shows average tumor volume on day 18 (left hand bar is untreated, middle bar is aPD1/aCTLA4, and right hand bar is aPD1/aCTLA4/Prednisolone). FIG. 2D shows a Kaplan-Meier plot for survival estimate of untreated mice and mice receiving aPD1+aCTLA4 with and without prednisolone. The table shows the number of surviving mice on days 0, 10, 20, 30, 40. FIG. 2E shows luminex for detecting levels of cytokines in peripheral blood serum collected at in-vivo endpoints (for each condition, left hand bar is untreated, middle bar is aPD1/aCTLA4, and right hand bar is aPD1/aCTLA4/Prednisolone). FIG. 2F shows the experiment design for examining the effect of anti-IL6 and anti-TNF antibodies on tumor and immune-related adverse event development. Either aIL6 or aTNFa was given biweekly starting day two at 200 μg per dose through intraperitoneal injections for two weeks on top of the regular dosage of aPDI and aCTLA4. FIG. 2G shows immune infiltration gradings of H&E-stained liver and lung harvested at an in-vivo endpoint (for each organ, from left to right bars represent untreated, aPD1/aCTLA4, aPD1/aCTLA4/aIL6, aPD1/aCTLA4/aTNFa). FIG. 2Hi shows tumor growth curves of different treatment groups (for each organ, from left to right bars represent untreated, aPD1/aCTLA4, aPD1/aCTLA4/aIL6, aPD1/aCTLA4/aTNFa). FIG. 2Hii shows the average tumor volumes on day 18 (from left to right bars represent untreated, aPD1/aCTLA4, aPD1/aCTLA4/aTNF6, aPD1/aCTLA4/aIL6). FIG. 2I shows KM plot of estimated survival probabilities of aPD1+aCTLA4 treated mice, aPD1+aCTLA4+aIL6 treated mice, and aPD1+aCTLA4+aTNFα treated mice within the 40 days treatment period.

FIGS. 3A-I show IL17RA as an alternative and advantageous target than IL25. FIG. 3A shows IL17 ligand and receptor family. Ligands-IL17A, IL17A/F, IL17F, IL17E (IL25), IL17B, IL17C, and IL17D—are either homo- or hetero-dimers. FIG. 3B shows a schematic representation of the experimental design for administering anti-IL17RA antibody (100 ug) twice in combination with aPD1+aCTLA4. FIG. 3Ci shows tumor growth curve of aIL17RA treated mice compared to those without aIL17RA treatment (treatment conditions are indicated by arrows). FIG. 3Cii shows average tumor volumes on day 18 (From the left hand side to the right is untreated, aPD1/aCTLA4, aPD1/aCTLA4/aIL17RA, aPD1/aCTLA4/Ail25). FIG. 3D shows gradings of immune infiltration on day 25. One case of pericarditis was observed and assigned a score of 2 (for each organ, from left to right bars represent untreated, aPD1/aCTLA4, aPD1/aCTLA4/aIL17RA). FIG. 3E shows flow cytometry plot of percent IL17RA+ in CD4+ T-cell and CD8+ T-cell from regular Lpr mice splenocytes (T cells=CD3+gate). FIG. 3F shows flow cytometry analysis of CD69 activation marker expression in IL17RA+ and IL17RA-CD4+t-cells. And flow cytometry analysis of IL17RA in central and effector CD4 t-cells. FIG. 3G shows flow cytometry analysis of IL17RA expression in activated (PD 1^{high} CD69+) and exhausted (PD1^{high} CD69−) CD4 t-cells. FIGS. 3H-I show comparison between naïve Lpr mice, Lpr untreated mice with MC38 tumor, tumor mice treated with aPD1+aCTLA4, and tumor mice treated with aPD1+aCTLA4+aIL17RA (from left hand to right). The percentage of exhausted cells among CD4 t-cells is illustrated.

FIGS. 4A-D shows that T cell IL17RA gene expression correlates with worse patient outcomes. FIG. 4A shows survival probability of patients receiving aPD1 immunotherapy predicted by IL17RA expression and IL17RA/CD3 gene expression ratio. FIG. 4B shows aPDI immunotherapy patient survival predicted by IL17RA/CD4 and IL17RA/CD8 gene expression ratio. FIGS. 4Ci, Cii, Ciii show probability of survival predicted by IL17RA/CD4 mRNA expression ratio in multiple malignancies. FIG. 4D shows colorectal and breast cancer patient survival predicted by the IL17RB/CD4 mRNA expression ratio. High dose treatment is indicated by a star in FIGS. 4A-D.

FIG. 5 shows the sequences for the CDRs for anti-L17RA variable heavy chain domain adapted from the International Patent Application No. WO2008054603.

FIG. 6 shows the sequences for the CDRs for anti-L17RA variable light chain domain adapted from the International Patent Application No. WO2008054603.

FIG. 7 shows types of irAEs adapted from Postow, M. A., R. Sidlow, and M. D. Hellmann, Immune-Related Adverse Events Associated with Immune Checkpoint Blockade, New England Journal of Medicine, 2018. 378 (2): p. 158-168.

FIG. 8 shows that Immune Checkpoint Inhibitors can lead to the development of inflammation nearly every organ adapted from Mor, A. and M. Strazza, Bridging the Gap: Connecting the Mechanisms of Immune-Related Adverse Events and Autoimmunity Through PD-1, Front Cell Dev Biol, 2021. 9: p. 790386.

FIGS. 9A-B shows that inhibition of ILRA promotes responsiveness to immune checkpoint inhibitor therapy. FIG. 9A shows that B6/lpr female mice of 7 & 10 weeks of age were inoculated with 2×10⁵ MC38 colon adenocarcinoma tumor cells in the right flank and tumor volumes were measured daily with mechanical caliper. Mice were treated with aPD-1+aCTLA-4, aPD-1+aCTLA+aIL-17RA, or untreated. 200 μg of anti-PD-1 and 200 μg of anti-CTLA-4 were given intra peritoneally starting on day 1, when tumor volumes were between 20 to 30 mm3, twice a week for six weeks (12 treatments in total). A single dose of anti-mouse IL-17RA was given intra peritoneally on day 6 at 20 mg/kg. FIG. 9B shows tumor growth curve of aIL17RA+aPD-1+aCTLA treated mice compared to those with only aIL17RA treatment and those without aIL17RA treatment.

FIG. 10 shows flow cytometry plot of percent IL17RA+ and IL17RB+ in MC38 mouse adenocarcinoma cells.

FIGS. 11A-11D shows that genetic silencing of IL17RA inhibits tumor growth. FIG. 11A shows a flow cytometry plot of percent IL17RA+ in parental and IL17RA KO MC38 mouse adenocarcinoma cells. B6/lpr female mice of 7 weeks of age are inoculated with either parental or knockout mice and tumor volumes were measured with mechanical calipers over the course of 22 days. FIG. 11B shows tumor growth curve of mice treated with parental MC38 tumor cells compared to those treated with IL17RA knockout cells. FIG. 11C shows that B6/lpr female mice of 7 weeks of age are inoculated with either parental or knockout mice and tumor volumes were measured with mechanical calipers over the course of 22 days, with anti-PD1 or no treatment therapy being administered periodically. FIG. 11D shows tumor growth curve of mice treated with parental MC38 tumor cells compared to those treated with IL17RA knockout cells in combination with anti-PD1 therapy or no treatment.

FIGS. 12A-B show binding curves to human-PD-1-His-coated plates quantified by ELISA. FIG. 12A shows binding curves for anti-PD-1 antibody clones 01, 02, 03, and 07, anti-human PD-1 antibody Penbio (pembrolizumab), anti-HEL-human IgG1 isotype control, and blank. FIG. 12B shows binding curves for anti-PD-1 antibody clones 09, 51, 55, 79, and 80, anti-human PD-1 antibody Penbio (pembrolizumab), anti-HEL-human IgG1 isotype control, and blank. EC50 values were calculated with GraphPad Prism (v10.2.1).

FIG. 13 shows binding of anti-PD-1 clones 01, 02, 03, 07, 09, 51, 55, 79, and 80 to cell-expressed PD-1 by flow cytometry.

FIG. 14 shows anti-PD-1 clones 01, 02, 03, 07, 09, 51, 55, 79, and 80 blocking the binding of rhPD-L2 to cell-expressed PD-1.

FIG. 15 shows IL-2 concentrations determined by ELISA following addition of anti-PD-1 clones 01, 02, 03, 07, 09, 51, 55, 79, or 80 and SEE (Staphylococcal Enterotoxin E) in Jurkat-Raji co-culture compared to no SEE and no antibody control and SEE and no antibody control.

FIG. 16 shows IL-2 concentrations determined by ELISA following addition of anti-PD-1 clones 01, 02, 03, 07, 09, 51, 55, 79, or 80 at different concentrations (10, 2, 0.5 or 0.1 μg/ml) and SEE (Staphylococcal Enterotoxin E) in Jurkat-Raji co-culture compared to no SEE and no antibody control and SEE and no antibody control.

FIG. 17 shows concentrations of IL-2, IFNγ, IL-6, IL-4 and IL-1ß determined by ELISA in PBMCs in the presence of anti-PD-1 clones 01, 02, 03, 07, 09, 51, 55, 79, or 80 and SEE (Staphylococcal Enterotoxin E).

FIG. 18 shows anti-PD1 antibody sequences with CDRs highlighted. Figure discloses SEQ ID NOS 313, 322, 314, 323, 315, 324, 316, 325, 317, 326, 318, 327, 319, 328, 320, 329, 321, and 330, respectively, in order of appearance.

FIGS. 19A-F show the Kaplan-Mayer curves of overall survival of high (star) and low (arrow) IL17RA expression defined by median expression for each cohort. FIG. 19A shows data from cervical cancer. FIG. 19B shows glioma. FIG. 19C shows ovarian cancer. FIG. 19D shows head and neck cancer. FIG. 19E shows liver cancer. FIG. 19F shows renal cancer.

FIG. 20 shows cluster plots derived from single-cell RNA sequencing data depicting the expression levels of IL17RA in CD4+, CD8+, and CD4+CD8+ T cells. The data shows that administration of anti-murine IL17RA antibodies resulted in decreased tumor size and prevention of irAEs.

FIG. 21 shows Serum cytokine levels and treatment response in irAE mice. Bars for each cytokine are shown from left to right as pretreatment, untreated, aPD-1/aCLTA-4, LNR125HD, LNR125LD, Prednisolone.

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein are antagonists of the interleukin 17 receptor A for cancer therapy and prevention of immune-related adverse events of checkpoint inhibitors. In some embodiments, specific anti-IL17RA, anti-CTLA-4, and anti-PD-1 antibodies can be used in the treatment of cancers and inhibition of irAEs.

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

The term “therapeutically effective amount,” as used herein, refers to an amount or a concentration of one or more compounds or a pharmaceutical composition described herein utilized for a period of time (including in vitro and in vivo acute or chronic administration and periodic or continuous administration) that is effective within the context of its administration for causing an intended effect or physiological outcome.

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein are methods of treating cancer (e.g., colon cancer, or breast cancer) while improving immune checkpoint inhibitors (ICI) tumor suppressive effects and reducing or inhibiting its induced immune-related adverse events (irAEs). Specifically, described herein are Il-17RA antagonists (e.g., anti-IL-17RA antibodies, siIL-17RA) in combination with ICIs (e.g., CTLA-4 antibody, anti-PDL-1 antibody, anti-LAG-3 antibody, an anti-PD-1 antibody, or antigen-binding fragment thereof, such as anti-PD1 antibodies described herein (clones 01, 02, 03, 04, 07, 09, 51, humanized 51, 55, 79, 80, or SEQ ID NOS: 311-312), or a combination thereof.

As used herein, “IL-17RA” refers to Interleukin-17A receptor.

### Immune Checkpoint Inhibitors as a Cancer Treatment

Immunotherapy drugs called immune checkpoint inhibitors (ICI) work by blocking checkpoint proteins from binding with their partner proteins. This prevents the “off” signal from being sent, allowing the T cells to kill cancer cells. One such drug acts against a checkpoint protein called CTLA-4. Other immune checkpoint inhibitors act against a checkpoint protein called PD-1 or its partner protein PD-L1. Some tumors turn down the T-cell response by producing lots of PD-L1. ICI therapy is approved for the treatment of several cancers including colon cancer. However, ICIs can cause side effects that include widespread inflammation. The benefits of ICIs can be offset by the production of an array of sometimes life-threatening immune-related adverse events (irAEs) and subsequently increase the risk for morbidity and mortality. There is currently no mechanistic understanding of irAEs, complexity and heterogeneity, nor ways to predict those patients who will develop an irAEs in response to ICIs. As ICIs approaches are expanded and used in increasingly effective combinations, controlling irAEs will be a critical goal of immunotherapy development.

ICIs are approved for indications across different cancer types and stages, with an estimated 233,790 cases eligible for treatment each year. Despite their powerful advance, there remain many challenges to the use of ICIs that must be met to best advance the next generation of therapies: increase responsiveness to PD-1 and CTLA-4 blockade, uncover new targets to optimize pathway blockade, and not less importantly, predict and effectively manage irAEs.

Immune checkpoint inhibitors (ICIs) have improved outcomes and extended patient survival in several tumor types. However, ICIs often induce immune-related adverse events (irAEs) that warrant therapy cessation, thereby limiting the overall effectiveness of this class of therapeutic agents. Currently, available therapies used to treat irAEs might also blunt the antitumor activity of the ICI themselves. Therefore, there is an urgent need to identify treatments that have the potential to be administered alongside ICI to optimize their use.

### Methods of Treatment

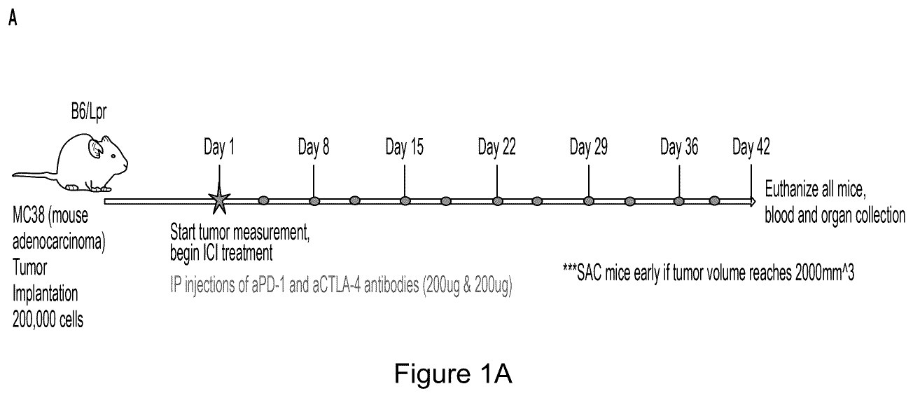

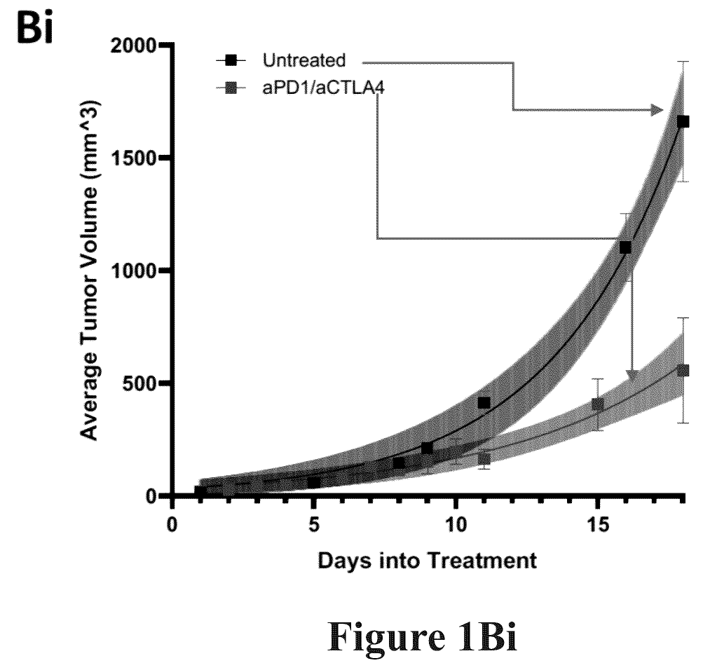

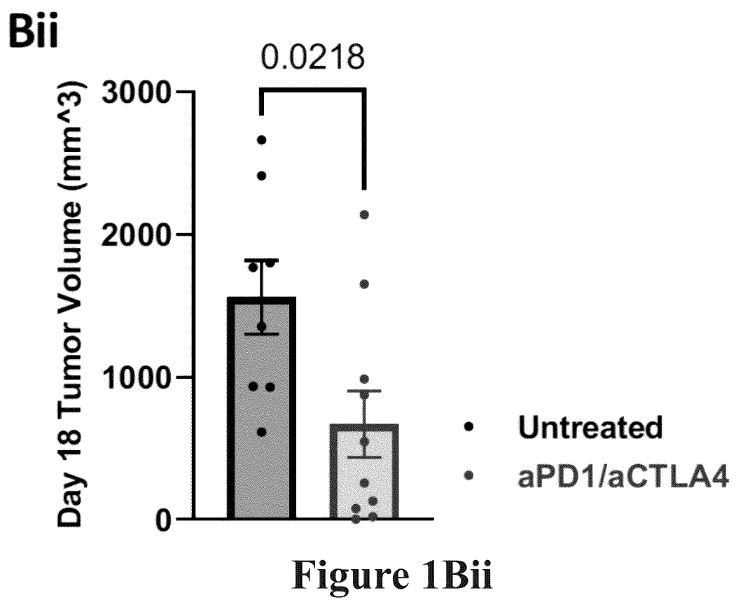

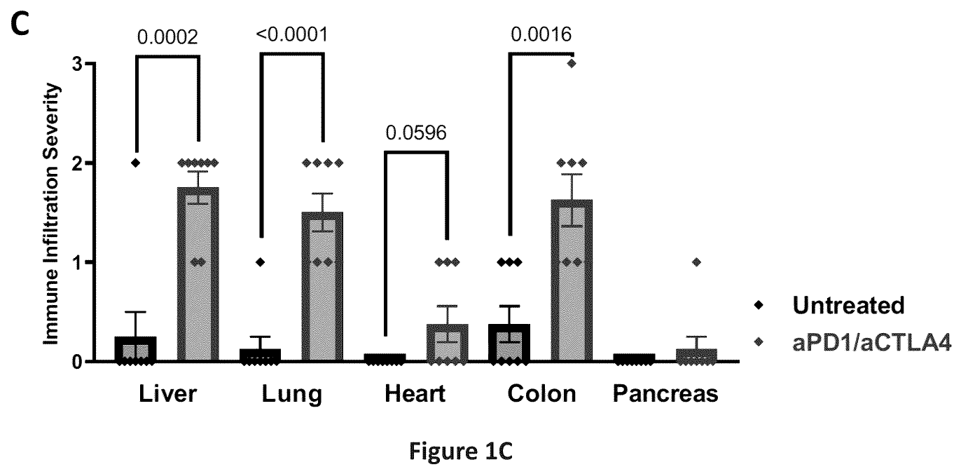

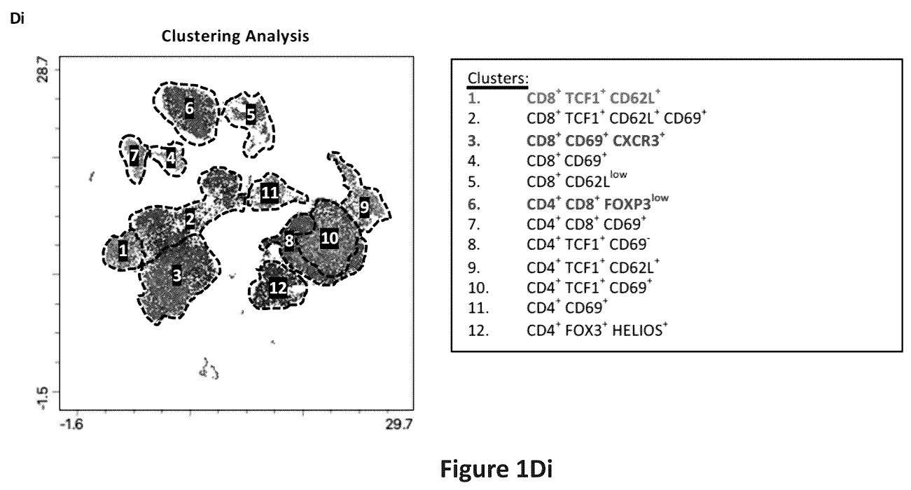

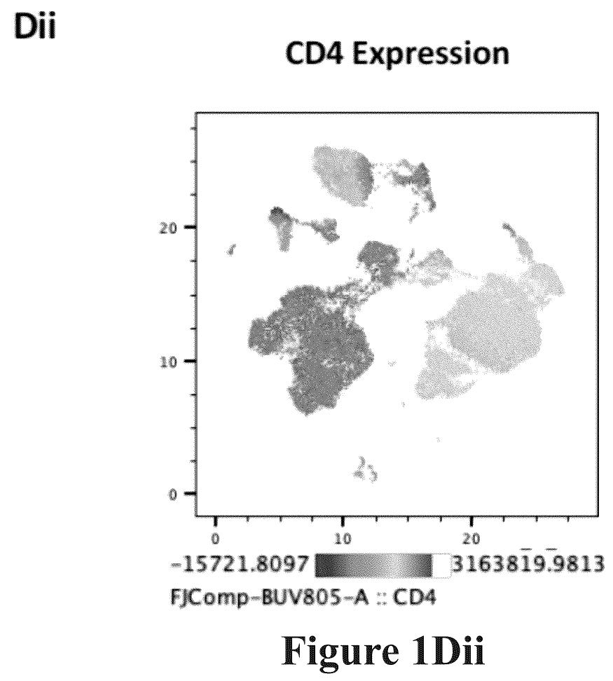

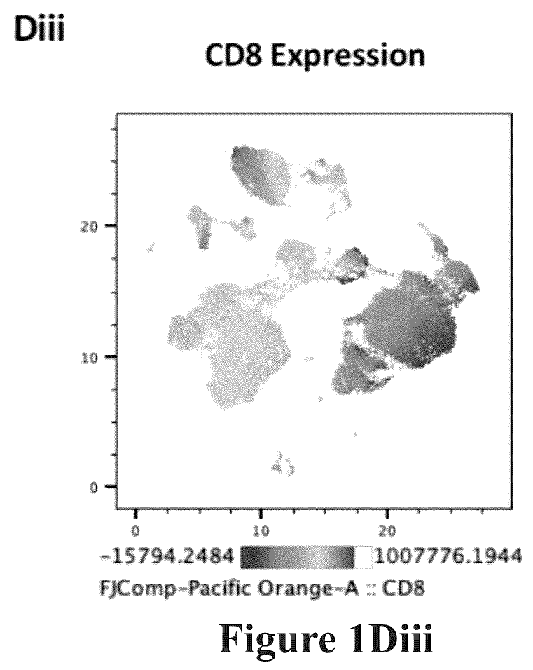

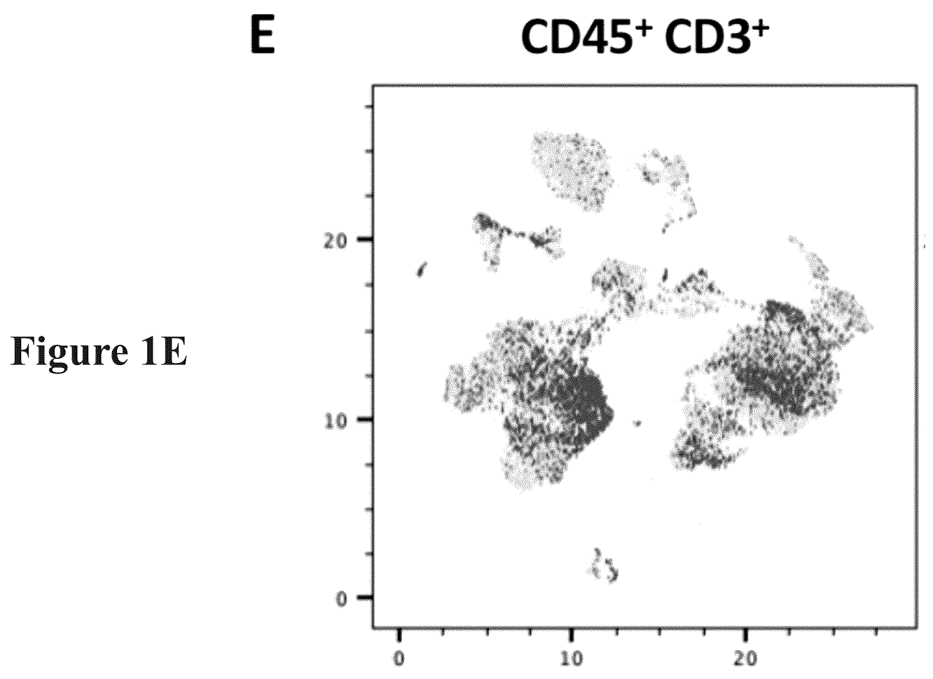

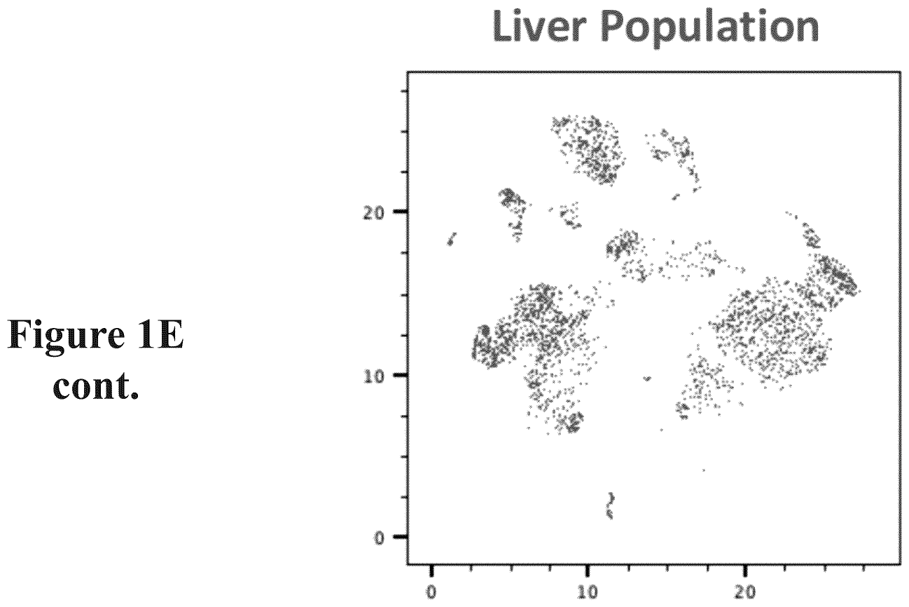

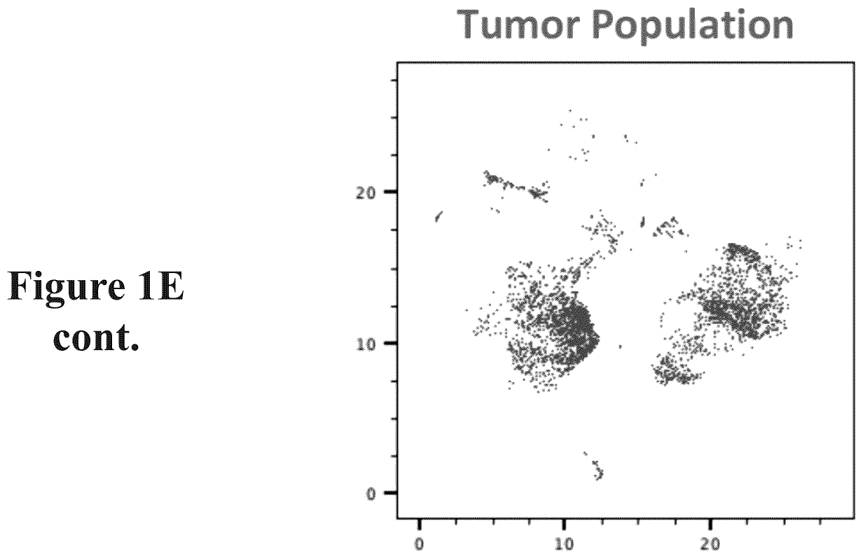

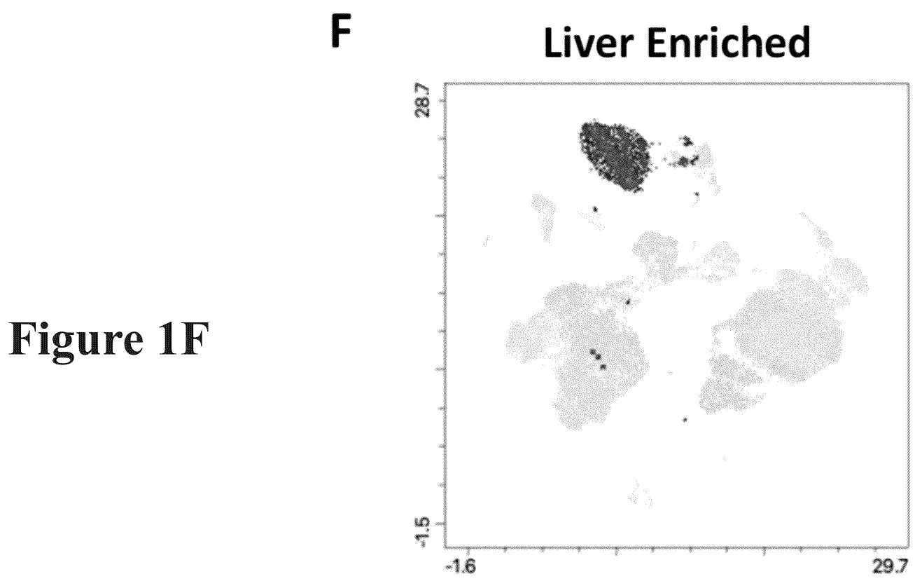

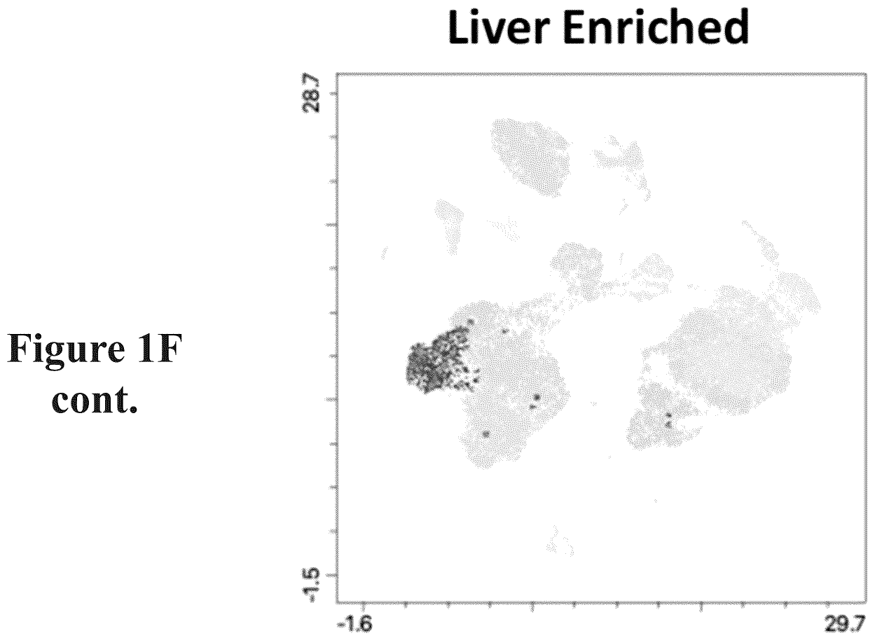

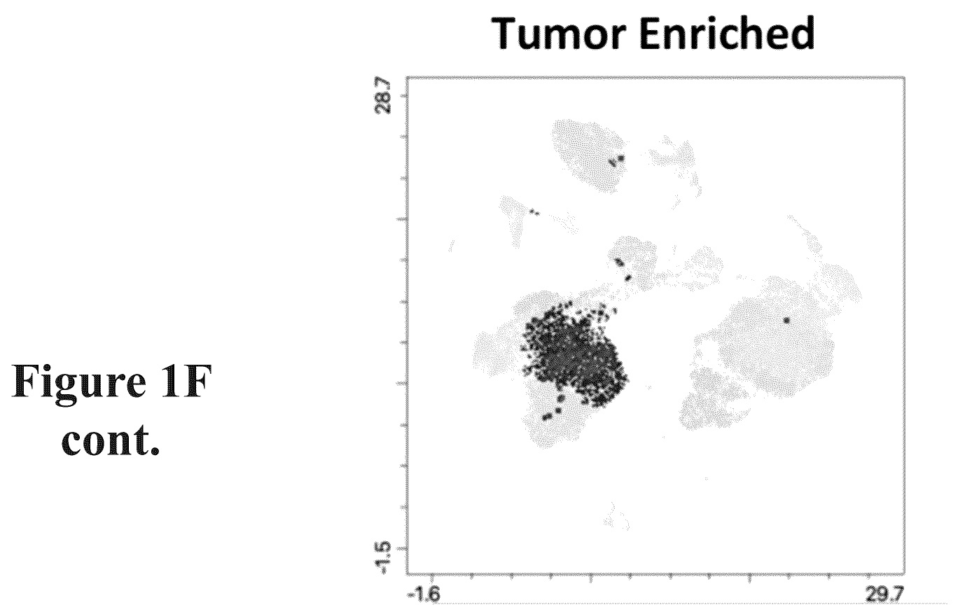

The interleukin-17 cytokine family consists of six cytokines (interleukins 17A to 17F) and five receptors (interleukins 17RA to 17RE). The interleukin 17A, 17F, and 17A/F heterodimer ligands share a common receptor subunit (interleukin-17RA) for signaling. 17E (also called IL25) binds to 17RA and 17RB for downstream signaling (FIG. 1). Brodalumab is a monoclonal antibody against IL17 receptor A (IL17RA). It's currently FDA-approved for treating moderate to severe plaque psoriasis in adult patients. Described herein is the role of 17E as a tumor suppressor. Studies previously published also showed that blocking 17A enhances tumor response to anti-PDI immunotherapy. See Liu, Chao et al. “Blocking IL17A enhances tumor response to anti-PD-1 immunotherapy in microsatellite stable colorectal cancer.” Journal for immunotherapy of cancer vol. 9, 1 (2021): e001895. doi: 10.1136/jitc-2020-001895. Described herein is blocking of the receptor 17RA can be a breakthrough immune checkpoint inhibitor therapy.

Without intending to be bound by any particular theory, it is hypothesized that Brodalumab (anti-IL17RA) antibody can be an effective treatment for cancer and in combination with ICIs described herein (e.g., comprising an anti-PD-1 antibody, or antigen-binding fragment thereof, such as anti-PD1 antibodies described herein (clones 01, 02, 03, 04, 07, 09, 51, humanized 51, 55, 79, 80, or SEQ ID NOS: 311-312)) can prevent irAEs. Brodalumab blocks the interaction between IL17RA and its ligand cytokine, interleukin 25 (IL25), ultimately blocking downstream activation of NF-KB and subsequent inflammation.

IL17 family cytokines (including but not limited to IL17A) are known to be regulating the tumor microenvironment and exhibits tangled effects on tumor growth. IL17 is known to mediate the protumoral and antitumoral effects through γδT-cells. Uncovering the functions of specific ligand/receptor in the IL17 family will shine light on new grounds for cancer therapy.

IL17 is also known to be mediating excessive inflammation in autoimmune diseases. irAEs. Even though biomarkers for irAEs have not been fully distinguished, risk of irAEs is correlated to predisposition to autoimmune diseases. There is a strong phenotypic and genotypic basis for targeting the IL17 family for immune-related adverse events.

Disclosed herein is an antagonist of IL 17 receptor A (IL17RA) as an effective therapy for tumors when used in combination with ICI therapy and is capable of reducing the irAEs of conventional ICI treatments. In some embodiments, the IL17RA antagonist is an anti-IL-17RA antibody. In some embodiments the ICI therapy comprises an anti-PD-1 antibody, or antigen-binding fragment thereof, such as anti-PD1 antibodies described herein (clones 01, 02, 04, 03, 07, 09, 51, humanized 51, 55, 79, 80, or SEQ ID NOS: 311-312).

In certain aspects, the present disclosure provides a method for treating cancer in a subject in need thereof comprising administering to the subject a therapeutically effective amount of an anti-IL-17RA antibody or antigen binding fragment thereof. In some embodiments, the treatment method further comprises administering to the subject a therapeutically effective amount of at least one immune checkpoint inhibitor (ICI). In certain embodiments, the present disclosure also provides a method for treating, reducing, or preventing iRAEs in a subject in need thereof comprising administering to the subject a composition comprising a therapeutically effective amount of an anti-IL-17RA antibody or antigen binding fragment thereof, wherein the iRAEs are caused by administration of at least one ICI to the subject.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof. In some embodiments, the anti-PD-1 antibody or antigen binding fragment thereof comprises Nivolumab, Pembrolizumab, Cemiplimab, Retifanlimab, Dostarlimab, Zimberelimab, Tiselelizumab, Camrelizumab, Sintilimab, Penpulimab or antigen binding fragment thereof. In some embodiments, the anti-PDL-1 antibody or antigen binding fragment thereof comprises Atezolimumab, Durvalumab and Avelumab, or a combination thereof. In some embodiments, the anti-CTLA-4 antibody or antigen binding fragment thereof comprises ipilimumab, tremelimumab, or a combination thereof. In some embodiments, the anti-LAG-3 antibody or antigen binding fragment thereof comprises BMS-986016, Relatimab, INCAGN02385, GSK2831781, or a combination thereof. In some embodiments, the anti-IL-17RA antibody or antigen binding fragment thereof is a monoclonal antibody or antigen binding fragment thereof.

In certain aspects, the present disclosure provides a method for treating cancer in a subject in need thereof comprising administering to the subject a therapeutically effective amount of an anti-IL-17RA antibody or antigen binding fragment thereof, the treatment method further comprises administering to the subject a therapeutically effective amount of at least one immune checkpoint inhibitor (ICI) comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312. In certain embodiments, the present disclosure also provides a method for treating, reducing, or preventing iRAEs in a subject in need thereof comprising administering to the subject a composition comprising a therapeutically effective amount of an anti-IL-17RA antibody or antigen binding fragment thereof, wherein the iRAEs are caused by administration of at least one ICI comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312 to the subject.

In some embodiments, the ICI comprises an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312. In some embodiments, the amino acid sequence that encodes the anti-PD-1 antibody heavy chain comprises of any of the sequences of Table 1, 4, 5, and SEQ ID NO: 308. In some embodiments, the amino acid sequence that encodes the anti-PD-1 antibody light chain comprises of any of the sequences of Table 2, 6, 7, and SEQ ID NO: 309.

In some embodiments, the amino acid sequence of the anti-PD-1 antibody in scFv format with VH and VL domains from clone 04 comprises SEQ ID NO: 311:

EVKLVESGGGLVQPGGSLKLSCAASGFTFSSYTMSWVRQTPEKRLEWVA
YISYGGGDTYYSDTVKGRFTISRDNAKNTLYLQMSSLKSEDTAMYYCAR
QKVDGYYVGMDYWGQGSSVTVSSGGGGSGGGGSGGGGSNIVMTQSPKSM
SMSVGERVTLSCKASENVGTNVSWYQQKPEQSPRLLIYGASNRYTGVPD
RFTGSGSVTDFTLTISSVQAEDLVDYHCGQSYSSPLTFGSGTKLELK.

In some embodiments, any of the CDRs or variable domains of clones 01, 02, 03, 04, 07, 09, 51, humanized 51, 55, 79, 80 are in scFv format.

In some embodiments, the anti-PD-1 antibody comprises a VH domain comprising SEQ ID NO: 306 (clone 04) (EVKLVESGGGLVQPGGSLKLSCAASGFTFSSYTMSWVRQTPEKRLEWVAYISYGGGDT YYSDTVKGRFTISRDNAKNTLYLQMSSLKSEDTAMYYCARQKVDGYYVGMDYWGQG SSVTVSS). In some embodiments, the anti-PD-1 antibody comprises a VL domain comprising SEQ ID NO: 307 (clone 04) (NIVMTQSPKSMSMSVGERVTLSCKASENVGTNVSWYQQKPEQSPRLLIYGASNRYTGV PDRFTGSGSVTDFTLTISSVQAEDLVDYHCGQSYSSPLTFGSGTKLELK).

In some embodiments, the amino acid sequence of the anti-PD-1 antibody in scFv format with humanized clone 51 VH and VL domains comprises SEQ ID NO: 312:

(SEQ ID NO: 312)
EVQLVESGGGLVKPGGSLRLSCAASGFTFSSYGMSWVRQAPGKGLEWVA
TISGGGRYTYYPDSVKGRFTISRDNAKNSLYLQMNSLRAEDTAVYYCAS
PYDGYYGAMDYWGQGTSVTVSSGGGGSGGGGSGGGGSEIVLTQSPATLS
LSPGERATLSCRASESVDNSGISFMNWYQQKPGQAPRLLIYAASNQGSG
IPARFSGSGSGTDFTLTISSLEPEDFAVYYCQQSKEVPWTFGGGTKLEI
KR.

In some embodiments, the anti-PD-1 antibody comprises a VH domain comprising SEQ ID NO: 308 (humanized clone 51) (EVOLVESGGGLVKPGGSLRLSCAASGFTFSSYGMSWVRQAPGKGLEWVATISGGGRY TYYPDSVKGRFTISRDNAKNSLYLQMNSLRAEDTAVYYCASPYDGYYGAMDYWGQGT SVTVSS). In some embodiments, the anti-PD-1 antibody comprises a VL domain comprising SEQ ID NO: 309 (humanized clone 51) (EIVLTQSPATLSLSPGERATLSCRASESVDNSGISFMNWYQQKPGQAPRLLIYAASNQGS GIPARFSGSGSGTDFTLTISSLEPEDFAVYYCQQSKEVPWTFGGGTKLEIKR).

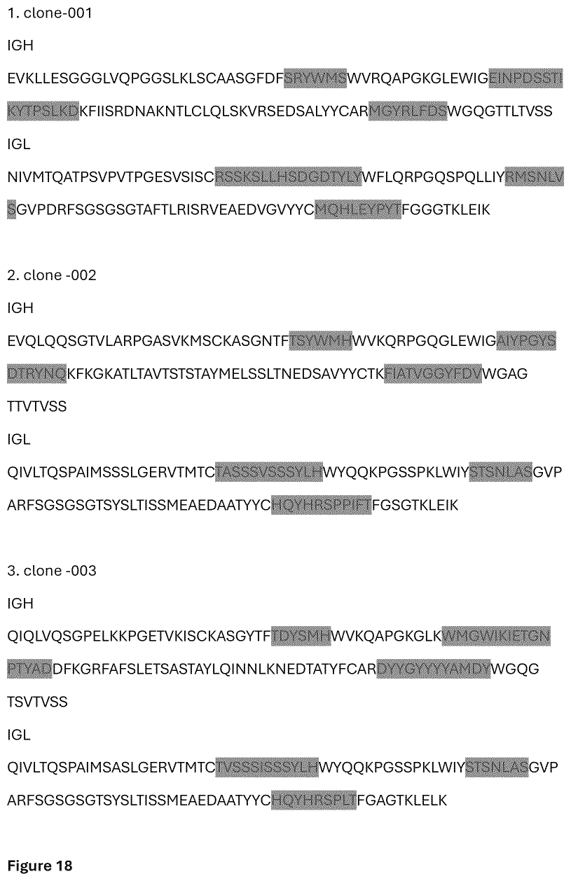

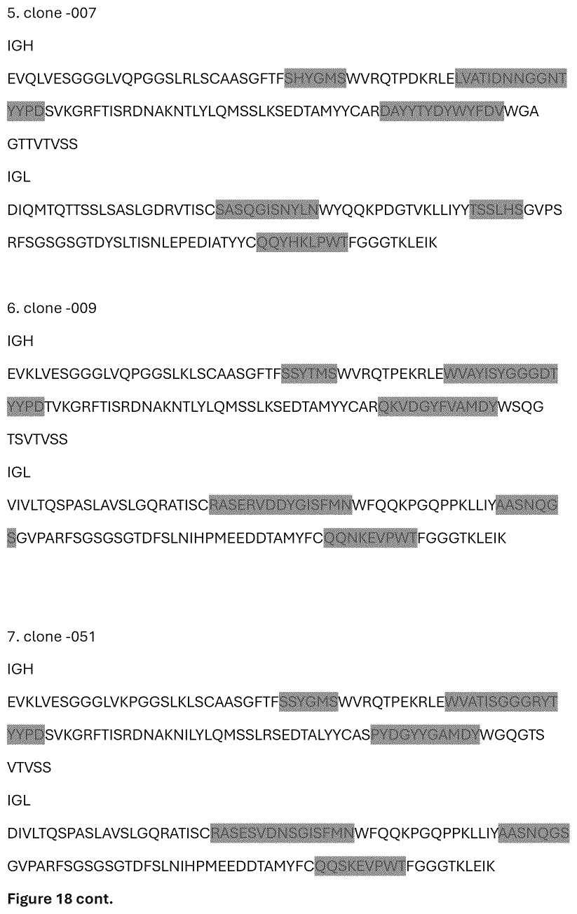

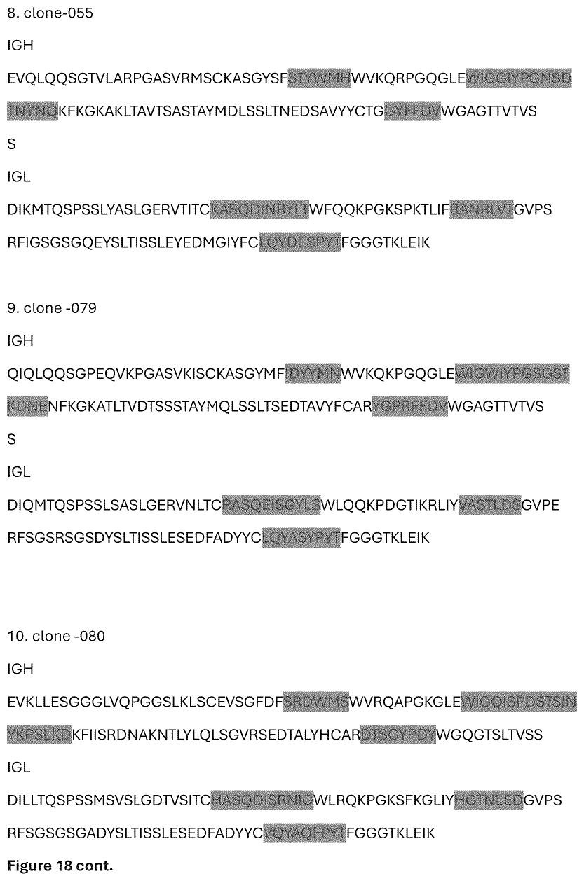

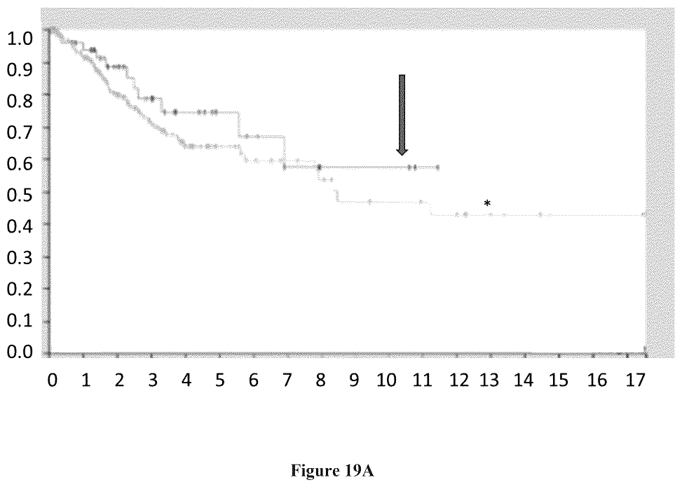

In some embodiments, the anti-PD-1 antibody comprises a VH domain with hypervariable regions HCDR1, HCDR2 and HCDR3 sequences disclosed as in either FIG. 18 or in Table 4, including combinations thereof (e.g. any HCDR1, HCDR2, HCDR3 of FIG. 18 or Table 4 in combination with any HCDR1, HCDR2, HCDR3 of FIG. 18 or Table 4). In some embodiments, the anti-PD-1 antibody comprises a FR region which comprises HFR regions HFR1, HFR2, HFR3, and HFR4 sequences disclosed in FIG. 18 or Table 5. In some embodiments, the anti-PD-1 antibody comprises a VL domain which comprises hypervariable regions LCDR1, LCDR2, and LCDR3 comprising the amino acid sequences disclosed in FIG. 18 or Table 6, including combinations thereof (e.g. any LCDR1, LCDR2, LCDR3 of FIG. 18 or Table 6 in combination with any LCDR1, LCDR2, LCDR3 of FIG. 18 or Table 6). In some embodiments, the anti-PD-1 antibody comprises a FR region which comprises LFR regions LFR1, LFR2, LFR3, and LFR4 sequences disclosed in FIG. 18 or Table 7.

In some embodiments, the CDRs of the variable heavy chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the CDRs of SEQ ID NO: 308. In some embodiments, the CDRsof the variable light chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the CDRs of SEQ ID NO: 309. In some embodiments, the CDRs of the variable heavy chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the CDRs of Table 4. In some embodiments, the CDRs of the variable light chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the CDRs of Table 6.

In some embodiments, the framework regions (FRs) of the variable heavy chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 308. In some embodiments, the framework regions (FRs) of the variable light chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 309. In some embodiments, the framework regions (FRs) of the variable heavy chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the FRs of Table 5. In some embodiments, the framework regions (FRs) of the variable light chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the FRs of Table 7.

TABLE 1

| SEQ ID No. | anti-PD-1 Heavy Chain. See FIG. 18 for CDRs |
|---|---|
| 313 (clone | EVKLLESGGGLVQPGGSLKLSCAASGFDFSRYWMSWVRQAPGKGL |
| 01) | EWIGEINPDSSTIKYTPSLKDKFIISRDNAKNTLCLQLSKVRSEDSALY |
| | YCARMGYRLFDSWGQGTTLTVSS |
| 314 (clone | EVQLQQSGTVLARPGASVKMSCKASGNTFTSYWMHWVKQRPGQG |
| 02 | LEWIGAIYPGYSDTRYNQKFKGKATLTAVTSTSTAYMELSSLTNEDS |
| | AVYYCTKFIATVGGYFDVWGAGTTVTVSS |
| 315 (clone | QIQLVQSGPELKKPGETVKISCKASGYTFTDYSMHWVKQAPGKGLK |
| 03 | WMGWIKIETGNPTYADDFKGRFAFSLETSASTAYLQINNLKNEDTA |
| | TYFCARDYYGYYYYAMDYWGQGTSVTVSS |
| 316 (clone | EVQLVESGGGLVQPGGSLRLSCAASGFTFSHYGMSWVRQTPDKRLE |
| 07) | LVATIDNNGGNTYYPDSVKGRFTISRDNAKNTLYLQMSSLKSEDTA |
| | MYYCARDAYYTYDYWYFDVWGAGTTVTVSS |
| 317 (clone | EVKLVESGGGLVQPGGSLKLSCAASGFTFSSYTMSWVRQTPEKRLE |
| 09) | WVAYISYGGGDTYYPDTVKGRFTISRDNAKNTLYLQMSSLKSEDTA |
| | MYYCARQKVDGYFVAMDYWSQGTSVTVSS |
| 318 (clone | EVKLVESGGGLVKPGGSLKLSCAASGFTFSSYGMSWVRQTPEKRLE |
| 51) | WVATISGGGRYTYYPDSVKGRFTISRDNAKNILYLQMSSLRSEDTAL |
| | YYCASPYDGYYGAMDYWGQGTSVTVSS |
| 319 (clone | EVQLQQSGTVLARPGASVRMSCKASGYSFSTYWMHWVKQRPGQG |
| 55) | LEWIGGIYPGNSDTNYNQKFKGKAKLTAVTSASTAYMDLSSLTNED |
| | SAVYYCTGGYFFDVWGAGTTVTVSS |
| 320 (clone | QIQLQQSGPEQVKPGASVKISCKASGYMFIDYYMNWVKQKPGQGL |
| 79 | EWIGWIYPGSGSTKDNENFKGKATLTVDTSSSTAYMQLSSLTSEDTA |
| | VYFCARYGPRFFDVWGAGTTVTVSS |
| 321 (clone | EVKLLESGGGLVQPGGSLKLSCEVSGFDFSRDWMSWVRQAPGKGL |
| 80) | EWIGQISPDSTSINYKPSLKDKFIISRDNAKNTLYLQLSGVRSEDTAL |
| | YHCARDTSGYPDYWGQGTSLTVSS |

TABLE 2

| SEQ ID No. | anti-PD-1 Light Chain See FIG. 18 for CDRs |
|---|---|
| 322 (clone 01) | NIVMTQATPSVPVTPGESVSISCRSSKSLLHSDGDTYLYWFLQRP |
| | GQSPQLLIYRMSNLVSGVPDRFSGSGSGTAFTLRISRVEAEDVGV |
| | YYCMQHLEYPYTFGGGTKLEIK |
| 323 (clone 02) | QIVLTQSPAIMSSSLGERVTMTCTASSSVSSSYLHWYQQKPGSSP |
| | KLWIYSTSNLASGVPARFSGSGSGTSYSLTISSMEAEDAATYYCH |
| | QYHRSPPIFTFGSGTKLEIK |
| 324 (clone 03) | QIVLTQSPAIMSASLGERVTMTCTVSSSISSSYLHWYQQKPGSSPK |
| | LWIYSTSNLASGVPARFSGSGSGTSYSLTISSMEAEDAATYYCHQ |
| | YHRSPLTFGAGTKLELK |
| 325 (clone 07) | DIQMTQTTSSLSASLGDRVTISCSASQGISNYLNWYQQKPDGTVK |
| | LLIYYTSSLHSGVPSRFSGSGSGTDYSLTISNLEPEDIATYYCQQYH |
| | KLPWTFGGGTKLEIK |
| 326 (clone 09) | VIVLTQSPASLAVSLGQRATISCRASERVDDYGISFMNWFQQKPG |
| | QPPKLLIYAASNQGSGVPARFSGSGSGTDFSLNIHPMEEDDTAMY |
| | FCQQNKEVPWTFGGGTKLEIK |
| 327 (clone 51) | DIVLTQSPASLAVSLGQRATISCRASESVDNSGISFMNWFQQKPG |
| | QPPKLLIYAASNQGSGVPARFSGSGSGTDFSLNIHPMEEDDTAMY |
| | FCQQSKEVPWTFGGGTKLEIK |
| 328 (clone 55) | DIKMTQSPSSLYASLGERVTITCKASQDINRYLTWFQQKPGKSPK |
| | TLIFRANRLVTGVPSRFIGSGSGQEYSLTISSLEYEDMGIYFCLQYD |
| | ESPYTFGGGTKLEIK |
| 329 (clone 79) | DIQMTQSPSSLSASLGERVNLTCRASQEISGYLSWLQQKPDGTIKR |
| | LIYVASTLDSGVPERFSGSRSGSDYSLTISSLESEDFADYYCLQYA |
| | SYPYTFGGGTKLEIK |
| 330 (clone 80) | DILLTQSPSSMSVSLGDTVSITCHASQDISRNIGWLRQKPGKSFKG |
| | LIYHGTNLEDGVPSRFSGSGSGADYSLTISSLESEDFADYYCVQY |
| | AQFPYTFGGGTKLEIK |

TABLE 4
Alternative HCDR Sequences for anti-PD1 portion

| Clone | Heavy Chain Sequence | HCDR1 | HCDR2 | HCDR3 |
|---|---|---|---|---|
| 001 | EVKLLESGGGLVQPGGSLKLSCAASGED | RYWMS | EINPDSSTIK | MGYRLFDS |
| | FSRYWMSWVRQAPGKGLEWIGEINPDS | (SEQ ID | YTPSLKD | (SEQ ID NO: |
| | STIKYTPSLKDKFIISRDNAKNTLCLQLSK | NO: 331) | (SEQ ID NO: | 333) |
| | VRSEDSALYYCARMGYRLFDSWGQGTT | | 332) | |
| | LTVSS (SEQ ID NO: 313) | | | |
| 002 | EVQLQQSGTVLARPGASVKMSCKASGN | SYWMH | AIYPGYSDT | FIATVGGYFD |
| | TFTSYWMHWVKQRPGQGLEWIGAIYPG | (SEQ ID | RYNQKFKG | V (SEQ ID |
| | YSDTRYNQKFKGKATLTAVTSTSTAYM | NO: 334) | (SEQ ID NO: | NO: 336) |
| | ELSSLTNEDSAVYYCTKFIATVGGYFDV | | 335) | |
| | WGAGTTVTVSS (SEQ ID NO: 314) | | | |
| 003 | QIQLVQSGPELKKPGETVKISCKASGYTF | DYSMH | WIKIETGNP | DYYGYYYY |
| | TDYSMHWVKQAPGKGLKWMGWIKIET | (SEQ ID | TYADDFKG | AMDY (SEQ |
| | GNPTYADDFKGRFAFSLETSASTAYLQI | NO: 337) | (SEQ ID NO: | ID NO: 339) |
| | NNLKNEDTATYFCARDYYGYYYYAMD | | 338) | |
| | YWGQGTSVTVSS (SEQ ID NO: 315) | | | |
| 004 | EVKLVESGGGLVQPGGSLKLSCAASGFT | SYTMS | YISYGGGDT | QKVDGYYV |
| | FSSYTMSWVRQTPEKRLEWVAYISYGG | (SEQ ID | YYSDTVKG | GMDY (SEQ |
| | GDTYYSDTVKGRFTISRDNAKNTLYLQ | NO: 340) | (SEQ ID NO: | ID NO: 342) |
| | MSSLKSEDTAMYYCARQKVDGYYVGM | | 341) | |
| | DYWGQGSSVTVSS (SEQ ID NO: 306) | | | |
| 007 | EVQLVESGGGLVQPGGSLRLSCAASGFT | HYGMS | TIDNNGGNT | DAYYTYDY |
| | FSHYGMSWVRQTPDKRLELVATIDNNG | (SEQ ID | YYPDSVKG | WYFDV (SEQ |
| | GNTYYPDSVKGRFTISRDNAKNTLYLQ | NO: 343) | (SEQ ID NO: | ID NO: 345) |
| | MSSLKSEDTAMYYCARDAYYTYDYWY | | 344) | |
| | FDVWGAGTTVTVSS (SEQ ID NO: 316) | | | |
| 009 | EVKLVESGGGLVQPGGSLKLSCAASGFT | SYTMS | YISYGGGDT | QKVDGYFVA |
| | FSSYTMSWVRQTPEKRLEWVAYISYGG | (SEQ ID | YYPDTVKG | MDY (SEQ ID |
| | GDTYYPDTVKGRFTISRDNAKNTLYLQ | NO: 340) | (SEQ ID NO: | NO: 347) |
| | MSSLKSEDTAMYYCARQKVDGYFVAM | | 346) | |
| | DYWSQGTSVTVSS (SEQ ID NO: 317) | | | |
| 051 | EVKLVESGGGLVKPGGSLKLSCAASGFT | GFTFSS | SGGGRY | CASPYDGYY |
| | FSSYGMSWVRQTPEKRLEWVATISGGG | Y (SEQ | (SEQ ID NO: | GAMDYW |
| | RYTYYPDSVKGRFTISRDNAKNILYLQM | ID NO: | 349) | (SEQ ID NO: |
| | SSLRSEDTALYYCASPYDGYYGAMDYW | 348) | | 350) |
| | GQGTSVTVSS (SEQ ID NO: 318) | | | |
| 055 | EVQLQQSGTVLARPGASVRMSCKASGY | TYWMH | GIYPGNSDT | GYFFDV |
| | SFSTYWMHWVKQRPGQGLEWIGGIYPG | (SEQ ID | NYNQKFKG | (SEQ ID NO: |
| | NSDTNYNQKFKGKAKLTAVTSASTAYM | NO: 351) | (SEQ ID NO: | 353) |
| | DLSSLTNEDSAVYYCTGGYFFDVWGAG | | 352) | |
| | TTVTVSS (SEQ ID NO: 319) | | | |
| 079 | QIQLQQSGPEQVKPGASVKISCKASGYM | DYYMN | WIYPGSGST | YGPRFFDV |
| | FIDYYMNWVKQKPGQGLEWIGWIYPGS | (SEQ ID | KDNENFKG | (SEQ ID NO: |
| | GSTKDNENFKGKATLTVDTSSSTAYMQ | NO: 354) | (SEQ ID NO: | 356) |
| | LSSLTSEDTAVYFCARYGPRFFDVWGAG | | 355) | |
| | TTVTVSS (SEQ ID NO: 320) | | | |
| 080 | EVKLLESGGGLVQPGGSLKLSCEVSGFD | RDWMS | QISPDSTSIN | DTSGYPDY |
| | FSRDWMSWVRQAPGKGLEWIGQISPDS | (SEQ ID | YKPSLKD | (SEQ ID NO: |
| | TSINYKPSLKDKFIISRDNAKNTLYLQLS | NO: 357) | (SEQ ID NO: | 359) |
| | GVRSEDTALYHCARDTSGYPDYWGQGT | | 358) | |
| | SLTVSS (SEQ ID NO: 321) | | | |

TABLE 5
Alternative HFR Sequences for anti-PD1 portion

| Clone | Heavy Chain Sequence | HFR1 | HFR2 | HFR3 | HFR4 |
|---|---|---|---|---|---|
| 001 | EVKLLESGGGLVQPGGSLK | EVKLLESG | WVRQAP | KFIISRDNA | WGQGTTLTV |
| | LSCAASGFDFSRYWMSWV | GGLVQPG | GKGLEWI | KNTLCLQL | SS (SEQ ID |
| | RQAPGKGLEWIGEINPDSST | GSLKLSCA | G (SEQ ID | SKVRSEDS | NO: 363) |
| | IKYTPSLKDKFIISRDNAKN | ASGFDFS | NO: 361) | ALYYCAR | |
| | TLCLQLSKVRSEDSALYYC | (SEQ ID | | (SEQ ID NO: | |
| | ARMGYRLFDSWGQGTTLT | NO: 360) | | 362) | |
| | VSS (SEQ ID NO: 313) | | | | |
| 002 | EVQLQQSGTVLARPGASVK | EVQLQQS | WVKQRP | KATLTAVT | WGAGTTVTV |
| | MSCKASGNTFTSYWMHWV | GTVLARP | GQGLEWI | STSTAYME | SS (SEQ ID |
| | KQRPGQGLEWIGAIYPGYS | GASVKMS | G (SEQ ID | LSSLTNEDS | NO: 367) |
| | DTRYNQKFKGKATLTAVTS | CKASGNT | NO: 365) | AVYYCTK | |
| | TSTAYMELSSLTNEDSAVY | FT (SEQ ID | | (SEQ ID NO: | |
| | YCTKFIATVGGYFDVWGA | NO: 364) | | 366) | |
| | GTTVTVSS (SEQ ID NO: | | | | |
| | 314) | | | | |
| 003 | QIQLVQSGPELKKPGETVKI | QIQLVQSG | WVKQAP | RFAFSLETS | WGQGTSVTV |
| | SCKASGYTFTDYSMHWVK | PELKKPGE | GKGLKW | ASTAYLQI | SS (SEQ ID |
| | QAPGKGLKWMGWIKIETG | TVKISCKA | MG (SEQ | NNLKNEDT | NO: 371) |
| | NPTYADDFKGRFAFSLETS | SGYTFT | ID NO: | ATYFCAR | |
| | ASTAYLQINNLKNEDTATY | (SEQ ID | 369) | (SEQ ID NO: | |
| | FCARDYYGYYYYAMDYW | NO: 368) | | 370) | |
| | GQGTSVTVSS (SEQ ID NO: | | | | |
| | 315) | | | | |
| 004 | EVKLVESGGGLVQPGGSLK | EVKLVES | WVRQTP | RFTISRDNA | WGQGSSVTV |
| | LSCAASGFTFSSYTMSWVR | GGGLVQP | EKRLEW | KNTLYLQ | SS (SEQ ID |
| | QTPEKRLEWVAYISYGGGD | GGSLKLSC | VA (SEQ | MSSLKSED | NO: 375) |
| | TYYSDTVKGRFTISRDNAK | AASGFTFS | ID NO: | TAMYYCA | |
| | NTLYLQMSSLKSEDTAMYY | (SEQ ID | 373) | R (SEQ ID | |
| | CARQKVDGYYVGMDYWG | NO: 372) | | NO: 374) | |
| | QGSSVTVSS (SEQ ID NO: | | | | |
| | 306) | | | | |
| 007 | EQLVESGGGLVQPGGSLR | EVQLVES | WVRQTP | RFTISRDNA | WGAGTTVTV |
| | LSCAASGFTFSHYGMSWVR | GGGLVQP | DKRLELV | KNTLYLQ | SS (SEQ ID |
| | QTPDKRLELVATIDNNGGN | GGSLRLSC | A (SEQ ID | MSSLKSED | NO: 367) |
| | TYYPDSVKGRFTISRDNAK | AASGFTFS | NO: 377) | TAMYYCA | |
| | NTLYLQMSSLKSEDTAMYY | (SEQ ID | | R (SEQ ID | |
| | CARDAYYTYDYWYFDVW | NO: 376) | | NO: 374) | |
| | GAGTTVTVSS (SEQ ID NO: | | | | |
| | 316) | | | | |
| 009 | EVKLVESGGGLVQPGGSLK | EVKLVES | WVRQTP | RFTISRDNA | WSQGTSVTVS |
| | LSCAASGFTFSSYTMSWVR | GGGLVQP | EKRLEW | KNTLYLQ | S (SEQ ID NO: |
| | QTPEKRLEWVAYISYGGGD | GGSLKLSC | VA (SEQ | MSSLKSED | 378) |
| | TYYPDTVKGRFTISRDNAK | AASGFTFS | ID NO: | TAMYYCA | |
| | NTLYLQMSSLKSEDTAMYY | (SEQ ID | 373) | R (SEQ ID | |
| | CARQKVDGYFVAMDYWS | NO: 372) | | NO: 374) | |
| | QGTSVTVSS (SEQ ID NO: | | | | |
| | 317) | | | | |
| 051 | EVKLVESGGGLVKPGGSLK | EVKLVES | GMSWVR | TYYPDSVK | GQGTSVTVSS |
| | LSCAASGFTFSSYGMSWVR | GGGLVKP | QTPEKRL | GRFTISRDN | (SEQ ID NO: |
| | QTPEKRLEWVATISGGGRY | GGSLKLSC | EWVATI | AKNILYLQ | 382) |
| | TYYPDSVKGRFTISRDNAK | AAS (SEQ | (SEQ ID | MSSLRSED | |
| | NILYLQMSSLRSEDTALYY | ID NO: 379) | NO: 380) | TALYY | |
| | CASPYDGYYGAMDYWGQ | | | (SEQ ID NO: | |
| | GTSVTVSS (SEQ ID NO: | | | 381) | |
| | 318) | | | | |
| 055 | EVQLQQSGTVLARPGASVR | EVQLQQS | WVKQRP | KAKLTAVT | WGAGTTVTV |
| | MSCKASGYSFSTYWMHWV | GTVLARP | GQGLEWI | SASTAYMD | SS (SEQ ID |
| | KQRPGQGLEWIGGIYPGNS | GASVRMS | G (SEQ ID | LSSLTNEDS | NO: 367) |
| | DTNYNQKFKGKAKLTAVT | CKASGYS | NO: 365) | AVYYCTG | |
| | SASTAYMDLSSLTNEDSAV | FS (SEQ ID | | (SEQ ID NO: | |
| | YYCTGGYFFDVWGAGTTV | NO: 383) | | 384) | |
| | TVSS (SEQ ID NO: 319) | | | | |
| 079 | QIQLQQSGPEQVKPGASVKI | QIQLQQSG | WVKQKP | KATLTVDT | WGAGTTVTV |
| | SCKASGYMFIDYYMNWVK | PEQVKPG | GQGLEWI | SSSTAYMQ | SS (SEQ ID |
| | QKPGQGLEWIGWIYPGSGS | ASVKISCK | G (SEQ ID | LSSLTSEDT | NO: 367) |
| | TKDNENFKGKATLTVDTSS | ASGYMFI | NO: 386) | AVYFCAR | |
| | STAYMQLSSLTSEDTAVYF | (SEQ ID | | (SEQ ID NO: | |
| | CARYGPRFFDVWGAGTTV | NO: 385) | | 387) | |
| | TVSS (SEQ ID NO: 320) | | | | |
| 080 | EVKLLESGGGLVQPGGSLK | EVKLLESG | WVRQAP | KFIISRDNA | WGQGTSLTVS |
| | LSCEVSGFDFSRDWMSWV | GGLVQPG | GKGLEWI | KNTLYLQL | S (SEQ ID NO: |
| | RQAPGKGLEWIGQISPDSTS | GSLKLSCE | G (SEQ ID | SGVRSEDT | 390) |
| | INYKPSLKDKFIISRDNAKN | VSGFDFS | NO: 361) | ALYHCAR | |
| | TLYLQLSGVRSEDTALYHC | (SEQ ID | | (SEQ ID NO: | |
| | ARDTSGYPDYWGQGTSLT | NO: 388) | | 389) | |
| | VSS (SEQ ID NO: 321) | | | | |

TABLE 6
Alternative LCDR Sequences for anti-PD1 portion

| Clone | Light Chain Sequence | LCDR1 | LCDR2 | LCDR3 |
|---|---|---|---|---|
| 001 | NIVMTQATPSVPVTPGESVSISCRS | RSSKSL | RMSNLVS (SEQ | MQHLEYPYT (SEQ |
| | SKSLLHSDGDTYLYWFLQRPGQS | LHSDG | ID NO: 392) | ID NO: 393) |
| | PQLLIYRMSNLVSGVPDRFSGSGS | DTYLY | | |
| | GTAFTLRISRVEAEDVGVYYCMQ | (SEQ ID | | |
| | HLEYPYTFGGGTKLEIK (SEQ ID | NO: 391) | | |
| | NO: 322) | | | |
| 002 | QIVLTQSPAIMSSSLGERVTMTCT | TASSSV | STSNLAS (SEQ | HQYHRSPPIFT |
| | ASSSVSSSYLHWYQQKPGSSPKL | SSSYLH | ID NO: 395) | (SEQ ID NO: 396) |
| | WIYSTSNLASGVPARFSGSGSGTS | (SEQ ID | | |
| | YSLTISSMEAEDAATYYCHQYHR | NO: 394) | | |
| | SPPIFTFGSGTKLEIK (SEQ ID | | | |
| | NO: 323) | | | |
| 003 | QIVLTQSPAIMSASLGERVTMTCT | TVSSSI | STSNLAS (SEQ | HQYHRSPLT (SEQ |
| | VSSSISSSYLHWYQQKPGSSPKLW | SSSYLH | ID NO: 395) | ID NO: 398) |
| | IYSTSNLASGVPARFSGSGSGTSYS | (SEQ ID | | |
| | LTISSMEAEDAATYYCHQYHRSP | NO: 397) | | |
| | LTFGAGTKLELK (SEQ ID NO: | | | |
| | 324) | | | |
| 004 | NIVMTQSPKSMSMSVGERVTLSC | KASEN | GASNRYT (SEQ | GQSYSSPLT (SEQ |
| | KASENVGTNVSWYQQKPEQSPRL | VGTNV | ID NO: 400) | ID NO: 401) |
| | LIYGASNRYTGVPDRFTGSGSVTD | S (SEQ | | |
| | FTLTISSVQAEDLVDYHCGQSYSS | ID NO: | | |
| | PLTFGSGTKLELK (SEQ ID NO: | 399) | | |
| | 307) | | | |
| 007 | DIQMTQTTSSLSASLGDRVTISCS | SASQGI | YTSSLHS (SEQ | QQYHKLPWT (SEQ |
| | ASQGISNYLNWYQQKPDGTVKLL | SNYLN | ID NO: 403) | ID NO: 404) |
| | IYYTSSLHSGVPSRFSGSGSGTDYS | (SEQ ID | | |
| | LTISNLEPEDIATYYCQQYHKLPW | NO: 402) | | |
| | TFGGGTKLEIK (SEQ ID NO: | | | |
| | 325) | | | |
| 009 | VIVLTQSPASLAVSLGQRATISCR | RASER | AASNQGS (SEQ | QQNKEVPWT (SEQ |
| | ASERVDDYGISFMNWFQQKPGQP | VDDYG | ID NO: 406) | ID NO: 407) |
| | PKLLIYAASNQGSGVPARFSGSGS | ISFMN | | |
| | GTDFSLNIHPMEEDDTAMYFCQQ | (SEQ ID | | |
| | NKEVPWTFGGGTKLEIK (SEQ ID | NO: 405) | | |
| | NO: 326) | | | |
| 051 | DIVLTQSPASLAVSLGQRATISCR | RASESV | AASNQGS (SEQ | CQQSKEVPWTF |
| | ASESVDNSGISFMNWFQQKPGQP | DNSGIS | ID NO: 406) | (SEQ ID NO: 409) |
| | PKLLIYAASNQGSGVPARFSGSGS | FMN | | |
| | GTDFSLNIHPMEEDDTAMYFCQQ | (SEQ ID | | |
| | SKEVPWTFGGGTKLEIK (SEQ ID | NO: 408) | | |
| | NO: 327) | | | |
| 055 | DIKMTQSPSSLYASLGERVTITCK | KASQDI | RANRLVT (SEQ | LQYDESPYT (SEQ |
| | ASQDINRYLTWFQQKPGKSPKTLI | NRYLT | ID NO: 411) | ID NO: 412) |
| | FRANRLVTGVPSRFIGSGSGQEYS | (SEQ ID | | |
| | LTISSLEYEDMGIYFCLQYDESPY | NO: 410) | | |
| | TFGGGTKLEIK (SEQ ID NO: | | | |
| | 328) | | | |
| 079 | DIQMTQSPSSLSASLGERVNLTCR | RASQEI | VASTLDS (SEQ | LQYASYPYT (SEQ |
| | ASQEISGYLSWLQQKPDGTIKRLI | SGYLS | ID NO: 414) | ID NO: 415) |
| | YVASTLDSGVPERFSGSRSGSDYS | (SEQ ID | | |
| | LTISSLESEDFADYYCLQYASYPY | NO: 413) | | |
| | TFGGGTKLEIK (SEQ ID NO: | | | |
| | 329) | | | |
| 080 | DILLTQSPSSMSVSLGDTVSITCHA | HASQDI | HGTNLED (SEQ | VQYAQFPYT (SEQ |
| | SQDISRNIGWLRQKPGKSFKGLIY | SRNIG | ID NO: 417) | ID NO: 418) |
| | HGTNLEDGVPSRFSGSGSGADYS | (SEQ ID | | |
| | LTISSLESEDFADYYCVQYAQFPY | NO: 416) | | |
| | TFGGGTKLEIK (SEQ ID NO: | | | |
| | 330) | | | |

TABLE 7
Alternative LFR Sequences for the anti-PD1 portion

| Clone | Light Chain Sequence | LFR1 | LFR2 | LFR3 | LFR4 |
|---|---|---|---|---|---|
| 001 | NIVMTQATPSVPVTPGESVS | NIVMTQA | WFLQRP | GVPDR | FGGGTKLEIK |
| | ISCRSSKSLLHSDGDTYLYW | TPSVPVTP | GQSPQLL | FSGSGS | (SEQ ID NO: 422) |
| | FLQRPGQSPQLLIYRMSNLV | GESVSISC | IY (SEQ | GTAFTL | |
| | SGVPDRFSGSGSGTAFTLRI | (SEQ ID | ID NO: | RISRVE | |
| | SRVEAEDVGVYYCMQHLE | NO: 419) | 420) | AEDVG | |
| | YPYTFGGGTKLEIK (SEQ ID | | | VYYC | |
| | NO: 322) | | | (SEQ ID | |
| | | | | NO: 421) | |
| 002 | QIVLTQSPAIMSSSLGERVT | QIVLTQSP | WYQQKP | GVPAR | FGSGTKLEIK |
| | MTCTASSSVSSSYLHWYQQ | AIMSSSLG | GSSPKLW | FSGSGS | (SEQ ID NO: 426) |
| | KPGSSPKLWIYSTSNLASGV | ERVTMTC | IY (SEQ | GTSYSL | |
| | PARFSGSGSGTSYSLTISSM | (SEQ ID | ID NO: | TISSME | |
| | EAEDAATYYCHQYHRSPPI | NO: 423) | 424) | AEDAA | |
| | FTFGSGTKLEIK (SEQ ID | | | TYYC | |
| | NO: 323) | | | (SEQ ID | |
| | | | | NO: 425) | |
| 003 | QIVLTQSPAIMSASLGERVT | QIVLTQSP | WYQQKP | GVPAR | FGAGTKLELK |
| | MTCTVSSSISSSYLHWYQQ | AIMSASLG | GSSPKLW | FSGSGS | (SEQ ID NO: 428) |
| | KPGSSPKLWIYSTSNLASGV | ERVTMTC | IY (SEQ | GTSYSL | |
| | PARFSGSGSGTSYSLTISSM | (SEQ ID | ID NO: | TISSME | |
| | EAEDAATYYCHQYHRSPLT | NO: 427) | 424) | AEDAA | |
| | FGAGTKLELK (SEQ ID NO: | | | TYYC | |
| | 324) | | | (SEQ ID | |
| | | | | NO: 425) | |
| 004 | NIVMTQSPKSMSMSVGERV | NIVMTQSP | WYQQKP | GVPDR | FGSGTKLELK |
| | TLSCKASENVGTNVSWYQ | KSMSMSV | EQSPRLLI | FTGSGS | (SEQ ID NO: 432) |
| | QKPEQSPRLLIYGASNRYTG | GERVTLS | Y (SEQ ID | VTDFTL | |
| | VPDRFTGSGSVTDFTLTISS | C (SEQ ID | NO: 430) | TISSVQ | |
| | VQAEDLVDYHCGQSYSSPL | NO: 429) | | AEDLV | |
| | TFGSGTKLELK (SEQ ID NO: | | | DYHC | |
| | 307) | | | (SEQ ID | |
| | | | | NO: 431) | |
| 007 | DIQMTQTTSSLSASLGDRVT | DIQMTQT | WYQQKP | GVPSRF | FGGGTKLEIK |
| | ISCSASQGISNYLNWYQQKP | TSSLSASL | DGTVKL | SGSGSG | (SEQ ID NO: 422) |
| | DGTVKLLIYYTSSLHSGVPS | GDRVTISC | LIY (SEQ | TDYSLT | |
| | RFSGSGSGTDYSLTISNLEPE | (SEQ ID | ID NO: | ISNLEP | |
| | DIATYYCQQYHKLPWTFGG | NO: 433) | 434) | EDIATY | |
| | GTKLEIK (SEQ ID NO: 325) | | | YC | |
| | | | | (SEQ ID | |
| | | | | NO: 435) | |
| 009 | VIVLTQSPASLAVSLGQRAT | VIVLTQSP | WFQQKP | GVPAR | FGGGTKLEIK |
| | ISCRASERVDDYGISFMNW | ASLAVSL | GQPPKLL | FSGSGS | (SEQ ID NO: 422) |
| | FQQKPGQPPKLLIYAASNQ | GQRATISC | IY (SEQ | GTDFSL | |
| | GSGVPARFSGSGSGTDFSLN | (SEQ ID | ID NO: | NIHPME | |
| | IHPMEEDDTAMYFCQQNKE | NO: 436) | 437) | EDDTA | |
| | VPWTFGGGTKLEIK (SEQ | | | MYFC | |
| | ID NO: 326) | | | (SEQ ID | |
| | | | | NO: 438) | |
| 051 | DIVLTQSPASLAVSLGQRAT | DIVLTQSP | WFQQKP | GVPAR | GGGTKLEIK |
| | ISCRASESVDNSGISFMNWF | ASLAVSL | GQPPKLL | FSGSGS | (SEQ ID NO: 441) |
| | QQKPGQPPKLLIYAASNQG | GQRATISC | IY (SEQ | GTDFSL | |
| | SGVPARFSGSGSGTDFSLNI | (SEQ ID | ID NO: | NIHPME | |
| | HPMEEDDTAMYFCQQSKE | NO: 439) | 437) | EDDTA | |
| | VPWTFGGGTKLEIK (SEQ | | | MYF | |
| | ID NO: 327) | | | (SEQ ID | |
| | | | | NO: 440) | |
| 055 | DIKMTQSPSSLYASLGERVT | DIKMTQSP | WFQQKP | GVPSRF | FGGGTKLEIK |
| | ITCKASQDINRYLTWFQQK | SSLYASLG | GKSPKTL | IGSGSG | (SEQ ID NO: 422) |
| | PGKSPKTLIFRANRLVTGVP | ERVTITC | IF (SEQ ID | QEYSLT | |
| | SRFIGSGSGQEYSLTISSLEY | (SEQ ID | NO: 443) | ISSLEY | |
| | EDMGIYFCLQYDESPYTFG | NO: 442) | | EDMGI | |
| | GGTKLEIK (SEQ ID NO: 328) | | | YFC | |
| | | | | (SEQ ID | |
| | | | | NO: 444) | |
| 079 | DIQMTQSPSSLSASLGERVN | DIQMTQSP | WLQQKP | GVPERF | FGGGTKLEIK |
| | LTCRASQEISGYLSWLQQK | SSLSASLG | DGTIKRL | SGSRSG | (SEQ ID NO: 422) |
| | PDGTIKRLIYVASTLDSGVP | ERVNLTC | IY (SEQ | SDYSLT | |
| | ERFSGSRSGSDYSLTISSLES | (SEQ ID | ID NO: | ISSLESE | |
| | EDFADYYCLQYASYPYTFG | NO: 445) | 446) | DFADY | |
| | GGTKLEIK (SEQ ID NO: 329) | | | YC | |
| | | | | (SEQ ID | |
| | | | | NO: 447) | |
| 080 | DILLTQSPSSMSVSLGDTVSI | DILLTQSP | WLRQKP | GVPSRF | FGGGTKLEIK |
| | TCHASQDISRNIGWLRQKP | SSMSVSL | GKSFKGL | SGSGSG | (SEQ ID NO: 422) |
| | GKSFKGLIYHGTNLEDGVP | GDTVSITC | IY (SEQ | ADYSL | |
| | SRFSGSGSGADYSLTISSLES | (SEQ ID | ID NO: | TISSLES | |
| | EDFADYYCVQYAQFPYTFG | NO: 448) | 449) | EDFAD | |
| | GGTKLEIK (SEQ ID NO: 330) | | | YYC | |
| | | | | (SEQ ID | |
| | | | | NO: 450) | |

In some embodiments, nucleic acids encoding VH and VL sequences of anti-PD1 variable domains disclosed herein are provided in Table 5.

TABLE 5
Exemplary nucleic acid sequences encoding an anti-PD1 portion

| Clone | Heavy Chain | Light Chain |
|---|---|---|
| 001 | GAGGTGAAGCTTCTCGAGTCTGGAGGT | AATATTGTGATGACTCAGGCTACAC |
| | GGCCTGGTGCAGCCTGGAGGATCCCTG | CCTCTGTACCTGTCACTCCTGGAGAG |
| | AAACTCTCCTGTGCAGCCTCAGGATTC | TCAGTATCCATCTCCTGCAGGTCTAG |
| | GATTTTAGTAGATACTGGATGAGTTGG | TAAGAGTCTCCTGCATAGTGATGGC |
| | GTCCGGCAGGCTCCAGGGAAAGGGCT | GACACTTACTTGTATTGGTTCCTGCA |
| | AGAATGGATTGGAGAAATTAATCCAG | GAGGCCAGGCCAGTCTCCTCAGCTC |
| | ATAGCAGTACGATAAAATACACGCCAT | CTGATATATCGGATGTCCAACCTTGT |
| | CTCTAAAGGATAAATTCATCATCTCCA | CTCAGGAGTCCCAGACAGGTTCAGT |
| | GAGACAACGCCAAAAATACGCTGTGC | GGCAGTGGGTCAGGAACTGCTTTCA |
| | CTGCAATTGAGCAAAGTGAGATCTGAG | CACTGAGAATCAGTAGAGTGGAGGC |
| | GACTCAGCCCTTTATTACTGTGCAAGA | TGAGGATGTGGGTGTTTATTACTGTA |
| | ATGGGCTATAGGCTCTTTGACTCCTGG | TGCAACATCTAGAATATCCGTACAC |
| | GGCCAAGGCACCACTCTCACAGTCTCC | GTTCGGAGGGGGGACCAAGCTGGAA |
| | TCA (SEQ ID NO: 451) | ATAAAA (SEQ ID NO: 452) |
| 002 | GAGGTTCAGCTCCAGCAGTCTGGGACT | CAAATTGTTCTCACCCAGTCTCCAGC |
| | GTGCTGGCAAGGCCTGGGGCTTCAGTG | AATCATGTCTTCATCTCTAGGGGAAC |
| | AAGATGTCCTGCAAGGCTTCTGGCAAC | GGGTCACCATGACCTGCACTGCCAG |
| | ACCTTTACCAGCTACTGGATGCACTGG | CTCAAGTGTAAGTTCCAGTTACTTGC |
| | GTAAAACAGAGGCCTGGACAGGGTCT | ACTGGTACCAGCAGAAGCCAGGATC |
| | GGAATGGATTGGCGCTATTTATCCTGG | CTCCCCCAAACTCTGGATTTATAGCA |
| | ATATAGTGATACTAGGTACAATCAGAA | CATCCAACCTGGCTTCTGGAGTCCCA |
| | GTTTAAGGGCAAGGCCACACTGACTGC | GCTCGCTTCAGTGGCAGTGGGTCTG |
| | AGTCACATCCACCAGCACTGCCTACAT | GGACCTCTTACTCTCTCACAATCAGC |
| | GGAGCTCAGCAGCCTGACAAATGAGG | AGCATGGAGGCTGAAGATGCTGCCA |
| | ACTCTGCGGTCTATTACTGTACAAAAT | CTTATTACTGCCACCAGTATCATCGT |
| | TTATTGCTACGGTAGGGGGGTACTTCG | TCCCCACCCATATTCACGTTCGGCTC |
| | ATGTCTGGGGCGCAGGGACCACGGTC | GGGGACAAAGTTGGAAATAAAA |
| | ACCGTCTCCTCA (SEQ ID NO: 453) | (SEQ ID NO: 454) |
| 003 | CAGATCCAGTTGGTGCAGTCTGGACCT | CAAATTGTTCTCACCCAGTCTCCAGC |
| | GAGCTGAAGAAGCCTGGAGAGACAGT | AATCATGTCTGCATCTCTAGGGGAA |
| | CAAGATCTCCTGCAAGGCTTCTGGTTA | CGGGTCACCATGACCTGCACTGTTA |
| | TACCTTCACAGACTATTCAATGCACTG | GCTCAAGTATAAGTTCCAGTTACTTG |
| | GGTGAAGCAGGCTCCAGGAAAGGGTT | CACTGGTACCAGCAGAAGCCAGGAT |
| | TAAAGTGGATGGGCTGGATAAAAATT | CCTCCCCCAAACTCTGGATTTATAGC |
| | GAGACTGGTAACCCAACATATGCAGAT | ACATCCAACCTGGCTTCTGGAGTCCC |
| | GACTTCAAGGGACGGTTTGCCTTCTCT | AGCTCGCTTCAGTGGCAGTGGGTCT |
| | TTGGAAACCTCTGCCAGCACTGCCTAT | GGGACCTCTTACTCTCTCACAATCAG |
| | TTGCAGATCAACAACCTCAAAAATGAG | CAGCATGGAGGCTGAAGATGCTGCC |
| | GACACGGCTACATATTTCTGTGCGAGA | ACTTATTACTGCCACCAGTATCATCG |
| | GACTACTATGGTTATTATTACTATGCT | TTCCCCGCTCACGTTCGGTGCTGGGA |
| | ATGGACTACTGGGGTCAAGGAACCTCA | CCAAGCTGGAGCTGAAA (SEQ ID NO: |
| | GTCACCGTCTCCTCA (SEQ ID NO: 455) | 456) |
| 004 | GAAGTGAAGCTGGTGGAGTCTGGGGG | AACATTGTAATGACCCAATCTCCCA |
| | AGGTTTAGTGCAGCCTGGAGGGTCCCT | AATCCATGTCCATGTCAGTAGGAGA |
| | GAAACTCTCCTGTGCAGCCTCTGGATT | GAGGGTCACCTTGAGCTGCAAGGCC |
| | CACTTTCAGTAGCTATACCATGTCTTG | AGCGAGAATGTGGGTACTAATGTAT |
| | GGTTCGCCAGACTCCAGAGAAGAGGC | CCTGGTATCAACAGAAACCAGAGCA |
| | TGGAGTGGGTCGCATACATTAGTTATG | GTCTCCTAGACTGCTGATATATGGGG |
| | GTGGAGGTGACACCTACTATTCAGACA | CTTCCAACCGGTACACTGGGGTCCCC |
| | CTGTAAAGGGCCGATTCACCATCTCCA | GATCGCTTCACAGGCAGTGGATCTG |
| | GAGACAATGCCAAGAACACCCTGTAC | TAACAGATTTCACTCTGACCATCAGC |
| | CTACAAATGAGCAGTCTGAAGTCTGAG | AGTGTGCAGGCTGAGGACCTTGTAG |
| | GACACGGCCATGTATTACTGTGCAAGA | ATTATCACTGTGGACAGAGTTACAG |
| | CAGAAGGTCGATGGTTACTACGTTGGT | CTCTCCGCTCACGTTCGGTTCTGGGA |
| | ATGGACTACTGGGGTCAAGGATCCTCA | CCAAGCTGGAGCTGAAA (SEQ ID NO: |
| | GTCACCGTCTCCTCA (SEQ ID NO: 457) | 458) |
| 007 | GAGGTGCAGCTGGTGGAGTCTGGGGG | GATATCCAGATGACACAGACTACAT |
| | AGGCTTAGTGCAGCCTGGAGGGTCCCT | CCTCCCTGTCTGCCTCTCTGGGAGAC |
| | GAGACTCTCCTGTGCAGCCTCTGGATT | AGAGTCACCATCAGTTGCAGTGCGA |
| | CACTTTCAGTCACTATGGCATGTCTTG | GTCAGGGCATTAGTAATTATTTAAAC |
| | GGTTCGCCAGACTCCAGACAAGAGGCT | TGGTATCAGCAGAAACCAGATGGAA |
| | GGAGTTGGTCGCAACCATTGATAATAA | CTGTGAAACTCCTGATCTATTACACA |
| | TGGTGGTAACACCTATTATCCAGACAG | TCAAGTTTACATTCAGGAGTCCCATC |
| | TGTGAAGGGCCGATTCACCATCTCCAG | AAGGTTCAGTGGCAGTGGGTCTGGG |
| | AGACAATGCCAAGAACACCCTGTACCT | ACAGATTATTCTCTCACCATCAGCAA |
| | GCAAATGAGCAGTCTGAAGTCTGAGG | CCTAGAACCTGAAGATATTGCCACTT |
| | ACACAGCCATGTATTACTGTGCAAGAG | ACTATTGTCAACAGTATCATAAGCTT |
| | ACGCCTACTATACTTACGACTACTGGT | CCGTGGACGTTCGGTGGAGGCACCA |
| | ACTTCGATGTCTGGGGCGCAGGGACCA | AGCTGGAAATCAAA (SEQ ID NO: 460) |
| | CGGTCACCGTCTCCTCA (SEQ ID NO: | |
| | 459) | |
| 009 | GAAGTGAAGCTGGTGGAGTCTGGGGG | GTCATTGTGCTGACCCAATCTCCAGC |
| | AGGTTTAGTGCAGCCTGGAGGGTCCCT | TTCTTTGGCTGTGTCTCTAGGGCAGA |
| | GAAACTCTCCTGTGCAGCCTCTGGATT | GGGCCACCATCTCCTGCAGAGCCAG |
| | CACTTTCAGTAGCTATACCATGTCTTG | CGAGCGTGTTGATGATTATGGCATTA |
| | GGTTCGCCAGACTCCAGAGAAGAGGC | GTTTTATGAACTGGTTCCAACAGAA |
| | TGGAGTGGGTCGCATACATTAGTTATG | ACCAGGACAGCCACCCAAACTCCTC |
| | GTGGAGGTGACACGTACTATCCAGACA | ATCTATGCTGCATCCAACCAAGGAT |
| | CTGTAAAGGGCCGATTCACCATCTCCA | CCGGGGTCCCTGCCAGGTTTAGTGG |
| | GAGACAATGCCAAGAACACCCTGTAC | CAGTGGGTCTGGGACAGACTTCAGC |
| | CTGCAAATGAGCAGTCTGAAGTCTGAG | CTCAACATCCATCCTATGGAGGAGG |
| | GACACGGCCATGTATTACTGTGCCAGA | ATGATACTGCAATGTATTTCTGTCAG |
| | CAGAAGGTCGATGGTTACTTCGTTGCT | CAAAATAAGGAGGTTCCGTGGACGT |
| | ATGGACTATTGGAGTCAAGGAACCTCA | TCGGTGGAGGCACCAAGCTGGAAAT |
| | GTCACCGTCTCCTCA (SEQ ID NO: 461) | CAAA (SEQ ID NO: 462) |
| 051 | GAAGTGAAGCTGGTGGAGTCTGGGGG | GACATTGTGCTGACCCAATCTCCAGC |
| | AGGCTTAGTGAAGCCTGGAGGGTCCCT | TTCTTTGGCTGTGTCTCTAGGGCAGA |
| | GAAACTCTCCTGTGCAGCCTCTGGATT | GGGCCACCATCTCCTGCAGAGCCAG |
| | CACTTTCAGTAGCTATGGCATGTCTTG | CGAAAGTGTTGATAATTCTGGCATTA |
| | GGTTCGCCAGACTCCGGAGAAGAGGC | GTTTTATGAACTGGTTCCAACAGAA |
| | TGGAGTGGGTCGCAACCATCAGTGGTG | ACCAGGACAGCCACCCAAACTCCTC |
| | GTGGTAGATACACCTACTATCCAGACA | ATCTATGCTGCATCCAACCAAGGAT |
| | GTGTGAAGGGGCGATTCACCATCTCCA | CCGGGGTCCCTGCCAGGTTTAGTGG |
| | GAGACAATGCCAAGAACATCCTGTACC | CAGTGGGTCTGGGACAGACTTCAGC |
| | TGCAAATGAGTAGTCTGAGGTCTGAGG | CTCAACATCCATCCTATGGAGGAGG |
| | ACACGGCCTTGTATTACTGTGCAAGCC | ATGATACTGCAATGTATTTCTGTCAG |
| | CCTATGATGGTTACTACGGTGCTATGG | CAAAGTAAGGAGGTTCCGTGGACGT |
| | ACTACTGGGGTCAAGGAACCTCAGTCA | TCGGTGGAGGCACCAAGCTGGAAAT |
| | CCGTCTCCTCA (SEQ ID NO: 463) | CAAA (SEQ ID NO: 464) |
| 055 | GAGGTTCAGCTCCAGCAGTCTGGGACT | GACATCAAGATGACCCAGTCTCCAT |
| | GTGCTGGCAAGGCCTGGGGCTTCCGTG | CTTCCTTGTATGCATCTCTAGGAGAG |
| | AGGATGTCCTGCAAGGCTTCTGGCTAC | AGAGTCACTATCACTTGCAAGGCGA |
| | AGCTTTTCCACCTACTGGATGCACTGG | GTCAGGACATTAATCGATATTTAACC |
| | GTAAAACAGAGGCCTGGACAGGGTCT | TGGTTCCAGCAGAAACCAGGGAAAT |
| | AGAATGGATTGGTGGTATTTATCCTGG | CTCCTAAGACCCTGATCTTTCGTGCA |
| | AAATAGTGACACTAACTACAACCAGA | AACAGATTGGTAACTGGGGTCCCAT |
| | AGTTCAAGGGCAAGGCCAAACTGACT | CAAGGTTCATTGGCAGTGGATCTGG |
| | GCAGTCACTTCCGCCAGCACTGCCTAC | GCAAGAATATTCTCTCACCATCAGC |
| | ATGGACCTCAGCAGCCTGACAAATGA | AGCCTGGAGTATGAAGACATGGGAA |
| | GGACTCTGCGGTCTATTACTGTACAGG | TTTATTTTTGTCTACAGTATGATGAG |
| | CGGTTACTTCTTCGATGTCTGGGGCGC | TCTCCGTACACGTTCGGAGGGGGGA |
| | AGGGACCACGGTCACCGTCTCCTCA | CCAAGTTGGAAATAAAA (SEQ ID NO: |
| | (SEQ ID NO: 465) | 466) |
| 079 | CAGATCCAGCTGCAGCAGTCTGGACCT | GACATCCAGATGACCCAGTCTCCAT |
| | GAGCAGGTGAAGCCTGGGGCTTCAGT | CCTCCTTATCTGCCTCTCTGGGAGAA |
| | GAAGATATCCTGCAAGGCTTCTGGCTA | AGAGTCAATCTCACTTGTCGGGCAA |
| | CATGTTCATCGACTACTATATGAACTG | GTCAGGAAATTAGTGGTTACTTAAG |
| | GGTGAAGCAGAAGCCTGGACAGGGAC | CTGGCTTCAGCAGAAACCAGATGGA |
| | TTGAGTGGATTGGATGGATTTATCCTG | ACTATTAAACGCCTGATCTACGTCGC |
| | GAAGCGGTAGTACTAAGGACAATGAG | ATCCACTTTAGATTCTGGTGTCCCAG |
| | AATTTCAAGGGCAAGGCCACATTGACT | AAAGGTTCAGTGGCAGTAGGTCTGG |
| | GTAGACACATCCTCCAGCACAGCCTAC | GTCAGATTATTCTCTCACCATCAGCA |
| | ATGCAGCTCAGTAGCCTGACATCTGAG | GCCTTGAGTCTGAGGATTTTGCAGAC |
| | GACACTGCTGTCTATTTCTGTGCAAGA | TATTACTGTCTACAATATGCTAGTTA |
| | TATGGGCCCAGGTTCTTCGATGTCTGG | TCCGTACACGTTCGGAGGGGGGACC |
| | GGCGCAGGGACCACGGTCACCGTCTCC | AAGCTGGAAATAAAA (SEQ ID NO: |
| | TCA (SEQ ID NO: 467) | 468) |
| 080 | GAGGTGAAGCTTCTCGAGTCTGGAGGT | GACATCCTGTTGACCCAATCTCCATC |
| | GGCCTGGTGCAGCCTGGAGGATCCCTG | CTCCATGTCTGTTTCTCTGGGAGACA |
| | AAACTCTCCTGTGAAGTCTCAGGATTC | CAGTCAGCATAACGTGTCATGCAAG |
| | GATTTTAGCAGAGACTGGATGAGTTGG | TCAGGACATTAGCAGGAATATAGGG |
| | GTCCGGCAGGCTCCAGGGAAAGGGCT | TGGTTGCGGCAGAAACCAGGGAAAT |
| | AGAGTGGATTGGACAAATTTCTCCAGA | CATTTAAGGGCCTGATCTATCATGGA |
| | TAGCACTTCGATAAACTATAAGCCATC | ACCAATTTGGAAGATGGAGTTCCAT |
| | TCTAAAGGATAAATTCATCATCTCCAG | CAAGGTTCAGCGGCAGTGGATCTGG |
| | AGACAACGCCAAAAATACGCTGTACCT | AGCAGATTATTCTCTCACCATCAGCA |
| | GCAATTGAGCGGAGTGAGATCTGAGG | GCCTGGAATCTGAAGATTTTGCAGA |
| | ACACAGCCCTTTATCACTGTGCAAGAG | CTATTACTGTGTGCAGTATGCTCAGT |
| | ACACCTCGGGCTACCCCGACTACTGGG | TTCCGTACACGTTCGGAGGGGGGAC |
| | GCCAAGGCACCAGTCTCACAGTCTCCT | CAAACTGGAAATAAAA (SEQ ID NO: |
| | CA (SEQ ID NO: 469) | 470) |

In some embodiments, the anti-IL-17RA antibody or antigen binding fragment thereof, comprises: a first arm comprising a first variable heavy chain domain and a first variable light chain domain, wherein a portion of the first arm is capable of binding to a portion of an IL-17RA; and a second arm comprising a second variable heavy chain domain and a second variable light chain domain, wherein a portion of the second arm is capable of binding to a portion of the IL-17RA protein wherein the first and second arms each further comprise a fragment, crystallizable (Fc) domain. In some embodiments, the first and second arms each further comprise a CH1 domain, a hinge domain, and a CL domain. In some embodiments, the portion of IL-17RA bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-17RA binding site and wherein the second variable heavy chain domain and second variable light chain domain form a second IL-17RA binding site.

In some embodiments, the first and second IL-17RA binding sites are the same. In some embodiments, the first and third polypeptide chain each further encode a hinge domain, a CHI domain, and the Fc domain, and wherein the second and fourth polypeptide chain each further encode a CL domain.

In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 146, HCDR2 comprising SEQ ID NO: 147, and HCDR3 comprising SEQ ID NOs: 148 and wherein the first and second variable light chain domain each comprises LCDR1 comprising SEQ ID NO: 224, LCDR2 comprising SEQ ID NO: 225, and LCDR3 comprising SEQ ID NO: 226.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

In some embodiments, the first and second variable heavy chain domain each comprises an amino acid sequence of SEQ ID NO: 300 and wherein the first and second variable light chain domain each comprises an amino acid sequence of SEQ ID NO: 301.

In some embodiments, the first and third polypeptide chain each comprises an amino acid sequence comprising SEQ ID NO: 300 and the second and fourth polypeptide chain each comprises an amino acid sequence comprising SEQ ID NO: 301.

In some embodiments, the first and second polypeptide chains are linked by one or more covalent disulfide bonds and the third and fourth polypeptide chains are linked by one or more covalent disulfide bonds. In some embodiments, the first and third polypeptide chains arc linked by one or more covalent disulfide bonds.

In some embodiments, the anti-IL17RA antibody is a human or humanized antibody. In some embodiments, the anti-IL17RA monoclonal antibody is Brodalumab.

In certain aspects, the present disclosure provides a method for treating cancer in a subject in need thereof comprising administering to the subject a composition comprising a therapeutically effective amount of an anti-IL-17RA antibody or antigen binding fragment thereof. In some embodiments, the method further comprises administering to the subject a therapeutically effective amount of at least one immune checkpoint inhibitor (ICI). Thus, in certain embodiments, the present disclosure also provides a method for treating, reducing, or preventing iRAEs in a subject in need thereof comprising administering to the subject a composition comprising a therapeutically effective amount of an anti-IL-17RA antibody or antigen binding fragment thereof, wherein the iRAEs are caused by administration of at least one ICI to the subject. In certain aspects, the present disclosure provides a method for treating cancer in a subject in need thereof comprising administering to the subject a composition comprising a therapeutically effective amount of an anti-IL-17RA antibody or antigen binding fragment thereof, the method further comprising administering to the subject a therapeutically effective amount of at least one immune checkpoint inhibitor (ICI) comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312. In some embodiments, immune-related adverse events (iRAEs) are treated, reduced, or prevented in the subject. Thus, in certain embodiments, the present disclosure also provides a method for treating, reducing, or preventing iRAEs in a subject in need thereof comprising administering to the subject a composition comprising a therapeutically effective amount of an anti-IL-17RA antibody or antigen binding fragment thereof, wherein the iRAEs are caused by administration of at least one ICI comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312 to the subject. The anti-IL17-RA antibody or antigen binding fragment and ICIs are described herein. In some embodiments, the treatment results in the subject having a reduction in cumulative steroid dosing. In some embodiments, the cumulative steroid dosing is measured in prednisone equivalents. In some embodiments, the treatment results in the subject having a decrease in steroid dose required for iRAE management. In some embodiments, the steroid dose is measured as the ratio of steroid dose over 7 days before administration of the composition to steroid dose over 7 days after administration of the composition. In some embodiments, the steroid dose over 7 days after administration of the composition is measured at 24 weeks after first administration of the composition. In some embodiments, the treatment results in the subject being tapered off of steroid treatment for at least one week. In some embodiments, the treatment results in a decrease in iRAEs by one or more grades. In some embodiments, the treatment results in a complete resolution of iRAEs. In some embodiments, the decrease in iRAEs by one or more grades or complete resolution of iRAEs is at 24 weeks post first administration of the composition. In some embodiments, iRAEs are measured using CTCAE criteria. In some embodiments, tumor burden of the subject is measured by RESIST criteria comparing CT or MRI scan before administration of the composition to CT or MRI scan after administration of the composition. In some embodiments, the CT or MRI scan after administration of the composition is measured at 24 weeks after first administration of the composition. In some embodiments, the treatment results in the subject having an improved quality of life as measured by the EQ-SD-3L. In some embodiments, the treatment results in the subject having increased progression-free survival. In some embodiments, the treatment is administered to a cohort of subjects and results in a cohort of said subjects having increased progression-free survival. In some embodiments, the treatment results in the subject having increased overall survival. In some embodiments, the treatment is administered to a cohort of subjects and results in a cohort of said subjects having increased overall survival. In some embodiments, the subject does not have a history of suicidality and/or depression. In some embodiments, the subject does not have a chronic infection and/or history of recurrent infection. In some embodiments, the subject does not have an active TB infection. In some embodiments, the subject does not have Chron's disease. In some embodiments, the subject does not have one or more of the exclusion criteria provided in Example 7. In some embodiments, before administration of the composition the subject has grade 3 or greater iRAEs. In some embodiments, the composition is administered subcutaneously. In some embodiments, the composition comprises 200-300 mg of brodalumab. In some embodiments, the composition comprises 210 mg of brodalumab. In some embodiments, the subject is administered multiple doses of a composition comprising an anti-IL-17RA antibody or antigen binding fragment thereof for at least 24 weeks. In some embodiments, the subject is administered multiple doses of a composition comprising an anti-IL-17RA antibody or antigen binding fragment thereof on a dosing schedule of three weekly doses followed by biweekly doses for a total of 24 weeks. In some embodiments, each does comprises 200-300 mg of brodalumab. In some embodiments, each dose comprises 210 mg of brodalumab.

In certain aspects, the present disclosure provides a method for treating cancer in a subject in need thereof comprising administering to the subject a composition comprising a therapeutically effective amount of an anti-IL17RA antagonist. In some embodiments, the IL17RA antagonist is an IL-17RA small interfering ribonucleic acid (siIL-17RA). In some embodiments, the siIL-17RA comprises the nucleic acid sequence of any of the siRNA sequences disclosed in Table 8. In some embodiments, the IL-17RA antagonist is an IL-17RA short-hairpin ribonucleic acid (shIL-17RA).

In some embodiments, the shIL-17RA comprises the nucleic acid sequence of any of the siIL-17RA sequences disclosed in Table 8. In some embodiments, the composition comprises a viral vector comprising a nucleic acid sequence encoding a shIL-17RA. In some embodiments, the viral vector is an adeno-associated vector (AAV).

TABLE 8

| SEQ. ID No. | gctgtgacggcgacgtccccgacct |
|---|---|
| 54 | aggcccaggggggctgcagtagacc |
| 55 | ccctctacgtggacgtggtcctgaa |
| 56 | accacctgcccaagcccatccctga |
| 57 | gctgtcgccaccaagtgcagatcca |
| 58 | gaccagaagagttccaccagcgatc |
| 59 | aaactgaggcatcaccacaggcggt |
| 60 | agcacgccaggatgaaggtaaccac |
| 61 | caaaggacctgcagatccagctgca |
| 62 | gagaaccacagttgctttgagcaca |

In various embodiments, the present application discloses a composition comprising IL-17RA siRNA. In various embodiments, the siRNA is at least 75%, 80%, 85%, 90%, 95%, 96%, 97%, 98% or 99% identical to the siRNA designs listed in Table 8. In some embodiments, the siRNA consists of a siRNA nucleic acid sequence of Table 8. In various embodiments, the present application discloses a composition comprising IL-17RA shRNA. For example, in various embodiments the composition is a vector encoding a shRNA wherein the shRNA comprises a nucleic acid sequence encoding the nucleic acid sequences provided in Table 8. In various embodiments, the shRNA comprises a nucleic acid sequence at least 75%, 80%, 85%, 90%, 95%, 96%, 97%, 98% or 99% identical to the nucleic acid sequence as provided in Table 8. In some embodiments, the shRNA consists of a nucleic acid sequence of Table 8. In various embodiments, the vector is a viral vector comprising a nucleic acid encoding a IL-17RA short-hairpin RNA (shRNA). In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10 or variants thereof. In various embodiments, the AAV is AAV8 or a variant thereof. In some embodiments, the AAV, including the AAV8, is a hepatocyte-targeted AAV. In some embodiments, the composition comprises hepatocyte-targeted AAV8 comprising a nucleic acid encoding IL-ILRA short-hairpin RNA (shRNA).

In some embodiments, the subject has a solid tumor. In some embodiments, the tumor is head-neck squamous cell carcinoma, sarcoma, liver hepatocellular carcinoma, gastric cancer, colorectal cancer, and breast cancer. In some embodiments, the cancer is breast cancer. In some embodiments, the cancer is melanoma. In some embodiments, the cancer is colon cancer.

In some embodiments, immune-related adverse events are treated, reduced, or prevented in the subject. In some embodiments, the immune-related adverse events comprise colitis, diarrhea, rash, pruritis, esophagitis, duodenitis, ileitis, neuritis, arthrhtis, vasculitis, nephritis, adrenal insufficiency, hepatitis, thrombocytopenia, anemia, pneumonitis, thyroiditis, hypophysitis, encephalitis, meningitis, uveitis, mucositis, rash, myocarditis, pericarditis, pancreatitis, colitis, enteritis, or any combination thereof.

In some embodiments, off-target immune infiltration of one or more untargeted organs in the subject is reduced or prevented. In some embodiments, CD3+ T cells are not detected or are not present at elevated levels in one or more untargeted organs in the subject.

In some embodiments, a tumor of the subject is reduced in volume. In some embodiments, growth of a tumor or cancer cells of the subject is inhibited.

In some embodiments, the combination of the IL-17RA antagonist, anti-IL17RA antibody or antigen-binding fragment thereof and the at least one ICI exhibits a synergistic effect on reducing a tumor volume, cancer treatment, or inhibiting tumor growth compared to the tumor volume reduction, cancer treatment effect, or tumor growth inhibition exhibited by administering a therapeutic dose of the one or more ICI alone or a therapeutic dose of the anti-IL-17RA antibody or antigen binding fragment thereof alone.

In some embodiments, the combination of the IL-17RA antagonist, anti-IL17RA antibody or antigen-binding fragment thereof and the at least one ICI comprising an anti-PDI antibody, or antigen-binding fragment thereof, comprising the CDRs or variable domains of clones 01, 02, 03, 04, 07, 09, 51, humanized 51, 55, 79, 80, or SEQ ID NOS: 311-312 exhibits a synergistic effect on reducing a tumor volume, cancer treatment, or inhibiting tumor growth compared to the tumor volume reduction, cancer treatment effect, or tumor growth inhibition exhibited by administering a therapeutic dose of the one or more ICI alone or a therapeutic dose of the anti-IL-17RA antibody or antigen binding fragment thereof alone.

In some embodiments, the IL-17RA antagonist, the anti-IL17RA antibody or antigen binding fragment thereof and the one or more ICI comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312 is administered concurrently as a single composition or as separate compositions.

In some embodiments, the IL-17RA antagonist, the anti-IL17RA antibody or antigen binding fragment thereof and the one or more ICI is administered concurrently as a single composition or as separate compositions.

In some embodiments, the IL-17RA antagonist, the anti-IL17RA antibody or antigen binding fragment thereof and the one or more ICI is administered sequentially.

In some embodiments, the IL-17RA antagonist, the anti-IL17RA antibody or antigen binding fragment thereof and the one or more ICI comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312 is administered sequentially.

In certain aspects, the subject matter disclosed herein relates to a preventive medical treatment started after following diagnosis of a disease (e.g., cancer) in order to prevent the disease from worsening or curing the disease. In one embodiment, the subject matter disclosed herein relates to prophylaxis of subjects who are believed to be at risk for moderate or severe disease associated with cancer or have previously been diagnosed with another disease, such as cancer. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method know in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (c) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding antibodies or mRNA-encoding antibodies and delivered by electroporation to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7 (2): 27.)

Inhibition of RNA encoding IL-17RA can effectively modulate the expression of these proteins. Inhibitors can include shRNAs encoding siRNAs, siRNA; interfering RNA or RNAi; dsRNA; RNA Polymerase III transcribed DNAs; ribozymes; Oligonucleotide (ASO) and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding an EGFR fusion molecule can be synthesized, e.g., by conventional phosphodiester techniques. Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector. Methods for producing and testing dsRNA or siRNA molecules are known in the art. A short hairpin RNA (shRNA) encodes an RNA molecule with a tight hairpin turn that can be used to silence target gene expression via RNA interference (RNAi). Expression of shRNA in cells is typically accomplished by delivery of plasmids or through viral or bacterial vectors.

RNA polymerase III transcribed DNAs contain promoters, such as the U6 promoter. These DNAs can be transcribed to produce small hairpin RNAs in the cell that can function as siRNA or linear RNAs, which can function as antisense RNA. The IL-17RA inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded.

As described herein, the methods of treatment refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. Methods described herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

A therapeutically effective amount of an agent or composition disclosed herein, for example, is one that is effective for preventing, ameliorating, treating or delaying the onset of a disease or condition.

Pharmaceutical compositions can be administered to any animal that can experience the beneficial effects of the agents of the invention. Such animals include humans and non-humans such as primates, pets and farm animals.

The present invention also comprises pharmaceutical compositions comprising the therapeutic agents described herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the pharmaceutical compositions in a local rather than systemic manner, for example, via injection of directly into the desired target site, often in a depot or sustained release formulation. Furthermore, one may administer the composition in a targeted drug delivery system.

One of ordinary skill in the art will appreciate that a method of administering pharmaceutically effective amounts of pharmaceutical compositions to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

The practice of aspects of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3rd Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986) and subsequent versions thereof. All patents, patent applications and references cited herein are incorporated by reference in their entireties.

One skilled in the art can obtain a protein in several ways, which include, but are not limited to, isolating the protein via biochemical means or expressing a nucleotide sequence encoding the protein of interest by genetic engineering methods.

### Compositions and Combinations

In certain aspects, the present disclosure provides a composition comprising therapeutically effect amounts of anti-IL-17RA antibody or antigen binding fragment thereof and the one or more ICIs.

In certain aspects, the present disclosure provides a composition comprising therapeutically effect amounts of anti-IL-17RA antibody or antigen binding fragment thereof and the one or more ICIs comprising an anti-PD1 antibody, or antigen-binding fragment thereof, comprising the CDRs or variable domains of clones 01, 02, 03, 04, 07, 09, 51, humanized 51, 55, 79, 80 or SEQ ID NOS: 311-312.

In certain aspects, the present disclosure provides a composition comprising therapeutically effect amounts of an anti-IL-17RA antibody or antigen binding fragment thereof and one or more ICIs.

In certain aspects, the present disclosure provides a composition comprising therapeutically effect amounts of an anti-IL-17RA antibody or antigen binding fragment thereof and one or more ICIs comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312.

In certain aspects, the present disclosure provides a combination in the form of a kit comprising two or more compositions, the first composition comprising a therapeutically effect amount of an anti-IL-17RA antibody or antigen binding fragment thereof and the second composition comprising a therapeutically effect amount of one or more ICIs.

In certain aspects, the present disclosure provides a combination in the form of a kit comprising two or more compositions, the first composition comprising a therapeutically effect amount of an anti-IL-17RA antibody or antigen binding fragment thereof and the second composition comprising a therapeutically effect amount of one or more ICIs comprising an anti-PD1 antibody, or antigen-binding fragment thereof, comprising the CDRs or variable domains of clones 01, 02, 03, 04, 07, 09, 51, humanized 51, 55, 79, 80, or SEQ ID NOS: 311-312.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

In some embodiments, the compositions further comprise one or more pharmaceutically acceptable excipients.

In some embodiments, the compositions further comprise a package insert or label providing directions for administering the compositions simultaneously, separately or sequentially.

In some embodiments, the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

In some embodiments, the anti-PD-1 antibody or antigen binding fragment thereof comprises Nivolumab, Pembrolizumab, Cemiplimab, Retifanlimab, Dostarlimab, Zimberclimab, Tisclelizumab, Camrelizumab, Sintilimab, Penpulimab or antigen binding fragment thereof. In some embodiments, the anti-PDL-1 antibody or antigen binding fragment thereof comprises Atezolimumab, Durvalumab and Avelumab, or antigen binding fragment thereof. In some embodiments, the anti-CTLA-4 antibody or antigen binding fragment thereof comprises ipilimumab, tremelimumab, or a combination thereof.

In some embodiments, the anti-LAG-3 antibody or antigen binding fragment thereof comprises BMS-986016, Relatimab, INCAGN02385, GSK2831781, or a combination thereof. In some embodiments, the anti-IL-17RA antibody or antigen binding fragment thereof is a monoclonal antibody or antigen binding fragment thereof.

In some embodiments, the anti-IL-17RA antibody or antigen binding fragment thereof, comprises: a first arm comprising a first variable heavy chain domain and a first variable light chain domain, wherein a portion of the first arm is capable of binding to a portion of an IL-17RA; and a second arm comprising a second variable heavy chain domain and a second variable light chain domain, wherein a portion of the second arm is capable of binding to a portion of the IL-17RA protein and the first and second arms each further comprise a fragment, crystallizable (Fc) domain.

In some embodiments, the first and second arms each further comprise a CHI domain, a hinge domain, and a CL domain. In some embodiments, the portion of IL-17RA bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-17RA binding site and the second variable heavy chain domain and second variable light chain domain form a second IL-17RA binding site.

In some embodiments, the first and second IL-17RA binding sites are the same. In some embodiments, the first and third polypeptide chain each further encode a hinge domain, a CHI domain, and the Fc domain, and wherein the second and fourth polypeptide chain each further encode a CL domain. In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 146, HCDR2 comprising SEQ ID NO: 147, and HCDR3 comprising SEQ ID NOs: 148 and wherein the first and second variable light chain domain each comprises LCDR1 comprising SEQ ID NO: 224, LCDR2 comprising SEQ ID NO: 225, and LCDR3 comprising SEQ ID NO: 226.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

In some embodiments, the first and second variable heavy chain domain each comprises an amino acid sequence of SEQ ID NO: 300 and wherein the first and second variable light chain domain each comprises an amino acid sequence of SEQ ID NO: 301.

In some embodiments, the first and third polypeptide chain each comprises an amino acid sequence comprising SEQ ID NO: 300 and the second and fourth polypeptide chain each comprises an amino acid sequence comprising SEQ ID NO: 301.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds. In some embodiments, the anti-IL17RA antibody is a human or humanized antibody. In some embodiments, the anti-IL17RA monoclonal antibody is Brodalumab.

In some embodiments, the IL17RA antagonist comprises several genome editing techniques such as RNAi (RNA interference), zinc finger nucleases (ZFNs), a TALE-effector domain nuclease (TALLEN), prime editing and base editing, CRISPR/Cas9 systems which are known in the art. In some embodiment, the CRISPR/Cas9 systems comprise a guide RNA (gRNA) or a single-molecule guide RNA (sgRNA). In some embodiment, the gRNA or sgRNA comprises a spacer sequence that is complementary to a portion of a nucleic acid sequence encoding IL17RA. In some embodiments, the IL17RA antagonist is an antisense RNA that specifically targets IL17RA, or a small molecule IL17RA antagonist.

In certain aspects, the present disclosure provides a method for treating cancer in a subject in need thereof comprising administering to the subject a composition comprising a therapeutically effective amount of an anti-IL17RA antagonist. In some embodiments, the IL17RA antagonist is an IL17RA small interfering ribonucleic acid (siIL 17RA). In some embodiments, the siIL 17RA comprises the nucleic acid sequence of any of the siRNA sequences of Table 1. In some embodiments, the IL17RA antagonist is an IL17RA short-hairpin ribonucleic acid (shIL17RA).

In some embodiments, the shIL17RA comprises the nucleic acid sequence of any of the siIL 17RA sequences Table 1. In some embodiments, the composition comprises a viral vector comprising a nucleic acid sequence encoding a shIL17RA. In some embodiments, the viral vector is an adeno-associated vector (AAV).

In various embodiments, the present application discloses a composition comprising IL17RA siRNA. In various embodiments, the siRNA is at least 75%, 80%, 85%, 90%, 95%, 96%, 97%, 98% or 99% identical to any of the sequences of the siIL-17RA in Table 1. In some embodiments, the siRNA consists of a siRNA nucleic acid sequence of any of the siIL 17RA sequences Table 1. In various embodiments, the present application discloses a composition comprising IL17RA shRNA. For example, in various embodiments the composition is a vector encoding a shRNA wherein the shRNA comprises a nucleic acid sequence encoding the nucleic acid sequences provided in Table 1. In various embodiments, the shRNA comprises a nucleic acid sequence at least 75%, 80%, 85%, 90%, 95%, 96%, 97%, 98% or 99% identical to the nucleic acid sequence as provided in Table 1. In some embodiments, the shRNA consists of a nucleic acid sequence of any of the siIL 17RA sequences Table 1. In various embodiments, the vector is a viral vector comprising a nucleic acid encoding a IL17RA short-hairpin RNA (shRNA). In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10 or variants thereof. In various embodiments, the AAV is AAV8 or a variant thereof. In some embodiments, the AAV, including the AAV8, is a hepatocyte-targeted AAV. In some embodiments, the composition comprises hepatocyte-targeted AAV8 comprising a nucleic acid encoding IL17RA short-hairpin RNA (shRNA).

### Methods of Prognosis

In certain aspects, the present disclosure provides a method of determining a cancer prognosis in a subject in need thereof comprising determining IL17RA gene expression levels in a sample from the subject.

In some embodiments, the IL17RA gene expression levels are IL17RA gene expression level of T cells of the subject. In some embodiments, the T cells are CD4 T cells, CD8 T cells, or both CD4 T cells and CD8 T cells. In some embodiments, the subject is determined to have a poor prognosis if the subject has an increased level of IL17RA gene expression as compared to the level of IL17RA gene expression in a healthy subject or cohort of healthy subjects.

In some embodiments, survival probability of a subject with cancer can be predicted by IL17RA expression and IL17RA/CD3 gene expression ratio. In some embodiments, the subject's survival can be predicted by IL17RA/CD4 and IL17RA/CD8 gene expression ratio. In some embodiments, the subject's survival can be predicted by IL17RA/CD4 mRNA expression ratio in multiple malignancies.

In some embodiments, the sample is biopsy tissue or blood. In some embodiments, the IL-17RA gene expression is measured using RNA-seq, gene chip data, or q-PCR. In some embodiments, the subject is a human.

In one embodiment, a biological sample comprises, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid), or from media (from cultured cells or cell lines). In one embodiment, a biological sample comprises, liver cells. The methods of detecting or quantifying a molecule include, but are not limited to, amplification-based assays with (signal amplification) hybridization based assays and combination amplification-hybridization assays. For detecting and quantifying a molecule, an exemplary method is an immunoassay that utilizes an antibody or other binding agents that specifically bind to protein or epitope of such, for example, Western blot or ELISA assays. In some embodiments, the level of gene expression is determined using single-cell RNA, RT-qPCR, RNA-seq, or gene chip data.

### IL17RA and PD-1 Antibodies

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety. The term “immunoglobulin” (Ig) is used interchangeably with “antibody” herein.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CHI and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

The antibodies of the various embodiments disclosed herein can include one or more of synthetic antibodies, monoclonal antibodies, oligoclonal or polyclonal antibodies, multiclonal antibodies, recombinantly produced antibodies, monospecific antibodies, monovalent antibodies, human antibodies, humanized antibodies, chimeric antibodies, CDR-grafted antibodies, primatized antibodies, single-chain Fv-Fcs (scFv-Fc)), bivalent with four scFv (scFv-Fc-scFv), IgG-scFv, IgM, IgA, trispecific, IgG-dAb, CrossMab 2:1 or 2:2, DVD-IgG, IgG (L)-scFv2, DVD-IgG, IgG (H)-scFv, scFv-(H) IgG, IgG (L)-scFv, scFv-(L) IgG, IgG (L,H)-Fv, IgG (H)-V, V (H)-IgG, IgG (L)-V, V (L)-IgG, KIH IgG-scFab, 2scFv-IgG, IgG-2scFv, scFv4-Ig, Zybody, DVI-IgG (four-in-one), scFv-KIH, and any other immunologically-reactive/antigen-binding molecules. In some embodiments, the antibody is a monoclonal antibody.

In some embodiments, the monoclonal antibody comprises a first, second, third and fourth chain. In some embodiments, the first and third chains each comprise a VH domain and the second and fourth chains each comprise a VL domain. In some embodiments, the first and third chains each further comprises a CH1 domain, a hinge domain, and a Fc domain. In some embodiments, the second and fourth chains each further comprises a CL domain. The pairing of the VH and VL of the first and second chains together forms a single antigen-binding site specific for an epitope on IL-17RA or PD-1 and the pairing of the VH and VL of the third and fourth chains together forms a single antigen-binding site specific for the same epitope. In some embodiments, the first and second chains are linked by one or more covalent disulfide bonds and the third and fourth chains are linked by one or more covalent disulfide bonds. In some embodiments, the first and third chains are linked by one or more disulfide bonds.

However, the antibodies disclosed herein are not limited to full-length IgG like antibodies. Other immunologically reactive/antigen-binding molecules including but not limited to, single-chain Fv-Fcs (scFv-Fc, bivalent with four scFv (scFv-Fc-scFv), IgG-scFv, IgM, IgA, trispecific, IgG-dAb, CrossMab 2:1 or 2:2, DVD-IgG, IgG (L)-scFv2, DVD-IgG, IgG (H)-scFv, scFv-(H) IgG, IgG (L)-scFv, scFv-(L) IgG, IgG (L,H)-Fv, IgG (H)-V, V (H)-IgG, IgG (L)-V, V (L)-IgG, KIH IgG-scFab, 2scFv-IgG, IgG-2scFv, scFv4-Ig, Zybody, DVI-IgG (four-in-one), and scFv-KIH are also contemplated herein and a person of skill in the art can readily synthesize such molecules using the sequences and identified domains of the heavy and light chains of the anti-IL-17RA and anti-PD-1 antibodies disclosed here. For example, in some embodiments, the monoclonal antibody comprises a first and second chain that associate together. In some embodiments, the first chain and second chain each comprises an scFv with specificity for an epitope on IL-17RA or PD-1 and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc antibody comprising a first and second chain that associate together, each chain comprising a variable heavy chain (VH) domain, a linker, a variable light chain (VL) domain, and an Fc domain.

For example, in some embodiments, the monoclonal antibody comprises a first and second chain that associate together. In some embodiments, the first chain and second chain each comprise two scFvs with specificity for an epitope on IL-17RA or PD-1 and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc-scFv antibody comprising a first and second chain that associate together, each chain comprising a first variable heavy chain (VH) domain, a first linker, a first variable light chain (VL) domain, an Fc domain, a second variable heavy chain (VH) domain, a second linker, and a second variable light chain (VL) domain.

In some embodiments, the monoclonal antibodies disclosed herein contain various modifications, substitutions, additions, or deletions to the variable or binding regions of one or more arms of an anti-IL-17RA or anti-PD-1 antibody disclosed herein. In some embodiments, the monoclonal antibodies disclosed herein may contain substitutions or modifications of the constant region (i.e., the Fc domain). The antibodies disclosed herein may contain one or more additional amino acid residue substitutions, mutations and/or modifications, which result in a compound with preferred characteristics including, but not limited to: altered pharmacokinetics, increased serum half-life, increase binding affinity, reduced binding affinity, reduced immunogenicity, increased production, altered Fc ligand binding, enhanced or reduced ADCC or CDC activity, altered glycosylation and/or disulfide bonds and modified binding specificity. IL17RA can be antagonized using antibodies specific to IL17RA, or antigen binding fragments thereof. In some embodiments the antibody is monoclonal. Non-limiting examples include brodalumab a human monoclonal antibody against IL17RA (AstraZeneca).

In some embodiments, the anti-IL17RA antibody or antigen binding fragment thereof for use in the methods of treatment disclosed herein comprises an antigen binding site comprising at least one immunoglobulin heavy chain variable domain (VH) which comprises in sequence hypervariable regions HCDR1, HCDR2 and HCDR3 and at least one immunoglobulin light chain variable domain (VL) which comprises in sequence hypervariable regions LCDR1, LCDR2, and LCDR3.

In some embodiments, the anti-IL-17RA antibody or antigen binding fragment thereof for use in the methods of treatment disclosed herein comprises an antigen binding site comprising at least one immunoglobulin heavy chain variable domain (VH) which comprises hypervariable regions HCDR1, HCDR2 and HCDR3 and at least one immunoglobulin light chain variable domain (VL) which comprises hypervariable regions LCDR1, LCDR2, and LCDR3.

In some embodiments, the anti-IL-17RA antibody for use in the methods of treatment disclosed herein comprises Brodalumab (an FDA approved anti-IL-17RA antibody) or an antigen binding fragment thereof as disclosed in the International patent publication WO2008054603, hereby incorporated in its entirety by reference. Brodalumab is a human IgG2 monoclonal antibody. The heavy chain variable region of brodalumab comprises the amino acid sequence: QVQLVQSGAEVKKPGASVKVSCKASGYTFTRYGISWVRQAPGQGLEWMGWISTYSGN TNYAQKLQGRVTMTTDTSTSTAYMELRSLRSDDTAVYYCARRQLYFDYWGQGTLVTV SS (SEQ ID NO: 300). The light chain variable region of brodalumab comprises the amino acid sequence:

(SEQ ID NO: 301)
EIVMTQSPATLSVSPGERATLSCRASQSVSSNLAWFQQKPGQAPRPLIY
DASTRATGVPARFSGSGSGTDFTLTISSLQSEDFAVYYCQQYDNWPLTF
GGGTKVEIK.

The HCDR1, HCDR2, and HCDR3 of brodalumab comprises SEQ ID NOS: 146, 147, and 148, respectively. The LCDR1, LCDR2, and LCDR3 of brodalumab comprises SEQ ID NOS: 224, 225, and 226 respectively. Thus, in other embodiments, the anti-IL-17RA antibody or antigen-binding fragment thereof comprises a light chain CDRI (SEQ ID NO:224), CDR2 (SEQ ID NO: 225), CDR3 (SEQ ID NO:226) and a heavy chain CDRI (SEQ ID NO: 146), CDR2 (SEQ ID NO: 147), CDR3 (SEQ ID NO: 148). In some embodiments, the anti-IL-17RA antibody or antigen binding fragment thereof for use in the methods of treatment disclosed herein comprises an antigen binding site comprising at least one immunoglobulin heavy chain variable domain comprising SEQ ID NO: 300 and at least one immunoglobulin light chain variable domain comprising SEQ ID NO: 301. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence of SEQ ID NO: 300 and/or a light chain amino acid sequence of SEQ ID NO: 301. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence of SEQ ID NO: 300 and comprising heavy chain CDRI (SEQ ID NO: 146), CDR2 (SEQ ID NO: 147), CDR3 (SEQ ID NO: 148) and/or a light chain amino acid sequence of SEQ ID NO: 301 and comprising light chain CDRI (SEQ ID NO:224), CDR2 (SEQ ID NO:225), CDR3 (SEQ ID NO:226).

In some embodiments, the anti-IL-17RA antibody or antigen binding fragment thereof comprises an antigen binding site comprising at least one immunoglobulin heavy chain variable domain with hypervariable regions HCDR1, HCDR2, and HCDR3; or HCDR equivalents thereof. In some embodiments, the anti-IL-17RA antibody or antigen binding fragment thereof can also comprise at least one immunoglobulin light chain variable domain which comprises hypervariable regions LCDR1, LCDR2, and LCDR3; or LCDR equivalents thereof.

In some embodiments, the anti-IL-17RA antibody or antigen-binding fragment thereof comprises a light chain LCDRI (SEQ ID NO: 185), LCDR2 (SEQ ID NO: 186), LCDR3 (SEQ ID NO: 187) and a heavy chain HCDRI (SEQ ID NO: 107), HCDR2 (SEQ ID NO: 108), HCDR3 (SEQ ID NO: 109). In other embodiments, the anti-IL-17RA antibody or antigen-binding fragment thereof comprises a light chain CDRI (SEQ ID NO; 188), CDR2 (SEQ ID NO: 189), CDR3 (SEQ ID NO: 190) and a heavy chain CDRI (SEQ ID NO: 110), CDR2 (SEQ ID NO: 111), CDR3 (SEQ ID NO: 112). In other embodiments, the anti-IL-17RA antibody or antigen-binding fragment thereof comprises a light chain CDRI (SEQ ID NO: 191), CDR2 (SEQ ID NO: 192), CDR3 (SEQ ID NO: 193) and a heavy chain CDRI (SEQ ID NO: 113), CDR2 (SEQ ID NO: 114), CDR3 (SEQ ID NO: 115). In other embodiments, the anti-IL-17RA antibody or antigen-binding fragment thereof comprises a light chain CDRI (SEQ ID NO: 194), CDR2 (SEQ ID NO: 195), CDR3 (SEQ ID NO: 196) and a heavy chain CDRI (SEQ ID NO: 116), CDR2 (SEQ ID NO: 117), CDR3 (SEQ ID NO: 118). In other embodiments, the anti-IL-17RA antibody or antigen-binding fragment thereof comprises a light chain CDRI (SEQ ID NO: 197), CDR2 (SEQ ID NO: 198), CDR3 (SEQ ID NO: 199) and a heavy chain CDRI (SEQ ID NO: 119), CDR2 (SEQ ID NO: 120), CDR3 (SEQ ID NO: 121). In other embodiments, the anti-IL-17RA antibody or antigen-binding fragment thereof comprises a light chain CDRI (SEQ ID NO: 200), CDR2 (SEQ ID NO:201), CDR3 (SEQ ID NO:202) and a heavy chain CDRI (SEQ ID NO: 122), CDR2 (SEQ ID NO: 123), CDR3 (SEQ ID NO: 124). In other embodiments, the anti-IL-17RA antibody or antigen-binding fragment thereof comprises a light chain CDRI (SEQ ID NO: 203), CDR2 (SEQ ID NO:204), CDR3 (SEQ ID NO:205) and a heavy chain CDRI (SEQ ID NO: 125), CDR2 (SEQ ID NO: 126), CDR3 (SEQ ID NO: 127). In other embodiments, the anti-IL-17RA antibody or antigen-binding fragment thereof comprises a light chain CDRI (SEQ ID NO: 206), CDR2 (SEQ ID NO:207), CDR3 (SEQ ID NO:208) and a heavy chain CDRI (SEQ ID NO: 128), CDR2 (SEQ ID NO: 129), CDR3 (SEQ ID NO: 130). In other embodiments, the anti-IL-17RA antibody or antigen-binding fragment thereof comprises a light chain CDRI (SEQ ID NO: 209), CDR2 (SEQ ID NO:210), CDR3 (SEQ ID NO:211) and a heavy chain CDRI (SEQ ID NO: 131), CDR2 (SEQ ID NO: 132), CDR3 (SEQ ID NO: 133). In other embodiments, the anti-IL-17RA antibody or antigen-binding fragment thereof comprises a light chain CDRI (SEQ ID NO: 212), CDR2 (SEQ ID NO:213), CDR3 (SEQ ID NO:214) and a heavy chain CDRI (SEQ ID NO: 134), CDR2 (SEQ ID NO: 135), CDR3 (SEQ ID NO: 136). In other embodiments, the anti-IL-17RA antibody or antigen-binding fragment thereof comprises a light chain CDRI (SEQ ID NO: 215), CDR2 (SEQ ID NO:216), CDR3 (SEQ ID NO:217) and a heavy chain CDRI (SEQ ID NO: 137), CDR2 (SEQ ID NO:138), CDR3 (SEQ ID NO: 139). In other embodiments, the anti-IL-17RA antibody or antigen-binding fragment thereof comprises light chain CDRI (SEQ ID NO: 218), CDR2 (SEQ ID NO:219), CDR3 (SEQ ID NO:220) and a heavy chain CDRI (SEQ ID NO: 140), CDR2 (SEQ ID NO: 141), CDR3 (SEQ ID NO: 142). In other embodiments, the anti-IL-17RA antibody or antigen-binding fragment thereof comprises a light chain CDRI (SEQ ID NO: 221), CDR2 (SEQ ID NO:222), CDR3 (SEQ ID NO:223) and a heavy chain CDRI (SEQ ID NO: 143), CDR2 (SEQ ID NO: 144), CDR3 (SEQ ID NO: 145). In other embodiments, the anti-IL-17RA antibody or antigen-binding fragment thereof comprises a light chain CDRI (SEQ ID NO: 227), CDR2 (SEQ ID NO:228), CDR3 (SEQ ID NO:229) and a heavy chain CDRI (SEQ ID NO: 149), CDR2 (SEQ ID NO: 150), CDR3 (SEQ ID NO: 151). In other embodiments, the anti-IL-17RA antibody or antigen-binding fragment thereof comprises a light chain CDRI (SEQ ID NO: 230), CDR2 (SEQ ID NO:231), CDR3 (SEQ ID NO:232) and a heavy chain CDRI (SEQ ID NO: 152), CDR2 (SEQ ID NO: 153), CDR3 (SEQ ID NO: 154). In other embodiments, the anti-IL-17RA antibody or antigen-binding fragment thereof comprises a light chain CDRI (SEQ ID NO: 233), CDR2 (SEQ ID NO:234), CDR3 (SEQ ID NO:235) and a heavy chain CDRI (SEQ ID NO: 155), CDR2 (SEQ ID NO: 156), CDR3 (SEQ ID NO: 157). In other embodiments, the anti-IL-17RA antibody or antigen-binding fragment thereof comprises a light chain CDRI (SEQ ID NO: 236), CDR2 (SEQ ID NO:237), CDR3 (SEQ ID NO:238) and a heavy chain CDRI (SEQ ID NO: 158), CDR2 (SEQ ID NO: 159), CDR3 (SEQ ID NO: 160). In other embodiments, the anti-IL-17RA antibody or antigen-binding fragment thereof comprises a light chain CDRI (SEQ ID NO: 239), CDR2 (SEQ ID NO:240), CDR3 (SEQ ID NO:241) and a heavy chain CDRI (SEQ ID NO: 161), CDR2 (SEQ ID NO: 162), CDR3 (SEQ ID NO: 163). In other embodiments, the anti-IL-17RA antibody or antigen-binding fragment thereof comprises a light chain CDRI (SEQ ID NO: 242), CDR2 (SEQ ID NO:243), CDR3 (SEQ ID NO:244) and a heavy chain CDRI (SEQ ID NO: 164), CDR2 (SEQ ID NO: 165), CDR3 (SEQ ID NO: 166). In other embodiments, the anti-IL-17RA antibody or antigen-binding fragment thereof comprises a light chain CDRI (SEQ ID NO: 245), CDR2 (SEQ ID NO:246), CDR3 (SEQ ID NO:247) and a heavy chain CDRI (SEQ ID NO: 167), CDR2 (SEQ ID NO: 168), CDR3 (SEQ ID NO: 169). In other embodiments, the anti-IL-17RA antibody or antigen-binding fragment thereof comprises a light chain CDRI (SEQ ID NO: 248), CDR2 (SEQ ID NO:249), CDR3 (SEQ ID NO:250) and a heavy chain CDRI (SEQ ID NO: 170), CDR2 (SEQ ID NO: 171), CDR3 (SEQ ID NO: 172). In other embodiments, the anti-IL-17RA antibody or antigen-binding fragment thereof comprises a light chain CDRI (SEQ ID NO: 251), CDR2 (SEQ ID NO:252), CDR3 (SEQ ID NO.253) and a heavy chain CDRI (SEQ ID NO: 173), CDR2 (SEQ ID NO: 174), CDR3 (SEQ ID NO: 175). In other embodiments, the anti-IL-17RA antibody or antigen-binding fragment thereof comprises a light chain CDRI (SEQ ID NO: 254), CDR2 (SEQ ID NO:255), CDR3 (SEQ ID NO:256) and a heavy chain CDRI (SEQ ID NO: 173), CDR2 (SEQ ID NO: 174), CDR3 (SEQ ID NO: 175). In other embodiments, the anti-IL-17RA antibody or antigen-binding fragment thereof comprises a light chain CDRI (SEQ ID NO: 257), CDR2 (SEQ ID NO:258), CDR3 (SEQ ID NO:259) and a heavy chain CDRI (SEQ ID NO: 176), CDR2 (SEQ ID NO: 177), CDR3 (SEQ ID NO: 178). In other embodiments, the anti-IL-17RA antibody or antigen-binding fragment thereof comprises a light chain CDRI (SEQ ID NO: 260), CDR2 (SEQ ID NO:261), CDR3 (SEQ ID NO:262) and a heavy chain CDRI (SEQ ID NO: 179), CDR2 (SEQ ID NO: 180), CDR3 (SEQ ID NO: 181). In other embodiments, the anti-IL-17RA antibody or antigen-binding fragment thereof comprises a light chain CDRI (SEQ ID NO: 263), CDR2 (SEQ ID NO:264), CDR3 (SEQ ID NO:265) and a heavy chain CDRI (SEQ ID NO: 182), CDR2 (SEQ ID NO: 183), CDR3 (SEQ ID NO: 184). In the various embodiments, the anti-IL-17RA antibody or antigen-binding fragment thereof comprises wherein said antibody specifically binds IL-17 receptor A.

TABLE 9
anti-IL17-RA antibody CDRs

| SEQ ID NO | Amino Acid Sequence |
|---|---|
| 107 | NYYWN |
| 108 | DIYYSGSTNYNPSLKS |
| 109 | DGELANYYGSGSYQFYYYYGMDV |
| 110 | GYYWS |
| 111 | EINHSGRTNYNPSLKS |
| 112 | GPYYFDSSGYLYYYYGLDV |
| 113 | SYGMH |
| 114 | VIWYDGSNKHYADSVKG |
| 115 | DTGVY |
| 116 | SYGMH |
| 117 | VIWYDGSNKHYADSVKG |
| 118 | DTGVY |
| 119 | SYYWS |
| 120 | RIYRSGNTIYNPSLKS |
| 121 | ENYSESSGLYYYYGMDV |
| 122 | RYGIS |
| 123 | WISAYNGNTNYAQKLQG |
| 124 | RDYDILTGYYNGFDP |
| 125 | RYGIS |
| 126 | WISAYNGNTNYAQKLQG |
| 127 | RDYDILTGYYNGFDP |
| 128 | GYGIS |
| 129 | WISAYNGNTNYAQNLQG |
| 130 | RDYDILTGYYNGFDP |
| 131 | RYGIS |
| 132 | WISAYNGNTNYAQKLQG |
| 133 | RDYDILTGYYNGFDP |
| 134 | SGGYYWS |
| 135 | YIYFSGSAYYNPSLKS |
| 136 | EYYDSSGYPDAFDI |
| 137 | SYGMH |
| 138 | VIWYDGSNKYYADSVKG |
| 139 | DTKDY |
| 140 | SYGIS |
| 141 | WISTYKGNTNYAQKLQG |
| 142 | KQLVFDY |
| 143 | SYGMQ |
| 144 | VIWYDGNKKYYADSVKG |
| 145 | GRVRDYYYGMDV |
| 146 | RYGIS |
| 147 | WISTYSGNTNYAQKLQG |
| 148 | RQLYFDY |
| 149 | SYGMQ |
| 150 | VIWYDGNKKYYADSVKG |
| 151 | GRVRDYYYGMDV |
| 152 | SYGIS |
| 153 | WISAYNGNTKYAQKLQG |
| 154 | KQLVFDY |
| 155 | SYGIS |
| 156 | WISAYSGNTKYAQKLQG |
| 157 | KQLVFDY |
| 158 | DYYMH |
| 159 | WMHPNSGGTDLAQRFQG |
| 160 | GGYCSTLSCSFYWYFDL |
| 161 | SYGIS |
| 162 | WISAYSGNTKYAQKFQG |
| 163 | RQLALDY |
| 164 | SYSMN |
| 165 | FISARSSTIYYADSVKG |
| 166 | PKVGGGMDV |
| 167 | SYSMN |
| 168 | IISSRSSIIHYADSVKG |
| 169 | PKVGGGMDV |
| 170 | RYGIS |
| 171 | WISAYSGNTNYAQKLQG |
| 172 | RQLYFDY |
| 173 | SYYWS |
| 174 | RIYPSGRTNYNPSLKS |
| 175 | EAYELQLGLYYYYGMDV |
| 176 | SYYWS |
| 177 | RIYPSGRTNYNPSLKS |
| 178 | EAYELQLGLYYYYGMDV |
| 179 | SGGYYWS |
| 180 | YSGNTYYNPSLRS |
| 181 | EAGGNSAYYYGMDV |
| 182 | DYYMS |
| 183 | YISSSGSTIYYADSVKG |
| 184 | DRTYYFGSGSYEGMDV |
| 185 | RASQGIRNDLG |
| 186 | AASSLQS |
| 187 | LQHNSNPFT |
| 188 | RASQSVSRNLV |
| 189 | GASTRAN |
| 190 | QQYKSWRT |
| 191 | RASQSISSYLN |
| 192 | AASSLQS |
| 193 | QQSYSTPFT |
| 194 | RASQSVSRNLA |
| 195 | GASTRAT |
| 196 | QQYNNWPTWT |
| 197 | RASQGIRNDLG |
| 198 | AASSFQS |
| 199 | LQHNSYPPT |
| 200 | RASQGIRNDLG |
| 201 | AASSLQS |
| 202 | LQHKSYPLT |
| 203 | RASQGIRNDLG |
| 204 | AASSLQS |
| 205 | LQHKSYPLT |
| 206 | RASQGIRNDLG |
| 207 | AASSLQS |
| 208 | LQHKSYPLT |
| 209 | RASQGIRNDLG |
| 210 | AASSLQS |
| 211 | LQHKSYPLT |
| 212 | RASQGIRSWLA |
| 213 | AASSLQS |
| 214 | QQANNFPRT |
| 215 | RASQSVSSNLA |
| 216 | GASTRAA |
| 217 | QHYINWPKWT |
| 218 | RASQSISSSLA |
| 219 | GASTRAT |
| 220 | QQYDNWPLT |
| 221 | KSSQSLLHSDGKTYLY |
| 222 | EVSTRES |
| 223 | MQSIQLPLT |
| 224 | RASQSVSSNLA |
| 225 | DASTRAT |
| 226 | QQYDNWPLT |
| 227 | RASQSVSSNLA |
| 228 | DASTRAA |
| 229 | QQYDNWPLT |
| 230 | RASQSISTSLA |
| 231 | GTSTRAT |
| 232 | QQYDIWPLT |
| 233 | RASQSVSSNLA |
| 234 | GASTRAT |
| 235 | QQYDNWPLT |
| 236 | KTSQSVLYSSKNKNFLA |
| 237 | WASTRES |
| 238 | QQYYSTPFT |
| 239 | RASQSISSNLA |
| 240 | GASTRAT |
| 241 | QQYDTWPLT |
| 242 | RASQGISNYLA |
| 243 | AASTLQS |
| 244 | QKYNRAPFT |
| 245 | RASQGISNYLA |
| 246 | AASTLQS |
| 247 | QKYNRAPFT |
| 248 | RASQSVSSNLA |
| 249 | DASTRAA |
| 250 | QQYDNWPLT |
| 251 | RASQGIINDLG |
| 252 | AASSLQS |
| 253 | LQHNSYPPT |
| 254 | RSSQSLVYSDGHTCLN |
| 255 | KVSNWDS |
| 256 | MQGTHWPLCS |
| 257 | RSSQSLVYSDGHTCLN |
| 258 | KVSNWDS |
| 259 | MQGTHWPLCS |
| 260 | RASQAISIYLA |
| 261 | AASSLQS |
| 262 | QQYSSYPRT |
| 263 | RASQSVYSNLA |
| 264 | GASTRAT |
| 265 | QQYYNWPWT |

In some embodiments, the anti-IL-17RA antibody or antigen binding fragment thereof for use in the methods of treatment disclosed herein comprises an antigen binding site comprising at least one immunoglobulin heavy chain variable domain comprising CDRs of SEQ ID NOs: 1-26, respectively.

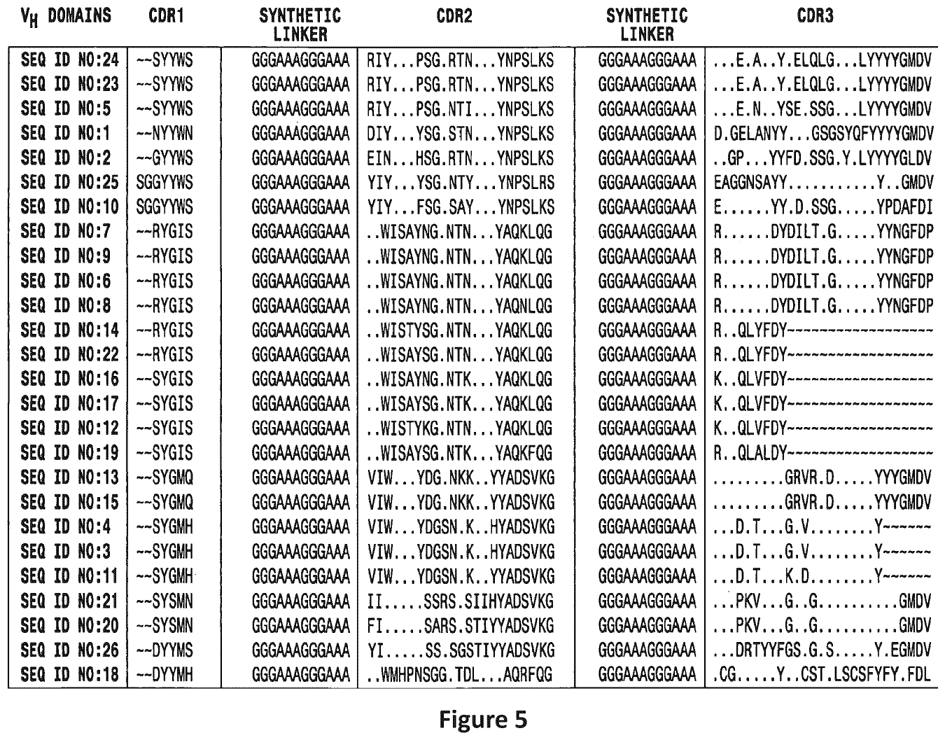

SEQ ID NOs: 1-26 are described in FIG. 5.

In some embodiments, the anti-IL-17RA antibody or antigen binding fragment thereof for use in the methods of treatment disclosed herein can also comprise at least one immunoglobulin light chain variable domain comprising CDRs of SEQ ID Nos: 27-53, respectively.

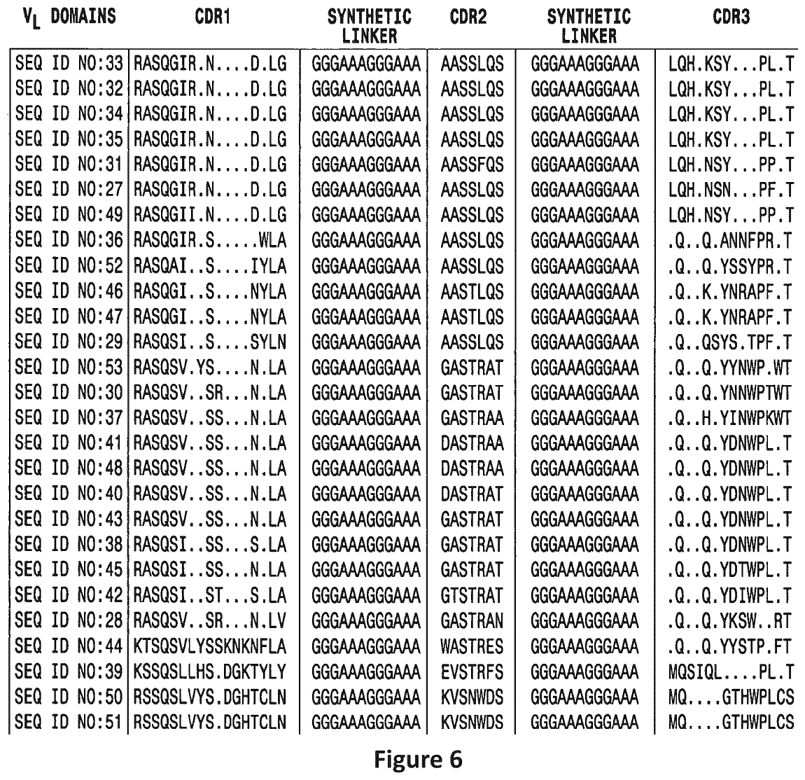

SEQ ID NOs: 27-53 are described in FIG. 6.

In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence comprising CDRs of SEQ ID NOs: 1-26 and/or a light chain amino acid sequence comprising CDRs of of SEQ ID NO: 27-53.

In some embodiments, the anti-IL-17RA antibody or antigen-binding fragment thereof for use in the methods of treatment disclosed herein comprises a light chain variable domain and a heavy chain variable domain comprising CDRs of SEQ ID NO:27 and SEQ ID NO: 1, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:28 and SEQ ID NO:2, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:29 and SEQ ID NO:3, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:30 and SEQ ID NO:4, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:31 and SEQ ID NO:5, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:32 and SEQ ID NO:6, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:33 and SEQ ID NO:7, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:34 and SEQ ID NO:8, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:35 and SEQ ID NO:9, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:36 and SEQ ID NO: 10, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:37 and SEQ ID NO: 11, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:38 and SEQ ID NO: 12, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:39 and SEQ ID NO: 13, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:40 and SEQ ID NO: 14, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:41 and SEQ ID NO:15, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:42 and SEQ ID NO: 16, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:43 and SEQ ID NO: 17, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:44 and SEQ ID NO: 18, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:45 and SEQ ID NO: 19, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:46 and SEQ ID NO:20, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:47 and SEQ ID NO:21, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:48 and SEQ ID NO:22, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 49 or SEQ ID NO:50 and SEQ ID NO:23, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:51 and SEQ ID NO:24, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:52 and SEQ ID NO:25, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:53 and SEQ ID NO:26, respectively. In some embodiments, the anti-IL-17RA antibody or antigen-binding fragment thereof for use in the methods of treatment disclosed herein comprises binds IL-17 receptor A.

In some embodiments, the anti-IL-17RA antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NOs: 1-26 and comprising HCDR1 comprising the amino acid sequence of SEQ ID NOs: 107, 110, 113, 116, 119, 122, 125, 128, 131, 134, 137, 140, 143, 146, 149, 152, 155, 158, 161, 164, 167, 170, 173, 176, 179, or 182, HCDR2 comprising SEQ ID NOs: 108, 111, 114, 117, 120, 123, 126, 129, 132, 135, 138, 141, 144, 147, 150, 153, 156, 159, 162, 165, 168, 171, 174, 177, 180, or183, and HCDR3 comprising SEQ ID NOs: 109, 112, 1115, 118, 121, 124, 127, 130, 133, 136, 139, 142, 145, 148, 151, 154, 157, 160, 163, 166, 169, 172, 175, 178, 181, or 184.

In some embodiments, the anti-IL-17RA antibody or antigen binding fragment thereof comprises an antigen binding site comprising at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NOs: 27-53 and comprising LCDR1 comprising the amino acid sequence SEQ ID NOs: 185, 188, 191, 194, 197, 200, 203, 206, 209, 212, 215, 218, 221, 224, 227, 230, 233, 236, 239, 242, 245, 248, 251, 254, 257, 260, or 263, LCDR2 comprising SEQ ID NOs: 186, 189, 192, 195, 198, 201, 204, 207, 210, 213, 216, 219, 222, 225, 228, 231, 234, 237, 240, 243, 246, 249, 252, 255, 258, 261, or 264, and LCDR3 comprising SEQ ID NOs: 187, 190, 193, 196, 199, 202, 205, 208, 211, 214, 217, 220, 223, 226, 229, 232, 235, 238, 241, 244, 247, 250, 253, 256, 259, 262, or 265.

In some embodiments, the anti-IL-17RA antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to the amino acid sequence of any of the heavy chain variable domains disclosed in the International patent publication WO2008054603 incorporated herein by reference. In some embodiments, the anti-IL-17RA antibody or antigen binding fragment thereof comprises an antigen binding site comprising at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to the amino acid sequence of any of the light chain variable domains disclosed in the International patent publication WO2008054603 incorporated herein by reference.

In some embodiments, the anti-PD-1 antibody or antigen binding fragment thereof for use in the methods of treatment disclosed herein comprises an antigen binding site comprising at least one immunoglobulin heavy chain variable domain (VH) which comprises in sequence hypervariable regions HCDR1, HCDR2 and HCDR3 of anti-PD-1 clones 01, 02, 03, 04, 07, 09, 51, humanized 51, 55, 79, or 80, or SEQ ID NOS: 311-312 and at least one immunoglobulin light chain variable domain (VL) which comprises in sequence hypervariable regions LCDR1, LCDR2, and LCDR3 of anti-PD-1 clones 01, 02, 03, 04, 07, 09, 51, humanized 51, 55, 79, or 80, or SEQ ID NOS: 311-312.

In some embodiments, the anti-IL-17RA antibody for use in the methods of treatment disclosed herein comprises a heavy chain variable region of brodalumab comprising SEQ ID NO: 1-9 and a light chain variable region comprising SEQ ID NO: 10-18. The HCDR1, HCDR2, and HCDR3 are provided in FIG. 5. The LCDR1, LCDR2, and LCDR3 are provided in FIG. 5. Thus, in other embodiments, the anti-IL-17RA antibody or antigen-binding fragment thereof comprises a light chain CDRI, CDR2, CDR3 and a heavy chain CDRI, CDR2, CDR3 of FIG. 5. In some embodiments, the anti-IL-17RA antibody or antigen binding fragment thereof for use in the methods of treatment disclosed herein comprises an antigen binding site comprising at least one immunoglobulin heavy chain variable domain comprising SEQ ID NO: 1-9 and at least one immunoglobulin light chain variable domain comprising SEQ ID NO: 10-18. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence of SEQ ID NOs: 1-9 and/or a light chain amino acid sequence of SEQ ID NO: 10-18. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain amino acid sequence of SEQ ID NO: 1-9 and comprising heavy chain CDR1, CDR2, CDR3 and/or a light chain amino acid sequence of SEQ ID NO: 10-18. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a light chain amino acid sequence of SEQ ID NOs: 10-18 and/or a heavy chain amino acid sequence of SEQ ID NO: 1-9. In some embodiments the anti-IL-17RA antibody or antigen-binding fragment thereof comprises an amino acid sequence that is of at least 80%, 81%, 82%, 83%, 84%, 85%, 86%, 87%, 88%, 89%, 90%, 91%, 92%, 93%, 94%, 95%, 96%, 97%, 98%, or 99% identical to a light chain amino acid sequence of SEQ ID NO: 10-18 and comprising light chain CDR1, CDR2, CDR3 and/or a light chain amino acid sequence of SEQ ID NO: 1-9.

In some embodiments, the anti-IL-17RA antibody or antigen binding fragment thereof comprises an antigen binding site comprising at least one immunoglobulin heavy chain variable domain with hypervariable regions HCDR1, HCDR2, and HCDR3; or HCDR equivalents thereof. In some embodiments, the anti-IL-17RA antibody or antigen binding fragment thereof can also comprise at least one immunoglobulin light chain variable domain which comprises hypervariable regions LCDR1, LCDR2, and LCDR3; or LCDR equivalents thereof.

In some embodiments, the anti-IL-17RA antibody or antigen-binding fragment thereof for use in the methods of treatment disclosed herein comprises a light chain variable domain and a heavy chain variable domain comprising CDRs of SEQ ID NO: 10 and SEQ ID NO: 1, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 11 and SEQ ID NO:2, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 12 and SEQ ID NO:3, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 13 and SEQ ID NO:4, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:14 and SEQ ID NO:5, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 15 and SEQ ID NO:6, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO:16 and SEQ ID NO:7, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 17 and SEQ ID NO:8, respectively. In some embodiments, the anti-IL-17RA antibody or antigen-binding fragment thereof for use in the methods of treatment disclosed herein comprises a light chain variable domain and a heavy chain variable domain comprising CDRs of SEQ ID NO: 18 and SEQ ID NO:9, respectively. In some embodiments, the anti-IL-17RA antibody or antigen-binding fragment thereof for use in the methods of treatment disclosed herein comprises binds PD1.

In some embodiments, the anti-PD-1 antibody or antigen-binding fragment thereof for use in the methods of treatment disclosed herein comprises a light chain variable domain and a heavy chain variable domain comprising CDRs of SEQ ID NO:307 and SEQ ID NO: 306, respectively. In some embodiments, the anti-PD-1 antibody or antigen-binding fragment thereof for use in the methods of treatment disclosed herein comprises a light chain variable domain and a heavy chain variable domain comprising CDRs of SEQ ID NO:309 and SEQ ID NO: 308, respectively.

In some embodiments, the anti-IL17RA or anti-PD-1 antibody or antigen binding fragment thereof for use in the methods of treatment disclosed herein comprises a human or humanized version of anti-IL17RA or anti-PD-1 antibody or an antigen binding fragment thereof. Humanized forms of non-human (e.g., murine) antibodies are chimeric immunoglobulins, immunoglobulin chains or fragments thereof (such as Fv, Fab, Fab′, F(ab′) 2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some embodiments, the anti-IL17RA or anti-PD-1 antibody is fully humanized wherein all the framework residues are derived from human immunoglobulins (recipient antibody). In some embodiments, the anti-IL17RA or anti-PD-1 antibody is partially humanized. In some instances, framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Methods for humanizing non-human antibodies are well known in the art.

In some embodiments, the antigen binding fragment of an anti-IL17RA antibody comprises fragments, such as F (ab′) 2, Fab′, Fab, Fv, sFv, dAb, complementarity determining region (CDR) fragments, single-chain antibodies (scFv), bivalent single-chain antibodies, diabodies, triabodies, tetrabodies, (poly) peptides that contain at least a fragment of an immunoglobulin that is sufficient to confer specific antigen binding to the (poly) peptide, etc., including hybrid fragments. Thus, fragments of the antibodies that retain the ability to bind IL17RA or anti-PD-1 specific antigens are provided. Such antibodies and fragments can be made by techniques known in the art and can be screened for specificity and activity according to the methods known in the art.

In some embodiments, the nucleic acid sequence of the anti-IL17RA or anti-PD-1 antibody codes for an amino acid sequence that comprises at least a variable heavy and variable light chain portions of the amino acid sequence of the anti-IL17RA or anti-PD-1 antibodies described herein. In some embodiments, the nucleic acid sequence encoding the anti-IL17RA or anti-PD-1 antibody codes for an amino acid sequence that comprises at least the CDRs of the variable heavy chain and the CDRs of the variable light chain portions of the amino acid sequence of the anti-IL17RA or anti-PD-1 antibodies described herein.

In some embodiments, the anti-IL17RA or anti-PD-1 antibody or antigen binding fragment thereof comprises conjugated antibodies or antibody fragments. Conjugated antibodies or fragments refer to antibodies or fragments that are operatively linked or otherwise physically or functionally associated with an effector moiety or tag, such as inter alia a toxic substance, a radioactive substance, fluorescent substance, a liposome, or an enzyme. In some embodiments, the anti-IL17RA or anti-PD-1 antibody or antigen binding fragment thereof is conjugated to a nanoparticle which can comprise a payload. Exemplary payloads include, but are not limited to, dexamethasone and budesonide, IL-2, and IL-15. In some embodiments, the dexamethasone or budesonide treats irAEs. In some embodiments, IL-2 or IL-15 treats cancer.

### Anti-PD-1 Antibodies

In some embodiments, the anti-PD-1 antibody comprises at least a portion of the amino acid sequence encoding the anti-PD-1 targeting portion of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the amino acid sequence of the anti-PD-1 antibody comprises at least a variable heavy and variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the amino acid sequence of the anti-PD-1 antibody comprises at least the CDRs of the variable heavy chain and the CDRs of the variable light chain portions of the amino acid sequence of Nivolumab, Pembrolizumab, Cemiplimab, Retifanlimab, Dostarlimab, Zimberclimab, Tiselelizumab, Camrelizumab, Sintilimab, Penpulimab, or any other anti-PD-1 antibody known in the art. In some embodiments, the nucleic acid sequence the anti-PD-1 antibody codes for an amino acid sequence that comprises at least a portion of the amino acid sequence of Nivolumab, Pembrolizumab, Cemiplimab, Retifanlimab, Dostarlimab, Zimberclimab, Tiselelizumab, Camrelizumab, Sintilimab, Penpulimab, or any other anti-PD-1 antibody known in the art. In some embodiments, the nucleic acid sequence of the anti-PD-1 antibody codes for an amino acid sequence that comprises at least a variable heavy and variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the nucleic acid sequence encoding the anti-PD-1 antibody codes for an amino acid sequence that comprises at least the CDRs of the variable heavy chain and the CDRs of the variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. The sequences of anti-PD-1 antibodies are described in the art and incorporated herein by reference as follows: Pembrolizumab (see U.S. Pat. Nos. 8,168,757, 8,354,509, 8,900,587, the contents of each of which is hereby incorporated by reference in its entirety), Cemiplimab (scc U.S. Pat. No. 9,987,500 the contents of which is hereby incorporated by reference in its entirety), Retifanlimab (see US2019/0127467 the contents of which is hereby incorporated by reference in its entirety), Dostarlimab (scc WO/2021/058711 the contents of which is hereby incorporated by reference in its entirety), Zimberelimab (see CN106432494 the contents of which is hereby incorporated by reference in its entirety), Tisclelizumab (see U.S. Pat. No. 8,735,553 the contents of which is hereby incorporated by reference in its entirety), Camrelizumab (see US2019/0309069 the contents of which is hereby incorporated by reference in its entirety), Sintilimab (see U.S. Pat. No. 10,316,089 the contents of which is hereby incorporated by reference in its entirety), Penpulimab (see US2019/0321466 the contents of which is hereby incorporated by reference in its entirety). The CDRs of certain anti-PD-1 antibodies are described in Jeong T J, Lee H T, Gu N, Jang Y J, Choi S B, Park U B, Lec S H, Heo Y S, The High-Resolution Structure Reveals Remarkable Similarity in PD-1 Binding of Cemiplimab and ostarlimab, the FDA-Approved Antibodies for Cancer Immunotherapy. Biomedicines, 2022 Dec. 6; 10 (12): 3154, the contents of which is hereby incorporated by reference in its entirety.

### Anti-PDL-1 Antibodies

In some embodiments, the anti-PDL-1 antibody comprises at least a portion of the amino acid sequence encoding the anti-PDL-1 targeting portion of Atezolimumab, Durvalumab and Avelumab, or any other anti-PDL-1 antibody known in the art. Keler et al. (2003) J. Immunol. 171:6251; Ribas et al. (2007) Oncologist 12:873. In some embodiments, the amino acid sequence of the anti-PDL-1 antibody comprises at least a variable heavy and variable light chain portions of the amino acid sequence of Atezolimumab, Durvalumab and Avelumab, or any other anti-PDL-1 antibody known in the art. In some embodiments, the amino acid sequence of the anti-PDL1 antibody comprises at least the CDRs of the variable heavy chain and the CDRs of the variable light chain portions of the amino acid sequence of Atezolimumab, Durvalumab and Avelumab, or any other anti-PDL-1 antibody known in the art. In some embodiments, the nucleic acid sequence the anti-PDL-1 antibody codes for an amino acid sequence that comprises at least a portion of the amino acid sequence of Atezolimumab, Durvalumab and Avelumab, or any other anti-PDL-1 antibody known in the art. In some embodiments, the nucleic acid sequence of the anti-PDL-1 antibody codes for an amino acid sequence that comprises at least a variable heavy and variable light chain portions of the amino acid sequence of Atezolimumab, Durvalumab and Avelumab, or any other anti-PDL-1 antibody known in the art. In some embodiments, the nucleic acid sequence encoding the anti-PDL-1 antibody codes for an amino acid sequence that comprises at least the CDRs of the variable heavy chain and the CDRs of the variable light chain portions of the amino acid sequence of Atezolimumab, Durvalumab and Avelumab, or any other anti-PDL-1 antibody known in the art. The sequences of anti-PDL-1 antibodies are described in the art and incorporated herein by reference as follows: Atezolimumab (see U.S. Pat. No. 8,217,149, the contents of each of which is hereby incorporated by reference in its entirety), Durvalumab (see U.S. Pat. Nos. 8,779,108 and 9,493,565, the contents of each of which is hereby incorporated by reference in its entirety), Avelumab (see IPN WO2013079174, where the antibody having the amino acid sequence of Avelumab is referred to as A09-246-2, the contents of each of which is hereby incorporated by reference in its entirety).

### Anti-CTLA-4 Antibodies

In some embodiments, the anti-CTLA-4 antibody comprises at least a portion of the amino acid sequence encoding the anti-CTLA-4 targeting portion of ipilimumab and tremelimumab, or any other anti-CTLA-4 antibody known in the art. In some embodiments, the amino acid sequence of the anti-CTLA-4 antibody comprises at least a variable heavy and variable light chain portions of the amino acid sequence of ipilimumab and tremelimumab, or any other anti-CTLA-4 antibody known in the art. In some embodiments, the amino acid sequence of the anti-CTLA-4 antibody comprises at least the CDRs of the variable heavy chain and the CDRs of the variable light chain portions of the amino acid sequence of ipilimumab and tremelimumab, or any other anti-CTLA-4 antibody known in the art. In some embodiments, the nucleic acid sequence the anti-CTLA-4 antibody codes for an amino acid sequence that comprises at least a portion of the amino acid sequence of ipilimumab and tremelimumab, or any other anti-CTLA-4 antibody known in the art. In some embodiments, the nucleic acid sequence of the anti-CTLA-4 antibody codes for an amino acid sequence that comprises at least a variable heavy and variable light chain portions of the amino acid sequence of ipilimumab and tremelimumab, or any other anti-CTLA-4 antibody known in the art. In some embodiments, the nucleic acid sequence encoding the anti-CTLA-4 antibody codes for an amino acid sequence that comprises at least the CDRs of the variable heavy chain and the CDRs of the variable light chain portions of the amino acid sequence of ipilimumab and tremelimumab, or any other anti-CTLA-4 antibody known in the art. The sequences of anti-CTLA-4 antibodies are described in the art and incorporated herein by reference as follows: ipilimumab (see USPPN 2002/0086014 and No. 2003/0086930, the contents of each of which is hereby incorporated by reference in its entirety), tremelimumab (see U.S. Pat. No. 6,682,736, the contents of each of which is hereby incorporated by reference in its entirety).

### Anti-LAG-3 Antibodies

In some embodiments, the anti-LAG-3 antibody comprises at least a portion of the amino acid sequence encoding the anti-LAG-3 targeting portion of ipilimumab and tremelimumab, or any other anti-LAG-3 antibody known in the art. In some embodiments, the amino acid sequence of the anti-LAG-3 antibody comprises at least a variable heavy and variable light chain portions of the amino acid sequence of favezelimab, INCAGN02385, IBI-110, sym-022, LBL-007, HLX 26, Icramilimab, fianlimab or any other anti-LAG-3 antibody known in the art. In some embodiments, the amino acid sequence of the anti-LAG-3 antibody comprises at least the CDRs of the variable heavy chain and the CDRs of the variable light chain portions of the amino acid sequence of favezelimab, INCAGN02385, IBI-110, sym-022, LBL-007, HLX 26, Ieramilimab, fianlimab, or any other anti-LAG-3 antibody known in the art. In some embodiments, the nucleic acid sequence the anti-LAG-3 antibody codes for an amino acid sequence that comprises at least a portion of the amino acid sequence of favezelimab, INCAGN02385, IBI-110, sym-022, LBL-007, HLX 26, Ieramilimab, fianlimab, or any other anti-LAG-3 antibody known in the art. In some embodiments, the nucleic acid sequence of the anti-LAG-3 antibody codes for an amino acid sequence that comprises at least a variable heavy and variable light chain portions of the amino acid sequence of favezelimab, INCAGN02385, IBI-110, sym-022, LBL-007, HLX 26, Ieramilimab, fianlimab, or any other anti-LAG-3 antibody known in the art. In some embodiments, the nucleic acid sequence encoding the anti-LAG-3 antibody codes for an amino acid sequence that comprises at least the CDRs of the variable heavy chain and the CDRs of the variable light chain portions of the amino acid sequence of favezelimab, INCAGN02385, IBI-110, sym-022, LBL-007, HLX 26, Ieramilimab, fianlimab, or any other anti-LAG-3 antibody known in the art. The sequences of anti-LAG-3 antibodies are described in the art and incorporated herein by reference.

### Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, antibodies are synthesized by the hybridoma culture method which results in antibodies that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N. Y., 1981)), recombinant DNA methods, phage-display technologies (scc, e.g., Clackson et al, Nature, 352:624-628 (1991); Marks et al, J. Mol Biol. 222:581-597 (1992); Sidhu et al, J. Mol Biol. 338 (2): 299-310 (2004); Lec et al, J. Mol Biol. 340 (5): 1073-1093 (2004); Fellouse, Proc. Natl. Acad. ScL USA 101 (34): 12467-12472 (2004); and Lee et al, J. Immunol. Methods 284 (1-2): 119-132 (2004), and technologies for producing human or humanlike antibodies in animals that have parts or all of the human immunoglobulin loci or genes encoding human immunoglobulin sequences (see, e.g., Lonberg et al, Nature 368:856-859 (1994); Morrison, Nature 368:812-813 (1994); Fishwild et al, Nature Biotechnol 14:845-851 (1996); Neuberger, Nature Biotechnol. 14:826 (1996); and Lonberg and Huszar, Intern. Rev. Immunol. 13:65-93 (1995).

In some embodiments, expression of an antibody comprises expression vector(s) containing a polynucleotide that encodes an anti-PD-1 or an anti-IL17RA antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an anti-PD-1- or an anti-IL17RA antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

The polynucleotide encoding the antibody may be modified, for example, by substituting the coding sequence for human heavy- and light-chain constant domains in place of the homologous murine sequences (U.S. Pat. No. 4,816,567; Morrison, et al, Proc. Natl Acad. ScL USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may by monovalent, the preparation of which is well known in the art. For example, one method involves recombinant expression of immunoglobulin light chain and a modified heavy chain. The heavy chain is truncated generally at any point in the Fc domain so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the antibodies disclosed herein are encoded in a vector for expression in a cell line. In some embodiments, a vector comprises a polynucleotide sequence that encodes an anti-IL-17RA or anti-PD-1 antibody and the vector is transfected into one or more cell lines for expression. In some embodiments, one or more vectors comprise polynucleotide sequences encoding a light chain and a heavy chain of the antibody. For example, in some embodiments, a first vector may comprise a polynucleotide sequence encoding a light chain, a second vector may comprise a polynucleotide sequence encoding a heavy chain, of anti-IL-17RA or anti-PD-1 antibody. In some embodiments, both vectors are transfected into one or more cell lines for expression. A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56 (2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available media can be utilized.

Antibodies disclosed herein can be purified from any human or non-human cell which expresses the antibody, including those which have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### IL-17RA Antagonists

In some embodiments, the composition comprises an IL-17RA small interfering ribonucleic acid (siIL-17RA). In some embodiments, the siIL-17RA comprises the sequences encoding the small interfering ribonucleic acid (siIL-17RA) of any of the sequences of Table 1. In various embodiments, the siRNA comprises a nucleic acid sequence at least 75%, 80%, 85%, 90%, 95%, 96%, 97%, 98% or 99% identical to any of the sequences of Table 1. In some embodiments, the siRNA consists of a siRNA nucleic acid sequence of any of the sequences of Table 8.

TABLE 8

| SEQ. ID No. | Sequence |
|---|---|
| 302 | gctgtgacggcgacgtccccgacct |
| 54 | aggcccaggggggctgcagtagacc |
| 55 | ccctctacgtggacgtggtcctgaa |
| 56 | accacctgcccaagcccatccctga |
| 57 | gctgtcgccaccaagtgcagatcca |
| 58 | gaccagaagagttccaccagcgatc |
| 59 | aaactgaggcatcaccacaggcggt |
| 60 | agcacgccaggatgaaggtaaccac |
| 61 | caaaggacctgcagatccagctgca |
| 62 | gagaaccacagttgctttgagcaca |

In some embodiments, the composition comprises an IL-17RA short-hairpin ribonucleic acid (shIL-17RA). In some embodiments, the shIL-17RA comprises a nucleic acid sequences of any of the sequences of Table 1. In some embodiments, the composition comprises a viral vector comprising a nucleic acid sequence encoding a shIL-17RA. In some embodiments, the viral vector is an adeno-associated vector (AAV). In various embodiments, the viral vector is a vector that preferentially targets the liver or liver cells. In various embodiments, the AAV is AAV 1, 2, 3, 4, 5, 6, 7, 8, 9, or 10 or variant thereof. In some embodiments, the viral vector is AAV8.

Inhibition of RNA encoding IL-17RA can effectively modulate the expression of these proteins. Inhibitors can include shRNAs encoding siRNAs, siRNA; interfering RNA or RNAi; dsRNA; RNA Polymerase III transcribed DNAs; ribozymes; Oligonucleotide (ASO) and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding an EGFR fusion molecule can be synthesized, e.g., by conventional phosphodiester techniques. Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector. Methods for producing and testing dsRNA or siRNA molecules are known in the art. A short hairpin RNA (shRNA) encodes an RNA molecule with a tight hairpin turn that can be used to silence target gene expression via RNA interference (RNAi). Expression of shRNA in cells is typically accomplished by delivery of plasmids or through viral or bacterial vectors.

RNA polymerase III transcribed DNAs contain promoters, such as the U6 promoter. These DNAs can be transcribed to produce small hairpin RNAs in the cell that can function as siRNA or linear RNAs, which can function as antisense RNA. The IL-25 inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded.

### Dosage

A prophylactically effective or therapeutically effective amount is typically dependent on the weight of the subject being treated, the subject's physical condition, the extensiveness of the condition to be treated, and the age of the subject being treated. In general, an anti-IL-17RA or anti-PD-1 antibody, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, or at least about 10 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 1 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

In some embodiments, brodalumab is be administered at a dose of 200-300 mg. In some embodiments, brodalumab is be administered at a dose of 210 mg.

In general, an anti-PD-1, anti-PDL-1, or anti-CTLA-4 antibody, or polynucleotides encoding one or more antibodies, disclosed herein may be administered in a therapeutically effective amount. Nivolumab can be administered at a dose between 200 mg and 500 mg. For example, Nivolumab can be administered at a dose of 240 mg every 2 weeks or 480 mg every four weeks for subjects weighing 40 kg or greater, 3 mg/kg every 2 weeks or 6 mg/kg every 4 weeks for subjects weighing less than 40 kg, or 360 mg with platinum-doublet chemotherapy on the same day every 3 weeks. Pembrolizumab can be administered at a dose between 100 mg and 500 mg. Pembrolizumab can be administered every three or six weeks. For example Pembrolizumab can be administered at a dose of 200 mg every three weeks or 400 mg every six weeks. In some embodiments, Pembrolizumab can be administered at 2 mg/kg every three weeks (up to a maximum of 200 mg). Cemiplimab can be administered at a dose between 300 mg and 400 mg. For example, cemiplimab can be administered at a dose of 350 mg every three weeks. Retifanlimab can be administered at a dose of 500 mg every 4 weeks. Dostarlimab can be administered at a dose between 400 mg and 1100 mg. For example, dostarlimab can be administered at at a dose of 500 mg every three weeks or 1000 mg every six weeks. Zimberelimab can be administered at a dose between 200 mg and 300 mg. For example, zimberelimab can be administered at a dose of 240 mg every 2 weeks. Tiselelizumab can be administered at a dose between 100 mg and 300 mg. For example, tiselelizumab can be administered at a dose of 200 mg every three weeks. Camrelizumab can be administered at a dose between 100 mg and 300 mg. For example, camrelizumab can be administered at a dose of 200 mg every 2 weeks. Sintilimab can be administered at a dose between 1 and 10 mg/kg or 100 mg and 300 mg. For example, Sintilimab can be administered at a dose of 1 mg/kg, 3 mg/kg, 10 mg/kg, or 200 mg every three weeks. Penpulimab can be administered at a dose between 100 mg and 300 mg. For example, penpulimab can be administered at a dose of 200 mg every two weeks. Atezolimumab can be administered at a dose between 740 mg and 1780 mg. For example, Atezolimumab can of be administered at a dose 840 mg every two weeks, 1200 mg every three weeks, or 1680 mg every four weeks. Durvalumab can be administered at a dose between 5 mg and 15 mg. Durvalumab can be administered at a dose of 10 mg/kg every two weeks or 1,500 mg every four weeks for subjects weighing 30 kg or greater, or 10 mg/kg every two weeks for subjects weighing less than 30 kg. Avelumab can be administered at a dose between 700 mg and 900 mg. For example, avelumab can be administered at a dose of 800 mg every two weeks. Ipilimumab can be administered at a dose between 0.5 mg/kg and 15 mg/kg. For example, ipilimumab can be administered at a dose 1 mg/kg, 3 mg/kg, or 10 mg/kg every three weeks. Tremelimumab can be administered at a dose between 50 mg and 400 mg or 0.5 mg/kg and 5 mg/kg. For example, tremelimumab can be administered at a dose of 75 mg or 300 mg for subjects weighing 30 kg or greater; or 1 mg/kg or 4 mg/kg for subjects weighing less than 30 kg.

Dosaging can also be administered in a patient-specific manner to provide a predetermined concentration of the agents in the blood, as determined by techniques accepted and routine in the art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

Immune checkpoint inhibitors (ICI) have improved outcomes and extended patient survival in several tumor types. However, ICI often induces immune-related adverse events (irAEs) that warrant therapy cessation, thereby limiting the overall effectiveness of this class of therapeutic agents. Currently, available therapies used to treat irAEs might also blunt the antitumor activity of the ICI themselves. Therefore, there is an urgent need to identify treatments that have the potential to be administered alongside ICI to optimize their use. Using a translationally relevant murine model of anti-PD-1 and anti-CTLA-4 antibodies-induced irAEs, the safety and efficacy of prednisolone, anti-IL6, anti-TNF, and anti-IL17RA administration to prevent irAEs and to reduce tumor size were compared. These findings suggest that IL17RA may serve dual roles in treating tumors responsive to ICI and allowing for extended ICI therapy by suppressing immune-related toxicities.

Immune checkpoint inhibitors (ICI) increase the survival of patients with several kinds of malignancies. Blocking the inhibitory receptors CTLA-4, PD-1, and PD-L1 (the ligand for PD-1) as monotherapy or combined with other agents has improved cancer treatment responses (1-4). CTLA-4 and PD-1 are T cell surface inhibitory receptors that prevent excessive T cell responses. Tumor cells have developed mechanisms to usurp those inhibitory mechanisms to prevent T cell-mediated tumor killing. To do that, tumor cells express inhibitory ligands such as PD-L1, and in some cases also PD-L2 (the second ligand for PD-1), to prevent T cell recognition and activation (5). Consequently, the therapeutic blockade of these checkpoints with ICI restores anti-tumor immunity. The PD-1-PD-LI interaction directly inhibits tumor-specific T cell responses, promotes peripheral effector T cell exhaustion, and enhances the development of regulatory T cell elements. The FDA has approved seven antibodies targeting inhibitory receptors for treating numerous cancers, including melanoma, renal cell carcinoma, squamous cell carcinoma, and Hodgkin lymphoma, to name a few. While ICI therapy has revolutionized cancer treatment, primary and secondary resistance is common. For example, ovarian cancer responds poorly to ICI therapy, with clinical trials reporting responses ranging from 6% to 22% (6-9).

One of the reasons for this is the presence of additional inhibitory immune checkpoint pathways in mammals. PD-LI is also the ligand of the receptor B7-1, and their interaction inhibits T cell proliferation and cytokine production (10-14). PD-1 has a second ligand, PD-L2, expressed in various immunosuppressive stromal cells, endothelial cells, macrophages, and, in some cases, tumor cells (15-17). PD-L2 binds to PD-1 with a 3-fold stronger affinity compared with PD-L1. PD-L2 also interacts with repulsive guidance molecule b (RGMb), an alternate receptor, and their interaction promotes respiratory immune tolerance by initiating immunoinhibitory signals (18). There are other mechanisms of tumor immune escape in the setting of ICI therapies, such as MHC downregulation, regulatory T cell deviation, and insufficient tumor-infiltrating lymphocytes (TIL) in tumors. In murine models, it has been shown that the presence of lymphocytes in different organs in mice treated with anti-CTLA-4 and anti-PD-1 antibodies varies and depends on multiple factors, including the genetic background of the mice.

The second limitation of ICI therapy is associated with irAEs (19-22). Since PD-1 is also expressed on non-tumor T cells, this on-target (PD-1) but off-tumor (irAEs organs) activation of exhausted autoimmune T cell clones is expected. Potentially, any organ can be injured as ICI disrupts self-tolerance to normal tissues. These irAEs range from mild to severe in various tissues, the most common of which include the skin, liver, lung, and gastrointestinal tract (23-25). Moderate irAEs require the temporary discontinuation of ICI and short-term use of corticosteroids with subsequent ICI treatment, limiting their efficacy. Severe irAEs often lead to the cessation of life-saving ICI therapy altogether. Other immunosuppressive drugs are usually required, such as high-dose prednisone, methotrexate, tocilizumab, and infliximab. Additionally, prolonged immune suppression may place the patients at risk of developing infections.

Given that similar immune mechanisms underpin both tumor control and irAEs, there remains a concern that steroids or other immunosuppressive agents used to treat irAEs may impede tumor response. Indeed, worse outcomes have been reported in patients treated with immunotherapy while receiving corticosteroids (26-30). Additionally, steroid-refractory colitis, myocarditis, and pneumonitis have been described and are associated with high mortality, and their optimal management strategies remain unclear. Unfortunately, since management decisions are often based on expert opinion and not mechanistic studies, a recent publication (31) suggests worse cancer outcomes in patients treated with anti-TNFα and anti-IL-6 agents. Controlled prospective trials coupled with emerging pathophysiological insight are needed to evaluate these issues properly.

### Example 1: Anti-PD-1 and Anti-CTLA-4 Antibodies Therapy Induces irAEs in Multiple Organs

For replicating clinical irAEs, a pharmacological model where anti-PD-1 and anti-CTLA-4 antibodies are injected into tumor-inoculated B6/Lpr mice biweekly for six weeks was utilized (FIG. 1A). Immune checkpoint blockade with anti-PD-1 and anti-CTLA-4 antibodies suppressed tumor growth (FIGS. 1Bi and 1Bii). However, compared to untreated mice, mice that underwent ICI therapy experienced increased levels of immune cell infiltration in the liver, lung, heart, and colon (FIG. 1C).

To uncover differences in the compositions of the immune infiltrates of the irAEs organ and the tumor, immune cells were isolated from the livers and the tumors of the ICI-treated mice. Next, a 33-plex flow cytometry panel was employed to generate a UMAP plot demonstrating clusters of CD45⁺ cells (FIG. 1Di). The CD4⁺ and the CD8⁺ T cells populated different clusters (FIGS. 1Dii and 1Diii). Remarkably, the immune cells isolated from the liver and from the tumor of the same mice differentially occupied other clusters (FIG. 1E). More specifically, clusters solely enriched in the liver or exclusively enriched in the tumor were identified (FIG. 1F). While CD4⁺CD8+FOXP3^{LOW} and CD8+TCF1⁺CD62L⁺ were mainly in the liver, CD8⁺CD69+CXCR3+ dominated the tumors. These data suggest that targeting specific T cell populations could inhibit irAEs inflammatory responses without interfering with the tumor inflammation.

### Example 2: Treatments for irAEs Counteract ICI's Anti-Tumor Effect

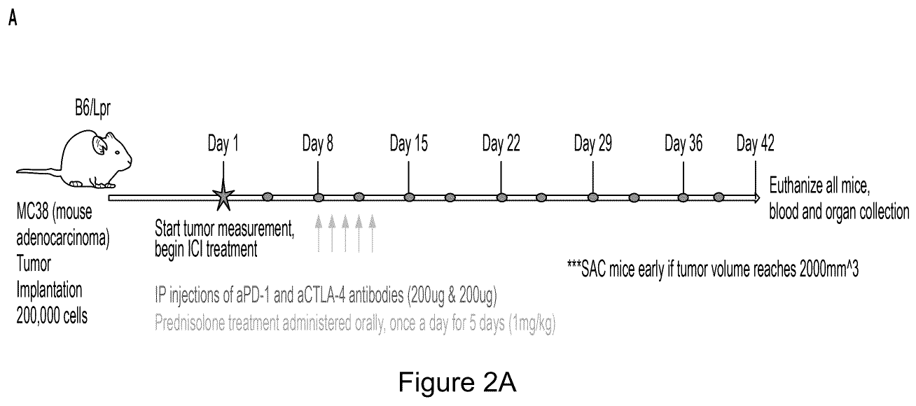

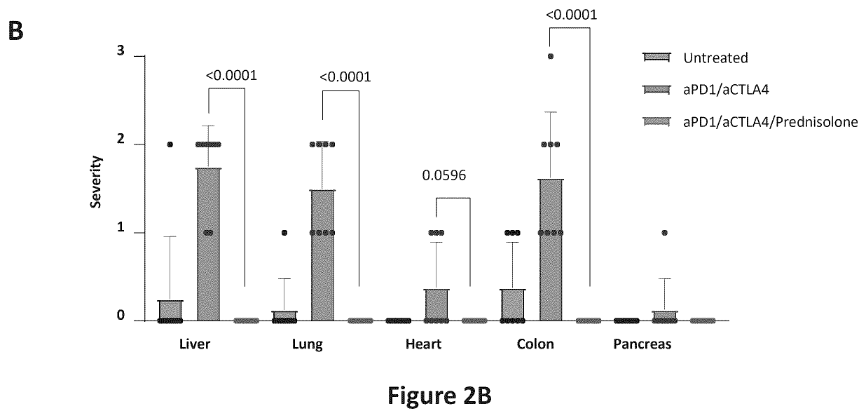

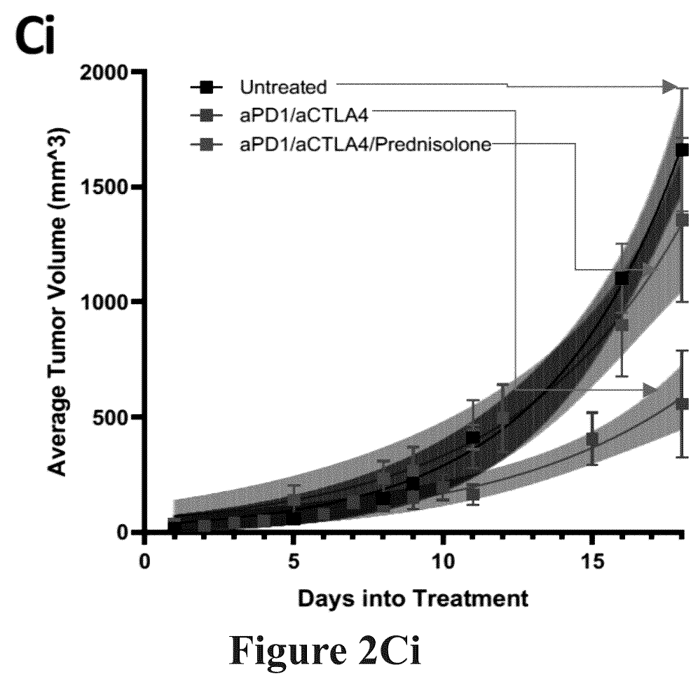

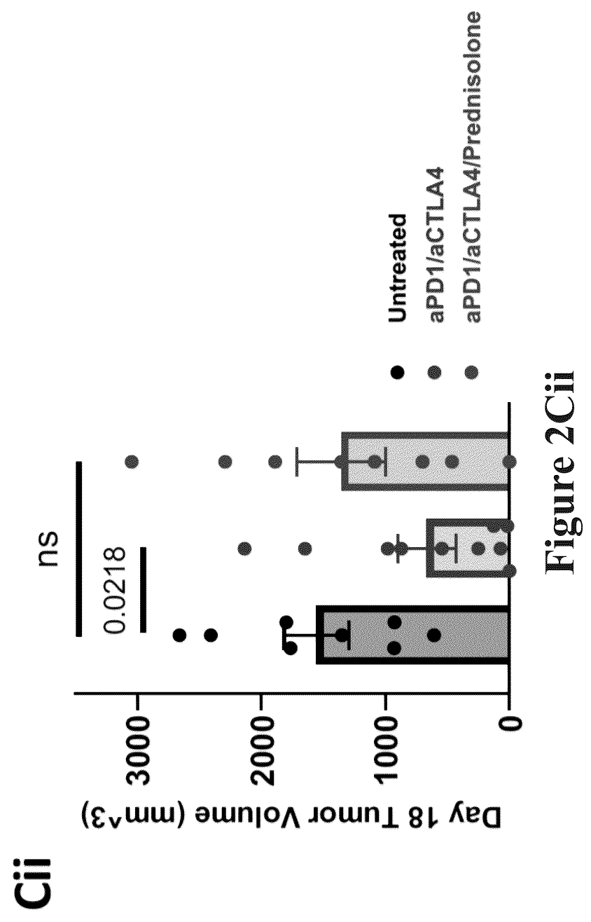

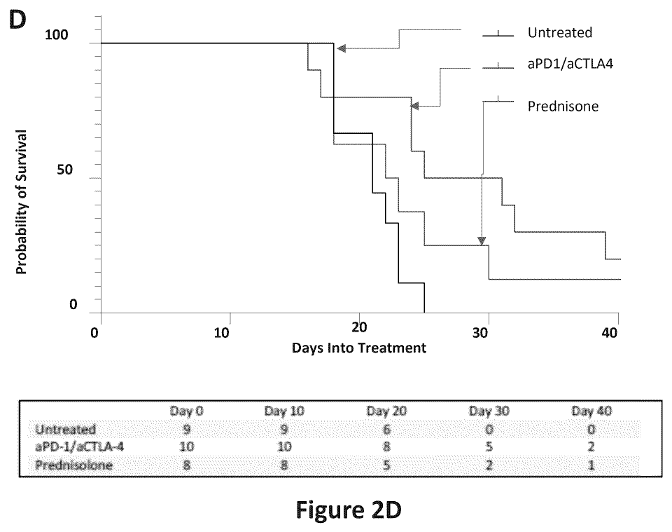

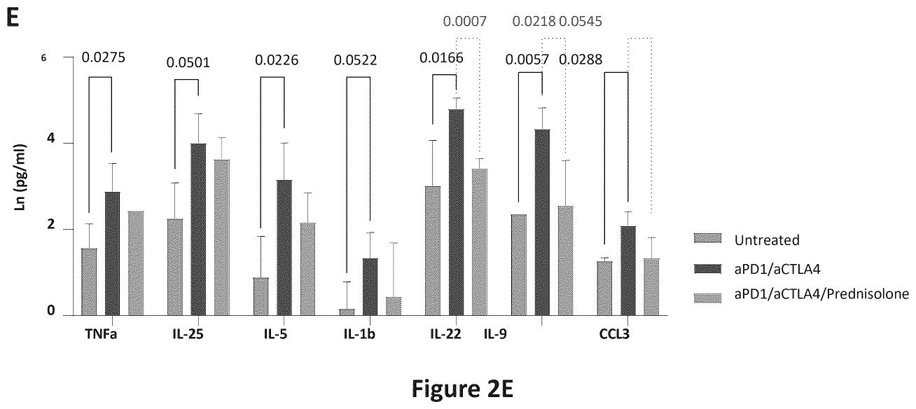

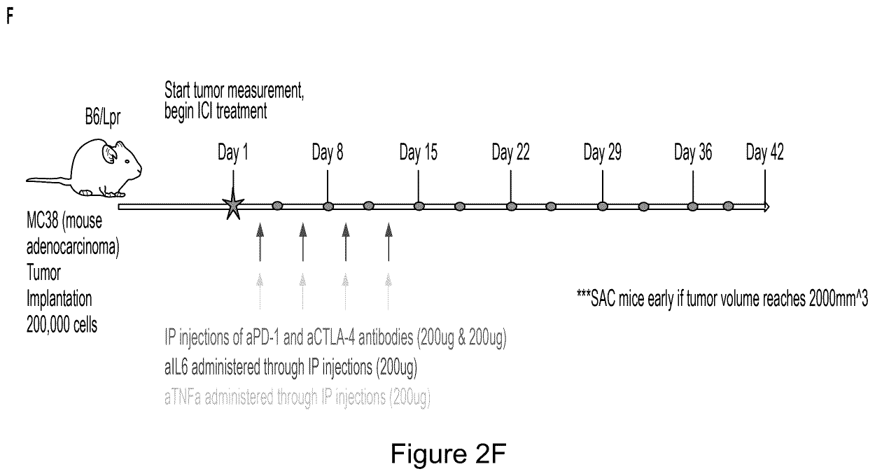

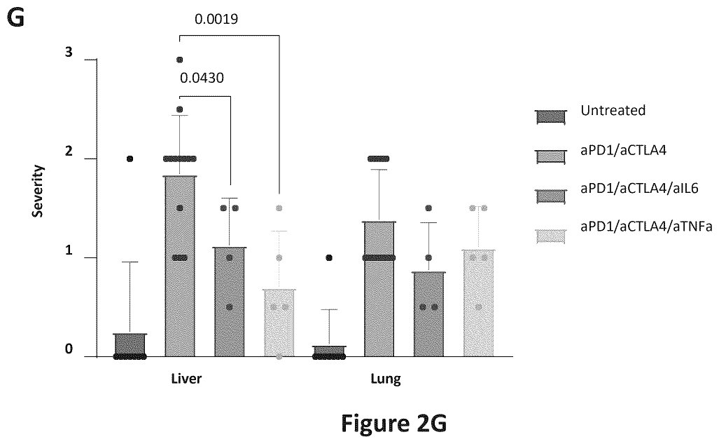

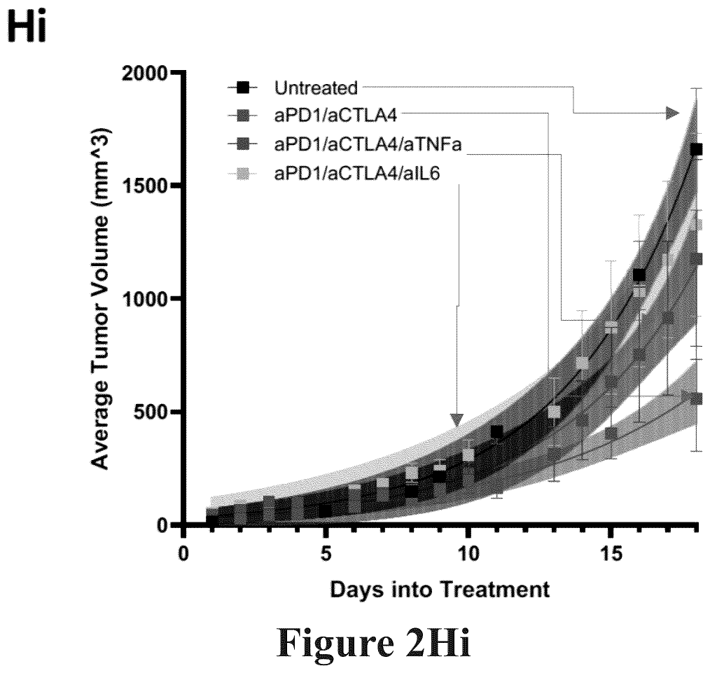

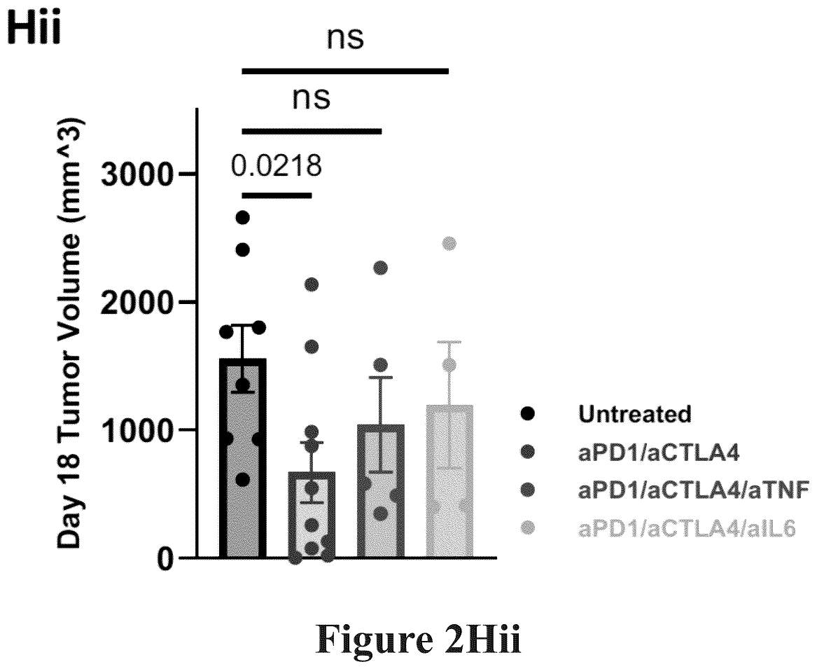

Prednisone is the first line of treatment for irAEs in the clinic. This care was simulated in vivo by providing prednisolone to irAEs mice also treated with anti-PD-1 and anti-CTLA-4 antibodies (FIG. 2A). Through H&E analysis of multi-organs collected at the endpoint of the experiment, Described herein is that prednisolone decreased immune-cell infiltration in the liver, lung, heart, and colon (FIG. 2B). However, prednisolone's anti-inflammatory property counteracts the anti-tumor effect of anti-PD-1 and anti-CTLA-4 antibodies and led to a notable increase in tumor growth and a decrease in survival probability (FIGS. 2Ci, 2Cii, and 2D). In search of an alternative drug target that could be neutralized to treat irAEs, a Luminex assay of serum samples of irAEs-treated mice was performed. As shown, prednisolone decreased IL22, IL9, and CCL3 levels. Yet, the decrease in the levels of the inflammatory cytokines TNFα, IL25, IL5, and ILlb was not notable (FIG. 2E). This suggested that neutralizing TNFα, as other groups have previously studied (33), or IL25 could potentially lower excessive off-tumor immune response through a pathway different from prednisolone treatment and be less detrimental to ICI's anti-tumor response.

Next the effects of anti-TNFα antibodies in the irAEs mice model, in comparison to anti-IL6 antibodies was examined (34), another agent used in irAEs clinical trials (FIG. 2F). As expected, theses results demonstrated a decrease in immune cells infiltrating the liver (FIG. 2G). However, anti-TNFα's and anti-IL6's anti-inflammatory effect was not confined to the liver and lung but also acted on the tumor, clashing with the desired immune activation of the ICI (FIGS. 2Hi and 2Hii). Anti-TNFα and anti-IL6 antibody treatments lowered mice's survival by accelerating tumor growth compared to anti-PD-1 and anti-CTLA-4 antibody therapy alone (FIG. 2I).

### Example 3: IL17RA is an Alternative and Advantageous Target than IL25

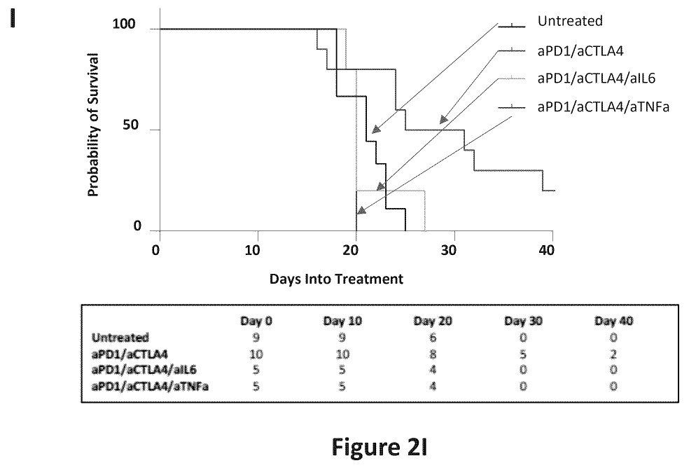

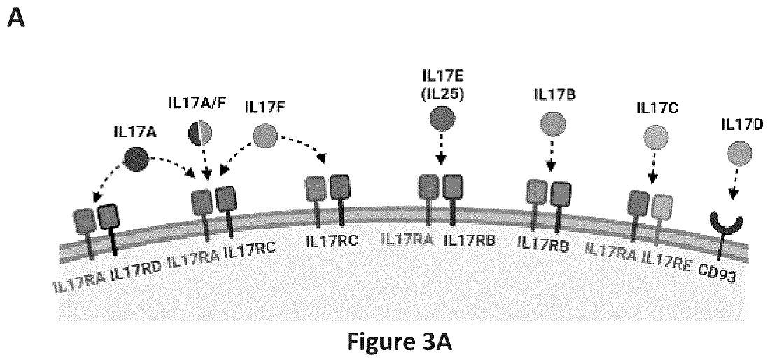

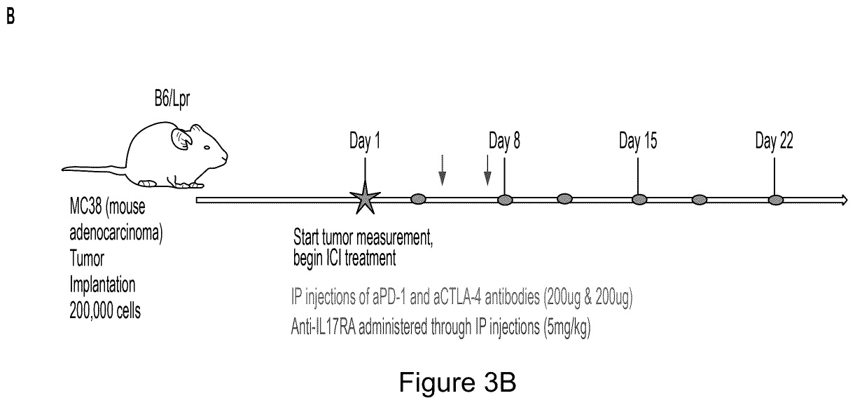

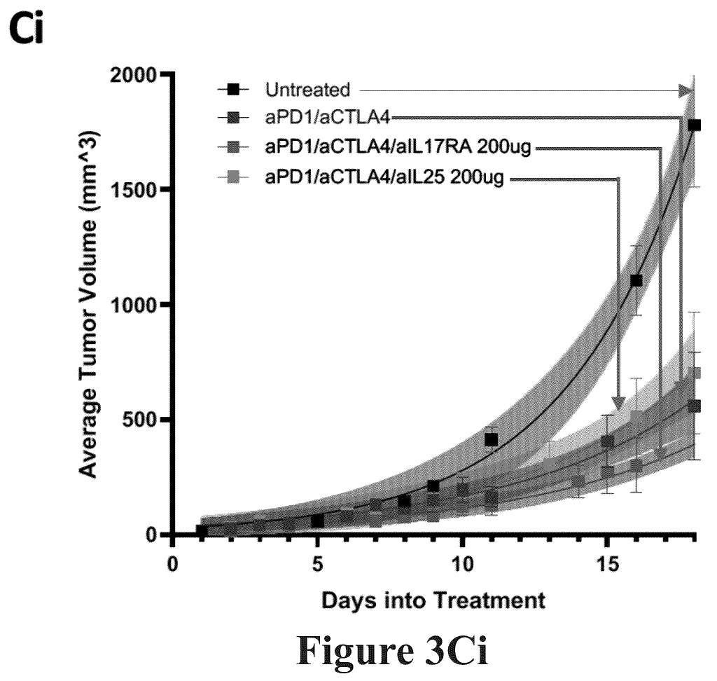

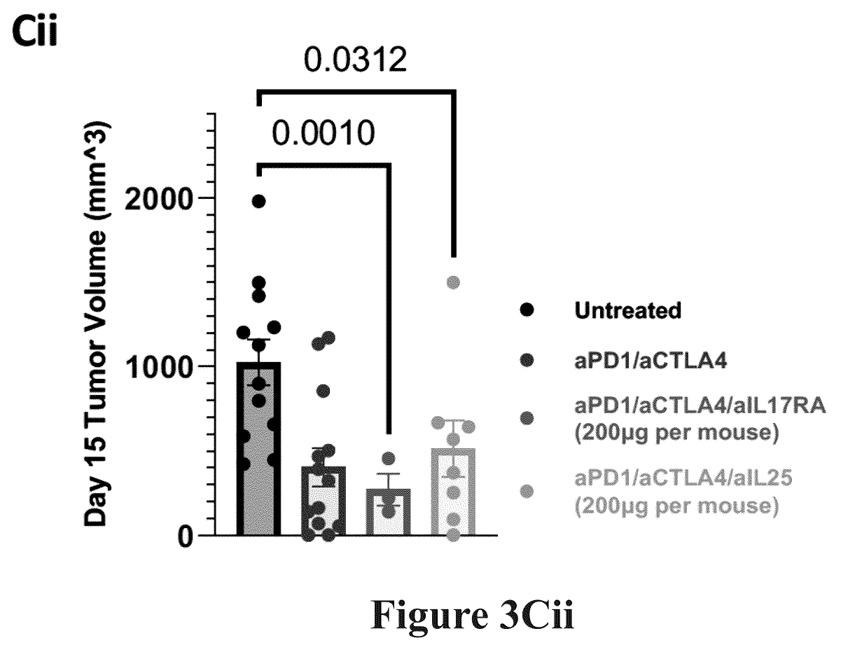

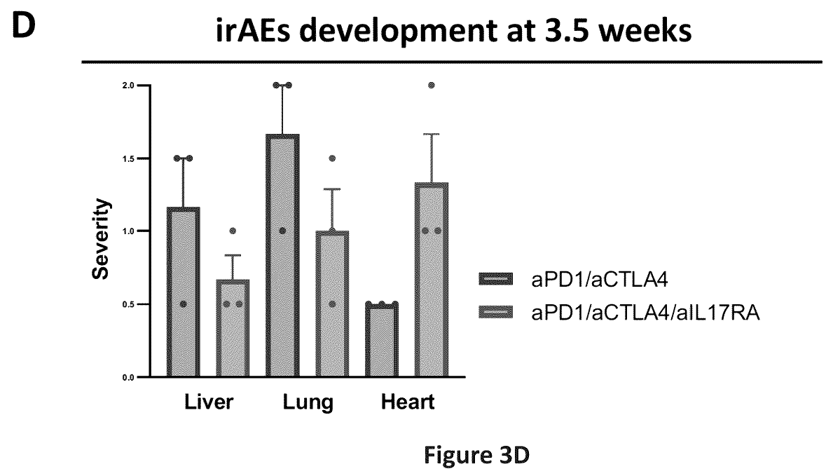

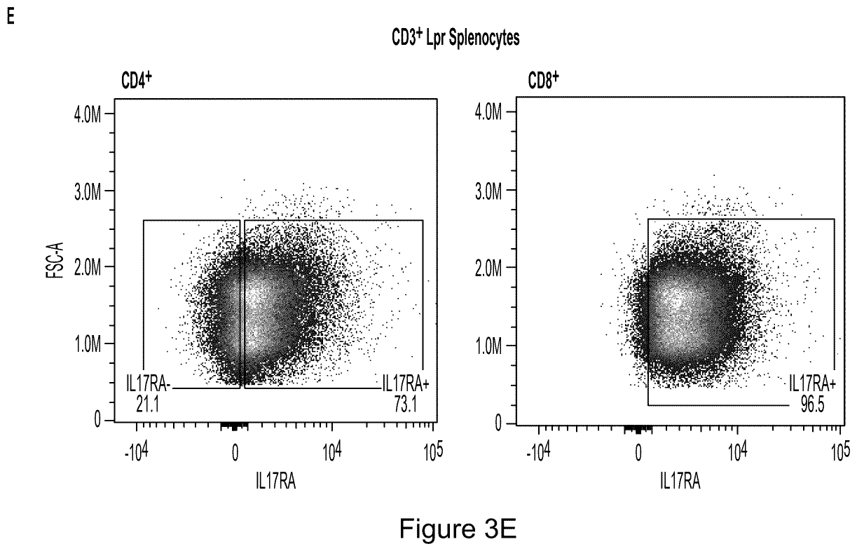

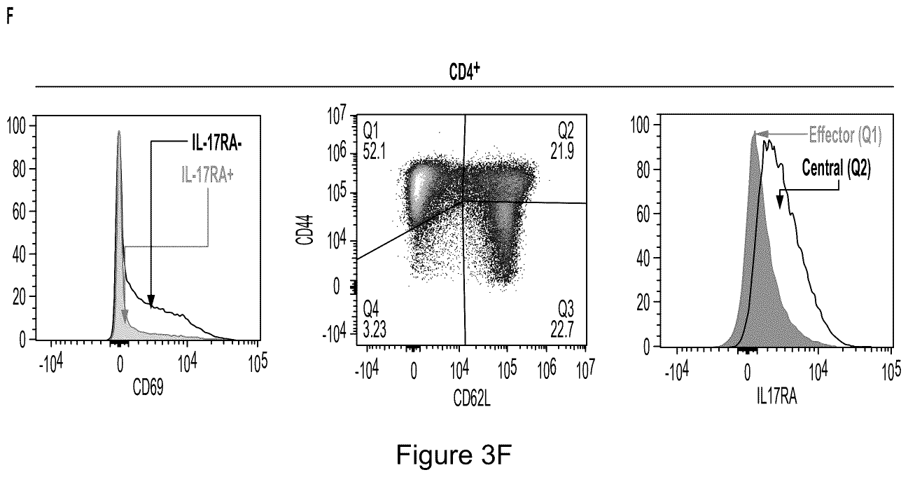

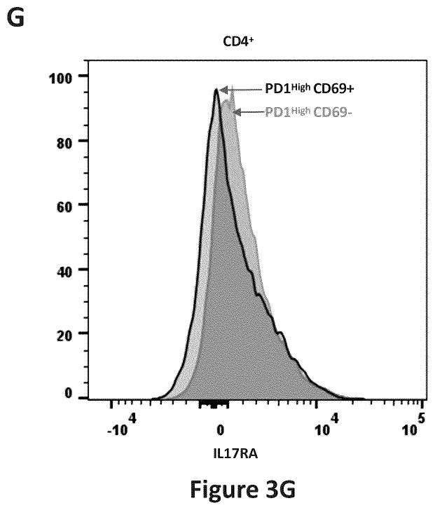

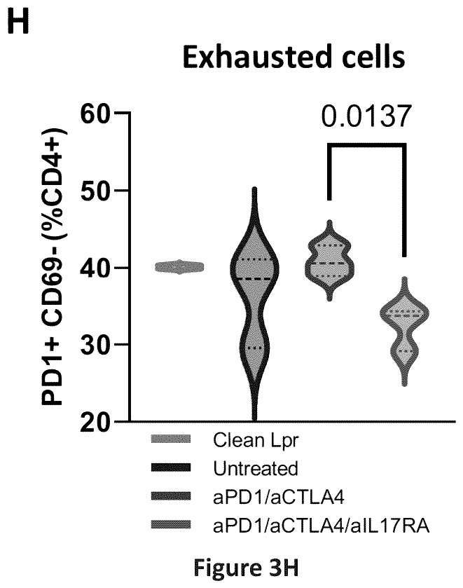

Illustrated in FIG. 3A is the IL17 ligand and receptor family (28-31). It has been previously reported that blocking IL17A enhanced the anti-tumor response of anti-PD-1 therapy (35). Combined with the data on neutralizing IL25, described herein is the aim to achieve higher efficacy by targeting IL17RA, a receptor for both IL17A and IL25 (IL17E). To test this hypothesis, an in-vivo experiment was performed using an anti-IL17RA neutralizing antibody (FIG. 3B). Tumor growth curves show enhanced anti-tumor response in mice treated with anti-IL17RA antibody in addition to anti-PD-1 and anti-CTLA-4 combination of antibodies (FIG. 3Ci). Additionally, compared to mice that received the same dose of anti-IL25 antibody, mice treated with anti-IL17RA had smaller tumors (FIG. 3Cii). irAEs analysis at 3.5 weeks reveals that anti-IL17RA treatment decreased immune infiltration in the liver and lung (FIG. 3D). Altogether, IL17RA neutralization promotes MC38 tumor regression while preventing immune checkpoint inhibitor induced hepatitis and pneumonitis in B6/lpr mice.

### Example 4: Neutralizing IL17RA Leads to More T Cell Activation and Less T Cell Exhaustion

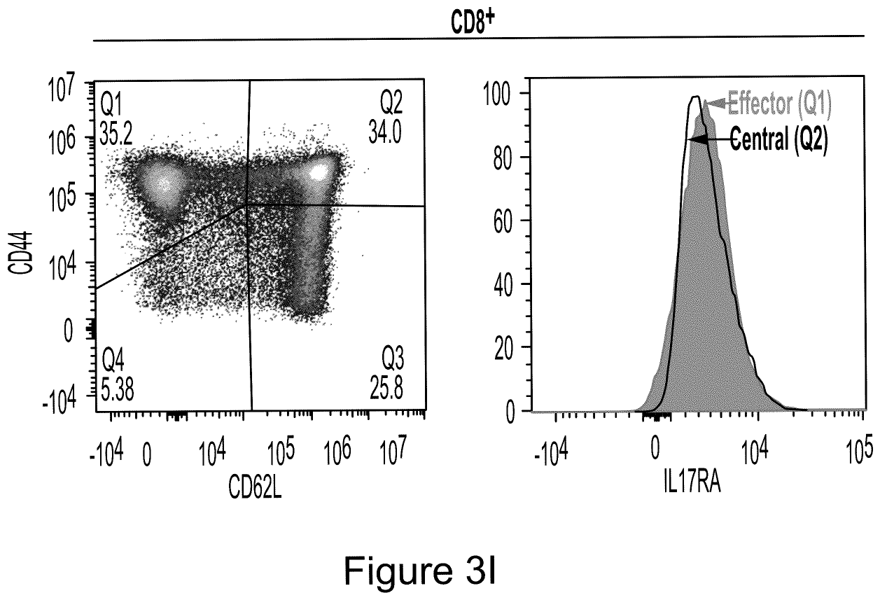

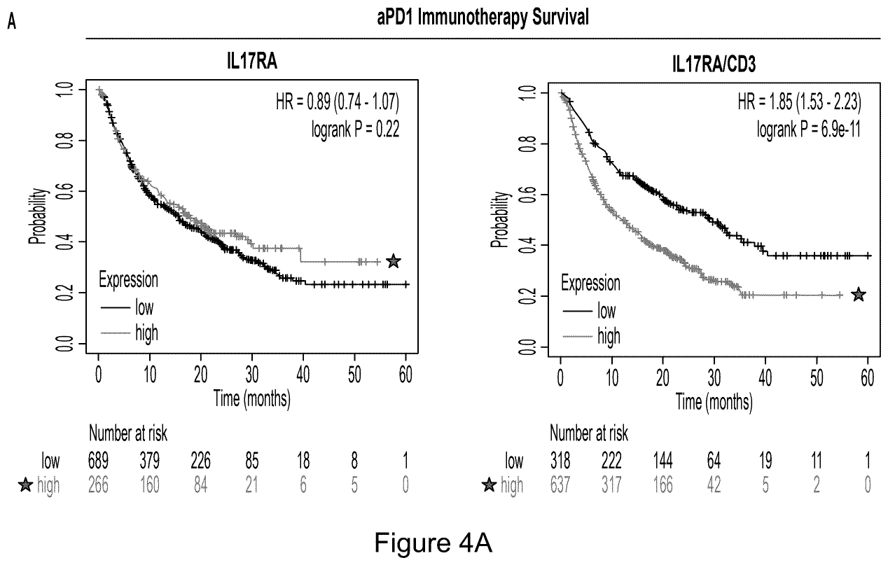

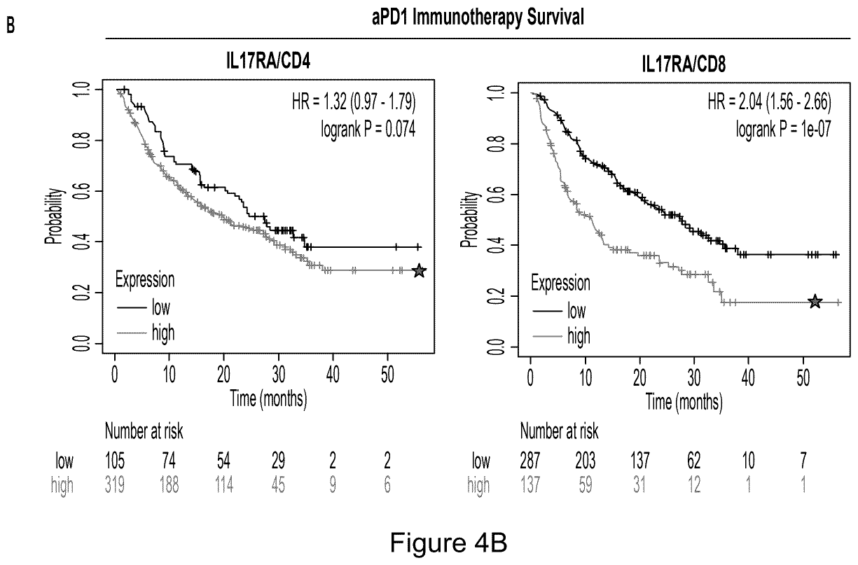

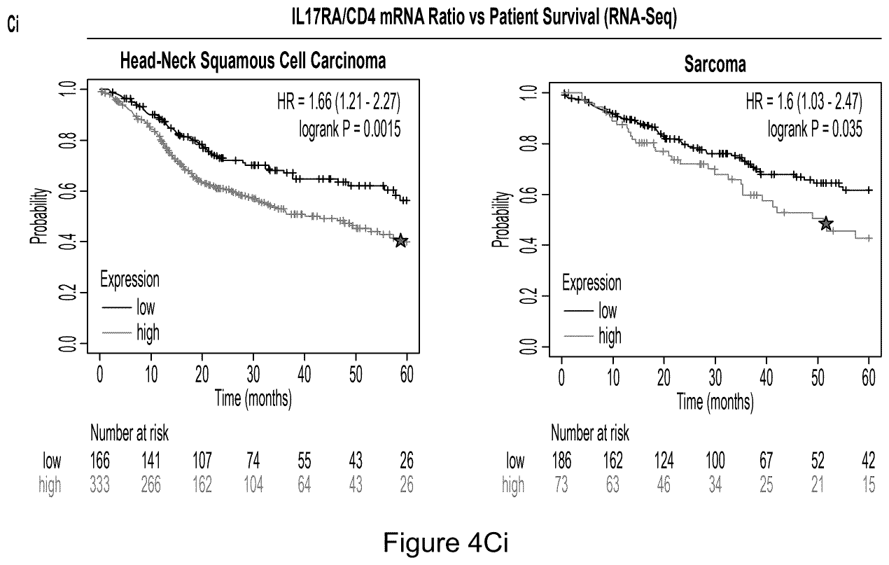

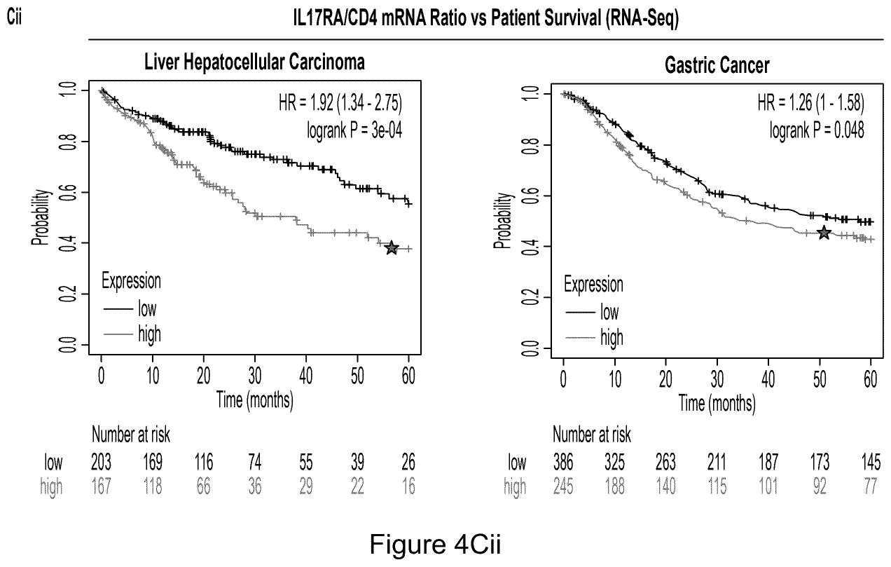

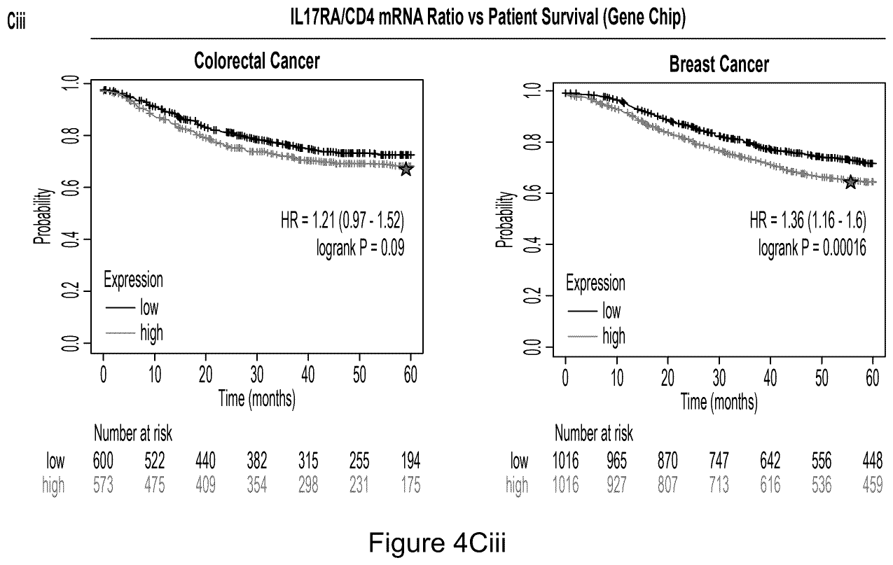

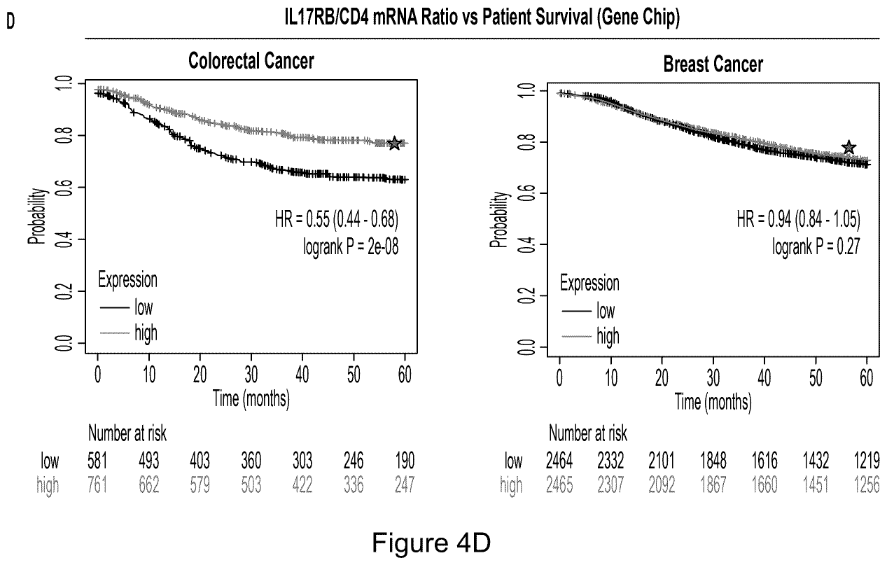

To characterize B6/lpr mice, flow cytometry was performed on splenocytes collected from B6/lpr mice. It was observed that 73% of the CD4⁺ T cells and 97% of the CD8⁺ T cells were positive for IL17RA (FIG. 3E). In the CD4⁺ T cells, it was discovered that IL17RA negative cells expressed more CD69 (activation marker) than IL17RA positive cells (FIG. 3F). Moreover, among the CD4⁺ positive T cells, effector memory cells had lower expression of IL17RA than central memory cells (FIG. 3F). also It was also found that exhausted cells express more IL17RA than activated cells (FIG. 3G). Flow cytometry splenocytes collected from the previously described in-vivo experiment reveal that neutralizing IL17RA offers protection against CD4⁺ T-cell exhaustion (FIG. 4H). The data (FIG. 3E), shows CD8⁺ T cells are 97% IL17RA positive. Hence, the differences between IL17RA positive and IL17RA negative cells was not evaluated in the CD8⁺ T cell population. Moreover, there is no difference in IL17RA expression between central and effector CD8⁺ T cells (FIG. 31).

### Example 5: T Cell IL17RA Gene Expression Correlates with Worse Patient Outcomes

The study of IL17RA to human subjects was extended. Analysis of anti-PD-1 immunotherapy patient survival data shows no significant survival difference between overall IL17RA high and IL17RA low patients; differences occur when looking at IL17RA in the T cell compartment (FIG. 4A). Both IL17RA in CD4 and IL17RA in CD8 T cells inversely correlate with anti-PD-1 immunotherapy patient survival (FIG. 4B). NextRNA-seq and gene chip data of IL17RA were evaluated in CD4 T cells and patient survival in multiple cancer types. Data consistently reveals a higher probability of survival in patients with lower IL17RA in CD4 T cells across head-neck squamous cell carcinoma, sarcoma, liver hepatocellular carcinoma, gastric cancer, colorectal cancer, and breast cancer (FIG. 4Ci, 4Cii, 4Ciii). Even though others have reported IL17RB expression is associated with poor prognosis in the oral cancer (36), the same tendency for IL17RB in CD4 T cells and colorectal or breast cancer patient survival was not observed (FIG. 4D). Altogether, data suggests cancer patients could benefit from IL17RA neutralization.

Cancer remains the second leading cause of death in the US, accounting for 25% of all deaths nationwide. ICI bolsters immune cells' ability to target cancer cells and has improved cancer treatments immensely. Anti-PD-1 and CTLA-4 antibodies provide excellent clinical efficacy, as evidenced by tumor regression and increased overall patient survival. While these therapies are highly successful in some patients, they fail or cause irAEs in others. More than fifty percent of the patients who receive ICI develop irAEs characterized by multiple organ inflammation with T cell infiltrates. irAEs may occur at any time during ICI treatment but are most commonly observed within the first three months (37). These clinically observed irAEs share similarities with primary autoimmune diseases. The contribution of PD-1 to peripheral tolerance is a significant mechanism for protection against the expansion of self-reactive T cell clones and autoimmune disease (38).

Several studies highlight cytokine dysregulation in irAE-affected tissues (34, 39, 40). Rahma et al., analyzed the levels of thirty-four cytokines in 52 melanoma patients receiving ICI who developed irAEs. There were no differences in cytokine levels between patients with grade 1-2 and grade 3-4 irAEs. Patients with irAEs dermatitis had higher baseline Angiopoictin and CD40L, and patients with pneumonitis had more elevated baseline IL17. They observed a fold-change increase in the levels of many cytokines in patients who developed irAEs before receiving steroids. Another study evaluated the efficacy of Tocilizumab, an anti-IL6, in twenty-two patients with irAEs. The average time to irAEs resolution was one week (range 1-93). Clinical improvement or benefit was demonstrated in most patients. However, prospective trials are required to evaluate its efficacy and, not less significantly, the impact on cancer outcomes compared with standard strategies. TNF inhibitors are commonly used to treat inflammatory diseases and have also been successfully adopted as second-line agents to treat irAEs refractory to steroids. Similarly, whether TNF inhibition can be safely used to treat irAEs without promoting cancer progression, either by compromising ICI therapy efficacy or via another route, remains an open question.

It has been previously reported that blocking IL17A enhanced the anti-tumor response of anti-PD-1 therapy. Combined with the data on neutralizing IL25, described herein is a method for achieving higher efficacy by targeting IL17RA, the receptor for both IL17A and IL25. Indeed, it is shown that inhibition of IL17RA ameliorated ICI-induced pneumonitis and promoted antitumor activity. Remarkably, analysis of data from the TCGA (www.cancer.gov/ccg/research/genome-sequencing/tcga) revealed that high expression of IL17RA is associated with worse prognosis and shorter survival in patients having multiple types of cancers. Thus, neutralizing IL17RA can be beneficial in treating patients with cancer and irAEs.

This work is significant for several reasons. Current treatments of irAEs are based on protocols used to treat phenotypically similar conditions, with corticosteroids being the first-line intervention. These nonspecific treatments are associated with toxicities, and increasing evidence suggests that they also interfere with the anti-tumor immune receptors induced by the ICI. This work defined the role of IL17RA in irAEs and has great significance because of the need to develop therapeutic approaches that exploit differences between anti-tumor immunity and the processes resulting in irAEs.

This data showed that neutralizing IL17RA with antibodies inhibited tumor growth and resolved irAEs. Described herein is the administration of anti-IL17RA antibodies to ameliorate irAEs and reduce tumor size. These findings suggest that IL17RA may serve dual roles in treating tumors responsive to ICI, allowing for extended ICI therapy by suppressing immune-related toxicities.

### Methods

| Material | Source | Catalog Number |
|---|---|---|
| Mice | | |
| B6 Lpr mouse | The Jackson | 000482 |
| | Laboratory | |
| B6 mouse | The Jackson | 000664 |
| | Laboratory | |
| In-Vivo Antibodies/Drugs | | |
| InVivoMab anti-mouse | BioXCell | BE0146 |
| PD-1 (CD279) | | |
| InVivoMab anti-mouse | BioXCell | BE0131 |
| CTLA-4 (CD152) | | |
| Predisolone | Sigma-Aldrich | P6004 |
| InVivoMAb anti-mouse | BioXCell | BE0058 |
| TNFα | | |
| InVivoMAb anti-mouse IL-6 | BioXCell | BE0046 |
| Mouse IL-17RA/IL-17R | R&D Systems | MAB4481 |
| Flow/IHC Antibodies | | |
| Alexa Fluor 488 anti-mouse | BioLegend | 100210 |
| CD3 | | |
| Brilliant Violet 510 anti- | BioLegend | 100553 |
| mouse CD4 | | |
| PerCP/Cy5.5 anti-mouse | BioLegend | 100734 |
| CD8a | | |
| Brilliant Violet 421 anti- | BioLegend | 103040 |
| mouse/human CD44 | | |
| Brilliant Violet 711 anti- | BioLegend | 104445 |
| mouse CD62L | | |
| PE/Cy7 anti-mouse CD279 | BioLegend | 109110 |
| (PD-1) | | |
| APC anti-mouse CD69 | BioLegend | 104514 |
| CD3 antibody [SP7] | GeneTex | GTX16669 |
| Reagents | | |
| DMEM, 1X | Corning | 10-013-CVvc |
| HI FBS | Gibco | 10438-026 |
| Penicillin Streptomycin | Corning | 30-001-CI |
| HEPES Buffer | Corning | 25-060-CI |
| PBS, 1X | Corning | 21-040-CV |
| 0.25% Trypsin | Corning | 25-053-CI |
| Dimethyl Sulfoxide | Fisher Bioreagents | BP231-1 |
| Lymphoprep | STEM CELL | 07801 |
| | Technologies | |
| RPMI 1640, 1X | Corning | 10-040-CVR |
| ACK Lysing Buffer | Gibco | A10492-01 |
| TruStain Monocyte Blocker | BioLegend | 426103 |
| TruStain FcX ™ PLUS (anti- | BioLegend | 156604 |
| mouse CD16/32) Antibody | | |
| TruStain FcX ™ (anti-mouse | BioLegend | 101320 |
| CD16/32) Antibody | | |
| Brilliant Stain Buffer | BD Horizon | 563794 |
| 10% Neutral Buffered | Thermo Scientific | 5701 |
| Formalin | | |
| Ethanol, Anhydrous | Fisher Bioreagents | A405P-4 |
| Kits | | |
| Mycoplasma Detection Kit | InvivoGen | rep-mys-50 |
| LIVE/DEAD ™ Fixable Blue | Invitrogen | L34962 |
| Dead Cell Stain Kit, for UV | | |
| excitation | | |
| True-Nuclear ™ Transcription | BioLegend | 424401 |
| Factor Buffer Set | | |
| Zombie UV ™ Fixable | BioLegend | 423107 |
| Viability Kit | | |
| IL-25 (IL-17E) Mouse | Invitrogen | EPX01A-26046-901 |
| ProcartaPlexTM Simplex Kit | | |
| Cytokine & Chemokine 36- | Invitrogen | EPX360-26092-901 |
| Plex Mouse 1 | | |
| ProcartaPlexTM Panel 1A | | |
| Software/Computer Based | | |
| Tools | | |
| HALO | Indica Labs | |
| FlowJo_V10 | BD Biosciences | |
| UMAP | Scikit-Learn | |
| Python | Python Software | |
| | Foundation | |
| Kaplan-Meier Plotter | KMPlot.com | |
| Prism 9 | GraphPad | |

### Cell Culture

The murine colon adenocarcinoma cancer cell line MC38 (Kerafast) was cultured in DMEM medium (Corning) with 10% FBS (Gibco) and 1% Pen-Strep (Corning). The murine breast cancer cell line E0771 (Robert F. Schwabe) was cultured in DMEM medium (Corning) with 10% FBS (Gibco), 1% Pen-Step (Corning), and 20 mM HEPES (Corning). Cells were grown in a 37° C. incubator and routinely examined for mycoplasma using mycoplasma detection kit (InvivoGen).

### Mice Breeding

B6 Lpr mice purchased from JAX (Cat #) were housed in the Columbia Institute of Comparative Medicine animal facility under protocol AC-AABO7553. Breeding cages were set up with two females and one male. Litters are routinely genotyped through PCR using Fas gene primers—oIMR1678 (5′-GTA AAT AAT TGT GCT TCG TCA G-3′ (SEQ ID NO: 303)) as common primer, oIMR1679 (5′-TAG AAA GGT GCA CGG GTG TG-3′ (SEQ ID NO: 304)) for Fas^{LPr} mutant, and oIMR 1680 (5′-CAA ATC TAG GCA TTA ACA GTG-3′ (SEQ ID NO: 305)) for FasWT. Mutant mice with homozygote alleles were recruited as new breeders or used for experiments. B6 WT mice were purchased from JAX and used directly.

In-Vivo Tumor and irAEs Model

For colon cancer tumor and irAEs combined studies, 2×10⁵ MC38 cells were suspended in cold, sterile PBS (Corning) and implanted subcutaneously into the right flank of 7-12 weeks B6 Lpr mice. Tumor width and length was measured using a digital caliper and calculate volume using the formula volume=(shorter dimension) 2×(larger dimension)/2. Mice with tumor smaller then 60 mm3 have been included and randomly assigned to different groups: untreated control group (n=12), aPD1/aCTLA4 induced control group (n=13), prednisolone treatment group (n=8), aIL6 treatment group (n=5), aTNFα treatment group (n=5), and aIL17RA treatment group (n=3). Anti-PD1 and anti-CTLA4 immune checkpoint inhibitor treatments are initiated once the tumor is visible. 200 μg anti-PD-1 (BioXCell) and 200 μg anti-CTLA-4 (BioXCell) antibodies are administered bi-weekly through intraperitoneal injections. In addition, oral prednisolone (Sigma), intraperitoneal anti-IL6 (BioXCell; MP5-20F3), intraperitoneal anti-TNFα (BioXCell; XT3.11), and intraperitoneal anti-IL17RA (R&D; 657603) were administered. The order of mice being treated and measured was random.

The cages were housed on the same rack to minimize potential confounder variables. Tumors and animal health have been closely monitored by researchers (not blinded) and veterinarians at Columbia Institute of Comparative Medicine. Humane endpoint euthanasia was provided when tumor volume reached over 2000 mm³ or when tumor became extremely ulcerated.

### Luminex Assay and Analysis

Peripheral blood was collected from the heart of the mice post-cuthanizing. Blood serum was isolated by centrifugation at 10,000×g for 10 minutes. Isolated serum was stored at −80° C. before the Luminex assay. Columbia Biomarkers Core Laboratory performed Luminex magnetic bead assay using 36-plex mouse panel (Invitrogen) and IL-25 simplex (Invitrogen) kits.

Each sample was run in duplicates. The coefficient of variation (CV) between duplicated samples was calculated. Repeated samples with CV>20% were eliminated. Models with cytokine/chemokine levels below the lower level of detection (LLOD) were assigned a value equal to LLOD/V2. Samples with cytokine/chemokine levels above the upper level of detection (ULOD) were given the value of ULOD. Cytokines/chemokines with more than 40% samples of undetectable values were discarded. Cytokine/chemokine values underwent a natural log transformation for normal distributions. Samples outside of their treatment groups' respective 95% CI were eliminated. The data presented only shows stratified data of interest.

### Histology Study

At the in-vivo endpoint, mice were transcardially perfused with 10 ml saline to clear blood. Heart, liver, lung, colon, pancreas, and tumor were collected and washed in 10 ml PBS before being transferred to 10 ml 10% formalin (Thermo Scientific). After >24 hours of fixation in formalin, the tissues were transferred to 70% histology-grade anhydrous ethanol (Fisher Bioreagents). Samples were then sent to Columbia Molecular Pathology Shared Resource (MPSR) for slicing, hematoxylin-cosin (H&E) stain, and paraffin embedding. H&E slides were viewed under a light microscope. Immune cell infiltration severity scores were graded by two trained experts on a scale of 0-3 (32).

### Immunohistochemistry

Immune-blank slides are made from paraffin-embedded blocks and stained with anti-CD3 (GencTex) by Histo Wiz. Scanned images of IHC slides are processed using HALO (Indica Labs) for artificial intelligence CD3⁺ T-cell labeling (41).

### Flow Cytometry and Gating Strategy

Livers and tumors were harvested at the endpoint of in-vivo experiments after perfusion. Livers were smashed through 100 μM filters using syringe plungers and collected in 20 ml of FACS buffer (2% FBS in PBS) in a 50 ml centrifuge tube. Liver cells pellet after centrifugation at 50×g for 2 minutes (brake off). The supernatant was collected and washed with PBS. Tumors were diced into small pieces using a scalpel blade and transferred to a 5 ml digestion mixture (500 ml PBS+500 mg collagenase D+25 ml FCS+10 mg DNasc) in 15 ml conical tubes. The samples were incubated in a 37° C. water bath for 30 minutes. Digested tissue samples were vortexed vigorously before being smashed through 45 μM filters and collected using 5 ml RPMI medium (Corning) containing 10% FBS and 1% Pen-Strep. Following centrifugation and collection, the cells were washed once with PBS. Liver and tumor lymphocytes were isolated using Lymphoprep (Stem Cell Technologies) following Lymphoprep's standard protocol. Isolated lymphocytes were washed with PBS and used immediately for flow cytometry. Lymphocytes isolated from the liver and tumor were stained for 34-plex flow cytometry using a protocol consisting of four parts: (I) live/dead staining, (II) surface staining, (III) fixation/permeabilization, and (iv) intracellular staining. Live/dead stain was performed with a live/dead fixable blue dead cell stain kit (Invitrogen). Following live/dead colors, Fc receptors, and monocytes were blocked using TruStain FcX PLUS (BioLegend) and TruStain Monocyte Blocker (BioLegend). Then, the cells were stained for cell surface proteins using fluorophore conjugated antibodies: TCR-B BUV395, CD103 BUV496, CD44 BUV563, PD-1 BUV615, Nrp1 BUV661, CD4 BUV805, CD39 BV421, IA-IE PacBluc, ST2 BV480, CD8 PacOrange, CD62L BV570, CD11c BV605, ICOS BV650, CXCR3 BV711, KLRGI BV750, PD-L1 BV785, CD45 A532, Sca-1 PerCP, Ly6C PerCP-Cy5.5, CD206 PerCPeF710, NK1.1 PE-Cy5, B220 PE/Fire 810, CD69 SN685, CD11b A700, F4/80 APC-Fire750, and CD38 APC-Fire810. Following cell surface staining, the cells were fixed/permeabilized using a Fixation/Permeabilization Buffer Set (BioLegend). Post-fix and perm, the cells were stained for intracellular proteins using fluorophore-conjugated antibodies: Ki67 BUV737, INOS FITC, TOX PE, Ly6G SYG593, Helios PE-Dazzle594, FoxP3 PE-Cy7, and TCF-1 APC. Data were acquired using Cytek 5L Aurora and analyzed using FlowJo and Python UMAP. Spleen was harvested at the endpoint of in-vivo experiments. Spleen tissue was smashed through 70 μM filters, washed twice in FACS, resuspended in ACK lysis buffer, and washed twice in FACS. Spleen-mixed white blood cells were used immediately for flow cytometry or frozen with 20% DMSO (Fisher Bioreagents) in FBS (Gibco). Dead cells in the mixed white blood cells isolated from the spleens were stained using Zombie UV™ Fixable Viability Kit, while the Fc receptors were blocked using TruStain FcX (BioLegend). Cell surface proteins were stained with C fluorophore-conjugated-antibodies, CD3 AF488 (BioLegend), CD4 BV510 (BioLegend), CD8 Percp/Cy5.5 (BioLegend), CD44 BV421 (BioLegend), CD62L BV711 (BioLegend), PD-1 PE-Cy7 (BioLegend), CD69 APC, and IL17RA PE, for CD4⁺/CD8⁺ T-cell identification, T_{Naive}/T_{CM}/T_{EM}/TEMRA subset differentiation, activation/exhaustion observation, and IL17RA expression assessment. Data were recorded using Cytek 5L Aurora and analyzed with FlowJo.

### Kaplan-Meier Plots

KM Plotter was used to investigate IL17RA's function in human subjects with cancer. IL17RA/CD4 mRNA expression ratio vs. survival was mapped for breast and colon cancer using gene chip data (60-month follow-up threshold). IL 17RA/CD4 mRNA expression ratio vs. survival was mapped for liver hepatocellular carcinoma, liver cancer, head-neck squamous cell carcinoma, and sarcoma using RNA sequencing data (60-month follow-up threshold). IL17RA/CD4 and IL17RA/CD8 gene expression ratio vs. survival was mapped for patients only receiving PD-1 immunotherapy regardless of which aPDI drug (Kaplan-Meier Plotter).

### Statistical Analysis

Data were analyzed using Prism 9 (GraphPad) and presented as mean or mean±SEM. For comparison analysis of immune infiltration/cytokine level/tumor volume on day 18 bar graphs, an unpaired two-tailed t-test was performed. Additionally, an unpaired t-test performed on tumor volume on day 18 used Welch's correction. Survival probabilities were analyzed using the Kaplan-Meier estimate.

### REFERENCES

1. Topalian, S. L., F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, and M. Sznol. 2012. Safety, activity, and immune correlates of anti-PD-1 antibody in cancer. N. Engl. J. Med. 366:2443-2454.
2. Powles, T., J. P. Eder, G. D. Fine, F. S. Braitch, Y. Loriot, C. Cruz, J. Bellmunt, H. A. Burris, D. P. Petrylak, S. Teng, X. Shen, Z. Boyd, P. S. Hegde, D. S. Chen, and N. J. Vogelzang. 2014. MPDL3280A (anti-PD-L1) treatment leads to clinical activity in metastatic bladder cancer. Nature 515:558-562.
3. Váraljai, R., L. Zimmer, Y. Al-Matary, P. Kaptein, L. J. Albrecht, B. Shannan, J. C. Brasc, D. Gusenleitner, T. Amaral, N. Wyss, J. Utikal, L. Flatz, F. Rambow, H. C. Reinhardt, J. Dick, D. R. Engel, S. Horn, S. Ugurel, W. Sondermann, E. Livingstone, A. Sucker, A. Paschen, F. Zhao, J. M. Placke, J. M. Klose, W. P. Fendler, D. S. Thommen, I. Helfrich, D. Schadendorf, and A. Roesch. 2023. Interleukin 17 signaling supports clinical benefit of dual CTLA-4 and PD-1 checkpoint inhibition in melanoma. Nat. Cancer 4:1292-1308.
4. Duan, J., L. Cui, X. Zhao, H. Bai, S. Cai, G. Wang, Z. Zhao, J. Zhao, S. Chen, J. Song, C. Qi, Q. Wang, M. Huang, Y. Zhang, D. Huang, Y. Bai, F. Sun, J. J. Lee, Z. Wang, and J. Wang. 2020. Use of Immunotherapy With Programmed Cell Death 1 vs Programmed Cell Death Ligand 1 Inhibitors in Patients With Cancer: A Systematic Review and Meta-analysis. JAMA Oncol. 6:375-384.
5. Kuol, N., L. Stojanovska, K. Nurgali, and V. Apostolopoulos. 2017. The mechanisms tumor cells utilize to evade the host's immune system. Maturitas 105:8-15.
6. Varga, A., S. A. Piha-Paul, P. A. Ott, J. M. Mehnert, D. Berton-Rigaud, A. Morosky, G. Q. Zhao, R. A. Rangwala, and D. Matei. 2017. Pembrolizumab in patients (pts) with PD-L1-positive (PD-L1⁺) advanced ovarian cancer: Updated analysis of KEYNOTE-028. J. Clin. Oncol. 35:5513-5513.
7. Matulonis, U. A., R. Shapira-Frommer, A. Santin, A. S. Lisyanskaya, S. Pignata, I. Vergote, F. Raspagliesi, G. S. Sonke, M. Birrer, D. M. Provencher, J. Schouli, N. Colombo, A. González-Martín, A. Oaknin, P. B. Ottevanger, V. Rudaitis, K. Katchar, Z. Wang, J. Ruman, and J. A. Ledermann. 2018. Antitumor activity and safety of pembrolizumab in patients with advanced recurrent ovarian cancer: Interim results from the phase 2 KEYNOTE-100 study. J. Clin. Oncol. 36:5511-5511.
8. Disis, M. L., M. R. Patel, S. Pant, E. P. Hamilton, A. C. Lockhart, K. Kelly, J. T. Beck, M. S. Gordon, G. J. Weiss, M. H. Taylor, J. Chaves, A. C. Mita, K. M. Chin, A. von Heydebreck, J.-M. Cuillerot, and J. L. Gulley. 2016. Avelumab (MSB0010718C; anti-PD-L1) in patients with recurrent/refractory ovarian cancer from the JAVELIN Solid Tumor phase Ib trial: Safety and clinical activity. J. Clin. Oncol. 34:5533-5533.
9. Liu, J. F., C. Herold, W. Luo, R. Penson, N. Horowitz, P. Konstantinopoulos, C. Castro, J. Curtis, U. A. Matulonis, S. Cannistra, and D. S. Dizon. 2018. A phase II trial of combination nivolumab and bevacizumab in recurrent ovarian cancer. Ann. Oncol. 29: viii334-viii335.
10. Butte, M. J., M. E. Keir, T. B. Phamduy, A. H. Sharpe, and G. J. Freeman. 2007. Programmed death-1 ligand 1 interacts specifically with the B7-1 costimulatory molecule to inhibit T cell responses. Immunity 27:111-122.
11. Butte, M. J., V. Peña-Cruz, M.-J. Kim, G. J. Freeman, and A. H. Sharpe. 2008. Interaction of human PD-L1 and B7-1. Mol. Immunol. 45:3567-3572.
12. Chaudhri, A., Y. Xiao, A. N. Klee, X. Wang, B. Zhu, and G. J. Freeman. 2018. PD-L1 Binds to B7-1 Only In Cis on the Same Cell Surface. Cancer Immunol. Res. 6:921-929.
13. Yang, J., L. V. Riella, S. Chock, T. Liu, X. Zhao, X. Yuan, A. M. Paterson, T. Watanabe, V. Vanguri, H. Yagita, M. Azuma, B. R. Blazar, G. J. Freeman, S. J. Rodig, A. H. Sharpe, A. Chandraker, and M. H. Sayegh. 2011. The novel costimulatory programmed death ligand 1/B7.1 pathway is functional in inhibiting alloimmune responses in vivo. J. Immunol. Baltim. Md 1950 187:1113-1119.
14. Paterson, A. M., K. E. Brown, M. E. Keir, V. K. Vanguri, L. V. Riella, A. Chandraker, M. H. Sayegh, B. R. Blazar, G. J. Freeman, and A. H. Sharpe. 2011. The programmed death-1 ligand 1: B7-1 pathway restrains diabetogenic effector T cells in vivo. J. Immunol. Baltim. Md 1950 187:1097-1105.
15. Ghiotto, M., L. Gauthier, N. Serriari, S. Pastor, A. Truneh, J. Nunès, and D. Olive. 2010. PD-L1 and PD-L2 differ in their molecular mechanisms of interaction with PD-1. Int. Immunol. 22:651-660.
16. Kuol, N., J. Godlewski, Z. Kmiec, S. Vogrin, S. Fraser, V. Apostolopoulos, and K. Nurgali. 2023. Cholinergic signaling influences the expression of immune checkpoint inhibitors, PD-L1 and PD-L2, and tumor hallmarks in human colorectal cancer tissues and cell lines. BMC Cancer 23:971.
17. Sumitomo, R., C.-L. Huang, M. Fujita, H. Cho, and H. Date. 2022. Differential expression of PD-L1 and PD-L2 is associated with the tumor microenvironment of TILs and M2 TAMs and tumor differentiation in non-small cell lung cancer. Oncol. Rep. 47:73.
18. Xiao, Y., S. Yu, B. Zhu, D. Bedoret, X. Bu, L. M. Francisco, P. Hua, J. S. Duke-Cohan, D. T. Umetsu, A. H. Sharpe, R. H. DeKruyff, and G. J. Freeman. 2014. RGMb is a novel binding partner for PD-L2 and its engagement with PD-L2 promotes respiratory tolerance. J. Exp. Med. 211:943-959.
19. Sandigursky, S., and A. Mor. 2018. Immune-Related Adverse Events in Cancer Patients Treated With Immune Checkpoint Inhibitors. Curr. Rheumatol. Rep. 20:65.
20. Das, S., and D. B. Johnson. 2019. Immune-related adverse events and anti-tumor efficacy of immune checkpoint inhibitors. J. Immunother. Cancer 7:306.
21. Arnaud-Coffin, P., D. Maillet, H. K. Gan, J.-J. Stelmes, B. You, S. Dalle, and J. Peron. 2019. A systematic review of adverse events in randomized trials assessing immune checkpoint inhibitors. Int. J. Cancer 145:639-648.
22. Mor, A., and M. Strazza. 2022. Bridging the Gap: Connecting the Mechanisms of Immune-Related Adverse Events and Autoimmunity Through PD-1. Front. Cell Dev. Biol. 9:790386.
23. Ribas, A., I. Puzanov, R. Dummer, D. Schadendorf, O. Hamid, C. Robert, F. S. Hodi, J. Schachter, A. C. Pavlick, K. D. Lewis, L. D. Cranmer, C. U. Blank, S. J. O'Day, P. A. Ascierto, A. K. S. Salama, K. A. Margolin, C. Loquai, T. K. Eigentler, T. C. Gangadhar, M. S. Carlino, S. S. Agarwala, S. J. Moschos, J. A. Sosman, S. M. Goldinger, R. Shapira-Frommer, R. Gonzalez, J. M. Kirkwood, J. D. Wolchok, A. Eggermont, X. N. Li, W. Zhou, A. M. Zernhelt, J. Lis, S. Ebbinghaus, S. P. Kang, and A. Daud. 2015. Pembrolizumab versus investigator-choice chemotherapy for ipilimumab-refractory melanoma (KEYNOTE-002): a randomised, controlled, phase 2 trial. Lancet Oncol. 16:908-918.
24. Gupta, A., K. M. De Felice, E. V. Loftus, and S. Khanna. 2015. Systematic review: colitis associated with anti-CTLA-4 therapy. Aliment. Pharmacol. Ther. 42:406-417.
25. Weber, J. S., K. C. Kähler, and A. Hauschild. 2012. Management of immune-related adverse events and kinetics of response with ipilimumab. J. Clin. Oncol. Off. J. Am. Soc. Clin. Oncol. 30:2691-2697.
26. Blum, S. M., S. J. Rouhani, and R. J. Sullivan. 2023. Effects of immune-related adverse events (irAEs) and their treatment on antitumor immune responses. Immunol. Rev. 318:167-178.
27. Paderi, A., E. Gambale, C. Botteri, R. Giorgione, D. Lavacchi, M. Brugia, F. Mazzoni, E. Giommoni, S. Bormioli, A. Amedei, S. Pillozzi, M. Matucci Cerinic, and L. Antonuzzo. 2021. Association of Systemic Steroid Treatment and Outcome in Patients Treated with Immune Checkpoint Inhibitors: A Real-World Analysis. Mol. Basel Switz. 26:5789.
28. Maslov, D. V., K. Tawagi, M. Kc, V. Simenson, H. Yuan, C. Parent, A. Bamnolker, R. Goel, Z. Blake, M. R. Matrana, and D. H. Johnson. 2021. Timing of steroid initiation and response rates to immune checkpoint inhibitors in metastatic cancer. J. Immunother. Cancer 9: c002261.
29. Leone, G. M., K. Mangano, M. C. Petralia, F. Nicoletti, and P. Fagone. 2023. Past, Present and (Foreseeable) Future of Biological Anti-TNF Alpha Therapy. J. Clin. Med. 12:1630.
30. Bar-Hai, N., G. Ben-Betzalel, R. Stoff, S. Grynberg, J. Schachter, R. Shapira-Frommer, and N. Asher. 2023. Better Late Than Never: The Impact of Steroidal Treatment on the Outcome of Melanoma Patients Treated with Immunotherapy. Cancers 15:3041.
31. Bass, A. R., N. Abdel-Wahab, P. D. Reid, J. A. Sparks, C. Calabrese, D. P. Jannat-Khah, N. Ghosh, D. Rajesh, C. A. Aude, L. Gedmintas, L. MacFarlane, S. Arabelovic, A. Falohun, K. Mushtaq, F. A. Haj, A. Diab, A. A. Shah, C. O. Bingham, K. K. Chan, and L. C. Cappelli. 2023. Comparative safety and effectiveness of TNF inhibitors, IL6 inhibitors and methotrexate for the treatment of immune checkpoint inhibitor-associated arthritis. Ann. Rheum. Dis. 82:920-926.
32. Adam, K., A. Iuga, A. S. Tocheva, and A. Mor. 2021. A novel mouse model for checkpoint inhibitor-induced adverse events. PloS One 16: e0246168.
33. Perez-Ruiz, E., L. Minute, I. Otano, M. Alvarez, M. C. Ochoa, V. Belsue, C. de Andrea, M. E. Rodriguez-Ruiz, J. L. Perez-Gracia, I. Marquez-Rodas, C. Llacer, M. Alvarez, V. de Luque, C. Molina, A. Teijeira, P. Berraondo, and I. Melero. 2019. Prophylactic TNF blockade uncouples efficacy and toxicity in dual CTLA-4 and PD-1 immunotherapy. Nature 569:428-432.
34. Zhou, Y., Y. B. Medik, B. Patel, D. B. Zamler, S. Chen, T. Chapman, S. Schneider, E. M. Park, R. L. Babcock, T. T. Chrisikos, L. M. Kahn, A. M. Dyevoich, J. E. Pineda, M. C. Wong, A. K. Mishra, S. H. Cass, A. P. Cogdill, D. H. Johnson, S. B. Johnson, K. Wani, D. A. Ledesma, C. W. Hudgens, J. Wang, M. A. Wadud Khan, C. B. Peterson, A. Y. Joon, W. Peng, H. S. Li, R. Arora, X. Tang, M. G. Raso, X. Zhang, W. C. Foo, M. T. Tetzlaff, G. E. Diehl, K. Clise-Dwyer, E. M. Whitley, M. M. Gubin, J. P. Allison, P. Hwu, N. J. Ajami, A. Diab, J. A. Wargo, and S. S. Watowich. 2023. Intestinal toxicity to CTLA-4 blockade driven by IL-6 and myeloid infiltration. J. Exp. Med. 220: e20221333.
35. Liu, C., R. Liu, B. Wang, J. Lian, Y. Yao, H. Sun, C. Zhang, L. Fang, X. Guan, J. Shi, S. Han, F. Zhan, S. Luo, Y. Yao, T. Zheng, and Y. Zhang. 2021. Blocking IL-17A enhances tumor response to anti-PD-1 immunotherapy in microsatellite stable colorectal cancer. J. Immunother. Cancer 9: e001895.
36. Yuan, S.-S. F., C.-W. Su, L.-P. Chan, H. D. H. Nguyen, Y.-K. Chen, J.-K. Du, K.-H. Cheng, and Y.-Y. Wang. 2023. IL17RB expression is associated with malignant cancer behaviors and poor prognosis in oral cancer. Oral Dis.
37. Villa-Crespo, L., S. Podlipnik, N. Anglada, C. Izquierdo, P. Giavedoni, P. Iglesias, M. Dominguez, F. Aya, A. Arance, J. Malvehy, S. Puig, and C. Carrera. 2022. Timeline of Adverse Events during Immune Checkpoint Inhibitors for Advanced Melanoma and Their Impacts on Survival. Cancers 14:1237.
38. Okazaki, T., and J. Wang. 2005. PD-1/PD-L pathway and autoimmunity. Autoimmunity 38:353-357.
39. Caldwell, R., G. Pina, P. Oderinde, A. Ali, J. Garrett, B. Siddiqui, S. Subudhi, C. Iliescu, S. W. Yusuf, E. Koutroumpakis, I. Hamzeh, S. Khalaf, A. Deswal, and N. Palaskas. 2022. 1262 Elevated serum TNF-α is a candidate prognostic biomarker for immune checkpoint inhibitor myocarditis. J. Immunother. Cancer 10.
40. Gergely, T. G., D. Kucsera, V. E. Tóth, T. Kovács, N. V. Sayour, Z. D. Drobni, M. Ruppert, B. Petrovich, B. Ágg, Z. Onódi, N. Fekete, É. Pállinger, E. I. Buzás, L. I. Yousif, W. C. Meijers, T. Radovits, B. Merkely, P. Ferdinandy, and Z. V. Varga. 2023. Characterization of immune checkpoint inhibitor-induced cardiotoxicity reveals interleukin-17A as a driver of cardiac dysfunction after anti-PD-1 treatment. Br. J. Pharmacol. 180:740-761.
41. Adam, K., and A. Mor. 2022. Immunohistochemistry of Immune Cells and Cells Bound to in vivo Administered Antibodies in Liver, Lung, Pancreas, and Colon of B6/lpr Mice. Bio-Protoc. 12: e4468.

### Example 6—In Vitro Binding of Anti-PD-1 Abs

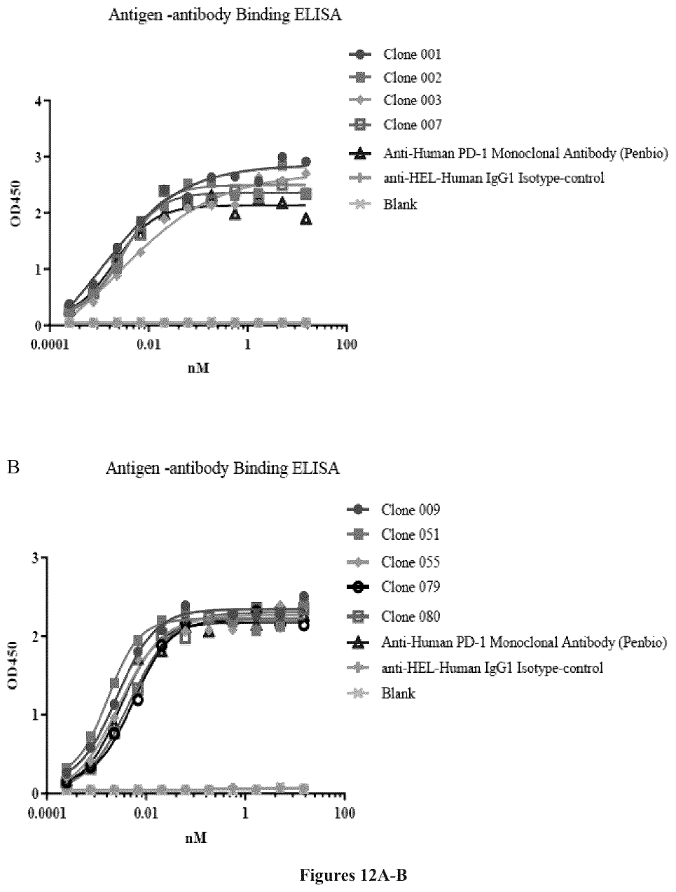

As shown in FIG. 12A-B, binding curves to human-PD-1-His-coated plates was quantified by ELISA. FIG. 12A shows binding curves for anti-PD1 antibody clones 01, 02, 03, and 07, anti-human PD-1 antibody Penbio (pembrolizumab), anti-HEL-human IgG1 isotype control, and blank. FIG. 12B shows binding curves for anti-PD1 antibody clones 09, 51, 55, 79, and 80, anti-human PD-1 antibody Penbio (pembrolizumab), anti-HEL-human IgG1 isotype control, and blank.

Table 4 shows EC50 values for anti-PD1 antibody clones 01, 02, 03, and 07, anti-human PD-1 antibody Penbio (pembrolizumab), anti-HEL-human IgG1 isotype control, and blank. Table 5 shows EC50 values for anti-PD1 antibody clones 09, 51, 55, 79, and 80, anti-human PD-1 antibody Penbio (pembrolizumab), anti-HEL-human IgG1 isotype control, and blank. EC50 values were calculated with GraphPad Prism (v10.2.1). These results show that the anti-PD1 antibody clones are capable of binding to PD-1, in some cases with lower or similar EC50 values when compared to the commercially available pembrolizumab.

| Antibody | EC₅₀(nM) |
|---|---|
| Clone 01 | 0.001054 |
| Clone 02 | 0.003222 |
| Clone 03 | 0.002326 |
| Clone 07 | 0.002939 |
| Anti-Human PD-1 Monoclonal Antibody (Penbio) | 0.002 |
| anti-HEL-Human IgG1 Isotype-control | No Binding |

| Antibody | EC₅₀(nM) |
|---|---|
| Clone 09 | 0.002673 |
| Clone 51 | 0.00176 |
| Clone 55 | 0.003264 |
| Clone 79 | 0.006451 |
| Clone 80 | 0.004642 |
| Anti-Human PD-1 Monoclonal Antibody (Penbio) | 0.003025 |
| anti-HEL-Human IgG1 Isotype-control | No Binding |

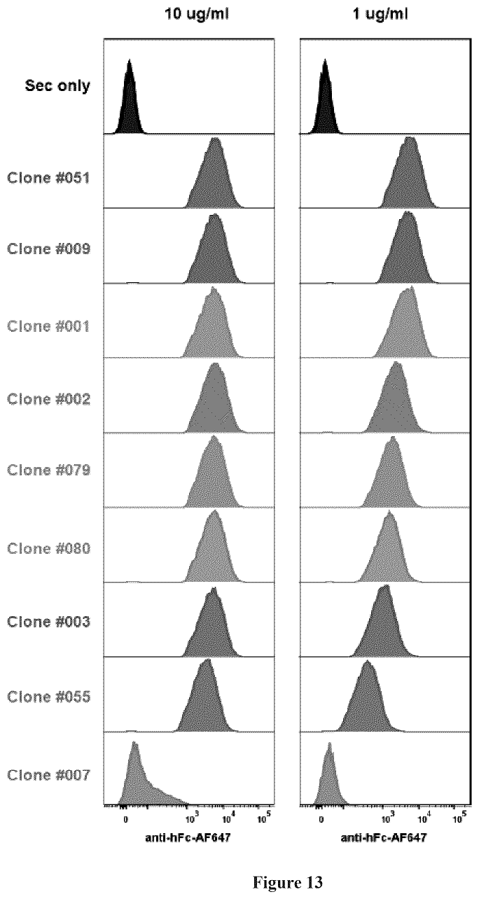

FIG. 13 shows binding of anti-PD-1 clones 01, 02, 03, 07, 09, 51, 55, 79, and 80 to cell-expressed PD-1 by flow cytometry. Jurkat T cells stably overexpressing human-PD-1 were incubated with 10 μg/ml (FIG. 13 left) or 1 μg/ml (FIG. 13 right) of each anti-PD-1 clone on ice for 30 minutes, washed twice, and then incubated for 30 minutes on ice with a fluorescent anti-human-Fc secondary antibody. Following two additional washes, cells were analyzed on a BD LSRFortessa™ flow cytometer. These results show that the anti-PD-1 clones bind to cell-expressed PD-1.

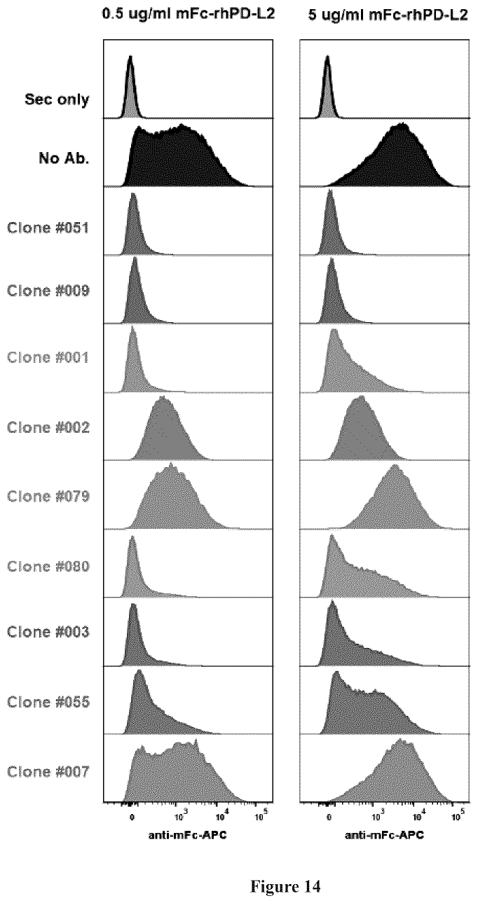

FIG. 14 shows anti-PD-1 clones 01, 02, 03, 07, 09, 51, 55, 79, and 80 blocking the binding of rhPD-L2 to cell-expressed PD-1. Jurkat T cells stably overexpressing human-PD-1 were incubated with 10 μg/ml of each anti-PD-1 clone on ice for 30 minutes, washed twice, and then incubated for 1 hour on ice with 0.5 μg/ml (FIG. 14 left) or 5 μg/ml (FIG. 14 right) of recombinant human PD-L2 (with mouse-Fc). Following two additional washes, the cells were incubated for 30 minutes on ice with a fluorescent anti-mouse-Fc secondary antibody, washed twice, and analyzed on a BD LSRFortessa™ flow cytometer. These results show that the anti-PD-1 clones block the binding of rhPD-L2 to cell-expressed PD-1.

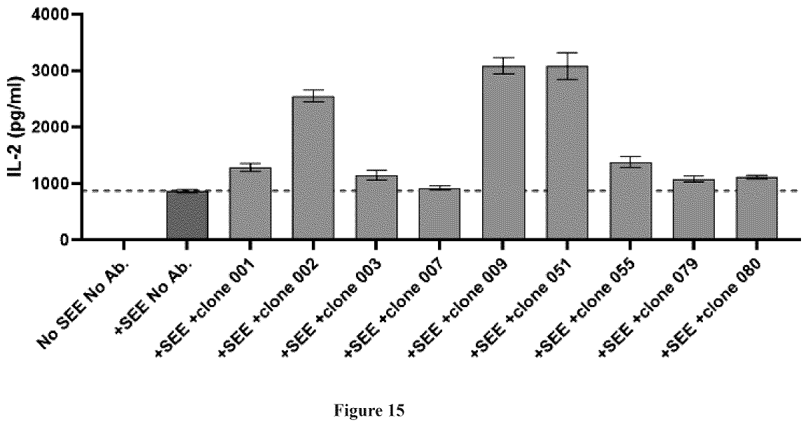

FIG. 15 shows IL-2 concentrations following addition of anti-PD-1 clones 01, 02, 03, 07, 09, 51, 55, 79, or 80 and SEE (Staphylococcal Enterotoxin E) in Jurkat-Raji co-culture compared to no SEE and no antibody control and SEE and no antibody control. Jurkat T cells stably overexpressing human-PD-1 and Raji B cells stably overexpressing human-PD-LI were co-cultured for 24 hours in the presence of 10 μg/ml of each anti-PD-1 clone and 100 μg/ml SEE (Staphylococcal Enterotoxin E). IL-2 concentrations in the supernatants were determined by ELISA. These results show that anti-PD-1 clones can increase IL-2 concentrations in the supernatants compared to no antibody controls.

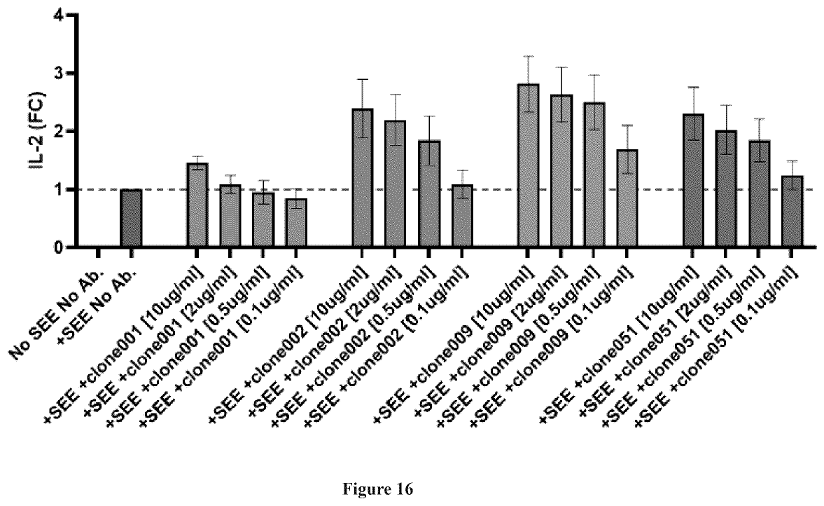

FIG. 16 shows IL-2 concentrations determined by ELISA following addition of anti-PD-1 clones 01, 02, 03, 07, 09, 51, 55, 79, or 80 at different concentrations (10, 2, 0.5 or 0.1 μg/ml) and SEE (Staphylococcal Enterotoxin E) in Jurkat-Raji co-culture compared to no SEE and no antibody control and SEE and no antibody control. Jurkat T cells stably overexpressing human-PD-1 and Raji B cells stably overexpressing human-PD-L1 were co-cultured for 24 hours in the presence of different concentrations (10, 2, 0.5 or 0.1 μg/ml) of each anti-PD-1 clone and 100 μg/ml SEE (Staphylococcal Enterotoxin E). These results show that anti-PD-1 clones can increase IL-2 concentrations in a dose dependent manner in the supernatants compared to no antibody controls.

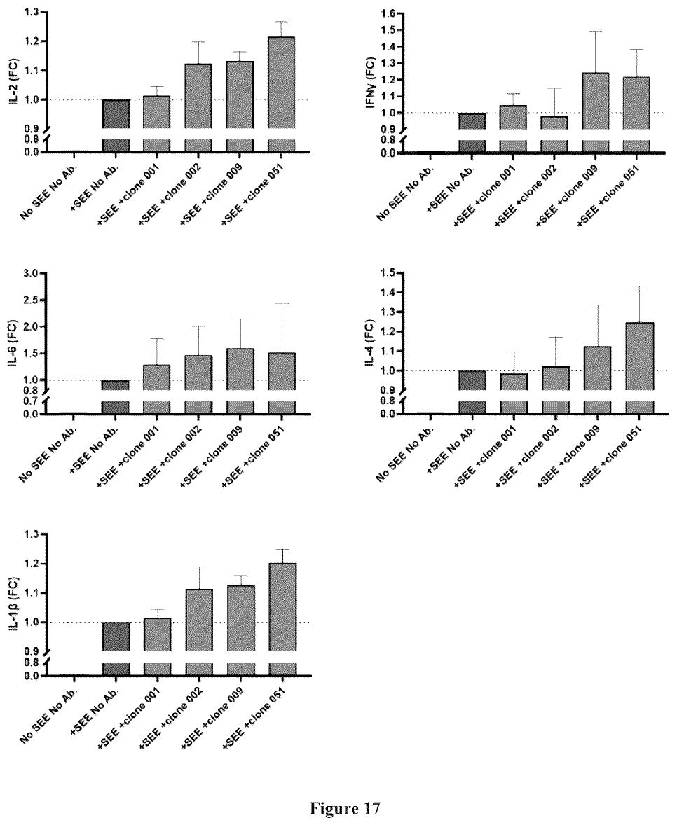

FIG. 17 shows concentrations of IL-2, IFNγ, IL-6, IL-4 and IL-1B determined by ELISA in PBMCs in the presence of anti-PD-1 clones 01, 02, 03, 07, 09, 51, 55, 79, or 80 and SEE (Staphylococcal Enterotoxin E). PBMCs from 3 healthy donors were incubated for 24 hours in the presence of 10 μg/ml of each anti-PD-1 clone and 100 μg/ml SEE (Staphylococcal Enterotoxin E). The concentrations of IL-2, IFNγ, IL-6, IL-4 and IL-1β in the supernatants were determined by ELISA. These results show that anti-PD-1 clones can increase IL-2, IFNγ, IL-6, IL-4 and IL-1β concentrations in the supernatants compared to no antibody controls.

### Example 7: Safety and Efficacy of Brodalumab in the Treatment of Immune-Related Adverse Events: A Pilot Study

This document is a protocol for a human research study. This study will be conducted according to US and international standards of Good Clinical Practice (FDA Title 21 part 312/812 and International Conference on Harmonization guidelines), applicable government regulations, and institutional research policies and procedures.

### Study Objectives

### Primary Objectives

To determine the safety of brodalumab in the treatment of immune-related adverse events (irAEs) in patients with solid tumors.

To determine the efficacy of brodalumab in treating immune-related adverse events, as measured by either reduction in cumulative steroid dosing or decreased irAEs by >=1 grade at 24 weeks.

### Secondary Objectives

To determine the proportion of patients who can be tapered completely off of steroids (and remain off for a minimum of | week).

To assess the percentage of patients with a net decrease in the average steroid dose required for irAEs management (defined by the ratio of the average steroid dose over the 7 days following enrollment compared to the average dose in the 7 days before study completion) 1

To identify the mean time to complete the resolution of irAE clinical manifestation (as defined by no longer meeting the relevant CTCAE criteria).

To determine the change in tumor burden as measured by RECIST criteria comparing CT/MRI scan at time of enrollment to CT/MRI scan at 24 weeks.

To determine the proportion of patients with ≥grade 3 infection (as per CTCAE criteria).

To calculate the cumulative steroid exposure (in prednisone equivalents) over 24 weeks.

To assess the change in the quality of life as measured by the EQ-5D-3L2.

To determine progression-free survival and overall survival.

### Cancer Immunotherapy

Cancer remains the second-leading cause of death in the US, accounting for 25% of all deaths nationwide³. In recent years, numerous immunomodulatory therapies have been developed to treat cancer. Specifically, the emergence of immune checkpoint inhibitors (ICIs); anti-cytotoxic T-lymphocyte antigen 4 (anti-CTLA-4), anti-programmed cell death 1 (anti-PD-1), and anti-programmed cell death ligand 1 (anti-PD-L1) antibodies have revolutionized cancer treatment. These antibodies, alone and in combination, are highly efficacious in treating multiple solid malignancies, as evidenced by tumor regression and prolonged overall survival. As a result, there has been significant interest in immune checkpoint inhibition with nearly 5,000 clinical trials for antibodies against PD-1 and its ligand, PD-L14. While these therapies are extremely successful in some patients', they can often cause significant complications due to immune-related adverse events⁵.

### Immune-Related Adverse Events

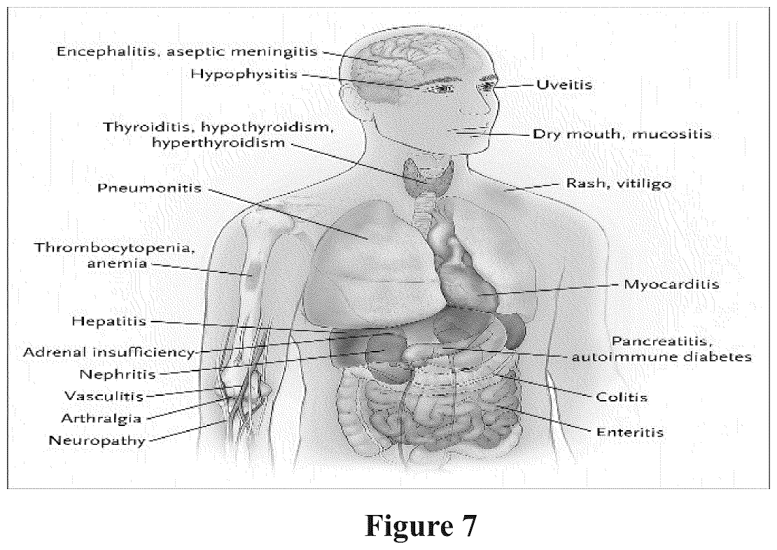

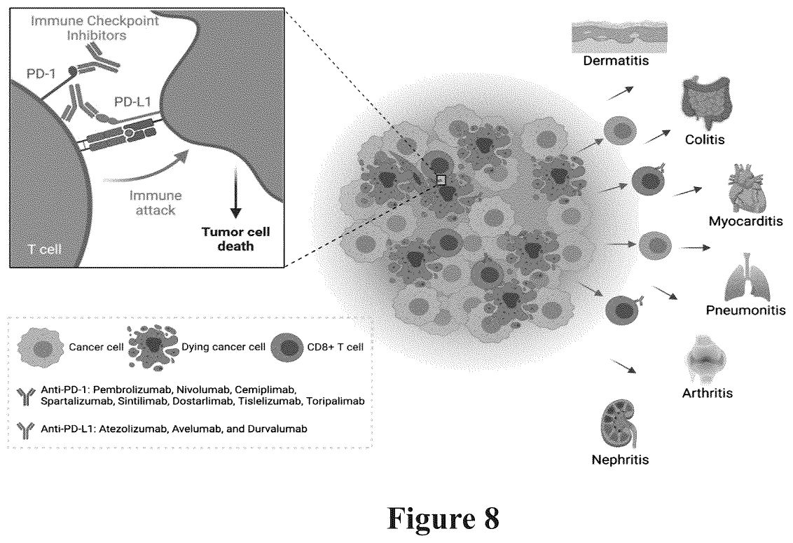

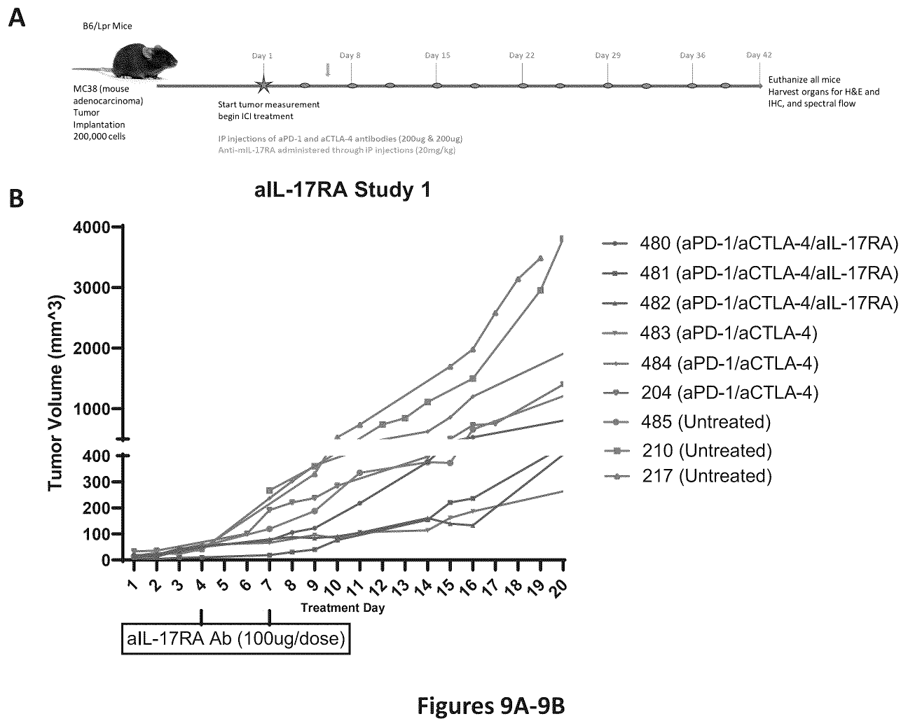

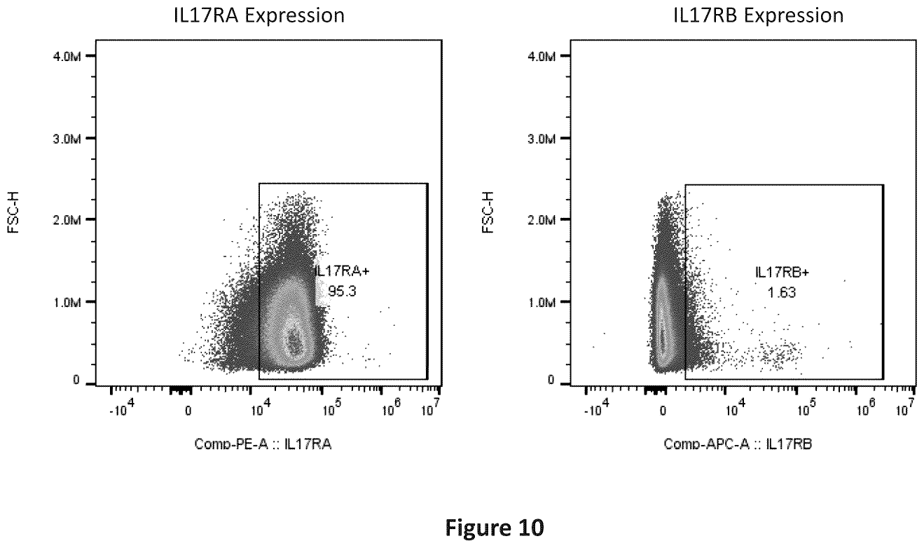

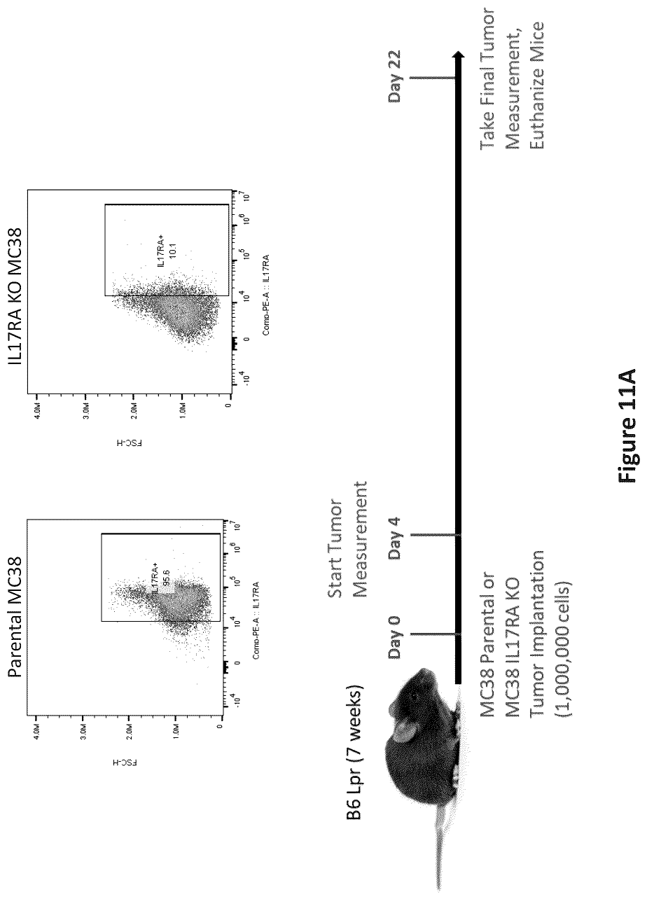

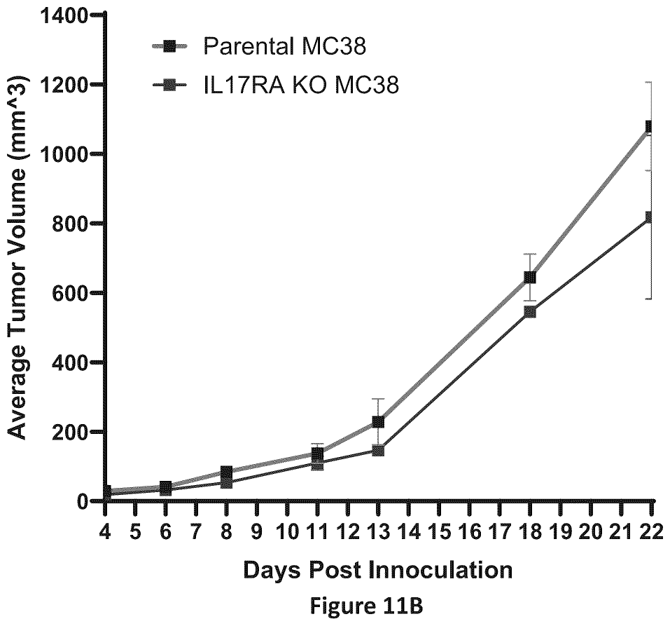

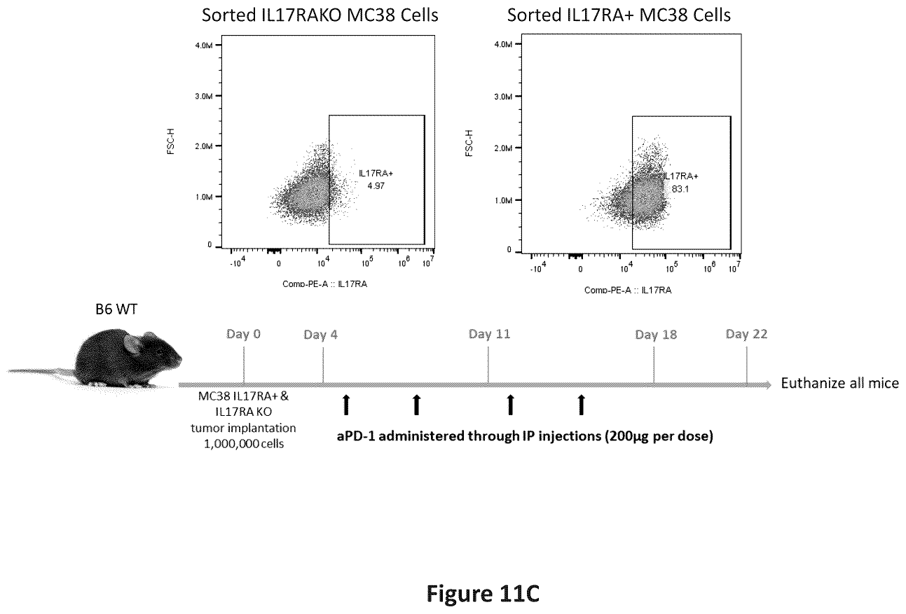

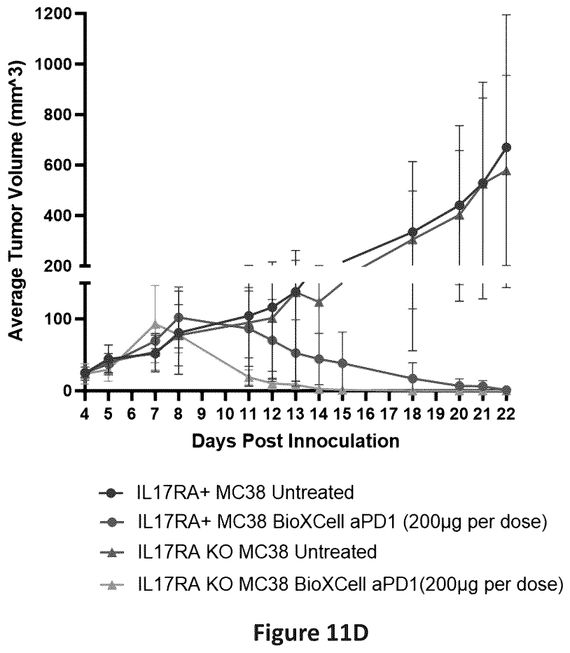

Immune-related adverse events are a spectrum of inflammatory toxicities due to the non-specific modulation of the immune system by immune checkpoint inhibitors. Clinically, irAEs resemble autoimmune disease (FIG. 7) and are often characterized by organ inflammation with T-cell infiltrates5. Other studies highlight cytokine dysregulation in irAE-affected tissues⁷ (FIG. 8). Most commonly, they affect the skin, gut, endocrine glands, liver, and lungs, but can potentially affect any organ⁸. Clinical data suggests that patients with NSCLC treated with single-agent PD-L1 therapy have rates of grade 3 or higher irAEs at approximately 25%, resulting in treatment discontinuation in 18% 9. In patients treated with combination blockade, grade 3 and higher irAEs occur in more than half of patients, leading to treatment discontinuation in approximately 40%, and the rates may be higher in those with early-stage cancers¹⁰. Initiation of appropriate irAE treatment is paramount to reduce the risk of sequelac. Thus, irAEs are frequently encountered in patients receiving immunotherapy, cause significant morbidity and mortality, and limit the ability to continue immunotherapy safely.

### Treatment of Immune-Related Adverse Events

Presently, most irAEs are empirically managed with corticosteroids¹¹. High-dose corticosteroids induce the resolution of symptoms in most patients, but the course of recovery may be prolonged. Data suggests that baseline corticosteroid use decreases the efficacy of checkpoint blockade.¹² This is postulated to be due to the immunosuppressive properties of steroids and their potential for blunting T cell responses¹³. In addition, irAEs can persist despite the use of corticosteroids and often recur during taper¹⁴. Given the numerous adverse effects of steroids, relapses during steroid withdrawal and the significant morbidity associated with immune-related adverse events, new therapies are needed to optimize irAE management.

The role of T_{H}17 and IL-17 in irAEs

Understanding of the pathophysiology behind irAEs is essential to optimizing treatment paradigms. To better elucidate the mechanisms underlying irAEs, single cell RNA sequencing was performed on peripheral blood from cancer patients treated with ICIs who developed irAEs. The presence of a subset of T_{H}17 cells at baseline predicted organ-specific irAEs. T_{H}17 cells are a specific population of T cells known to produce IL-17, a highly inflammatory cytokinc. Drugs neutralizing IL-17 are FDA-approved for psoriasis and psoriatic arthritis¹⁷. Interestingly, IL-17 is also known to regulate the tumor microenvironment, and higher levels of IL-17RA (Interleukin 17 Receptor A), one of the receptors for IL-17A, are associated with increased tumor growth and metastasis¹⁸. Furthermore, in the analysis, IL-17RA expression was correlated with worse overall survival in patients with solid malignancies.

With this strong rationale for targeting IL-17 signaling in irAEs, a mouse model that is predisposed to developing irAEs¹⁹ was used and administered anti-IL17 receptor monoclonal antibodies. As expected, this intervention prevented hepatitis, pneumonitis, myocarditis, and pancreatitis in the mice. But even more exciting was that therapy with IL17RA monoclonal antibodies augmented the ability of PD-1 blockade to inhibit tumor growth. Thus, targeting IL17RA prevented both the tumor growth and irAEs, indicating use for improving outcomes in patients with irAEs.

### Investigational Agent

### The Rationale for the Selection of Investigational Product or Intervention

Brodalumab is a fully human anti-IL-17 receptor A monoclonal antibody. In contrast to anti-IL-17A ligand antibodies (secukinumab and ixekizumab), brodalumab, inhibits the activity of IL-17A, IL-17F, IL-17A/F, and IL-17E (also known as IL-25).

Patients with IL-17-driven autoimmune conditions such as psoriasis and psoriatic arthritis are known to benefit from treatment with brodalumab.²⁰ While certain irAEs occur in cancer patients following treatment with immune-based therapies that phenotypically resemble brodalumab's approved indications, its efficacy has not yet been confirmed for this population. A clinical trial will evaluate the preliminary safety and efficacy of brodalumab in treating IL-17-mediated irAEs.

### Preclinical Data

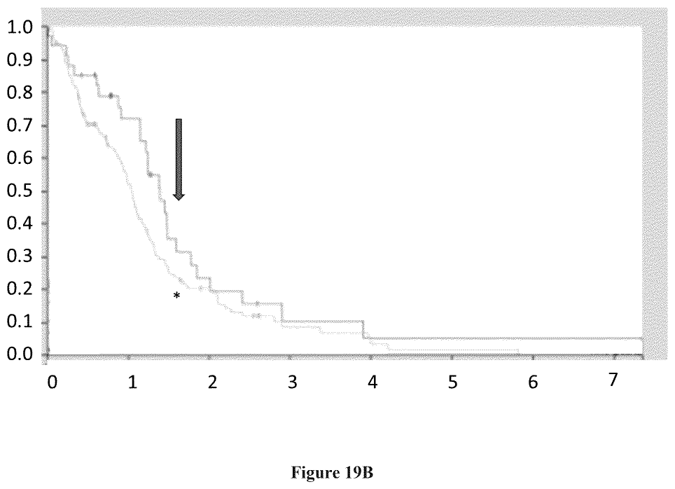

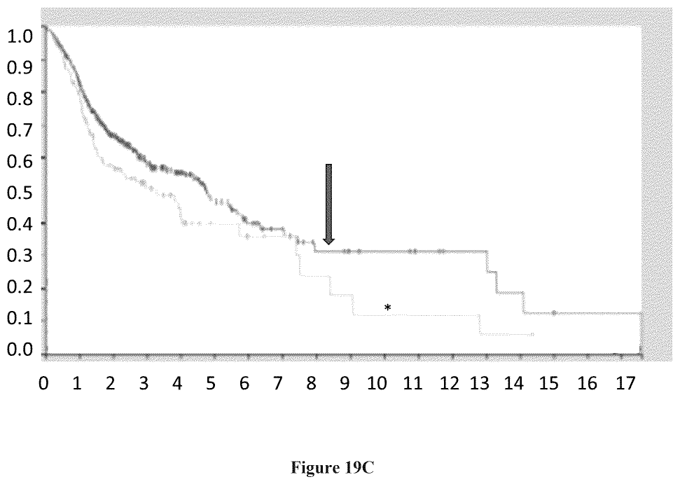

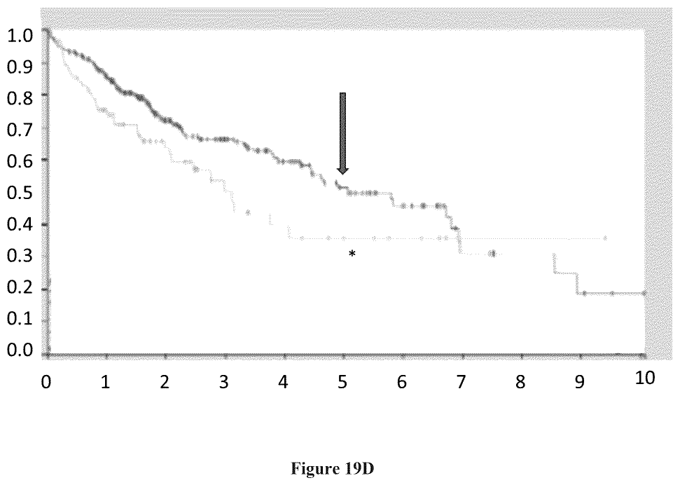

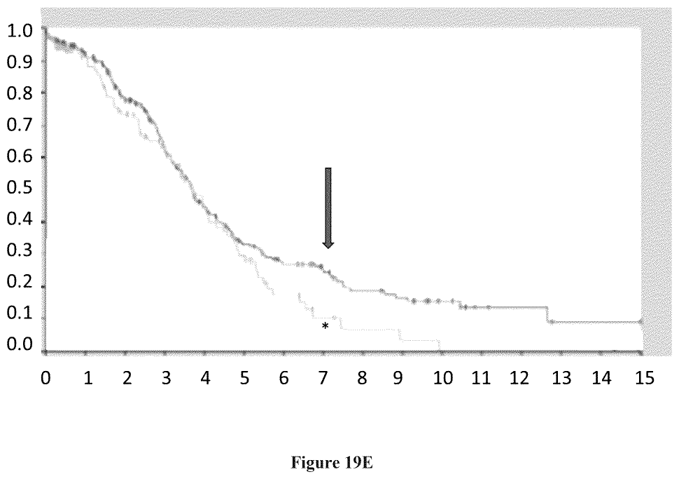

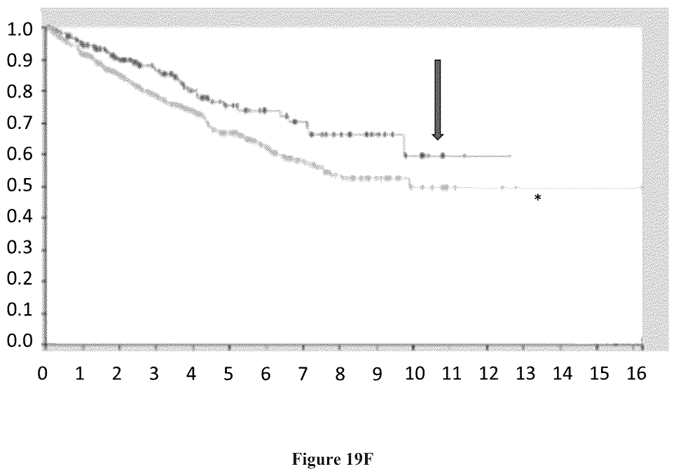

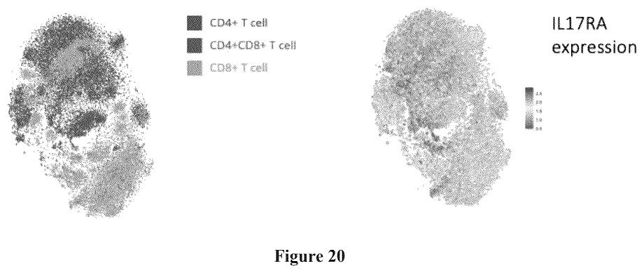

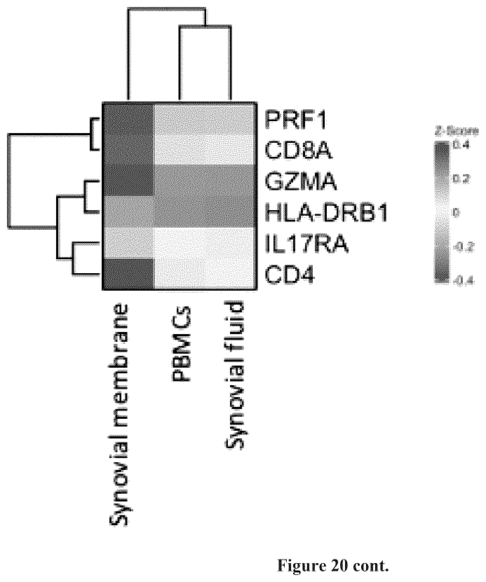

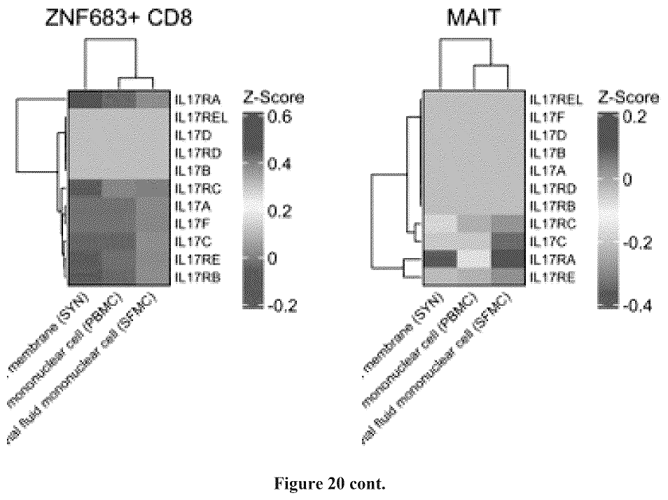

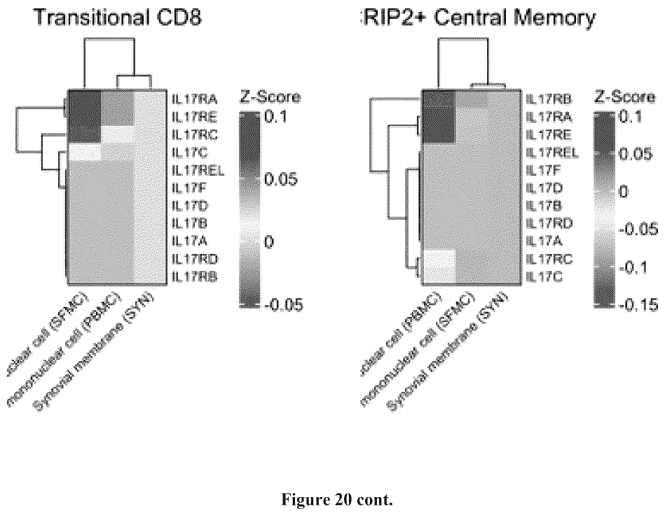

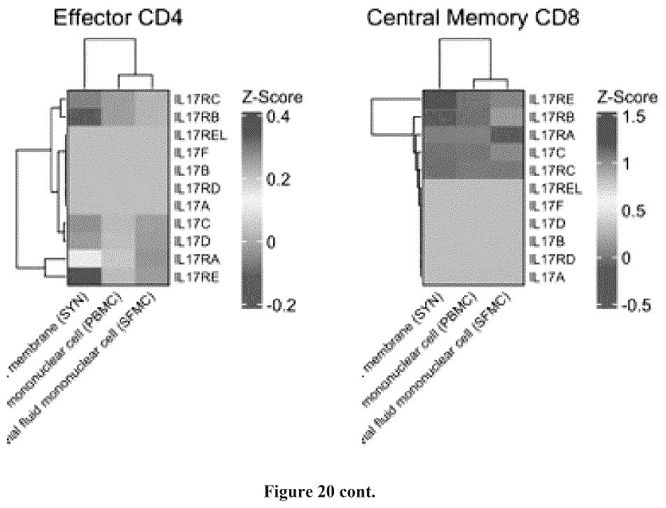

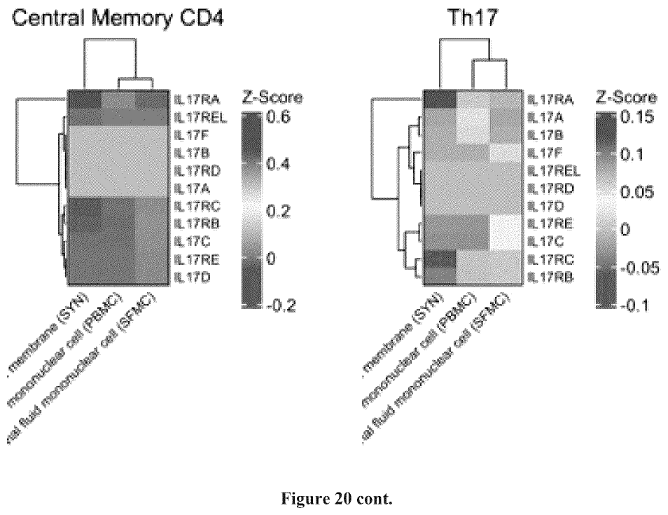

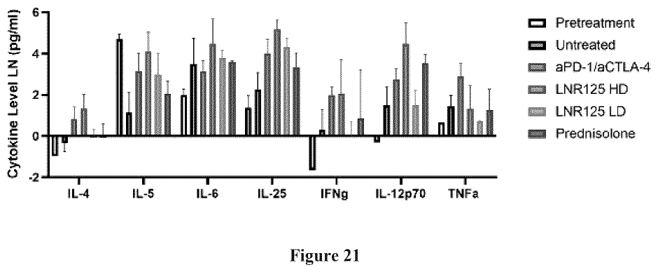

Data from The Cancer Genome Atlas (TGCA) database was analyzed²¹ to show that IL-17RA expression is associated with worse prognosis in multiple malignancies. TCGA data displayed in FIGS. 19A-F shows the Kaplan-Mayer survival curves of high and low IL17RA expression defined by the median expression for each cohort. Data from cervical (A), glioma (B), ovarian (C), head and neck (D), liver (E), and renal (E) cancers were analyzed²². Using publicly available sequencing data of patients with inflammatory arthritis, it was shown that IL-17RA expression is higher in patients with inflammatory arthritis. Cluster plots derived from single-cell RNA sequencing data depicting the expression levels of IL17RA in CD4+, CD8+, and CD4+CD8+ T cells are presented in FIG. 20. Heat map analysis of single-cell gene expression shows that, like other inflammatory genes, IL17RA levels are higher in synovial membranes of patients with psoriatic arthritis compared to peripheral blood mononuclear cells (PBMCs) or synovial fluid from the same patients. In addition to IL17RA, the expression levels of some other members of the IL-17 signaling system are also highly expressed in likely pathogenic and clonally expanded subsets of T cells isolated from the synovial membrane of patients with inflammatory arthritis. In the irAEs murine model and using an MC38 syngeneic tumor model, it was shown that administration of anti-murine IL17RA antibodies resulted in decreased tumor size and prevention of irAEs (FIG. 20). Moreover, these data correlated with scrum IL-17 levels of the same mice (FIG. 21). Altogether, this data supports the hypothesis that anti-IL17RA therapy should prevent irAEs in cancer patients and, at the same time, enhance the anti-tumoral effect of ICIs.

### Clinical Data

Brodalumab has been explored in clinical trials as a potential treatment for psoriasis, psoriatic arthritis, rheumatoid arthritis, and inflammatory bowel disease. Initial data from November 2014 showed superior skin clearance in a Phase III trial of patients with psoriasis compared to ustekinumab and placebo²³. Brodalumab was approved by the US Federal Drug Administration (FDA) in 2017 to treat moderate to severe plaque psoriasis²⁴. This study will use the dosing scheme approved by the FDA for the treatment of psoriasis. Specifically, patients will receive 210 mg subcutaneous injection on weeks 0, 1, 2 followed by administration every 2 weeks until week 24. Given that patients with irAEs can clinically present similarly to those with psoriasis and psoriatic arthritis, it is anticipated that anti-IL-17RA therapy will also have similar efficacy in these patients.

Suicidal ideation and behavior have been reported with brodalumab. Four completed suicides occurred in subjects treated with brodalumab in the psoriasis clinical trials compared to none in the placebo arm in the 12-week-controlled trials. Participants with a history of suicidality or depression had an increased incidence of suicidal ideation and behavior as compared to users without such a history. Investigators will weigh the potential risks and benefits before using brodalumab in patients with a history of depression or suicidality. Participants with new or worsening symptoms of depression or suicidality will be referred to a mental health professional. All participants will be counseled to seek medical attention for manifestations of suicidal ideation and behavior, new/worsening depression, anxiety, and mood changes. Investigators should also evaluate the risks/benefits of continuing treatment should a participant report new/worsening depression or suicidal ideation.

Brodalumab may increase the risk of infection. In clinical trials, subjects treated with brodalumab had a higher rate of infections, including serious infections, compared to subjects treated with placebo (0.5% versus 0.2%) and an increased rate of fungal infections (2.4% versus 0.9%). One case of cryptococcal meningitis was reported in a subject treated with brodalumab during the 12-week randomized treatment period, leading to discontinuation of therapy. During the clinical trials for plaque psoriasis, the exposure-adjusted rates for infections and serious infections were similar in the subjects treated with brodalumab and those treated with ustekinumab. In participants with a chronic infection/history of recurrent infection, investigators should consider the risks and benefits of brodalumab. Participants should be counseled to seek medical help if they experience signs or symptoms of worsening chronic or acute infection. Participants who develop a serious infection should be discontinued from brodalumab.

Participants will be evaluated for tuberculosis (TB) infection prior to initiating treatment with brodalumab. Brodalumab should not be administered to participants with active TB infection. Participants should be closely monitored for signs and symptoms of active TB during and after treatment.

In clinical trials for plaque psoriasis, Crohn's disease occurred in 1 subject receiving brodalumab, leading to discontinuation of therapy. Other trials have reported exacerbation of Crohn's disease observed with brodalumab use. Brodalumab is contraindicated in patients with Crohn's disease. Brodalumab should be discontinued in participants who are diagnosed with Crohn's disease during the course of the study.

Live vaccines should be avoided patients treated with brodalumab. No data are available on the ability of live or inactive vaccines in use with brodalumab.²⁷

### Benefits of Brodalumab

Potential benefits include improved control of immune-related adverse events and cancer control due to exposure to brodalumab. This can decrease morbidity and mortality experienced from irAEs, high dose steroid therapy and/or malignancy.

### Other Agent(s)

Corticosteroids will be administered in accordance with clinical practice guidelines. It is well known that prolonged corticosteroid use is associated with significant toxicity including hypertension, adrenal insufficiency, weight gain, myopathy, dyslipidemia, diabetic mellitus, gastritis, osteoporosis, increased risk of opportunistic infections, and psychiatric disturbances, which can lead to treatment discontinuation.²⁹ Every effort will be made to reduce the steroid doses in patients safely and it is expected that brodalumab will assist with steroid tapering.

### Study Design

### General Design

Brodalumab will be used for the treatment of irAEs for patients with solid tumors in 11 patients. The proposed study will evaluate the safety and efficacy with regards improvement and resolution of the irAE in patients treated with Brodalumab. All subjects will meet the National Cancer Institute Common Terminology Criteria for Adverse Events (NCI CTCAEv5.0) definition of ≥grade 3 irAEs. Subjects will receive subcutaneous brodalumab for 24 weeks. Peripheral blood will be collected at all in-person study visits for mechanistic studies.

Participants will be evaluated at week 0, 1, 2, 4 and then every 4 weeks after until week 24 as dictated by the standard of care using a combination of telemedicine and face-to-face evaluations. Additional safety follow-up visits will occur at week 28 and 36. A follow up safety visit will be conducted at weeks 28 and 36. All patients will have the Columbia Suicide Severity Rating Scale (C-SSRS), and Patient Health Questionnaire-9 (PHQ-9) administered at all visits.

The treatment protocol consists of subcutaneous brodalumab 210 mg administered at baseline and then at weeks 0, 1, 2, then bi-weekly for a total of 24 weeks (the current FDA-approved dosing for plaque psoriasis). Glucocorticoids may be used at baseline at the discretion of the investigators, but all subjects treated with glucocorticoids will be tapered to 0 mg of steroids by week 4. Assessments will be done every month as dictated by the standard of care using a combination of telemedicine and face-to-face evaluations.

The primary objectives will be to assess the safety and efficacy of brodalumab in patients by measuring the percentage of patients meeting Common Terminology Criteria for Adverse Events (CTCAE v5.0)³⁰ grade 3 and above during the study and the change in irAE grade from enrollment to 24 weeks based upon CTCAE v5.0 grading.³¹

Imaging studies, guided by organ involvement at baseline, will be repeated at 24 weeks in order to assess disease activity at the time of the primary endpoint. Imaging studies can also be performed at other time points at the discretion of the investigating physician.

Peripheral blood mononuclear cells will be collected at baseline, four weeks, 12, and 24 weeks. B and T cell subsets will be quantified using a 25-parameter flow cytometry panel.

### Dose Limiting Toxicities

No dose adjustments will be permitted in this study.

### Number of Participants

11 patients will be enrolled, all of whom have a ≥grade 3 immune-related adverse event which is presumed to be IL-17 related per CTCAEv5 criteria in the opinion of the treating physician and/or study investigators and who have a solid malignancy that has been treated with immunotherapy.

### Subject Selection and Withdrawal

Inclusion Criteria: (1) Ability to provide written informed consent by subject or guardian; (2) Individuals >18 years of age; (3) Diagnosis of >/=grade 3 irAE clinically suspected to be IL-17 mediated; (4) Histology-proven primary solid organ malignancy.

Exclusion Criteria: (1): Estimated creatinine clearance <40 mg/min; (2) Severe depression is defined as PHQ 9 score >20; (3) Active suicidal ideation or severe depression at the time of enrollment; (4) In the opinion of the investigator, the patient requires additional immunosuppressive treatment (other than corticosteroids); (5) Prior therapy with anti-CD20 or anti-IL-6 agents within three months; (6) Known hypersensitivity or contraindication to brodalumab corticosteroids or any components of these drug products; (7) Prior treatment with brodalumab; (8) Pregnancy, breastfeeding, or use of a nonreliable method of contraception; (9) Chronic or current severe infection; (10) Evidence of active hepatitis B, C, or tuberculosis; (11) History of or active Crohn's disease; (12) Any medical condition or treatment for a condition that, in the opinion of the investigator, might interfere with participation in the study or affect the reliability of clinician assessment or patient self-report; (13) Other known clinically significant active medical conditions, such as: Severe cardiovascular disease, including congestive heart failure; Liver dysfunction unrelated to irAE (aspartate aminotransferase-AST, alanine aminotransferase-ALT, or bilirubin greater than 4 times the upper limit of normal, measured on at least two separate occasions); Bone marrow insufficiency unrelated to the irAE (according to investigator judgment) with White Blood Cell (WBC)<2000/mm³, absolute neutrophil count <1500/mm³, thrombocytopenia (platelet count)<50,000/mm³, hemoglobin >9.0 g/dL; (14) Participation in another clinical trial and/or receipt of investigational drugs within 4 weeks prior to the screening visit; (15) Previous diagnosis of an autoimmune disease or administration of immunosuppressants in a time frame that would impede interpretation of brodalumab administration; (16) Administration of live-virus vaccines within last 4 weeks

### Inclusion of Women and Minorities

Both men and women of all races and ethnic groups are eligible for this trial.

### Early Withdrawal of Subjects

### When and How to Withdraw Subjects

If at any time a subject develops suicidal ideation or new severe depression the patient will be referred for further care and removed from the study.

If at any time a subject develops unacceptable toxicity (as defined by the investigators), he/she will be removed from study.

Suppose at any time the patient is found to be ineligible for the protocol designated in the Criteria for Patient/Subject Eligibility (i.e., a change in diagnosis). In that case, the patient will be removed from the study.

If the patient fails to comply with the defined treatment plan and follow-up evaluations (as per above), the patient will be removed from the study.

If the patient withdraws consent for continued participation, he/she will be removed from study.

Subjects will also be withdrawn if in the opinion of the investigator a therapy other than steroids is needed to adequately manage their immune related adverse event.

### Data Collection and Follow-Up for Withdrawn Subjects

Even though subjects may be withdrawn prematurely from the study, it is imperative to collect at least survival data on such subjects throughout the protocol-defined follow-up period. Such data is important to the integrity of the final study analysis since early withdrawal could be related to the safety profile of the study drug. Suppose a subject withdraws consent to participate in the study. In that case, attempts will be made to obtain consent from the subject to record at least survival data up to the protocol-described end of the subject follow-up period. Obtaining at least survival data on all subjects lost to follow-up must be a high priority. Multiple attempts will be made to contact the patient, and attempts will also be made to contact next of kin available in the medical record and letters will be sent to their address on file. Subjects withdrawn because of unacceptable adverse events will be followed until resolution or stabilization of the adverse event.

### Treatment and Imaging Plan

### Agent Administration

Treatment will be administered according to the package insert. Reported adverse events and potential risks for broadlumab and steroids are described herein. No investigational or commercial agents or therapies other than those described below may be administered with the intent to treat the patient's malignancy.

Patients will receive 210 mg subcutaneous injection on weeks 0, 1, 2 followed by administration every 2 weeks until week 24.

### Administration Plan for Investigational Agent(s)

No prophylaxis or supportive care therapies will be administered routinely along with the investigational agent. Qualified and trained staff will administer the subcutaneous injection. If the patient has an active infection, discussion should be held with a primary investigator to confirm that it is safe to proceed.

### Administration Plan for Other Agent(s)

Patients will take or be administered steroids as per their treating physician's instructions. If it is anticipated that patients will be on >20 mg of prednisone (or equivalent) for >1 month, pneumocystis jirovecii prophylaxis should be administered.

### Other Modality(ies) or Procedures

### General Concomitant Medication and Supportive Care Guidelines

Immunosuppressive therapies other than steroids and brodalumab will not be permitted.

### Duration of Therapy

In the absence of treatment delays due to adverse events, treatment may continue for 24 weeks or until one of the following criteria applies: Intercurrent illness that prevents further administration of treatment; Unacceptable adverse events(s); Patient decides to withdraw from the study; General or specific changes in the patient's condition render the patient unacceptable for further treatment in the judgment of the investigator.

Treatment beyond the study period must be discussed with the Sponsor-Investigator in cases where ongoing benefit to the patient is observed.

### Duration of Follow Up

Participants will be followed for 12 weeks after completion or removal from study or until death, whichever occurs first. Participants removed from study for unacceptable adverse events will be followed until resolution or stabilization of the adverse event.

A participant will be considered lost to follow-up if he or she fails to return for 3 scheduled visits and is unable to be contacted by the study site staff.

Criteria for Removal from Study: (1) Severe worsening of the initial irAE that in the opinion of the investigator requires treatment with a different immunosuppressant; (2) Severe, life-threatening infections; (3) Any adverse event that in the opinion of the investigator require study discontinuation; (4) Severe depression as defined as PHQ-9 score >20 and/or suicidal ideation (an action plan will be in place for patients who develop severe depression and/or suicidal ideation); (5) Pregnancy; (6) Study therapy may also be prematurely discontinued for any participant if the investigator believes that the study treatment is no longer in the best interest of the participant.

Participants will be removed from study when any of the criteria listed applies.

### Dosing Delays/Dose Modifications

Dose modifications will not be allowed. Treatment may be delayed if a patient has an active infection or illness and resumed when deemed safe to do so by a clinical investigator. In this situation, the planned number of doses will still be administered beginning from the time that the patient receives the missed dose.

### Pharmaceutical Information

### Study Drugs

### Description of Treatment Regimen

Brodalumab is an anti-IL-17RA monoclonal antibody which is supplied in a prefilled single-dose syringe containing 210 mg of drug in 1.5 mL of sterile, preservative-free solution.

### Treatment Regimen

The dose of brodalumab is 210 mg at week 0, 1, and 2 followed by 210 mg every 2 weeks to week 24.

### Preparation and Administration of Study Drug

Brodalumab prefilled syringe should be brought to room temperature approximately 30 minutes before injecting. Brodalumab should not warm in any other way. Do not remove the gray needle cap on the prefilled syringe while allowing it to reach room temperature.

Brodalumab is administered subcutaneously. Each prefilled syringe is for single-dose only. It should not be injected in areas where skin is tender, bruised, red, hardened, thick, scaly or affected by psoriasis or rash.

### Subject Compliance Monitoring

At each visit, adherence will be documented. Patients who miss an appointment will have their appointment re-scheduled as soon as clinically feasible but ideally within 5 days. Patients who miss a particular visit will still be able to receive all study drug doses. If patients miss 3 sequential visits they will be removed from the study.

### Prior and Concomitant Therapy

All treatments that the investigator considers necessary for a subject's welfare may be administered at the discretion of the investigator in keeping with the community standards of medical care. All concomitant medication will be recorded on the case report form (CRF) including all prescription, over-the-counter (OTC), herbal supplements, and intravenous (IV) medications and fluids. If changes occur during the trial period, documentation of drug dosage, frequency, route, and date may also be included on the CRF.

All medications received within 28 days before the first dose of trial treatment and 30 days after the last dose of trial treatment should be recorded. All chemotherapies taken within 6 weeks of the trial must be noted. Concomitant medications administered after 30 days after the last dose of trial treatment should be recorded for SAEs.

Subjects are prohibited from receiving the following therapies during the screening and treatment phase of this trial: Non-biologic immunosuppressive or immune-modulating drug (e.g., methotrexate, azathioprinc, cyclosporine, hydroxychloroquine, penicillamine); Investigational agents including rituximab, tocilizumab, infliximab, vedolizumab, mycophenolate mofetil; Live vaccines within 4 weeks prior to the first dose of trial treatment and while participating in the trial. Examples of live vaccines include, but are not limited to, the following: measles, mumps, rubella, chicken pox, yellow fever, rabies, BCG, and typhoid (oral) vaccine. Seasonal influenza vaccines for injection are generally killed virus vaccines and are allowed; however intranasal influenza vaccines (e.g., Flu-Mist®) are live attenuated vaccines, and are not allowed; Cancer directed immunotherapy is not permitted; Chemotherapy will not be permitted while a patient is enrolled in the trial; Glucocorticoids are permitted on this trial.

Subjects who, in the assessment by the investigator, require the use of any of the aforementioned treatments for clinical management should be removed from the trial. Subjects may receive other medications that the investigator deems to be medically necessary.

The Exclusion Criteria describes other medications, which are prohibited in this trial.

### Packaging

The drug will be supplied as cartons of two 210 mg/1.5 mL single-dose prefilled syringes.

### Storage

Brodalumab must be stored refrigerated at 2° C. to 8° C. (36° F. to 46° F.) in the original carton to protect from light and physical damage during storage. When necessary, prefilled syringes can be stored at room temperature up to a maximum of 77° F. (25° C.) in the original carton for a maximum single period of 14 days with protection from light and sources of heat. Once the prefilled syringe has reached room temperature, brodalumab may not be placed back into the refrigerator. Brodalumab should be discarded after 14 days at room temperature.

For further information please see package insert.³⁴

### Other Agent(s)

Steroid medications will be dispensed from patient's respective pharmacies or from CUIMC inpatient pharmacy in accordance with standard policies and procedures.

### Study Calendar

Schedule of Activities for Part I

| | Week | | | | | | | | | | | |
|---|---|---|---|---|---|---|---|---|---|---|---|---|
| | −4 | 0 | 1 | 2 ^{a} | 4 | 8 ^{a} | 12 | 16 | 20 ^{a} | 24 | 28 ^{a} | 36 |
| | Visit | | | | | | | | | | | |
| | −1 | 0^{b} | 1 | 2 | 3 | 4 | 5 | 6 | 7 | 8 | 9 | 10 |
| Informed Consent | x | | | | | | | | | | | |
| Eligibility Criteria | x | | | | | | | | | | | |
| Demographics | x | | | | | | | | | | | |
| Brodalumab | | x | x | x | x | x | x | x | x | x | | |
| Adherence Assessment | | | x | x | x | x | x | x | x | x | | |
| Medical History | x | | | | | | | | | | | |
| Adverse Events | | x | x | x | x | x | x | x | x | x | x | x |
| Concomitant Medications | x | x | x | x | x | x | x | x | x | x | x | x |
| Vital Signs | x | x | x | x | x | x | x | x | x | x | x | x |
| Comprehensive Physical | x | x | | | x | | x | | | x | | x |
| Limited Physical | | | x | x | | x | | x | x | | x | x |
| Examination | | | | | | | | | | | | |
| Adverse Events | | x | x | x | x | x | x | x | x | x | x | x |
| Drug Accountability | | x | x | x | x | x | x | x | x | x | x | x |
| Safety follow up phone | x | x | x | x | x | x | x | x | x | x | x | x |
| call, as needed in between | | | | | | | | | | | | |
| study visits | | | | | | | | | | | | |
| Patient Global Assessment | x | x | x | x | x | x | x | x | x | x | x | x |
| Physician Global | x | x | x | x | x | x | x | x | x | x | x | x |
| Assessment | | | | | | | | | | | | |
| Organ Specific irAE | x | x | x | x | x | x | x | x | x | x | x | x |
| Assessment | | | | | | | | | | | | |
| Patient Quality of Life | x | x | x | x | x | x | x | x | x | x | x | x |
| Assessment | | | | | | | | | | | | |
| C-SSRS and PHQ-9 | x | x | x | x | x | x | x | x | x | x | x | x |
| Labs per standard of care | x | x | x | x | x | x | x | x | x | x | x | x |
| (CBC, CMP, urinalysis, | | | | | | | | | | | | |
| irAE specific evalutions, | | | | | | | | | | | | |
| lymphocyte panel, urine | | | | | | | | | | | | |
| pregnancy, hepatitis B | | | | | | | | | | | | |
| antibodies, HCV | | | | | | | | | | | | |
| antibodies, | | | | | | | | | | | | |
| QuantiFERON/PPD) | | | | | | | | | | | | |
| Imaging to evaluate the | x | | | | | | | | | x | | |
| irAE (if indicated) | | | | | | | | | | | | |
| Tumor Burden | x | | | | | | | | | x | | |
| Assessment | | | | | | | | | | | | |
| Mechanistic Samples | x | x | x | x | x | x | x | x | x | x | x | x |

^{a} Option for telemedicine visit, labs to be completed if visit conducted in person
^{b}May occur on same day as screening if patient meets all eligibility criteria
^{c} For individuals of childbearing potential

### Measurement of Effect

Treatment effect will be measured by determining the irAE grade as per CTCAE v5.0 criteria at each visit and comparison will be made between irAE grade at enrollment and at the conclusion of brodalumab treatment.

### Antitumor Effect—Solid Tumors

For the purposes of this study, participants will undergo a scan at baseline (i.e., within 4 weeks of receiving the first dose of therapy), at 8 weeks, and thereafter every 12 weeks. A+/−7-day window will be permitted for scans. Imaging tests to be used will include standard of care imaging for the patient's tumor (i.e., typically CT or MRI scans of affected organs, PET-CT).

Response and progression will be evaluated in this study using the international criteria proposed by the revised Response Evaluation Criteria in Solid Tumors (RECIST) guideline (version 1.1).³⁵ Changes in the largest diameter (unidimensional measurement) of the tumor lesions and the shortest diameter in the case of malignant lymph nodes are used in the RECIST criteria.

Evaluable for toxicity: All participants will be evaluable for toxicity from the time of their first treatment with brodalumab.

Evaluable for objective response: Only those participants who have measurable disease present at baseline, have received at least 8 weeks of therapy, and have had their disease re-evaluated will be considered evaluable for response. These participants will have their response classified according to the definitions stated below. (Note: Participants who exhibit objective disease progression prior to the end of cycle 1 will also be considered evaluable.)

Evaluable Non-Target Disease Response: Participants who have lesions present at baseline that are evaluable but do not meet the definitions of measurable disease, have received at least one cycle of therapy, and have had their disease re-evaluated will be considered evaluable for non-target disease. The response assessment is based on the presence, absence, or unequivocal progression of the lesions.

### Disease Parameters

Measurable disease: Measurable lesions are defined as those that can be accurately measured in at least one dimension (longest diameter to be recorded for non-nodal lesions and short axis for nodal lesions) as ≥20 mm by chest x-ray, as ≥10 mm with CT scan, or ≥10 mm with calipers by clinical exam. All tumor measurements must be recorded in millimeters (or decimal fractions of centimeters).

Malignant lymph nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

Non-measurable disease: All other lesions (or sites of disease), including small lesions (longest diameter <10 mm or pathological lymph nodes with ≥10 to <15 mm short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable.

Note: Cystic lesions that meet the criteria for radiographically defined simple cysts should not be considered as malignant lesions (neither measurable nor non-measurable) since they are, by definition, simple cysts. Cystic lesions thought to represent cystic metastases can be considered as measurable lesions, if they meet the definition of measurability described above. However, if non-cystic lesions are present in the same patient, these are preferred for selection as target lesions.

Target lesions: All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions: All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

### Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam.

Clinical lesions: Clinical lesions will only be considered measurable when they are superficial (e.g., skin nodules and palpable lymph nodes) and ≥10 mm diameter as assessed using calipers (e.g., skin nodules). In the case of skin lesions, documentation by color photography, including a ruler to estimate the size of the lesion, is recommended.

Conventional CT and MRI: This guideline has defined measurability of lesions on CT scan based on the assumption that CT slice thickness is 5 mm or less. If CT scans have slice thickness greater than 5 mm, the minimum size for a measurable lesion should be twice the slice thickness. MRI is also acceptable in certain situations (e.g., for body scans).

Use of MRI remains a complex issue. MRI has excellent contrast, spatial, and temporal resolution; however, there are many image acquisition variables involved in MRI, which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up should be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used and the image acquisition protocol should be followed as closely as possible to prior scans. Body scans should be performed with breath-hold scanning techniques, if possible.

FDG-PET: While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

Negative FDG-PET at baseline, with a positive FDG-PET at follow-up is a sign of PD based on a new lesion.

No FDG-PET at baseline and a positive FDG-PET at follow-up: If the positive FDG-PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.

FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease-specific medical literature for the indication. However, it must be acknowledged that both approaches may lead to false positive CR due to limitations of FDG-PET and biopsy resolution/sensitivity.

PrimNote: A ‘positive’ FDG-PET scan lesion means one which is FDG avid with an uptake greater than twice that of the surrounding tissue on the attenuation corrected image.

### Response Criteria

### Evaluation of Target Lesions

Other standard disease-specific response criteria can to substituted for the suggested text.

Complete Response (CR): Disappearance of all target lesions. Any pathological lymph nodes (whether target or non-target) must have reduction in short axis to <10 mm.

Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

Stable Disease (SD): Neither sufficient shrinkage to qualify for PR nor sufficient increase to qualify for PD, taking as reference the smallest sum diameters while on study. Evaluation of Non-Target Lesions

Complete Response (CR): Disappearance of all non-target lesions and normalization of tumor marker level. All lymph nodes must be non-pathological in size (<10 mm short axis).

Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase. Although a clear progression of “non-target” lesions only is exceptional, the opinion of the treating physician should prevail in such circumstances, and the progression status should be confirmed at a later time by the review panel (or Principal Investigator).

### Evaluation of Best Overall Response

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

For Participants with Measurable Disease (e.g., Target Disease)

| | | | | Best Overall |
|---|---|---|---|---|
| | | | | Response when |
| Target | Non-Target | New | Overall | Confirmation |
| Lesions | Lesions | Lesions | Response | is Required* |
| CR | CR | No | CR | ≥4 wks. |
| | | | | Confirmation** |
| CR | Non-CR/Non-PD | No | PR | ≥4 wks. |
| CR | Not evaluated | No | PR | Confirmation** |
| PR | Non-CR/Non- | No | PR | |
| | PD/not evaluated | | | |
| SD | Non-CR/Non- | No | SD | documented at least |
| | PD/not evaluated | | | once ≥4 wks. from |
| | | | | baseline** |
| PD | Any | Yes or | PD | no prior SD, PR or CR |
| | | No | | |
| Any | PD*** | Yes or | PD | |
| | | No | | |
| Any | Any | Yes | PD | |

*See RECIST 1.1 manuscript for further details on what is evidence of a new lesion.
**Only for non-randomized trials with response as primary endpoint.
***In exceptional circumstances, unequivocal progression in non-target lesions may be accepted as disease progression.
Note:
Participants with a global deterioration of health status requiring discontinuation of treatment without objective evidence of disease progression at that time should be reported as “symptomatic deterioration.” Every effort should be made to document the objective progression even after discontinuation of treatment.

For Participants with Non-Measurable Disease (e.g., Non-Target Disease)

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

*‘Non-CR/non-PD’ is preferred over ‘stable disease’ for non-target disease since SD is increasingly used as an endpoint for assessment of efficacy in some trials so to assign this category when no lesions can be measured is not advised

### Duration of Response

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, takinexg as reference the smallest measurements recorded since the treatment started, including the baseline measurements.

### Progression-Free Survival

PFS is defined as the duration of time from start of treatment to time of progression or death, whichever occurs first.

### Response Review

Response will be evaluated by a board certified radiologist using the above RECIST criteria.

### Statistical Considerations

### Study Design/Endpoints

This is a prospective, 24-week, open-label, single-center, proof-of-concept study in 11 patients to evaluate the safety and potential efficacy of brodalumab in subjects with active irAEs. All subjects will meet the NCI CTCAEv5 definition of >=grade 3 irAEs. Subjects will receive subcutaneous brodalumab for 24 weeks, regardless of irAE resolution. Concurrent glucocorticoid treatment will be permitted. Participants will be evaluated at week 0, 1, 2, 4 and then every 4 weeks after until week 24. A follow up safety visit will be conducted at weeks 28 and 36. A formal assessment of efficacy and safety in the study population will be compared to historical AE rates monthly as dictated by the standard of care. Brodalumab dosing will be based on the FDA-approved regimen for treatment of moderate-severe psoriasis, 210 mg subcutaneous injection on weeks 0, 1, 2 and every 2 weeks after.

### Size/Accrual Rate

The trial will include 11 participants. Accrual of 1-2 participants per month is anticipated.

### Evaluation of Toxicity

All participants will be evaluable for toxicity from the time of their first treatment with the study drug.

### Evaluation of Response

All participants included in the study must be assessed for response to treatment, even if there are major protocol treatment deviations or if they are ineligible. Each patient will be assigned one of the following categories: 1) complete response, 2) partial response, 3) stable disease, 4) progressive disease, 5) early death from malignant disease, 6) early death from toxicity, 7) early death because of other cause, or 9) unknown (not assessable, insufficient data).

All of the participants who met the eligibility criteria (with the exception of those who received no study medication) should be included in the main analysis of the response rate. Participants in response categories 4-9 should be considered to have a treatment failure (disease progression). Thus, an incorrect treatment schedule or drug administration does not result in exclusion from the analysis of the response rate.

All conclusions should be based on all eligible participants. Sub analyses may then be performed on the basis of a subset of participants, excluding those for whom major protocol deviations have been identified (e.g., early death due to other reasons, early discontinuation of treatment, major protocol violations, etc.). However, these sub analyses may not serve as the basis for drawing conclusions concerning treatment efficacy, and the reasons for excluding participants from the analysis should be clearly reported. The 95% confidence intervals should also be provided.

### REFERENCES FOR EXAMPLE 7

1. van Vollenhoven R F, Petri M, Wallace D J, Roth D A, Molta C T, Hammer A E, Tang Y, Thompson A. Cumulative Corticosteroid Dose Over Fifty-Two Weeks in Patients With Systemic Lupus Erythematosus: Pooled Analyses From the Phase III Belimumab Trials. Arthritis Rheumatol. 2016 September; 68 (9): 2184-92.
2. EuroQol Research Foundation. EQ-5D Instrument: euroqol.org/eq-5d-instruments/sample-demo/. Accessed Oct. 27, 2023.
3. Siegel, R. L., et al., Cancer statistics, 2022. CA: A Cancer Journal for Clinicians, 2022. 72 (1): p. 7-33.
4. ClinicalTrials.gov. 2023 [cited 2023 Apr. 22]; Available from: https://clinicaltrials.gov/ct2/results?cond=&term=PDL1&cntry=&state=&city=&dist=
5. Postow, M. A., R. Sidlow, and M. D. Hellmann. Immune-Related Adverse Events Associated with Immune Checkpoint Blockade. New England Journal of Medicine, 2018. 378 (2): p. 158-168.
6. Conroy, M., Naidoo, J. Immune-related adverse events and the balancing act of immunotherapy. Nat Commun 13, 392 (2022). doi.org/10.1038/s41467-022-27960-2.
7. Mor, A. and M. Strazza, Bridging the Gap: Connecting the Mechanisms of Immune-Related Adverse Events and Autoimmunity Through PD-1. Front Cell Dev Biol, 2021. 9: p. 790386.
8. Michot, J. M., et al., Immune-related adverse events with immune checkpoint blockade: a comprehensive review. Eur J Cancer, 2016. 54: p. 139-148.
9. Gettinger, S., et al., Five-Year Follow-Up of Nivolumab in Previously Treated Advanced Non-Small-Cell Lung Cancer: Results From the CA209-003 Study. J Clin Oncol, 2018. 36 (17): p. 1675-1684.
10. Larkin, J., F. S. Hodi, and J. D. Wolchok, Combined Nivolumab and Ipilimumab or Monotherapy in Untreated Melanoma. N Engl J Med, 2015. 373 (13): p. 1270-1.
11. Boutros, C., et al., Safety profiles of anti-CTLA-4 and anti-PD-1 antibodies alone and in combination. Nat Rev Clin Oncol, 2016. 13 (8): p. 473-86.
12. Brahmer, J. R., et al., Management of Immune-Related Adverse Events in Patients Treated With Immune Checkpoint Inhibitor Therapy: American Society of Clinical Oncology Clinical Practice Guideline. J Clin Oncol, 2018. 36 (17): p. 1714-1768.
13. Arbour, K. C., et al., Impact of Baseline Steroids on Efficacy of Programmed Cell Death-1 and Programmed Death-Ligand 1 Blockade in Patients With Non-Small-Cell Lung Cancer. J Clin Oncol, 2018. 36 (28): p. 2872-2878.
14. Libert, C. and L. Dejager, How steroids steer T cells. Cell Rep, 2014. 7 (4): p. 938-9.
15. Bluml, S., et al., B-cell targeted therapeutics in clinical development. Arthritis Res Ther, 2013. 15 Suppl 1: p. S4.
16. Postow, M. A., R. Sidlow, and M. D. Hellmann, Immune-Related Adverse Events Associated with Immune Checkpoint Blockade. New England Journal of Medicine, 2018. 378 (2): p. 158-168.
17. Mor, A. and M. Strazza, Bridging the Gap: Connecting the Mechanisms of Immune-Related Adverse Events and Autoimmunity Through PD-1. Front Cell Dev Biol, 2021. 9: p. 790386.
18. Wang J, Wang C, Liu L, Hong S, Ru Y, Sun X, Chen J, Zhang M, Lin N, Li B, Li X. Adverse events associated with anti-IL-17 agents for psoriasis and psoriatic arthritis: a systematic scoping review. Front Immunol. 2023 Jan. 31; 14:993057. doi: 10.3389/fimmu.2023.993057. PMID: 36817423; PMCID: PMC9928578.
19. Zhao J, Chen X, Herjan T, Li X. The role of interleukin-17 in tumor development and progression. J Exp Med. 2020; 217 (1): e20190297.
20. Adam K, Iuga A, Tocheva A S, Mor A. A novel mouse model for checkpoint inhibitor-induced adverse events. PLOS One. 2021 Feb. 11; 16 (2): c0246168
21. Wang J, Wang C, Liu L, Hong S, Ru Y, Sun X, Chen J, Zhang M, Lin N, Li B, Li X. Adverse events associated with anti-IL-17 agents for psoriasis and psoriatic arthritis: a systematic scoping review. Front Immunol. 2023 Jan. 31; 14:993057.
22. National Cancer Institue Center for Cancer Genomics. The Cancer Genome Atlas Program. www.cancer.gov/ccg/research/genome-sequencing/tcga. Accessed Oct. 28, 2023.
23. The Human Protein Atlas Project. The Human Protein Atlast. www.proteinatlas.org/news/tag/tcga. Accessed Oct. 28, 2023.
24. Puig L, Lebwohl M, Bachelez H, Sobell J, Jacobson A A. Long-term efficacy and safety of brodalumab in the treatment of psoriasis: 120-week results from the randomized, double-blind, placebo- and active comparator-controlled phase 3 AMAGINE-2 trial. J Am Acad Dermatol. 2020; 82 (2): 352-359.
25. Brodalumab Package Insert www.accessdata.fda.gov/drugsatfda_docs/label/2017/7610321bl.pdf
26. American Psychological Association. Physician Health Questionnaire 9. www.apa.org/depression-guideline/patient-health-questionnaire.pdf
27. The Research Foundation for Mental Hygiene, Inc. Columbia Suicide Severity Rating Scale. www.cms.gov/files/document/cssrs-screen-version-instrument.pdf
28. Brodalumab (Siliq) Package Insert. www.accessdata.fda.gov/drugsatfda_docs/label/2017/7610321bl.pdf
29. Coutinho, A. E. and K. E. Chapman, The anti-inflammatory and immunosuppressive effects of glucocorticoids, recent developments and mechanistic insights. Mol Cell Endocrinol, 2011. 335 (1): p. 2-13.
30. National Cancer Institue Cancer Therapy Evaluation Program. Common Terminology Criteria for Adverse Events (CTCAE) v5.0. Available at: ctep.cancer.gov/protocoldevelopment/electronic_applications/ctc.htm #ctc_60. Accessed Oct. 26, 2023.
31. CTCAE v5 ctep.cancer.gov/protocoldevelopment/electronic_applications/ctc.htm #ctc_60
32. van Vollenhoven R F, Petri M, Wallace D J, Roth D A, Molta C T, Hammer A E, Tang Y, Thompson A. Cumulative Corticosteroid Dose Over Fifty-Two Weeks in Patients With Systemic Lupus Erythematosus: Pooled Analyses From the Phase III Belimumab Trials. Arthritis Rheumatol. 2016 September; 68 (9): 2184-92.
33. EuroQol Research Foundation. EQ-5D Instrument: euroqol.org/eq-5d-instruments/sample-demo/. Accessed Oct. 27, 2023.
34. Lexicomp Inc. Prednisone: Drug Information. www.uptodate.com/contents/prednisone-drug-information. Accessed Oct. 27, 2023.
35. Brodalumab (Siliq) Package Insert. www.accessdata.fda.gov/drugsatfda_docs/label/2017/761032lbl.pdf
36. Eisenhauer E A, Therasse P, Bogaerts J, et al. New response evaluation criteria in solid tumours: revised RECIST guideline (version 1.1). Eur J Cancer. 2009; 45 (2): 228-247.

What is claimed is:

1. A method for treating cancer in a subject in need thereof comprising administering to the subject a therapeutically effective amount of an anti-IL-17RA antibody or antigen binding fragment thereof.

2. The method of claim 1, further comprising administering to the subject a therapeutically effective amount of at least one immune checkpoint inhibitor (ICI).

3. The method of claim 2, wherein the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

4. The method of claim 3, wherein the anti-PD-1 antibody or antigen binding fragment thereof comprises Nivolumab, Pembrolizumab, Cemiplimab, Retifanlimab, Dostarlimab, Zimberelimab, Tiselelizumab, Camrelizumab, Sintilimab, Penpulimab or antigen binding fragment thereof.

5. The method of claim 3, wherein the anti-PDL-1 antibody or antigen binding fragment thereof comprises Atezolimumab, Durvalumab and Avelumab, or a combination thereof.

6. The method of claim 3, wherein the anti-CTLA-4 antibody or antigen binding fragment thereof comprises ipilimumab, tremelimumab, or a combination thereof.

7. The method of claim 3, wherein the anti-LAG-3 antibody or antigen binding fragment thereof comprises BMS-986016, Relatimab, INCAGN02385, GSK2831781, or a combination thereof.

8. The method of claims 1-7, wherein the anti-IL-17RA antibody or antigen binding fragment thereof is a monoclonal antibody or antigen binding fragment thereof.

9. The method of claims 1-7, wherein the anti-IL-17RA antibody or antigen binding fragment thereof, comprises:
a first arm comprising a first variable heavy chain domain and a first variable light chain domain, wherein a portion of the first arm is capable of binding to a portion of an IL-17RA; and
a second arm comprising a second variable heavy chain domain and a second variable light chain domain, wherein a portion of the second arm is capable of binding to a portion of the IL-17RA protein
wherein the first and second arms each further comprise a fragment, crystallizable (Fc) domain.

10. The method of claim 9, wherein the first and second arms each further comprise a CH1 domain, a hinge domain, and a CL domain.

11. The method of claims 9-10, wherein the portion of IL-17RA bound by the first arm and second arm is the same.

12. The method of claims 9-10, wherein:
the first variable heavy chain domain of the first arm is encoded by a first polypeptide chain;
the first variable light chain domain of the first arm is encoded by a second polypeptide chain;
the second variable heavy chain domain of the second arm is encoded by a third polypeptide chain;
the second variable light chain domain of the second arm is encoded by a fourth polypeptide chain; and
the first variable heavy chain domain and first variable light chain domain form a first IL-17RA binding site and wherein the second variable heavy chain domain and second variable light chain domain form a second IL-17RA binding site.

13. The method of claim 12, wherein the first and second IL-17RA binding sites are the same.

14. The method of claim 12, wherein the first and third polypeptide chain each further encode a hinge domain, a CH1 domain, and the Fc domain, and wherein the second and fourth polypeptide chain each further encode a CL domain.

15. The method of claim 12-14, wherein the first and third polypeptide chains comprise the same sequence and the second and fourth polypeptide chains comprise the same sequence.

16. The method of claims 9-15, wherein the first and second variable heavy chain domain each comprises HCDR1 comprising SEQ ID NO: 146, HCDR2 comprising SEQ ID NO: 147, and HCDR3 comprising SEQ ID NOs: 148 and wherein the first and second variable light chain domain each comprises LCDR1 comprising SEQ ID NO: 224, LCDR2 comprising SEQ ID NO:225, and LCDR3 comprising SEQ ID NO: 226.

17. The method of claim 16, wherein the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

18. The method of claims 9-15, wherein the first and second variable heavy chain domain each comprises an amino acid sequence of SEQ ID NO: 300 and wherein the first and second variable light chain domain each comprises an amino acid sequence of SEQ ID NO: 301.

19. The method of claims 9-15, wherein the first and third polypeptide chain each comprises an amino acid sequence comprising SEQ ID NO: 300 and the second and fourth polypeptide chain each comprises an amino acid sequence comprising SEQ ID NO: 301.

20. The method of claims 12-15 and 18-19, wherein the first and second polypeptide chains are linked by one or more covalent disulfide bonds and the third and fourth polypeptide chains are linked by one or more covalent disulfide bonds.

21. The method of claims 12-15 and 19-20, wherein the first and third polypeptide chains are linked by one or more covalent disulfide bonds.

22. The method of claims 1-21, wherein the anti-IL17RA antibody is a human or humanized antibody.

23. The method of claim 22, wherein the anti-IL17RA monoclonal antibody is Brodalumab.

24. The method of claims 1-23, wherein the subject has a solid tumor.

25. The method of claim 24, wherein the tumor is head-neck squamous cell carcinoma, sarcoma, liver hepatocellular carcinoma, gastric cancer, colorectal cancer, and breast cancer.

26. The method of claim 24, wherein the cancer is breast cancer.

27. The method of claim 24, wherein the cancer is melanoma.

28. The method of claim 24, wherein the cancer is colon cancer.

29. The method of claims 1-28, wherein immune-related adverse events are treated, reduced, or prevented in the subject.

30. The method of claim 29, wherein the immune-related adverse events comprise colitis, diarrhea, rash, pruritis, esophagitis, duodenitis, ileitis, neuritis, arthrhtis, vasculitis, nephritis, adrenal insufficiency, hepatitis, thrombocytopenia, anemia, pneumonitis, thyroiditis, hypophysitis, encephalitis, meningitis, uveitis, mucositis, rash, myocarditis, pericarditis, pancreatitis, colitis, enteritis, or any combination thereof.

31. The method of claims 1-30, wherein off-target immune infiltration of one or more untargeted organs in the subject is reduced or prevented.

32. The method of claims 1-30, wherein CD3⁺ T cells are not detected or are not present at elevated levels in one or more untargeted organs in the subject.

33. The method of claims 1-32, wherein a tumor of the subject is reduced in volume.

34. The method of claims 1-32, wherein growth of a tumor or cancer cells of the subject is inhibited.

35. The method of claims 1-34, wherein the combination of the anti-IL17RA antibody or antigen binding fragment thereof and the at least one ICI exhibits a synergistic effect on reducing a tumor volume, cancer treatment, or inhibiting tumor growth compared to the tumor volume reduction, cancer treatment effect, or tumor growth inhibition exhibited by administering a therapeutic dose of the one or more ICI alone or a therapeutic dose of the anti-IL-17RA antibody or antigen binding fragment thereof alone.

36. The method of claims 1-34, wherein the anti-IL17RA antibody or antigen binding fragment thereof and the one or more ICI is administered concurrently as a single composition or as separate compositions.

37. The method of claims 1-34, wherein the anti-IL17RA antibody or antigen binding fragment thereof and the one or more ICI is administered sequentially.

38. A method of determining a cancer prognosis in a subject in need thereof comprising determining IL17RA gene expression levels in a sample from the subject.

39. The method of claim 38, wherein the IL17RA gene expression levels are IL17RA gene expression level of T cells of the subject.

40. The method of claim 39, wherein the T cells are CD4 T cells, CD8 T cells, or both CD4 T cells and CD8 T cells.

41. The method of claims 38-40, wherein the subject is determined to have a poor prognosis if the subject has an increased level of IL17RA gene expression as compared to the level of IL17RA gene expression in a healthy subject or cohort of healthy subjects.

42. The method of claims 38-41 wherein the sample is biopsy tissue or blood.

43. The method of claims 38-42, wherein the IL-17RA gene expression is measured using RNA-seq, gene chip data, or q-PCR.

44. The method of claims 1-43, wherein the subject is a human.

45. A composition comprising therapeutically effect amounts of an anti-IL-17RA antibody or antigen binding fragment thereof and one or more ICIs.

46. A combination in the form of a kit comprising two or more compositions, the first composition comprising a therapeutically effect amount of an anti-IL-17RA antibody or antigen binding fragment thereof and the second composition comprising a therapeutically effect amount of one or more ICIs.

47. The compositions of claims 45-46, wherein the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

48. The compositions of claims 46-47, further comprising one or more pharmaceutically acceptable excipients.

49. The compositions of claims 46-48, further comprising a package insert or label providing directions for administering the compositions simultaneously, separately or sequentially.

50. The compositions of claims 46-49, wherein the at least one ICI comprises an anti-CTLA-4 antibody or antigen binding fragment thereof, anti-PD-1 antibody or antigen binding fragment thereof, anti-PDL-1 antibody or antigen binding fragment thereof, anti-LAG-3 antibody or antigen binding fragment thereof or a combination thereof.

51. The compositions of claim 50, wherein the anti-PD-1 antibody or antigen binding fragment thereof comprises Nivolumab, Pembrolizumab, Cemiplimab, Retifanlimab, Dostarlimab, Zimberelimab, Tiselelizumab, Camrelizumab, Sintilimab, Penpulimab or antigen binding fragment thereof.

52. The compositions of claim 50, wherein the anti-PDL-1 antibody or antigen binding fragment thereof comprises Atezolimumab, Durvalumab and Avelumab, or antigen binding fragment thereof.

53. The compositions of claim 50, wherein the anti-CTLA-4 antibody or antigen binding fragment thereof comprises ipilimumab, tremelimumab, or a combination thereof.

54. The compositions of claim 52, wherein the anti-LAG-3 antibody or antigen binding fragment thereof comprises BMS-986016, Relatimab, INCAGN02385, GSK2831781, or a combination thereof.

55. The compositions of claims 45-54, wherein the anti-IL-17RA antibody or antigen binding fragment thereof is a monoclonal antibody or antigen binding fragment thereof.

56. The compositions of claims 45-54, wherein the anti-IL-17RA antibody or antigen binding fragment thereof, comprises:
a first arm comprising a first variable heavy chain domain and a first variable light chain domain, wherein a portion of the first arm is capable of binding to a portion of an IL-17RA; and
a second arm comprising a second variable heavy chain domain and a second variable light chain domain, wherein a portion of the second arm is capable of binding to a portion of the IL-17RA protein
wherein the first and second arms each further comprise a fragment, crystallizable (Fc) domain.

57. The compositions of claim 56, wherein the first and second arms each further comprise a CHI domain, a hinge domain, and a CL domain.

58. The compositions of claims 56-57, wherein the portion of IL-17RA bound by the first arm and second arm is the same.

59. The compositions of claims 56-57, wherein:
the first variable heavy chain domain of the first arm is encoded by a first polypeptide chain;
the first variable light chain domain of the first arm is encoded by a second polypeptide chain;
the second variable heavy chain domain of the second arm is encoded by a third polypeptide chain;
the second variable light chain domain of the second arm is encoded by a fourth polypeptide chain; and
the first variable heavy chain domain and first variable light chain domain form a first IL-17RA binding site and wherein the second variable heavy chain domain and second variable light chain domain form a second IL-17RA binding site.

60. The compositions of claim 59, wherein the first and second IL-17RA binding sites are the same.

61. The compositions of claim 59, wherein the first and third polypeptide chain each further encode a hinge domain, a CH1 domain, and the Fc domain, and wherein the second and fourth polypeptide chain each further encode a CL domain.

62. The compositions of claim 59-61, wherein the first and third polypeptide chains comprise the same sequence and the second and fourth polypeptide chains comprise the same sequence.

63. The compositions of claims 56-62, wherein the first and second variable heavy chain domain each comprises HCDR1 comprising SEQ ID NO: 146, HCDR2 comprising SEQ ID NO: 147, and HCDR3 comprising SEQ ID NO: 148 and wherein the first and second variable light chain domain each comprises LCDR1 comprising SEQ ID NO: 224, LCDR2 comprising SEQ ID NO: 225, and LCDR3 comprising SEQ ID NOs: 226.

64. The compositions of claim 63, wherein the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 300 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 301.

65. The compositions of claims 56-62, wherein the first and second variable heavy chain domain each comprises an amino acid sequence of SEQ ID NO: 300 and wherein the first and second variable light chain domain each comprises an amino acid sequence of SEQ ID NO: 301.

66. The compositions of claims 56-62, wherein the first and third polypeptide chain each comprises an amino acid sequence comprising SEQ ID NO: 300 and the second and fourth polypeptide chain each comprises an amino acid sequence comprising SEQ ID NO: 301.

67. The compositions of claims 59-62 and 65-66, wherein the first and second polypeptide chains are linked by one or more covalent disulfide bonds and the third and fourth polypeptide chains are linked by one or more covalent disulfide bonds.

68. The compositions of claims 59-62 and 65-67, wherein the first and third polypeptide chains are linked by one or more covalent disulfide bonds.

69. The compositions of claims 1-68, wherein the anti-IL17RA antibody is a human or humanized antibody.

70. The compositions of claim 69, wherein the anti-IL17RA monoclonal antibody is Brodalumab.


SEQUENCE LISTING (470 sequences)

SEQ ID NO: 1 - protein, 68 aa, synthetic construct
NYYWNGGGAAAGGGAAADIYYSGSTNYNPSLKSGGGAAAGGGAAADGELANYYGSGSYQFYYYYGMDV

SEQ ID NO: 2 - protein, 64 aa, synthetic construct
GYYWSGGGAAAGGGAAAEINHSGRTNYNPSLKSGGGAAAGGGAAAGPYYFDSSGYLYYYYGLDV

SEQ ID NO: 3 - protein, 51 aa, synthetic construct
SYGMHGGGAAAGGGAAAVIWYDGSNKHYADSVKGGGGAAAGGGAAADTGVY

SEQ ID NO: 4 - protein, 51 aa, synthetic construct; identical to SEQ ID NO: 3

SEQ ID NO: 5 - protein, 62 aa, synthetic construct
SYYWSGGGAAAGGGAAARIYPSGNTIYNPSLKSGGGAAAGGGAAAENYSESSGLYYYYGMDV

SEQ ID NO: 6 - protein, 61 aa, synthetic construct
RYGISGGGAAAGGGAAAWISAYNGNTNYAQKLQGGGGAAAGGGAAARDYDILTGYYNGFDP

SEQ ID NO: 7 - protein, 61 aa, synthetic construct; identical to SEQ ID NO: 6

SEQ ID NO: 8 - protein, 61 aa, synthetic construct
RYGISGGGAAAGGGAAAWISAYNGNTNYAQNLQGGGGAAAGGGAAARDYDILTGYYNGFDP

SEQ ID NO: 9 - protein, 61 aa, synthetic construct; identical to SEQ ID NO: 6

SEQ ID NO: 10 - protein, 61 aa, synthetic construct
SGGYYWSGGGAAAGGGAAAYIYFSGSAYYNPSLKSGGGAAAGGGAAAEYYDSSGYPDAFDI

SEQ ID NO: 11 - protein, 51 aa, synthetic construct
SYGMHGGGAAAGGGAAAVIWYDGSNKYYADSVKGGGGAAAGGGAAADTKDY

SEQ ID NO: 12 - protein, 53 aa, synthetic construct
SYGISGGGAAAGGGAAAWISTYKGNTNYAQKLQGGGGAAAGGGAAAKQLVFDY

SEQ ID NO: 13 - protein, 58 aa, synthetic construct
SYGMQGGGAAAGGGAAAVIWYDGNKKYYADSVKGGGGAAAGGGAAAGRVRDYYYGMDV

SEQ ID NO: 14 - protein, 53 aa, synthetic construct
RYGISGGGAAAGGGAAAWISTYSGNTNYAQKLQGGGGAAAGGGAAARQLYFDY

SEQ ID NO: 15 - protein, 58 aa, synthetic construct; identical to SEQ ID NO: 13

SEQ ID NO: 16 - protein, 53 aa, synthetic construct
SYGISGGGAAAGGGAAAWISAYNGNTKYAQKLQGGGGAAAGGGAAAKQLVFDY

SEQ ID NO: 17 - protein, 53 aa, synthetic construct
SYGISGGGAAAGGGAAAWISAYSGNTKYAQKLQGGGGAAAGGGAAAKQLVFDY

SEQ ID NO: 18 - protein, 63 aa, synthetic construct
DYYMHGGGAAAGGGAAAWMHPNSGGTDLAQRFQGGGGAAAGGGAAACGYCSTLSCSFYFYFDL

SEQ ID NO: 19 - protein, 53 aa, synthetic construct
SYGISGGGAAAGGGAAAWISAYSGNTKYAQKFQGGGGAAAGGGAAARQLALDY

SEQ ID NO: 20 - protein, 55 aa, synthetic construct
SYSMNGGGAAAGGGAAAFISARSSTIYYADSVKGGGGAAAGGGAAAPKVGGGMDV

SEQ ID NO: 21 - protein, 55 aa, synthetic construct
SYSMNGGGAAAGGGAAAIISSRSSIIHYADSVKGGGGAAAGGGAAAPKVGGGMDV

SEQ ID NO: 22 - protein, 53 aa, synthetic construct
RYGISGGGAAAGGGAAAWISAYSGNTNYAQKLQGGGGAAAGGGAAARQLYFDY

SEQ ID NO: 23 - protein, 62 aa, synthetic construct
SYYWSGGGAAAGGGAAARIYPSGRTNYNPSLKSGGGAAAGGGAAAEAYELQLGLYYYYGMDV

SEQ ID NO: 24 - protein, 62 aa, synthetic construct; identical to SEQ ID NO: 23

SEQ ID NO: 25 - protein, 61 aa, synthetic construct
SGGYYWSGGGAAAGGGAAAYIYYSGNTYYNPSLRSGGGAAAGGGAAAEAGGNSAYYYGMDV

SEQ ID NO: 26 - protein, 62 aa, synthetic construct
DYYMSGGGAAAGGGAAAYISSSGSTIYYADSVKGGGGAAAGGGAAADRTYYFGSGSYEGMDV

SEQ ID NO: 27 - protein, 51 aa, synthetic construct
RASQGIRNDLGGGGAAAGGGAAAAASSLQSGGGAAAGGGAAALQHNSNPFT

SEQ ID NO: 28 - protein, 50 aa, synthetic construct
RASQSVSRNLVGGGAAAGGGAAAGASTRANGGGAAAGGGAAAQQYKSWRT

SEQ ID NO: 29 - protein, 51 aa, synthetic construct
RASQSISSVLNGGGAAAGGGAAAAASSLQSGGGAAAGGGAAAQQSYSTPFT

SEQ ID NO: 30 - protein, 52 aa, synthetic construct
RASQSVSRNLAGGGAAAGGGAAAGASTRATGGGAAAGGGAAAQQYNNWPTWT

SEQ ID NO: 31 - protein, 51 aa, synthetic construct
RASQGIRNDLGGGGAAAGGGAAAAASSFQSGGGAAAGGGAAALQHNSYPPT

SEQ ID NO: 32 - protein, 51 aa, synthetic construct
RASQGIRNDLGGGGAAAGGGAAAAASSLQSGGGAAAGGGAAALQHKSYPLT

SEQ ID NO: 33 - protein, 51 aa, synthetic construct; identical to SEQ ID NO: 32

SEQ ID NO: 34 - protein, 51 aa, synthetic construct; identical to SEQ ID NO: 32

SEQ ID NO: 35 - protein, 51 aa, synthetic construct; identical to SEQ ID NO: 32

SEQ ID NO: 36 - protein, 51 aa, synthetic construct
RASQGIRSWLAGGGAAAGGGAAAAASSLQSGGGAAAGGGAAAQQANNFPRT

SEQ ID NO: 37 - protein, 52 aa, synthetic construct
RASQSVSSNLAGGGAAAGGGAAAGASTRAAGGGAAAGGGAAAQHYINWPKWT

SEQ ID NO: 38 - protein, 51 aa, synthetic construct
RASQSISSSLAGGGAAAGGGAAAGASTRATGGGAAAGGGAAAQQYDNWPLT

SEQ ID NO: 39 - protein, 56 aa, synthetic construct
KSSQSLLHSDGKTYLYGGGAAAGGGAAAEVSTRFSGGGAAAGGGAAAMQSIQLPLT

SEQ ID NO: 40 - protein, 51 aa, synthetic construct
RASQSVSSNLAGGGAAAGGGAAADASTRATGGGAAAGGGAAAQQYDNWPLT

SEQ ID NO: 41 - protein, 51 aa, synthetic construct
RASQSVSSNLAGGGAAAGGGAAADASTRAAGGGAAAGGGAAAQQYDNWPLT

SEQ ID NO: 42 - protein, 51 aa, synthetic construct
RASQSISTSLAGGGAAAGGGAAAGTSTRATGGGAAAGGGAAAQQYDIWPLT

SEQ ID NO: 43 - protein, 51 aa, synthetic construct
RASQSVSSNLAGGGAAAGGGAAAGASTRATGGGAAAGGGAAAQQYDNWPLT

SEQ ID NO: 44 - protein, 57 aa, synthetic construct
KTSQSVLYSSKNKNFLAGGGAAAGGGAAAWASTRESGGGAAAGGGAAAQQYYSTPFT

SEQ ID NO: 45 - protein, 51 aa, synthetic construct
RASQSISSNLAGGGAAAGGGAAAGASTRATGGGAAAGGGAAAQQYDTWPLT

SEQ ID NO: 46 - protein, 51 aa, synthetic construct
RASQGISNYLAGGGAAAGGGAAAAASTLQSGGGAAAGGGAAAQKYNRAPFT

SEQ ID NO: 47 - protein, 51 aa, synthetic construct; identical to SEQ ID NO: 46

SEQ ID NO: 48 - protein, 51 aa, synthetic construct; identical to SEQ ID NO: 41

SEQ ID NO: 49 - protein, 51 aa, synthetic construct
RASQGIINDLGGGGAAAGGGAAAAASSLQSGGGAAAGGGAAALQHNSYPPT

SEQ ID NO: 50 - protein, 57 aa, synthetic construct
RSSQSLVYSDGHTCLNGGGAAAGGGAAAKVSNWDSGGGAAAGGGAAAMQGTHWPLCS

SEQ ID NO: 51 - protein, 57 aa, synthetic construct; identical to SEQ ID NO: 50

SEQ ID NO: 52 - protein, 51 aa, synthetic construct
RASQAISIYLAGGGAAAGGGAAAAASSLQSGGGAAAGGGAAAQQYSSYPRT

SEQ ID NO: 53 - protein, 51 aa, synthetic construct
RASQSVYSNLAGGGAAAGGGAAAGASTRATGGGAAAGGGAAAQQYYNWPWT

SEQ ID NO: 54 - DNA, 25 bp, synthetic construct
  source 1..25: mol_type = other DNA
aggcccaggggggctgcagtagacc

SEQ ID NO: 55 - DNA, 25 bp, synthetic construct
  source 1..25: mol_type = other DNA
ccctctacgtggacgtggtcctgaa

SEQ ID NO: 56 - DNA, 25 bp, synthetic construct
  source 1..25: mol_type = other DNA
accacctgcccaagcccatccctga

SEQ ID NO: 57 - DNA, 25 bp, synthetic construct
  source 1..25: mol_type = other DNA
gctgtcgccaccaagtgcagatcca

SEQ ID NO: 58 - DNA, 25 bp, synthetic construct
  source 1..25: mol_type = other DNA
gaccagaagagttccaccagcgatc

SEQ ID NO: 59 - DNA, 25 bp, synthetic construct
  source 1..25: mol_type = other DNA
aaactgaggcatcaccacaggcggt

SEQ ID NO: 60 - DNA, 25 bp, synthetic construct
  source 1..25: mol_type = other DNA
agcacgccaggatgaaggtaaccac

SEQ ID NO: 61 - DNA, 25 bp, synthetic construct
  source 1..25: mol_type = other DNA
caaaggacctgcagatccagctgca

SEQ ID NO: 62 - DNA, 25 bp, synthetic construct
  source 1..25: mol_type = other DNA
gagaaccacagttgctttgagcaca

SEQ ID NO: 63-106 - skipped (44 entries)

SEQ ID NO: 107 - protein, 5 aa, synthetic construct
NYYWN

SEQ ID NO: 108 - protein, 16 aa, synthetic construct
DIYYSGSTNYNPSLKS

SEQ ID NO: 109 - protein, 23 aa, synthetic construct
DGELANYYGSGSYQFYYYYGMDV

SEQ ID NO: 110 - protein, 5 aa, synthetic construct
GYYWS

SEQ ID NO: 111 - protein, 16 aa, synthetic construct
EINHSGRTNYNPSLKS

SEQ ID NO: 112 - protein, 19 aa, synthetic construct
GPYYFDSSGYLYYYYGLDV

SEQ ID NO: 113 - protein, 5 aa, synthetic construct
SYGMH

SEQ ID NO: 114 - protein, 17 aa, synthetic construct
VIWYDGSNKHYADSVKG

SEQ ID NO: 115 - protein, 5 aa, synthetic construct
DTGVY

SEQ ID NO: 116 - protein, 5 aa, synthetic construct
SYGMH

SEQ ID NO: 117 - protein, 17 aa, synthetic construct
VIWYDGSNKHYADSVKG

SEQ ID NO: 118 - protein, 5 aa, synthetic construct
DTGVY

SEQ ID NO: 119 - protein, 5 aa, synthetic construct
SYYWS

SEQ ID NO: 120 - protein, 16 aa, synthetic construct
RIYRSGNTIYNPSLKS

SEQ ID NO: 121 - protein, 17 aa, synthetic construct
ENYSESSGLYYYYGMDV

SEQ ID NO: 122 - protein, 5 aa, synthetic construct
RYGIS

SEQ ID NO: 123 - protein, 17 aa, synthetic construct
WISAYNGNTNYAQKLQG

SEQ ID NO: 124 - protein, 15 aa, synthetic construct
RDYDILTGYYNGFDP

SEQ ID NO: 125 - protein, 5 aa, synthetic construct
RYGIS

SEQ ID NO: 126 - protein, 17 aa, synthetic construct
WISAYNGNTNYAQKLQG

SEQ ID NO: 127 - protein, 15 aa, synthetic construct
RDYDILTGYYNGFDP

SEQ ID NO: 128 - protein, 5 aa, synthetic construct
GYGIS

SEQ ID NO: 129 - protein, 17 aa, synthetic construct
WISAYNGNTNYAQNLQG

SEQ ID NO: 130 - protein, 15 aa, synthetic construct
RDYDILTGYYNGFDP

SEQ ID NO: 131 - protein, 5 aa, synthetic construct
RYGIS

SEQ ID NO: 132 - protein, 17 aa, synthetic construct
WISAYNGNTNYAQKLQG

SEQ ID NO: 133 - protein, 15 aa, synthetic construct
RDYDILTGYYNGFDP

SEQ ID NO: 134 - protein, 7 aa, synthetic construct
SGGYYWS

SEQ ID NO: 135 - protein, 16 aa, synthetic construct
YIYFSGSAYYNPSLKS

SEQ ID NO: 136 - protein, 14 aa, synthetic construct
EYYDSSGYPDAFDI

SEQ ID NO: 137 - protein, 5 aa, synthetic construct
SYGMH

SEQ ID NO: 138 - protein, 17 aa, synthetic construct
VIWYDGSNKYYADSVKG

SEQ ID NO: 139 - protein, 5 aa, synthetic construct
DTKDY

SEQ ID NO: 140 - protein, 5 aa, synthetic construct
SYGIS

SEQ ID NO: 141 - protein, 17 aa, synthetic construct
WISTYKGNTNYAQKLQG

SEQ ID NO: 142 - protein, 7 aa, synthetic construct
KQLVFDY

SEQ ID NO: 143 - protein, 5 aa, synthetic construct
SYGMQ

SEQ ID NO: 144 - protein, 17 aa, synthetic construct
VIWYDGNKKYYADSVKG

SEQ ID NO: 145 - protein, 12 aa, synthetic construct
GRVRDYYYGMDV

SEQ ID NO: 146 - protein, 5 aa, synthetic construct
RYGIS

SEQ ID NO: 147 - protein, 17 aa, synthetic construct
WISTYSGNTNYAQKLQG

SEQ ID NO: 148 - protein, 7 aa, synthetic construct
RQLYFDY

SEQ ID NO: 149 - protein, 5 aa, synthetic construct
SYGMQ

SEQ ID NO: 150 - protein, 17 aa, synthetic construct
VIWYDGNKKYYADSVKG

SEQ ID NO: 151 - protein, 12 aa, synthetic construct
GRVRDYYYGMDV

SEQ ID NO: 152 - protein, 5 aa, synthetic construct
SYGIS

SEQ ID NO: 153 - protein, 17 aa, synthetic construct
WISAYNGNTKYAQKLQG

SEQ ID NO: 154 - protein, 7 aa, synthetic construct
KQLVFDY

SEQ ID NO: 155 - protein, 5 aa, synthetic construct
SYGIS

SEQ ID NO: 156 - protein, 17 aa, synthetic construct
WISAYSGNTKYAQKLQG

SEQ ID NO: 157 - protein, 7 aa, synthetic construct
KQLVFDY

SEQ ID NO: 158 - protein, 5 aa, synthetic construct
DYYMH

SEQ ID NO: 159 - protein, 17 aa, synthetic construct
WMHPNSGGTDLAQRFQG

SEQ ID NO: 160 - protein, 17 aa, synthetic construct
GGYCSTLSCSFYWYFDL

SEQ ID NO: 161 - protein, 5 aa, synthetic construct
SYGIS

SEQ ID NO: 162 - protein, 17 aa, synthetic construct
WISAYSGNTKYAQKFQG

SEQ ID NO: 163 - protein, 7 aa, synthetic construct
RQLALDY

SEQ ID NO: 164 - protein, 5 aa, synthetic construct
SYSMN

SEQ ID NO: 165 - protein, 17 aa, synthetic construct
FISARSSTIYYADSVKG

SEQ ID NO: 166 - protein, 9 aa, synthetic construct
PKVGGGMDV

SEQ ID NO: 167 - protein, 5 aa, synthetic construct
SYSMN

SEQ ID NO: 168 - protein, 17 aa, synthetic construct
IISSRSSIIHYADSVKG

SEQ ID NO: 169 - protein, 9 aa, synthetic construct
PKVGGGMDV

SEQ ID NO: 170 - protein, 5 aa, synthetic construct
RYGIS

SEQ ID NO: 171 - protein, 17 aa, synthetic construct
WISAYSGNTNYAQKLQG

SEQ ID NO: 172 - protein, 7 aa, synthetic construct
RQLYFDY

SEQ ID NO: 173 - protein, 5 aa, synthetic construct
SYYWS

SEQ ID NO: 174 - protein, 16 aa, synthetic construct
RIYPSGRTNYNPSLKS

SEQ ID NO: 175 - protein, 17 aa, synthetic construct
EAYELQLGLYYYYGMDV

SEQ ID NO: 176 - protein, 5 aa, synthetic construct
SYYWS

SEQ ID NO: 177 - protein, 16 aa, synthetic construct
RIYPSGRTNYNPSLKS

SEQ ID NO: 178 - protein, 17 aa, synthetic construct
EAYELQLGLYYYYGMDV

SEQ ID NO: 179 - protein, 7 aa, synthetic construct
SGGYYWS

SEQ ID NO: 180 - protein, 13 aa, synthetic construct
YSGNTYYNPSLRS

SEQ ID NO: 181 - protein, 14 aa, synthetic construct
EAGGNSAYYYGMDV

SEQ ID NO: 182 - protein, 5 aa, synthetic construct
DYYMS

SEQ ID NO: 183 - protein, 17 aa, synthetic construct
YISSSGSTIYYADSVKG

SEQ ID NO: 184 - protein, 16 aa, synthetic construct
DRTYYFGSGSYEGMDV

SEQ ID NO: 185 - protein, 11 aa, synthetic construct
RASQGIRNDLG

SEQ ID NO: 186 - protein, 7 aa, synthetic construct
AASSLQS

SEQ ID NO: 187 - protein, 9 aa, synthetic construct
LQHNSNPFT

SEQ ID NO: 188 - protein, 11 aa, synthetic construct
RASQSVSRNLV

SEQ ID NO: 189 - protein, 7 aa, synthetic construct
GASTRAN

SEQ ID NO: 190 - protein, 8 aa, synthetic construct
QQYKSWRT

SEQ ID NO: 191 - protein, 11 aa, synthetic construct
RASQSISSYLN

SEQ ID NO: 192 - protein, 7 aa, synthetic construct
AASSLQS

SEQ ID NO: 193 - protein, 9 aa, synthetic construct
QQSYSTPFT

SEQ ID NO: 194 - protein, 11 aa, synthetic construct
RASQSVSRNLA

SEQ ID NO: 195 - protein, 7 aa, synthetic construct
GASTRAT

SEQ ID NO: 196 - protein, 10 aa, synthetic construct
QQYNNWPTWT

SEQ ID NO: 197 - protein, 11 aa, synthetic construct
RASQGIRNDLG

SEQ ID NO: 198 - protein, 7 aa, synthetic construct
AASSFQS

SEQ ID NO: 199 - protein, 9 aa, synthetic construct
LQHNSYPPT

SEQ ID NO: 200 - protein, 11 aa, synthetic construct
RASQGIRNDLG

SEQ ID NO: 201 - protein, 7 aa, synthetic construct
AASSLQS

SEQ ID NO: 202 - protein, 9 aa, synthetic construct
LQHKSYPLT

SEQ ID NO: 203 - protein, 11 aa, synthetic construct
RASQGIRNDLG

SEQ ID NO: 204 - protein, 7 aa, synthetic construct
AASSLQS

SEQ ID NO: 205 - protein, 9 aa, synthetic construct
LQHKSYPLT

SEQ ID NO: 206 - protein, 11 aa, synthetic construct
RASQGIRNDLG

SEQ ID NO: 207 - protein, 7 aa, synthetic construct
AASSLQS

SEQ ID NO: 208 - protein, 9 aa, synthetic construct
LQHKSYPLT

SEQ ID NO: 209 - protein, 11 aa, synthetic construct
RASQGIRNDLG

SEQ ID NO: 210 - protein, 7 aa, synthetic construct
AASSLQS

SEQ ID NO: 211 - protein, 9 aa, synthetic construct
LQHKSYPLT

SEQ ID NO: 212 - protein, 11 aa, synthetic construct
RASQGIRSWLA

SEQ ID NO: 213 - protein, 7 aa, synthetic construct
AASSLQS

SEQ ID NO: 214 - protein, 9 aa, synthetic construct
QQANNFPRT

SEQ ID NO: 215 - protein, 11 aa, synthetic construct
RASQSVSSNLA

SEQ ID NO: 216 - protein, 7 aa, synthetic construct
GASTRAA

SEQ ID NO: 217 - protein, 10 aa, synthetic construct
QHYINWPKWT

SEQ ID NO: 218 - protein, 11 aa, synthetic construct
RASQSISSSLA

SEQ ID NO: 219 - protein, 7 aa, synthetic construct
GASTRAT

SEQ ID NO: 220 - protein, 9 aa, synthetic construct
QQYDNWPLT

SEQ ID NO: 221 - protein, 16 aa, synthetic construct
KSSQSLLHSDGKTYLY

SEQ ID NO: 222 - protein, 7 aa, synthetic construct
EVSTRFS

SEQ ID NO: 223 - protein, 9 aa, synthetic construct
MQSIQLPLT

SEQ ID NO: 224 - protein, 11 aa, synthetic construct
RASQSVSSNLA

SEQ ID NO: 225 - protein, 7 aa, synthetic construct
DASTRAT

SEQ ID NO: 226 - protein, 9 aa, synthetic construct
QQYDNWPLT

SEQ ID NO: 227 - protein, 11 aa, synthetic construct
RASQSVSSNLA

SEQ ID NO: 228 - protein, 7 aa, synthetic construct
DASTRAA

SEQ ID NO: 229 - protein, 9 aa, synthetic construct
QQYDNWPLT

SEQ ID NO: 230 - protein, 11 aa, synthetic construct
RASQSISTSLA

SEQ ID NO: 231 - protein, 7 aa, synthetic construct
GTSTRAT

SEQ ID NO: 232 - protein, 9 aa, synthetic construct
QQYDIWPLT

SEQ ID NO: 233 - protein, 11 aa, synthetic construct
RASQSVSSNLA

SEQ ID NO: 234 - protein, 7 aa, synthetic construct
GASTRAT

SEQ ID NO: 235 - protein, 9 aa, synthetic construct
QQYDNWPLT

SEQ ID NO: 236 - protein, 17 aa, synthetic construct
KTSQSVLYSSKNKNFLA

SEQ ID NO: 237 - protein, 7 aa, synthetic construct
WASTRES

SEQ ID NO: 238 - protein, 9 aa, synthetic construct
QQYYSTPFT

SEQ ID NO: 239 - protein, 11 aa, synthetic construct
RASQSISSNLA

SEQ ID NO: 240 - protein, 7 aa, synthetic construct
GASTRAT

SEQ ID NO: 241 - protein, 9 aa, synthetic construct
QQYDTWPLT

SEQ ID NO: 242 - protein, 11 aa, synthetic construct
RASQGISNYLA

SEQ ID NO: 243 - protein, 7 aa, synthetic construct
AASTLQS

SEQ ID NO: 244 - protein, 9 aa, synthetic construct
QKYNRAPFT

SEQ ID NO: 245 - protein, 11 aa, synthetic construct
RASQGISNYLA

SEQ ID NO: 246 - protein, 7 aa, synthetic construct
AASTLQS

SEQ ID NO: 247 - protein, 9 aa, synthetic construct
QKYNRAPFT

SEQ ID NO: 248 - protein, 11 aa, synthetic construct
RASQSVSSNLA

SEQ ID NO: 249 - protein, 7 aa, synthetic construct
DASTRAA

SEQ ID NO: 250 - protein, 9 aa, synthetic construct
QQYDNWPLT

SEQ ID NO: 251 - protein, 11 aa, synthetic construct
RASQGIINDLG

SEQ ID NO: 252 - protein, 7 aa, synthetic construct
AASSLQS

SEQ ID NO: 253 - protein, 9 aa, synthetic construct
LQHNSYPPT

SEQ ID NO: 254 - protein, 16 aa, synthetic construct
RSSQSLVYSDGHTCLN

SEQ ID NO: 255 - protein, 7 aa, synthetic construct
KVSNWDS

SEQ ID NO: 256 - protein, 10 aa, synthetic construct
MQGTHWPLCS

SEQ ID NO: 257 - protein, 16 aa, synthetic construct
RSSQSLVYSDGHTCLN

SEQ ID NO: 258 - protein, 7 aa, synthetic construct
KVSNWDS

SEQ ID NO: 259 - protein, 10 aa, synthetic construct
MQGTHWPLCS

SEQ ID NO: 260 - protein, 11 aa, synthetic construct
RASQAISIYLA

SEQ ID NO: 261 - protein, 7 aa, synthetic construct
AASSLQS

SEQ ID NO: 262 - protein, 9 aa, synthetic construct
QQYSSYPRT

SEQ ID NO: 263 - protein, 11 aa, synthetic construct
RASQSVYSNLA

SEQ ID NO: 264 - protein, 7 aa, synthetic construct
GASTRAT

SEQ ID NO: 265 - protein, 9 aa, synthetic construct
QQYYNWPWT

SEQ ID NO: 266-299 - skipped (34 entries)

SEQ ID NO: 300 - protein, 116 aa, synthetic construct
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 301 - protein, 107 aa, synthetic construct
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 302 - DNA, 25 bp, synthetic construct
  source 1..25: mol_type = other DNA
gctgtgacggcgacgtccccgacct

SEQ ID NO: 303 - DNA, 22 bp, synthetic construct
  source 1..22: mol_type = other DNA
gtaaataattgtgcttcgtcag

SEQ ID NO: 304 - DNA, 20 bp, synthetic construct
  source 1..20: mol_type = other DNA
tagaaaggtgcacgggtgtg

SEQ ID NO: 305 - DNA, 21 bp, synthetic construct
  source 1..21: mol_type = other DNA
caaatctaggcattaacagtg

SEQ ID NO: 306 - protein, 121 aa, synthetic construct
EVKLVESGGGLVQPGGSLKLSCAASGFTFSSYTMSWVRQTPEKRLEWVAYISYGGGDTYYSDTVKGRFTISRDNAKNTLYLQMSSLKSEDTAMYYCARQKVDGYYVGMDYWGQGSSVTVSS

SEQ ID NO: 307 - protein, 107 aa, synthetic construct
NIVMTQSPKSMSMSVGERVTLSCKASENVGTNVSWYQQKPEQSPRLLIYGASNRYTGVPDRFTGSGSVTDFTLTISSVQAEDLVDYHCGQSYSSPLTFGSGTKLELK

SEQ ID NO: 308 - protein, 120 aa, synthetic construct
EVQLVESGGGLVKPGGSLRLSCAASGFTFSSYGMSWVRQAPGKGLEWVATISGGGRYTYYPDSVKGRFTISRDNAKNSLYLQMNSLRAEDTAVYYCASPYDGYYGAMDYWGQGTSVTVSS

SEQ ID NO: 309 - protein, 112 aa, synthetic construct
EIVLTQSPATLSLSPGERATLSCRASESVDNSGISFMNWYQQKPGQAPRLLIYAASNQGSGIPARFSGSGSGTDFTLTISSLEPEDFAVYYCQQSKEVPWTFGGGTKLEIKR

SEQ ID NO: 310 - skipped

SEQ ID NO: 311 - protein, 243 aa, synthetic construct
EVKLVESGGGLVQPGGSLKLSCAASGFTFSSYTMSWVRQTPEKRLEWVAYISYGGGDTYYSDTVKGRFTISRDNAKNTLYLQMSSLKSEDTAMYYCARQKVDGYYVGMDYWGQGSSVTVSSGGGGSGGGGSGGGGSNIVMTQSPKSMSMSVGERVTLSCKASENVGTNVSWYQQKPEQSPRLLIYGASNRYTGVPDRFTGSGSVTDFTLTISSVQAEDLVDYHCGQSYSSPLTFGSGTKLELK

SEQ ID NO: 312 - protein, 247 aa, synthetic construct
EVQLVESGGGLVKPGGSLRLSCAASGFTFSSYGMSWVRQAPGKGLEWVATISGGGRYTYYPDSVKGRFTISRDNAKNSLYLQMNSLRAEDTAVYYCASPYDGYYGAMDYWGQGTSVTVSSGGGGSGGGGSGGGGSEIVLTQSPATLSLSPGERATLSCRASESVDNSGISFMNWYQQKPGQAPRLLIYAASNQGSGIPARFSGSGSGTDFTLTISSLEPEDFAVYYCQQSKEVPWTFGGGTKLEIKR

SEQ ID NO: 313 - protein, 117 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSS

SEQ ID NO: 314 - protein, 120 aa, synthetic construct
EVQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSS

SEQ ID NO: 315 - protein, 121 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSS

SEQ ID NO: 316 - protein, 122 aa, synthetic construct
EVQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSS

SEQ ID NO: 317 - protein, 121 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSS

SEQ ID NO: 318 - protein, 120 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSS

SEQ ID NO: 319 - protein, 115 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSS

SEQ ID NO: 320 - protein, 117 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSS

SEQ ID NO: 321 - protein, 117 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSS

SEQ ID NO: 322 - protein, 112 aa, synthetic construct
NIVMTQATPSVPVTPGESVSISCRSSKSLLHSDGDTYLYWFLQRPGQSPQLLIYRMSNLVSGVPDRFSGSGSGTAFTLRISRVEAEDVGVYYCMQHLEYPYTFGGGTKLEIK

SEQ ID NO: 323 - protein, 110 aa, synthetic construct
QIVLTQSPAIMSSSLGERVTMTCTASSSVSSSYLHWYQQKPGSSPKLWIYSTSNLASGVPARFSGSGSGTSYSLTISSMEAEDAATYYCHQYHRSPPIFTFGSGTKLEIK

SEQ ID NO: 324 - protein, 108 aa, synthetic construct
QIVLTQSPAIMSASLGERVTMTCTVSSSISSSYLHWYQQKPGSSPKLWIYSTSNLASGVPARFSGSGSGTSYSLTISSMEAEDAATYYCHQYHRSPLTFGAGTKLELK

SEQ ID NO: 325 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYHKLPWTFGGGTKLEIK

SEQ ID NO: 326 - protein, 111 aa, synthetic construct
VIVLTQSPASLAVSLGQRATISCRASERVDDYGISFMNWFQQKPGQPPKLLIYAASNQGSGVPARFSGSGSGTDFSLNIHPMEEDDTAMYFCQQNKEVPWTFGGGTKLEIK

SEQ ID NO: 327 - protein, 111 aa, synthetic construct
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 328 - protein, 107 aa, synthetic construct
DIKMTQSPSSLYASLGERVTITCKASQDINRYLTWFQQKPGKSPKTLIFRANRLVTGVPSRFIGSGSGQEYSLTISSLEYEDMGIYFCLQYDESPYTFGGGTKLEIK

SEQ ID NO: 329 - protein, 107 aa, synthetic construct
DIQMTQSPSSLSASLGERVNLTCRASQEISGYLSWLQQKPDGTIKRLIYVASTLDSGVPERFSGSRSGSDYSLTISSLESEDFADYYCLQYASYPYTFGGGTKLEIK

SEQ ID NO: 330 - protein, 107 aa, synthetic construct
DILLTQSPSSMSVSLGDTVSITCHASQDISRNIGWLRQKPGKSFKGLIYHGTNLEDGVPSRFSGSGSGADYSLTISSLESEDFADYYCVQYAQFPYTFGGGTKLEIK

SEQ ID NO: 331 - protein, 5 aa, synthetic construct
RYWMS

SEQ ID NO: 332 - protein, 17 aa, synthetic construct
EINPDSSTIKYTPSLKD

SEQ ID NO: 333 - protein, 8 aa, synthetic construct
MGYRLFDS

SEQ ID NO: 334 - protein, 5 aa, synthetic construct
SYWMH

SEQ ID NO: 335 - protein, 17 aa, synthetic construct
AIYPGYSDTRYNQKFKG

SEQ ID NO: 336 - protein, 11 aa, synthetic construct
FIATVGGYFDV

SEQ ID NO: 337 - protein, 5 aa, synthetic construct
DYSMH

SEQ ID NO: 338 - protein, 17 aa, synthetic construct
WIKIETGNPTYADDFKG

SEQ ID NO: 339 - protein, 12 aa, synthetic construct
DYYGYYYYAMDY

SEQ ID NO: 340 - protein, 5 aa, synthetic construct
SYTMS

SEQ ID NO: 341 - protein, 17 aa, synthetic construct
YISYGGGDTYYSDTVKG

SEQ ID NO: 342 - protein, 12 aa, synthetic construct
QKVDGYYVGMDY

SEQ ID NO: 343 - protein, 5 aa, synthetic construct
HYGMS

SEQ ID NO: 344 - protein, 17 aa, synthetic construct
TIDNNGGNTYYPDSVKG

SEQ ID NO: 345 - protein, 13 aa, synthetic construct
DAYYTYDYWYFDV

SEQ ID NO: 346 - protein, 17 aa, synthetic construct
YISYGGGDTYYPDTVKG

SEQ ID NO: 347 - protein, 12 aa, synthetic construct
QKVDGYFVAMDY

SEQ ID NO: 348 - protein, 7 aa, synthetic construct
GFTFSSY

SEQ ID NO: 349 - protein, 6 aa, synthetic construct
SGGGRY

SEQ ID NO: 350 - protein, 15 aa, synthetic construct
CASPYDGYYGAMDYW

SEQ ID NO: 351 - protein, 5 aa, synthetic construct
TYWMH

SEQ ID NO: 352 - protein, 17 aa, synthetic construct
GIYPGNSDTNYNQKFKG

SEQ ID NO: 353 - protein, 6 aa, synthetic construct
GYFFDV

SEQ ID NO: 354 - protein, 5 aa, synthetic construct
DYYMN

SEQ ID NO: 355 - protein, 17 aa, synthetic construct
WIYPGSGSTKDNENFKG

SEQ ID NO: 356 - protein, 8 aa, synthetic construct
YGPRFFDV

SEQ ID NO: 357 - protein, 5 aa, synthetic construct
RDWMS

SEQ ID NO: 358 - protein, 17 aa, synthetic construct
QISPDSTSINYKPSLKD

SEQ ID NO: 359 - protein, 8 aa, synthetic construct
DTSGYPDY

SEQ ID NO: 360 - protein, 30 aa, synthetic construct
EVKLLESGGGLVQPGGSLKLSCAASGFDFS

SEQ ID NO: 361 - protein, 14 aa, synthetic construct
WVRQAPGKGLEWIG

SEQ ID NO: 362 - protein, 32 aa, synthetic construct
KFIISRDNAKNTLCLQLSKVRSEDSALYYCAR

SEQ ID NO: 363 - protein, 11 aa, synthetic construct
WGQGTTLTVSS

SEQ ID NO: 364 - protein, 30 aa, synthetic construct
EVQLQQSGTVLARPGASVKMSCKASGNTFT

SEQ ID NO: 365 - protein, 14 aa, synthetic construct
WVKQRPGQGLEWIG

SEQ ID NO: 366 - protein, 32 aa, synthetic construct
KATLTAVTSTSTAYMELSSLTNEDSAVYYCTK

SEQ ID NO: 367 - protein, 11 aa, synthetic construct
WGAGTTVTVSS

SEQ ID NO: 368 - protein, 30 aa, synthetic construct
QIQLVQSGPELKKPGETVKISCKASGYTFT

SEQ ID NO: 369 - protein, 14 aa, synthetic construct
WVKQAPGKGLKWMG

SEQ ID NO: 370 - protein, 32 aa, synthetic construct
RFAFSLETSASTAYLQINNLKNEDTATYFCAR

SEQ ID NO: 371 - protein, 11 aa, synthetic construct
WGQGTSVTVSS

SEQ ID NO: 372 - protein, 30 aa, synthetic construct
EVKLVESGGGLVQPGGSLKLSCAASGFTFS

SEQ ID NO: 373 - protein, 14 aa, synthetic construct
WVRQTPEKRLEWVA

SEQ ID NO: 374 - protein, 32 aa, synthetic construct
RFTISRDNAKNTLYLQMSSLKSEDTAMYYCAR

SEQ ID NO: 375 - protein, 11 aa, synthetic construct
WGQGSSVTVSS

SEQ ID NO: 376 - protein, 30 aa, synthetic construct
EVQLVESGGGLVQPGGSLRLSCAASGFTFS

SEQ ID NO: 377 - protein, 14 aa, synthetic construct
WVRQTPDKRLELVA

SEQ ID NO: 378 - protein, 11 aa, synthetic construct
WSQGTSVTVSS

SEQ ID NO: 379 - protein, 25 aa, synthetic construct
EVKLVESGGGLVKPGGSLKLSCAAS

SEQ ID NO: 380 - protein, 19 aa, synthetic construct
GMSWVRQTPEKRLEWVATI

SEQ ID NO: 381 - protein, 38 aa, synthetic construct
TYYPDSVKGRFTISRDNAKNILYLQMSSLRSEDTALYY

SEQ ID NO: 382 - protein, 10 aa, synthetic construct
GQGTSVTVSS

SEQ ID NO: 383 - protein, 30 aa, synthetic construct
EVQLQQSGTVLARPGASVRMSCKASGYSFS

SEQ ID NO: 384 - protein, 32 aa, synthetic construct
KAKLTAVTSASTAYMDLSSLTNEDSAVYYCTG

SEQ ID NO: 385 - protein, 30 aa, synthetic construct
QIQLQQSGPEQVKPGASVKISCKASGYMFI

SEQ ID NO: 386 - protein, 14 aa, synthetic construct
WVKQKPGQGLEWIG

SEQ ID NO: 387 - protein, 32 aa, synthetic construct
KATLTVDTSSSTAYMQLSSLTSEDTAVYFCAR

SEQ ID NO: 388 - protein, 30 aa, synthetic construct
EVKLLESGGGLVQPGGSLKLSCEVSGFDFS

SEQ ID NO: 389 - protein, 32 aa, synthetic construct
KFIISRDNAKNTLYLQLSGVRSEDTALYHCAR

SEQ ID NO: 390 - protein, 11 aa, synthetic construct
WGQGTSLTVSS

SEQ ID NO: 391 - protein, 16 aa, synthetic construct
RSSKSLLHSDGDTYLY

SEQ ID NO: 392 - protein, 7 aa, synthetic construct
RMSNLVS

SEQ ID NO: 393 - protein, 9 aa, synthetic construct
MQHLEYPYT

SEQ ID NO: 394 - protein, 12 aa, synthetic construct
TASSSVSSSYLH

SEQ ID NO: 395 - protein, 7 aa, synthetic construct
STSNLAS

SEQ ID NO: 396 - protein, 11 aa, synthetic construct
HQYHRSPPIFT

SEQ ID NO: 397 - protein, 12 aa, synthetic construct
TVSSSISSSYLH

SEQ ID NO: 398 - protein, 9 aa, synthetic construct
HQYHRSPLT

SEQ ID NO: 399 - protein, 11 aa, synthetic construct
KASENVGTNVS

SEQ ID NO: 400 - protein, 7 aa, synthetic construct
GASNRYT

SEQ ID NO: 401 - protein, 9 aa, synthetic construct
GQSYSSPLT

SEQ ID NO: 402 - protein, 11 aa, synthetic construct
SASQGISNYLN

SEQ ID NO: 403 - protein, 7 aa, synthetic construct
YTSSLHS

SEQ ID NO: 404 - protein, 9 aa, synthetic construct
QQYHKLPWT

SEQ ID NO: 405 - protein, 15 aa, synthetic construct
RASERVDDYGISFMN

SEQ ID NO: 406 - protein, 7 aa, synthetic construct
AASNQGS

SEQ ID NO: 407 - protein, 9 aa, synthetic construct
QQNKEVPWT

SEQ ID NO: 408 - protein, 15 aa, synthetic construct
RASESVDNSGISFMN

SEQ ID NO: 409 - protein, 11 aa, synthetic construct
CQQSKEVPWTF

SEQ ID NO: 410 - protein, 11 aa, synthetic construct
KASQDINRYLT

SEQ ID NO: 411 - protein, 7 aa, synthetic construct
RANRLVT

SEQ ID NO: 412 - protein, 9 aa, synthetic construct
LQYDESPYT

SEQ ID NO: 413 - protein, 11 aa, synthetic construct
RASQEISGYLS

SEQ ID NO: 414 - protein, 7 aa, synthetic construct
VASTLDS

SEQ ID NO: 415 - protein, 9 aa, synthetic construct
LQYASYPYT

SEQ ID NO: 416 - protein, 11 aa, synthetic construct
HASQDISRNIG

SEQ ID NO: 417 - protein, 7 aa, synthetic construct
HGTNLED

SEQ ID NO: 418 - protein, 9 aa, synthetic construct
VQYAQFPYT

SEQ ID NO: 419 - protein, 23 aa, synthetic construct
NIVMTQATPSVPVTPGESVSISC

SEQ ID NO: 420 - protein, 15 aa, synthetic construct
WFLQRPGQSPQLLIY

SEQ ID NO: 421 - protein, 32 aa, synthetic construct
GVPDRFSGSGSGTAFTLRISRVEAEDVGVYYC

SEQ ID NO: 422 - protein, 10 aa, synthetic construct
FGGGTKLEIK

SEQ ID NO: 423 - protein, 23 aa, synthetic construct
QIVLTQSPAIMSSSLGERVTMTC

SEQ ID NO: 424 - protein, 15 aa, synthetic construct
WYQQKPGSSPKLWIY

SEQ ID NO: 425 - protein, 32 aa, synthetic construct
GVPARFSGSGSGTSYSLTISSMEAEDAATYYC

SEQ ID NO: 426 - protein, 10 aa, synthetic construct
FGSGTKLEIK

SEQ ID NO: 427 - protein, 23 aa, synthetic construct
QIVLTQSPAIMSASLGERVTMTC

SEQ ID NO: 428 - protein, 10 aa, synthetic construct
FGAGTKLELK

SEQ ID NO: 429 - protein, 23 aa, synthetic construct
NIVMTQSPKSMSMSVGERVTLSC

SEQ ID NO: 430 - protein, 15 aa, synthetic construct
WYQQKPEQSPRLLIY

SEQ ID NO: 431 - protein, 32 aa, synthetic construct
GVPDRFTGSGSVTDFTLTISSVQAEDLVDYHC

SEQ ID NO: 432 - protein, 10 aa, synthetic construct
FGSGTKLELK

SEQ ID NO: 433 - protein, 23 aa, synthetic construct
DIQMTQTTSSLSASLGDRVTISC

SEQ ID NO: 434 - protein, 15 aa, synthetic construct
WYQQKPDGTVKLLIY

SEQ ID NO: 435 - protein, 32 aa, synthetic construct
GVPSRFSGSGSGTDYSLTISNLEPEDIATYYC

SEQ ID NO: 436 - protein, 23 aa, synthetic construct
VIVLTQSPASLAVSLGQRATISC

SEQ ID NO: 437 - protein, 15 aa, synthetic construct
WFQQKPGQPPKLLIY

SEQ ID NO: 438 - protein, 32 aa, synthetic construct
GVPARFSGSGSGTDFSLNIHPMEEDDTAMYFC

SEQ ID NO: 439 - protein, 23 aa, synthetic construct
DIVLTQSPASLAVSLGQRATISC

SEQ ID NO: 440 - protein, 31 aa, synthetic construct
GVPARFSGSGSGTDFSLNIHPMEEDDTAMYF

SEQ ID NO: 441 - protein, 9 aa, synthetic construct
GGGTKLEIK

SEQ ID NO: 442 - protein, 23 aa, synthetic construct
DIKMTQSPSSLYASLGERVTITC

SEQ ID NO: 443 - protein, 15 aa, synthetic construct
WFQQKPGKSPKTLIF

SEQ ID NO: 444 - protein, 32 aa, synthetic construct
GVPSRFIGSGSGQEYSLTISSLEYEDMGIYFC

SEQ ID NO: 445 - protein, 23 aa, synthetic construct
DIQMTQSPSSLSASLGERVNLTC

SEQ ID NO: 446 - protein, 15 aa, synthetic construct
WLQQKPDGTIKRLIY

SEQ ID NO: 447 - protein, 32 aa, synthetic construct
GVPERFSGSRSGSDYSLTISSLESEDFADYYC

SEQ ID NO: 448 - protein, 23 aa, synthetic construct
DILLTQSPSSMSVSLGDTVSITC

SEQ ID NO: 449 - protein, 15 aa, synthetic construct
WLRQKPGKSFKGLIY

SEQ ID NO: 450 - protein, 32 aa, synthetic construct
GVPSRFSGSGSGADYSLTISSLESEDFADYYC

SEQ ID NO: 451 - DNA, 351 bp, synthetic construct
  source 1..351: mol_type = other DNA
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaaatacacgccatctctaaaggataaattcatcatctccagagacaacgccaaaaatacgctgtgcctgcaattgagcaaagtgagatctgaggactcagccctttattactgtgcaagaatgggctataggctctttgactcctggggccaaggcaccactctcacagtctcctca

SEQ ID NO: 452 - DNA, 336 bp, synthetic construct
  source 1..336: mol_type = other DNA
aatattgtgatgactcaggctacaccctctgtacctgtcactcctggagagtcagtatccatctcctgcaggtctagtaagagtctcctgcatagtgatggcgacacttacttgtattggttcctgcagaggccaggccagtctcctcagctcctgatatatcggatgtccaaccttgtctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaa

SEQ ID NO: 453 - DNA, 360 bp, synthetic construct
  source 1..360: mol_type = other DNA
gaggttcagctccagcagtctgggactgtgctggcaaggcctggggcttcagtgaagatgtcctgcaaggcttctggcaacacctttaccagctactggatgcactgggtaaaacagaggcctggacagggtctggaatggattggcgctatttatcctggatatagtgatactaggtacaatcagaagtttaagggcaaggccacactgactgcagtcacatccaccagcactgcctacatggagctcagcagcctgacaaatgaggactctgcggtctattactgtacaaaatttattgctacggtaggggggtacttcgatgtctggggcgcagggaccacggtcaccgtctcctca

SEQ ID NO: 454 - DNA, 330 bp, synthetic construct
  source 1..330: mol_type = other DNA
caaattgttctcacccagtctccagcaatcatgtcttcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccatattcacgttcggctcggggacaaagttggaaataaaa

SEQ ID NO: 455 - DNA, 363 bp, synthetic construct
  source 1..363: mol_type = other DNA
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaaaattgagactggtaacccaacatatgcagatgacttcaagggacggtttgccttctctttggaaacctctgccagcactgcctatttgcagatcaacaacctcaaaaatgaggacacggctacatatttctgtgcgagagactactatggttattattactatgctatggactactggggtcaaggaacctcagtcaccgtctcctca

SEQ ID NO: 456 - DNA, 324 bp, synthetic construct
  source 1..324: mol_type = other DNA
caaattgttctcacccagtctccagcaatcatgtctgcatctctaggggaacgggtcaccatgacctgcactgttagctcaagtataagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaagctggagctgaaa

SEQ ID NO: 457 - DNA, 363 bp, synthetic construct
  source 1..363: mol_type = other DNA
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagttatggtggaggtgacacctactattcagacactgtaaagggccgattcaccatctccagagacaatgccaagaacaccctgtacctacaaatgagcagtctgaagtctgaggacacggccatgtattactgtgcaagacagaaggtcgatggttactacgttggtatggactactggggtcaaggatcctcagtcaccgtctcctca

SEQ ID NO: 458 - DNA, 321 bp, synthetic construct
  source 1..321: mol_type = other DNA
aacattgtaatgacccaatctcccaaatccatgtccatgtcagtaggagagagggtcaccttgagctgcaaggccagcgagaatgtgggtactaatgtatcctggtatcaacagaaaccagagcagtctcctagactgctgatatatggggcttccaaccggtacactggggtccccgatcgcttcacaggcagtggatctgtaacagatttcactctgaccatcagcagtgtgcaggctgaggaccttgtagattatcactgtggacagagttacagctctccgctcacgttcggttctgggaccaagctggagctgaaa

SEQ ID NO: 459 - DNA, 366 bp, synthetic construct
  source 1..366: mol_type = other DNA
gaggtgcagctggtggagtctgggggaggcttagtgcagcctggagggtccctgagactctcctgtgcagcctctggattcactttcagtcactatggcatgtcttgggttcgccagactccagacaagaggctggagttggtcgcaaccattgataataatggtggtaacacctattatccagacagtgtgaagggccgattcaccatctccagagacaatgccaagaacaccctgtacctgcaaatgagcagtctgaagtctgaggacacagccatgtattactgtgcaagagacgcctactatacttacgactactggtacttcgatgtctggggcgcagggaccacggtcaccgtctcctca

SEQ ID NO: 460 - DNA, 321 bp, synthetic construct
  source 1..321: mol_type = other DNA
gatatccagatgacacagactacatcctccctgtctgcctctctgggagacagagtcaccatcagttgcagtgcgagtcagggcattagtaattatttaaactggtatcagcagaaaccagatggaactgtgaaactcctgatctattacacatcaagtttacattcaggagtcccatcaaggttcagtggcagtgggtctgggacagattattctctcaccatcagcaacctagaacctgaagatattgccacttactattgtcaacagtatcataagcttccgtggacgttcggtggaggcaccaagctggaaatcaaa

SEQ ID NO: 461 - DNA, 363 bp, synthetic construct
  source 1..363: mol_type = other DNA
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagttatggtggaggtgacacgtactatccagacactgtaaagggccgattcaccatctccagagacaatgccaagaacaccctgtacctgcaaatgagcagtctgaagtctgaggacacggccatgtattactgtgccagacagaaggtcgatggttacttcgttgctatggactattggagtcaaggaacctcagtcaccgtctcctca

SEQ ID NO: 462 - DNA, 333 bp, synthetic construct
  source 1..333: mol_type = other DNA
gtcattgtgctgacccaatctccagcttctttggctgtgtctctagggcagagggccaccatctcctgcagagccagcgagcgtgttgatgattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaaataaggaggttccgtggacgttcggtggaggcaccaagctggaaatcaaa

SEQ ID NO: 463 - DNA, 360 bp, synthetic construct
  source 1..360: mol_type = other DNA
gaagtgaagctggtggagtctgggggaggcttagtgaagcctggagggtccctgaaactctcctgtgcagcctctggattcactttcagtagctatggcatgtcttgggttcgccagactccggagaagaggctggagtgggtcgcaaccatcagtggtggtggtagatacacctactatccagacagtgtgaaggggcgattcaccatctccagagacaatgccaagaacatcctgtacctgcaaatgagtagtctgaggtctgaggacacggccttgtattactgtgcaagcccctatgatggttactacggtgctatggactactggggtcaaggaacctcagtcaccgtctcctca

SEQ ID NO: 464 - DNA, 333 bp, synthetic construct
  source 1..333: mol_type = other DNA
gacattgtgctgacccaatctccagcttctttggctgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 465 - DNA, 345 bp, synthetic construct
  source 1..345: mol_type = other DNA
gaggttcagctccagcagtctgggactgtgctggcaaggcctggggcttccgtgaggatgtcctgcaaggcttctggctacagcttttccacctactggatgcactgggtaaaacagaggcctggacagggtctagaatggattggtggtatttatcctggaaatagtgacactaactacaaccagaagttcaagggcaaggccaaactgactgcagtcacttccgccagcactgcctacatggacctcagcagcctgacaaatgaggactctgcggtctattactgtacaggcggttacttcttcgatgtctggggcgcagggaccacggtcaccgtctcctca

SEQ ID NO: 466 - DNA, 321 bp, synthetic construct
  source 1..321: mol_type = other DNA
gacatcaagatgacccagtctccatcttccttgtatgcatctctaggagagagagtcactatcacttgcaaggcgagtcaggacattaatcgatatttaacctggttccagcagaaaccagggaaatctcctaagaccctgatctttcgtgcaaacagattggtaactggggtcccatcaaggttcattggcagtggatctgggcaagaatattctctcaccatcagcagcctggagtatgaagacatgggaatttatttttgtctacagtatgatgagtctccgtacacgttcggaggggggaccaagttggaaataaaa

SEQ ID NO: 467 - DNA, 351 bp, synthetic construct
  source 1..351: mol_type = other DNA
cagatccagctgcagcagtctggacctgagcaggtgaagcctggggcttcagtgaagatatcctgcaaggcttctggctacatgttcatcgactactatatgaactgggtgaagcagaagcctggacagggacttgagtggattggatggatttatcctggaagcggtagtactaaggacaatgagaatttcaagggcaaggccacattgactgtagacacatcctccagcacagcctacatgcagctcagtagcctgacatctgaggacactgctgtctatttctgtgcaagatatgggcccaggttcttcgatgtctggggcgcagggaccacggtcaccgtctcctca

SEQ ID NO: 468 - DNA, 321 bp, synthetic construct
  source 1..321: mol_type = other DNA
gacatccagatgacccagtctccatcctccttatctgcctctctgggagaaagagtcaatctcacttgtcgggcaagtcaggaaattagtggttacttaagctggcttcagcagaaaccagatggaactattaaacgcctgatctacgtcgcatccactttagattctggtgtcccagaaaggttcagtggcagtaggtctgggtcagattattctctcaccatcagcagccttgagtctgaggattttgcagactattactgtctacaatatgctagttatccgtacacgttcggaggggggaccaagctggaaataaaa

SEQ ID NO: 469 - DNA, 351 bp, synthetic construct
  source 1..351: mol_type = other DNA
gaggtgaagcttctcgagtctggaggtggcctggtgcagcctggaggatccctgaaactctcctgtgaagtctcaggattcgattttagcagagactggatgagttgggtccggcaggctccagggaaagggctagagtggattggacaaatttctccagatagcacttcgataaactataagccatctctaaaggataaattcatcatctccagagacaacgccaaaaatacgctgtacctgcaattgagcggagtgagatctgaggacacagccctttatcactgtgcaagagacacctcgggctaccccgactactggggccaaggcaccagtctcacagtctcctca

SEQ ID NO: 470 - DNA, 321 bp, synthetic construct
  source 1..321: mol_type = other DNA
gacatcctgttgacccaatctccatcctccatgtctgtttctctgggagacacagtcagcataacgtgtcatgcaagtcaggacattagcaggaatatagggtggttgcggcagaaaccagggaaatcatttaagggcctgatctatcatggaaccaatttggaagatggagttccatcaaggttcagcggcagtggatctggagcagattattctctcaccatcagcagcctggaatctgaagattttgcagactattactgtgtgcagtatgctcagtttccgtacacgttcggaggggggaccaaactggaaataaaa